(19) United States
(12) Patent Application Publication
(10) Pub. No.: US 2009/0082464 A1
(43) Pub. Date: Mar. 26, 2009

(54) EXTERNALLY MASKED NEOPENTYL SULFONYL ESTER CYCLIZATION RELEASE PRODRUGS OF ACAMPROSATE, COMPOSITIONS THEREOF, AND METHODS OF USE

(76) Inventors: Bernd Jandeleit, Menlo Park, CA (US); Yunxiao Li, Sunnyvale, CA (US); Mark A. Gallop, Santa Clara, CA (US); Noa Zerangue, Belmont, CA (US); Peter A. Virsik, Portola Valley, CA (US); Wolf-Nicolas Fischer, Sunnyvale, CA (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(21) Appl. No.: 12/205,275

(22) Filed: Sep. 5, 2008

Related U.S. Application Data
(60) Provisional application No. 60/970,928, filed on Sep. 7, 2007.

(51) Int. Cl. A61K 31/16; C07C 233/00

(52) U.S. Cl. 514/625; 564/199; 564/215

(57) ABSTRACT

Masked nitrogen-substituted and oxygen-substituted neopentyl sulfonyl ester prodrugs of acamprosate, pharmaceutical compositions comprising such prodrugs, and methods of using such prodrugs and compositions thereof for treating diseases are disclosed. In particular, acamprosate prodrugs exhibiting enhanced oral bioavailability and methods of using acamprosate prodrugs to treat neurodegenerative disorders, psychotic disorders, mood disorders, anxiety disorders, somatoform disorders, movement disorders, substance abuse disorders, binge eating disorder, cortical spreading depression related disorders, tinnitus, sleeping disorders, multiple sclerosis, and pain are disclosed.

This application claims the benefit under 35 U.S.C. § 119(e) of U.S. Provisional Application Ser. No. 60/970,928 filed Sep. 7, 2007, which is incorporated by reference in its entirety.

### FIELD

Disclosed herein are masked nitrogen-substituted and oxygen-substituted neopentyl sulfonyl ester prodrugs of acamprosate that exhibit enhanced oral bioavailability, pharmaceutical compositions comprising such prodrugs, and methods of using such prodrugs and compositions thereof for treating diseases such as neurodegenerative disorders, psychotic disorders, mood disorders, anxiety disorders, somatoform disorders, movement disorders, substance abuse disorders, binge eating disorder, cortical spreading depression related disorders, sleeping disorders, tinnitus, multiple sclerosis, and pain.

### BACKGROUND

Prodrugs are derivatized forms of drugs that following administration are converted or metabolized to an active form of the drug in vivo. Prodrugs are used to modify one or more aspects of the pharmacokinetics of a drug in a manner that enhances the therapeutic efficacy of a drug. For example, prodrugs are often used to enhance the oral bioavailability of a drug. To be therapeutically effective, drugs exhibiting poor oral bioavailability may require frequent dosing, large administered doses, or may need to be administered by other than oral routes, such as intravenously. In particular, many drugs with sulfonic acid groups exhibit poor oral bioavailability.

Intramolecular cyclization prodrug strategies have been used to modify the pharmacokinetics of drugs (Bundgaard in “A Textbook of Drug Design and Development,” Krogsgaard-Larsen and Bundgaard Eds., Harwood Academic, Philadelphia, 1991, pp. 113-192; Bungaard and Nielsen, U.S. Pat. No. 5,073,641; Santos et al., Bioorganic & Medicinal Chemistry Letters, 2005, 15, 1595-1598; Papot et al., Curr Med Chem—Anti-Cancer Agents, 2002, 2, 155-185; and Shan et al., J Pharm Sciences 1997, 86(7), 765-767). Intramolecular cyclization prodrug strategies have been applied to drugs containing sulfonic acid functional groups. Prodrugs comprising a substituted neopentyl sulfonate ester derivative in which the neopentyl group is removed in vivo by unmasking a nucleophilic heteroatom bonded to a substituted neopentyl moiety followed by intramolecular cyclization to generate the parent drug in the sulfonic acid or sulfonic acid salt form have been described (Roberts and Patch, U.S. Pat. No. 5,596,095; and Roberts et al., Tetrahedron Lett 1997, 38(3), 355-358). In such prodrugs the nucleophilic heteroatom can be nitrogen or oxygen and that the nitrogen or oxygen nucleophile can be masked with any amine or alcohol protecting group, respectively, capable of being deprotected in vivo. Roberts and Patch also disclose that the masked nucleophilic group can be a carboxylic ester, e.g., —OCOR where R can be aryl, substituted aryl, heteroaryl, C₁₋₈ alkyl, arylalkyl, or heteroarylalkyl. However, Roberts and Patch do not provide biological or pharmacological data to indicate which if any of the substituted neopentyl sulfonate esters release the prodrug in vivo and would therefore be useful for enhancing the oral bioavailability of the corresponding drug.

3-(Acetylamino)propylsulfonic acid (also referred to as N-acetylhomotaurine), acamprosate,

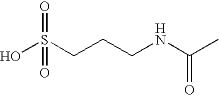

is a derivative of homotaurine, a naturally occurring structural analog of γ-aminobutyric acid (GABA) that appears to affect multiple receptors in the central nervous system (CNS). As an antiglutamatergic agent, acamprosate is believed to exert a neuropharmacological effect as an antagonist of N-methyl-D-aspartate (NMDA) receptors. The mechanism of action is believed to include blocking of the Ca²⁺ channel to slow Ca²⁺ influx and reduce the expression of c-fos, leading to changes in messenger RNA transcription and the concomitant modification to the subunit composition of NMDA receptors in selected brain regions (Zornoza et al., CNS Drug Reviews, 2003, 9(4), 359-374; and Rammes et al., Neuropharmacology 2001, 40, 749-760). In addition, acamprosate may block GABA_{B} receptors (Daost, et al., Pharmacol Biochem Behav. 1992, 41, 669-74; and Johnson et al., Psychopharmacology 2000, 149, 327-344). Similar mechanisms are believed to be associated with the activity of other glutamate modulators such as riluzole, N-acetylcysteine, β-lactams, amantadine, lamictal, memantine, neramexane, remacemide, ifenprodil, and dextromethorphan.

Other diseases or disorders known to be associated with modulation of NMDA activity and for which modulators of NMDA receptor activity are clinically useful include psychotic disorders such as schizophrenia and schizoaffective disorder; mood disorders such as anxiety disorders including posttraumatic stress disorder and obsessive-compulsive disorder, depression, mania, bipolar disorder; and somatoform disorders such as somatization disorder, conversion disorder, hypochondriasis, and body dysmorphic disorder; movement disorders such as Tourette's syndrome, focal dystonia, Huntington's disease, Parkinson's disease, Syndeham's chorea, systemic lupus erythematosus, drug-induced movement disorders, tardive dyskinesia, blepharospasm, tic disorder, and spasticity; substance abuse disorders such as alcohol abuse disorders, narcotic abuse disorders, and nicotine abuse disorders; cortical spreading depression related disorders such as migraine, cerebral damage, epilepsy, and cardiovascular; sleeping disorders such as sleep apnea; multiple sclerosis; and neurodegenerative disorders such as Parkinson's disease, Huntington's disease, Alzheimer's disease, and amyotrophic lateral sclerosis. Recently, acamprosate has been found to be effective in treating tinnitus, or noise originating in the ear, a common disorder (de Azevedo et al., 109^{th} Meeting and OTO EXPO of the Am. Acad. Otolaryngology—Head and Neck Foundation, Los Angeles, Calif., Sep. 25-28, 2005; Azevedo et al, Rev. Bras. Otorrinolaringol. Engl. Ed., 2005, 71, 618-623; and Azevedo et al., WO 2007/082561 A2). Acamprosate analogs (Berthelon et al., U.S. Pat. No. 6,265,437) and salt forms of acamprosate analogs (Durlach, U.S. Pat. No. 4,355,043) are also reported to have therapeutic use.

There is also evidence that acamprosate may interact with excitatory glutamatergic neurotransmission in general and as an antagonist of the metabotropic glutamate receptor subtype 5 (mGluR5) in particular (De Witte et al., CNS Drugs 2005, 19(6), 517-37). The glutamatergic mechanism of action of acamprosate may explain the effects of acamprosate on alcohol dependence and suggests other activities such as in neuroprotection. Dysregulation of the mGluR5 receptor has been implicated in a number of diseases and mGluR5 antagonists have been shown to be effective in treating depression pain, (anxiety disorders, alcohol abuse disorders, drug abuse disorders, nicotine abuse disorders, neurodegenerative disorders such as Parkinson's disease, diabetes, schizophrenia, and gastrointestinal reflux disease.

Acamprosate is a polar molecule that lacks the requisite physicochemical characteristics for effective passive permeability across cellular membranes. Intestinal absorption of acamprosate is mainly by passive diffusion and to a lesser extent by an active transport mechanism such as via an amino acid transporter (Más-Serrano et al., Alcohol 2000, 4(3); and 324-330; Saivin et al., Clin Pharmacokinet 1998, 35, 331-345). As a consequence, the oral bioavailability of acamprosate in humans is only about 11%. The mean elimination half-life of acamprosate following intravenous infusion (15 min) is 3.2±0.2 h. Efforts to enhance the gastrointestinal absorption and oral bioavailability of acamprosate include co-administrating the drug with polyglycolysed glycerides (Saslawski et al., U.S. Pat. No. 6,514,524). Acamprosate prodrugs exhibiting enhanced absorption from the lower gastrointestinal tract have the potential to increase the oral bioavailability of the drug and to facilitate administration of acamprosate using sustained release oral dosage forms.

### SUMMARY

Thus, there is a need for new prodrugs of acamprosate with demonstrated enhanced oral bioavailability. In particular, masked nitrogen-substituted and oxygen-substituted neopentylsulfonate ester prodrugs of acamprosate that exhibit enhanced absorption throughout the gastrointestinal tract and especially in the large intestine/colon and hence that are suitable for sustained release oral formulations, can enhance the convenience (by reducing the dose and dosing frequency), efficacy, and side effect profile of acamprosate.

In a first aspect, compounds of Formula (I) are provided:

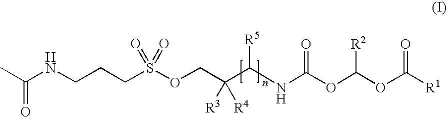

or a pharmaceutically acceptable salt thereof; wherein:

n is chosen from 0, 1, 2, and 3;

R¹ is chosen from C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;

R² is chosen from hydrogen, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, and substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;

R³ and R⁴ are independently chosen from C₁₋₄ alkyl and substituted C₁₋₄ alkyl; or R³ and R⁴ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and

each R⁵ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

In a second aspect, compounds of Formula (II) are provided:

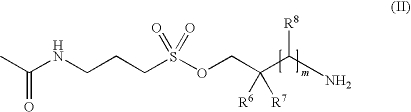

or a pharmaceutically acceptable salt thereof; wherein:

m is chosen from 0, 1, 2, and 3;

R⁶ and R⁷ are independently chosen from C₁₋₄ alkyl and substituted C₁₋₄ alkyl; or R⁶ and R⁷ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and

each R⁸ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

In a third aspect, compounds of Formula (III) are provided:

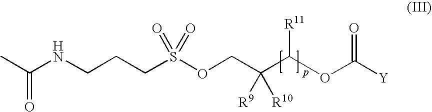

or a pharmaceutically acceptable salt thereof; wherein:

p is chosen from 0, 1, 2, and 3;

Y is chosen from R¹², —OR¹², and —NR¹²₂, wherein:
each R¹² is independently chosen from C₁₋₈ alkyl, substituted C₁₋₈ alkyl, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;

R⁹ and R¹⁰ are independently chosen from C₁₋₄ alkyl and substituted C₁₋₄ alkyl; or R⁹ and R¹⁰ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and

each R¹¹ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl; and

with the proviso that when

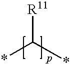

is C₁₋₈ alkyldiyl, and Y is chosen from R¹² and —OR¹²; then R¹² is not chosen from C₁₋₈ alkyl, C₆₋₁₀ aryl, substituted C₆₋₁₀ aryl, C₅₋₁₀ heteroaryl, substituted C₅₋₁₀ heteroaryl, C₇₋₁₈ arylalkyl, and C₆₋₁₈ heteroarylalkyl.

In a fourth aspect compounds of Formula (IV) are provided.

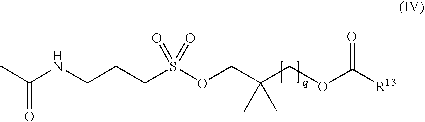

or a pharmaceutically acceptable salt thereof, wherein:

q is chosen from 0, 1, 2, and 3; and

R¹³ is chosen from ethoxy, phenyl, —CH₂NH₂, and C₁₋₆ alkyl.

In a fifth aspect, compounds of Formula (V) are provided:

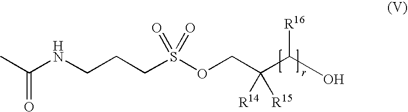

or a pharmaceutically acceptable salt thereof; wherein:

r is chosen from 0, 1, 2, and 3;

R¹⁴ and R¹⁵ are independently chosen from C₁₋₄ alkyl and substituted C₁₋₄ alkyl; or R¹⁴ and R¹⁵ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and

each R¹⁶ is independently chosen from hydrogen, halogen, —OH, —CN, —CF₃, —OCF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

In a sixth aspect, pharmaceutical compositions are provided comprising at least one pharmaceutically acceptable vehicle and at least one compound chosen from Formula (I), Formula (III), Formula (IV), and a pharmaceutically acceptable salt of any of the foregoing.

In a seventh aspect, methods of treating a disease in a patient comprising administering to a patient in need of such treatment a therapeutically effective amount of a compound chosen from Formula (I), Formula (III), Formula (IV), and a pharmaceutically acceptable salt of any of the foregoing. In certain embodiments, the disease is chosen from a neurodegenerative disorder, a psychotic disorder, a mood disorder, an anxiety disorder, a somatoform disorder, a movement disorder, a substance abuse disorder, binge eating disorder, a cortical spreading depression related disorder, tinnitus, a sleeping disorder, multiple sclerosis, and pain.

### DETAILED DESCRIPTION

### Definitions

A dash (“-”) that is not between two letters or symbols is used to indicate a point of attachment for a moiety or substituent. For example, —CONH₂ is attached through the carbon atom.

“Alkyl” by itself or as part of another substituent refers to a saturated or unsaturated, branched, or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group can have from 1 to 20 carbon atoms, in certain embodiments, from 1 to 10 carbon atoms, in certain embodiments from 1 to 8 carbon atoms, in certain embodiments, from 1 to 6 carbon atoms, in certain embodiments from 1 to 4 carbon atoms, and in certain embodiments, from 1 to 3 carbon atoms.

“Alkyldiyl” refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Examples of alkyldiyl groups include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. In certain embodiments, an alkyldiyl group is C₁₋₂₀ alkyldiyl, C₁₋₁₀ alkyldiyl, C₁₋₈ alkyldiyl, and in certain embodiments, C₁₋₄ alkyldiyl. Also, in certain embodiments, an alkyldiyl group is a saturated acyclic alkanyldiyl group in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkoxy” by itself or as part of another substituent refers to a radical —OR³¹ where R³¹ is chosen from alkyl, heteroalkyl, cycloalkyl, heterocycloalkyl, cycloalkylalkyl, heterocycloalkylalkyl, aryl, heteroaryl, arylalkyl, and heteroarylalkyl, as defined herein. Examples of alkoxy groups include, but are not limited to, methoxy, ethoxy, propoxy, butoxy, cyclohexyloxy, and the like. In certain embodiments, an alkoxy group is C₁₋₁₈ alkoxy, in certain embodiments, C₁₋₁₂ alkoxy, in certain embodiments, C₁₋₈ alkoxy, in certain embodiments, C₁₋₆ alkoxy, in certain embodiments, C₁₋₄ alkoxy, and in certain embodiments, C₁₋₃ alkoxy.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms (C₆₋₂₀), from 6 to 12 carbon atoms (C₆₋₁₂), and in certain embodiments, from 6 to 10 carbon atoms (C₆₋₁₀).

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₇₋₂₀, in certain embodiments, an arylalkyl group is C₆₋₁₈ arylalkyl, e.g., the alkanyl, alkenyl or alkynyl moiety of the arylalkyl group is C₁₋₈ and the aryl moiety is C₆₋₁₀.

“AUC” is the area under a curve representing the concentration of a compound or metabolite thereof in a biological fluid in a patient as a function of time following administration of the compound to the patient. In certain embodiments provided by the present disclosure, the compound is a prodrug of Formula (I), Formula (III), and Formula (IV), and the drug is acamprosate. Examples of biological fluids include plasma and blood. The AUC may be determined by measuring the concentration of a compound or metabolite thereof in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the present disclosure, an AUC for acamprosate or metabolite thereof may be determined by measuring over time the concentration of acamprosate or metabolite thereof in the plasma, blood, or other biological fluid or tissue of a patient following administration of a corresponding prodrug of Formula (I), Formula (III), or Formula (IV) to the patient.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to maximum concentration (Tₘₐₓ), and the maximum drug concentration (Cₘₐₓ), where Cₘₐₓ is the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Cₘₐₓ” is the maximum concentration of a drug in the plasma or blood of a patient following administration of a dose of the drug or prodrug to the patient.

“Tₘₐₓ” is the time to the maximum (peak) concentration (Cₘₐₓ) of a drug in the plasma or blood of a patient following administration of a dose of the drug or prodrug to the patient.

“Compounds” of Formula (I)-(V) disclosed herein include any specific compounds within these formulae. Compounds may be identified either by their chemical structure and/or chemical name. When the chemical structure and chemical name conflict, the chemical structure is determinative of the identity of the compound. The compounds described herein may comprise one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

Compounds of Formula (I)-(V) include, but are not limited to, optical isomers of compounds of Formula (I)-(V), racemates thereof, and other mixtures thereof. In such embodiments, the single enantiomers or diastereomers, i.e., optically active forms, can be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column. In addition, compounds of Formula (I)-(V) include Z- and E-forms (or cis- and trans-forms) of compounds with double bonds. Compounds of Formula (I)-(V) may also exist in several tautomeric forms including the enol form, the keto form, and mixtures thereof. Accordingly, the chemical structures depicted herein encompass all possible tautomeric forms of the illustrated compounds. Compounds of Formula (I)-(V) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds as referred to herein may be free acid, salt, hydrated, solvated, or N-oxide forms of the compounds. Thus, when reference is made to compounds of the present disclosure, such as compounds of Formula (I)-(V), it is understood that a compound also implicitly refers to salts, solvates, hydrates, and combinations of any of the foregoing. Certain compounds may exist in multiple crystalline, cocrystalline, or amorphous forms. Compounds of Formula (I)-(V) include pharmaceutically acceptable solvates of a free acid or salt form of any of the foregoing, hydrates of a free acid or salt form of any of the foregoing, as well as crystalline forms of any of the foregoing.

Compounds of Formula (I)-(V) also include solvates. The term “solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

Further, when partial structures of the compounds are illustrated, an asterisk (*) indicates the point of attachment of the partial structure to the rest of the molecule.

“Cycloalkyl” by itself or as part of another substituent refers to a saturated or partially unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, C₃₋₁₀ cycloalkyl and in certain embodiments, C₃₋₈ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂. In certain embodiments, a cycloalkylalkyl group is C₄₋₁₈ cycloalkylalkyl.

“Disease” refers to a disease, disorder, condition, or symptom of any of the foregoing.

“Drug” as defined under 21 U.S.C. § 321(g)(1) means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals . . . ”

“Halogen” refers to a fluoro, chloro, bromo, or iodo group. In certain embodiments, halogen is fluoro, and in certain embodiments, halogen is chloro.

“Heteroalkyl” by itself or as part of another substituent refer to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—, —NR³⁷, ═N—N═, —N═N—, —N═N—NR³⁷—, —PR³⁷—, —P(O)₂—, —POR³⁷—, —O—P(O)₂—, —SO—, —SO₂—, —Sn(R³⁷)₂—, and the like, where each R³⁷ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatoms, groups having four carbon atoms, and groups having two heteroatoms, etc. In certain embodiments, each R³⁷ is independently chosen from hydrogen and C₁₋₃ alkyl. In certain embodiments, a heteroatomic group is chosen from —O—, —S—, —NH—, —N(CH₃)—, and —SO₂—.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. Heteroaryl encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; and 5- to 14-membered bicyclic rings containing one or more, for example, from 1 to 4, or in certain embodiments, from 1 to 3, heteroatoms chosen from N, O, and S, with the remaining ring atoms being carbon, wherein at least one of the rings is an aromatic ring, and wherein at least one heteroatom is present in the at least one aromatic ring. For example, heteroaryl includes a 5- to 7-membered heteroaromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. In certain embodiments, a heteroaryl group is C₅₋₁₂ heteroaryl, C₅₋₁₀ heteroaryl, and in certain embodiments, C₅₋₆ heteroaryl. The ring of a C₅₋₁₀ heteroaryl has from 4 to 9 carbon atoms, with the remainder of the atoms in the ring being heteroatoms.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, in certain embodiments from 5- to I O-membered heteroaryl, and in certain embodiments from 6- to 8-heteroaryl. In certain embodiments heteroaryl groups are those derived from thiophene, pyrrole, benzothiophene, benzofuran, indole, pyridine, quinoline, imidazole, oxazole, or pyrazine.

“Heteroarylalkyl” by itself or as part of another substituent refers to an acyclic alkyl radical in which one of the hydrogen atoms bonded to a carbon atom, is replaced with a heteroaryl group. Typically a terminal or sp³ carbon atom is the atom replaced with the heteroaryl group. Where specific alkyl moieties are intended, the nomenclature “heteroarylalkanyl,” “heteroarylalkenyl,” and “heterorylalkynyl” is used. In certain embodiments, a heteroarylalkyl group is a 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl, and in certain embodiments, 6- to 14-membered heteroarylalkyl, e.g., the alkanyl, alkenyl, or alkynyl moiety of the heteroarylalkyl is 1- to 4-membered and the heteroaryl moiety is a 5- to 12-membered heteroaryl. In certain embodiments, a heteroarylalkyl group is C₆₋₁₈ heteroaryl alkyl and in certain embodiments, C₆₋₁₀ heteroarylalkyl.

“Heterocycloalkyl” by itself or as part of another substituent refers to a saturated or partially unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. In certain embodiments, a heterocycloalkyl group is a C₃₋₁₂ heterocycloalkylalkyl, C₃₋₁₀ heterocycloalkylalkyl, and in certain embodiments C₃₋₈ heterocyclalkyalkyl.

“Heterocycloalkyalkyl” by itself or as part of another substituent refers to an acyclic alkyl radical in which one of the hydrogen atoms bonded to a carbon atom, is replaced with a cycloalkyl group as defined herein. In certain embodiments, a heterocycloalkylalkyl group is a C₄₋₁₈ heterocycloalkylalkyl, C₄₋₁₂ heterocycloalkylalkyl, and in certain embodiments C₄₋₁₀ heterocyclalkyalkyl.

“Metabolic intermediate” refers to a compound that is formed in vivo by metabolism of a parent compound and that further undergoes reaction in vivo to release an active agent. Compounds of Formula (I), Formula (III), and Formula (IV) are protected amine or oxygen nucleophile prodrugs of acamprosate that are metabolized in vivo to provide the corresponding metabolic intermediates of Formula (II) or Formula (V). Metabolic intermediates of Formula (II) and Formula (V) undergo nucleophilic cyclization to release acamprosate and one or more reaction products. It is desirable that the reaction products or metabolites thereof not be toxic.

“Neopentyl” refers to a radical in which a methylene carbon is bonded to a carbon atom, which is bonded to three non-hydrogen substituents. Examples of non-hydrogen substituents include carbon, oxygen, nitrogen, and sulfur. In certain embodiments, each of the three non-hydrogen substituents is carbon. In certain embodiments, two of the three non-hydrogen substituents is carbon, and the third non-hydrogen substituent is chosen from oxygen and nitrogen. In certain embodiments, a neopentyl group has the structure:

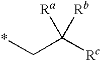

where R^{a} and R^{b} are independently chosen from C₁₋₄ alkyl, substituted C₁₋₄ alkyl, C₁₋₄ alkoxy, and substituted C₁₋₄ alkoxy; or R³ and R⁴ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and R^{c} is chosen from carbon, nitrogen, and oxygen. In certain embodiments, each of R^{a} and R^{b} is methyl; and R^{c} is chosen from carbon, nitrogen, and oxygen. In certain embodiments, each of R^{a} and R^{b} is methyl; and R^{c} is carbon; in certain embodiments, nitrogen; and in certain embodiments, oxygen.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom in such a way as to maintain the continuous π (pi)-electron system characteristic of aromatic systems and a number or out-of-plane π (pi)-electrons corresponding to the Hickel rule (4n+1). Examples of heteroatoms to replace the carbon atoms include, but are not limited to, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, pharmaceutically acceptable addition salts include metal salts such as sodium, potassium, aluminum, calcium, magnesium and zinc salts, and ammonium salts such as isopropylamine, diethylamine, and diethanolamine salts. In certain embodiments, a pharmaceutically acceptable salt is the hydrochloride salt. In certain embodiments, a pharmaceutically acceptable salt is the sodium salt. Pharmaceutically acceptable salts may be prepared by the skilled chemist, by treating a compound of Formula (I) with an appropriate base in a suitable solvent, followed by crystallization and filtration.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to at least one compound of Formula (I), Formula (III), or Formula (IV) and at least one pharmaceutically acceptable vehicle, with which the at least one compound of Formula (I), Formula (III), or Formula (IV) is administered to a patient.

“Prodrug” refers to a derivative of a drug molecule that requires a transformation within the body to release the active drug. Prodrugs are frequently, although not necessarily, pharmacologically inactive until converted to the parent drug. Prodrugs may be obtained by bonding a promoiety (defined herein) typically via a functional group, to a drug. For example, referring to compounds of Formula (I), the promoiety is bonded to the drug via the sulfonic acid functional group of acamprosate. Compounds of Formula (I), Formula (III), and Formula (IV) are prodrugs of acamprosate that can be metabolized within a patient's body to release acamprosate.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a patient to which the prodrug is administered or the acidic conditions of the stomach or the agent may be supplied exogenously. For example, for a prodrug of Formula (I), the drug is acamprosate (1) and the promoiety has the structure:

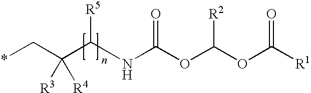

and for a prodrug of Formula (III), the drug is acamprosate (1) and the promoiety has the structure:

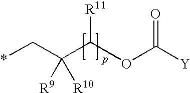

where n, p, R¹, R², R³, R⁴, R⁵, R⁹, R¹⁰, R¹¹, and Y are is defined herein.

“Protecting group” refers to a grouping of atoms, which when attached to a reactive group in a molecule masks, reduces, or prevents that reactivity. Examples of amino protecting groups include, but are not limited to, formyl, acetyl, trifluoroacetyl, benzyl, benzyloxycarbonyl (CBZ), tert-butoxycarbonyl (Boc), trimethylsilyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like. Examples of hydroxy protecting groups include, but are not limited to, those in which the hydroxy group is either acylated or alkylated such as benzyl, and trityl ethers as well as alkyl ethers, tetrahydropyranyl ethers, trialkylsilyl ethers, and allyl ethers.

“Salt” refers to a chemical compound consisting of an assembly of cations and anions. Salts of a compound of the present disclosure include stoichiometric and non-stoichiometric forms of the salt. In certain embodiments, because of its potential use in medicine, salts of a compound of Formula (I) are pharmaceutically acceptable salts.

“Substituted” refers to a group in which one or more hydrogen atoms are independently replaced with the same or different substituent group(s). Examples of substituent groups include, but are not limited to, -M, —R⁶⁰, —O, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, and —C(NR⁶²)NR⁶⁰R⁶¹ where M is independently a halogen; R⁶⁰, R⁶¹, R⁶², and R⁶³ are independently chosen from hydrogen, alkyl, alkoxy, cycloalkyl, heterocycloalkyl, aryl, and heteroaryl, or R⁶⁰ and R⁶¹ together with the nitrogen atom to which they are bonded form a ring chosen from a heterocycloalkyl ring. In certain embodiments, R⁶⁰, R⁶¹, R⁶², and R⁶³ are independently chosen from hydrogen, C₁₋₆ alkyl, C₁₋₆ alkoxy, C₃₋₁₂ cycloalkyl, C₃₋₁₂ heterocycloalkyl, C₆₋₁₂ aryl, and C₆₋₁₂ heteroaryl. In certain embodiments, each substituent group is independently chosen from halogen, —OH, —CN, —CF₃, ═O, —NO₂, C₁₋₃ alkoxy, C₁₋₃ alkyl, —COOR⁶⁴ wherein R⁶⁴ is chosen from hydrogen and C₁₋₃ alkyl, and —NR⁶⁵₂ wherein each R⁶⁵ is independently chosen from hydrogen and C₁₋₃ alkyl. In certain embodiments, each substituent group is independently chosen from halogen, —OH, —CN, —CF₃, —OCF₃, ═O, —NO₂, C₁₋₆ alkoxy, C₁₋₆ alkyl, —COOR²⁶, —NR²⁷₂, and —CONR²⁸₂; wherein each of R²⁶, R²⁷, and R²⁸ is independently chosen from hydrogen and C₁₋₆ alkyl.

In certain embodiments, each substituent group is independently chosen from halogen, —OH, —CN, —CF₃, ═O, —NO₂, C₁₋₃ alkoxy, C₁₋₃ alkyl, —COOR¹² wherein R¹² is chosen from hydrogen and C₁₋₃ alkyl, and —NR¹²₂ wherein each R¹² is independently chosen from hydrogen and C₁₋₃ alkyl. In certain embodiments, each substituent group is independently chosen from halogen, —OH, —CN, —CF₃, —OCF₃, ═O, —NO₂, C₁₋₆ alkoxy, C₁₋₆ alkyl, —COOR¹², —NR¹²₂, and —CONR²; wherein each R¹² is independently chosen from hydrogen and C₁₋₆ alkyl. In certain embodiments, each substituent group is chosen from C₁₋₄ alkyl, —OH, and —NH₂.

“Sustained release” refers to release of a compound from a dosage form of a pharmaceutical composition at a rate effective to achieve a therapeutic or prophylactic concentration of the compound or active metabolite thereof, in the systemic circulation of a patient over a prolonged period of time relative to that achieved by administration of an immediate release formulation of the same compound by the same route of administration. In some embodiments, release of a compound occurs over a time period of at least about 4 hours, such as at least about 8 hours, at least about 12 hours, at least about 16 hours, at least about 20 hours, and in some embodiments, at least about 24 hours.

“Treating” or “treatment” of any disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or at least one or more symptoms thereof in a patient which may be exposed to or predisposed to a disease or disorder even though that patient does not yet experience or display symptoms of the disease.

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

Reference is now made in detail to certain embodiments of compounds, compositions, and methods. The disclosed embodiments are not intended to be limiting of the claims. To the contrary, the claims are intended to cover all alternatives, modifications, and equivalents.

### Compounds

Certain embodiments provide a compound of Formula (I):

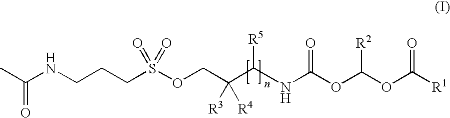

or a pharmaceutically acceptable salt thereof; wherein:

n is chosen from 0, 1, 2, and 3;

R¹ is chosen from C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;

R² is chosen from hydrogen, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;

R³ and R⁴ are independently chosen from C₁₋₄ alkyl and substituted C₁₋₄ alkyl; or R³ and R⁴ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and

each R⁵ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

In certain embodiments of a compound of Formula (III), R⁵ is not substituted C₁₋₈ heteroalkyl. In certain embodiments of a compound of Formula (III), R⁵ is not substituted C₁₋₈heteroalkyl, wherein the one or more substituent groups is ═O.

In certain embodiments of compounds of Formula (I), each substituent group is independently chosen from halogen, —OH, —CN, —CF₃, —OCF₃, ═O, —NO₂, C₁₋₆ alkoxy, C₁₋₆ alkyl, —COOR²⁶, —NR²⁷₂, and —CONR²⁸₂; wherein each of R²⁶, R²⁷, and R²⁸ is independently chosen from hydrogen and C₁₋₆ alkyl. In certain embodiments, each substituent group is chosen from —OH, C₁₋₃ alkoxy, and C₁₋₃ alkyl.

In certain embodiments of compounds of Formula (I), R¹ is chosen from C₁₋₆ alkyl, C₁₋₆ alkoxy, C₃₋₆ cycloalkyl, substituted C₃₋₆ cycloalkyl, phenyl, and substituted phenyl.

In certain embodiments of compounds of Formula (I), R¹ is chosen from C₁₋₆ alkyl, C₁₋₆ alkoxy, phenyl, and substituted phenyl.

In certain embodiments of compounds of Formula (I), R¹ is —OR²⁰ wherein R²⁰ is chosen from C₁₋₄ alkyl, substituted C₁₋₄ alkyl, C₃₋₆ cycloalkyl, substituted C₃₋₆ cycloalkyl, phenyl, substituted phenyl, C₄₋₁₀ cycloalkylalkyl, substituted C₄₋₁₀cycloalkyalkyl, C₇₋₁₀ phenylalkyl, substituted C₇₋₁₀ phenylalkyl, C₁₋₄ heteroalkyl, substituted C₁₋₄ heteroalkyl, C₃₋₆ heterocycloalkyl, substituted C₃₋₆ heterocycloalkyl, C₅₋₆ heteroaryl, substituted C₅₋₆ heteroaryl, C₄₋₁₀ heterocycloalkylalkyl, substituted C₄₋₁₀ heterocycloalkyalkyl, C₆₋₁₀ heteroaryl, and substituted C₆₋₁₀ heteroaryl. In certain embodiments of compounds of Formula (I), R¹ is —OR²⁰ wherein R²⁰ is chosen from C₁₋₄ alkyl, C₃₋₆ cycloalkyl, phenyl, C₄₋₁₀ cycloalkylalkyl, C₇₋₁₀ phenylalkyl, C₁₋₄ heteroalkyl, C₃₋₆ heterocycloalkyl, C₅₋₆ heteroaryl, C₄₋₁₀heterocycloalkylalkyl, and C₆₋₁₀heteroaryl. In certain embodiments of compounds of Formula (I), R¹ is —OR²⁰ wherein R²⁰ is chosen from C₁₋₄alkyl, C₃₋₆ cycloalkyl, phenyl, C₄₋₁₀ cycloalkylalkyl, and C₇₋₁₀ phenylalkyl. In certain embodiments of compounds of Formula (I), R¹ is —OR²⁰ wherein R²⁰ is chosen from C₁₋₄ alkyl, cyclohexyl, phenyl, benzyl, and cyclohexylmethyl.

In certain embodiments of compounds of Formula (I), R² is chosen from hydrogen, C₁₋₆ alkyl, cyclohexyl, and phenyl; in certain embodiments, R² is hydrogen; in certain embodiments R² is C₁₋₆ alkyl; and in certain embodiments R² is C₁₋₄ alkyl. In certain embodiments of compounds of Formula (I), R² is chosen from hydrogen, methyl, ethyl, n-propyl, isopropyl, cyclohexyl, and phenyl.

In certain embodiments of a compound of Formula (I), the stereochemistry of the carbon atom to which R² is bonded is of the (R) configuration. In certain embodiments of a compound of Formula (I), the stereochemistry of the carbon atom to which R² is bonded is of the (S) configuration.

In certain embodiments of compounds of Formula (I), R² is chosen from hydrogen, methyl, ethyl, n-propyl, isopropyl, cyclohexyl, and phenyl; and the stereochemistry of the carbon atom to which R² is bonded is of the (R) configuration.

In certain embodiments of compounds of Formula (I), R² is chosen from hydrogen, methyl, ethyl, n-propyl, isopropyl, cyclohexyl, and phenyl; and the stereochemistry of the carbon atom to which R² is bonded is of the (S) configuration.

In certain embodiments of compounds of Formula (I), each of R³ and R⁴ is methyl.

In certain embodiments of compounds of Formula (I), each R⁵ is hydrogen.

In certain embodiments of compounds of Formula (I), n is chosen from 0, 1, and 2; in certain embodiments n is chosen from 0 and 2; in certain embodiments n is 0; n is 1; and in certain embodiments, n is 2.

In certain embodiments of compounds of Formula (I), R¹ is chosen from C₁₋₆ alkyl, C₁₋₆alkoxy, phenyl, and substituted phenyl; R² is chosen from hydrogen and C₁₋₆ alkyl; each of R³ and R⁴ is methyl; each R⁵ is hydrogen; and n is chosen from 0, 1, and 2.

In certain embodiments of compounds of Formula (I), R¹ is chosen from C₁₋₆ alkyl, C₁₋₆ alkoxy, cyclohexyl, substituted cyclohexyl, phenyl, and substituted phenyl; R² is chosen from hydrogen, C₁₋₆ alkyl, cyclohexyl, and phenyl; each of R³ and R⁴ is methyl; each R⁵ is hydrogen; and n is chosen from 0, 1, and 2.

In certain embodiments of compounds of Formula (I), the compound is chosen from:
[N-(4-{[3-(acetylamino)propyl]sulfonyloxy}-3,3-dimethylbutyl)carbamoyloxy]ethyl 2-methylpropanoate;
[N-(4-{[3-(acetylamino)propyl]sulfonyloxy}-3,3-dimethylbutyl)carbamoyloxy]ethyl benzoate;
[N-(5-{[3-(acetylamino)propyl]sulfonyloxy}-4,4-dimethylpentyl)carbamoyloxy]ethyl 2-methylpropanoate;
[N-(5-{[3-(acetylamino)propyl]sulfonyloxy}-4,4-dimethylpentyl)carbamoyloxy]methyl benzoate;
[N-(3-{[3-(acetylamino)propyl]sulfonyloxy}-2,2-dimethylpropyl)carbamoyloxy]ethyl 2-methylpropanoate;
[N-(2-{[3-(acetylamino)propyl]sulfonyloxy}-tert-butyl)carbamoyloxy]ethyl 2-methylpropanoate; and

a pharmaceutically acceptable salt of any of the foregoing.

Certain embodiments provide a compound of Formula (II):

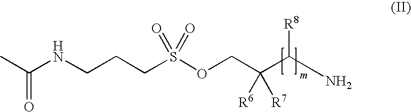

or a pharmaceutically acceptable salt thereof; wherein:

m is chosen from 0, 1, 2, and 3;

R⁶ and R⁷ are independently chosen from C₁₋₄ alkyl and substituted C₁₋₄ alkyl; or R⁶ and R⁷ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and

each R⁸ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

In certain embodiments of a compound of Formula (II),

when each of R⁶ and R⁷ is methyl, and m is 2; then both of R⁸ are not hydrogen.

In certain embodiments of compounds of Formula (II), each substituent group is independently chosen from halogen, —OH, —CN, —CF₃, —OCF₃, ═O, —NO₂, C₁₋₆ alkoxy, C₁₋₆ alkyl, —COOR²⁶, —NR²⁷₂, and —CONR²⁸₂; wherein each of R²⁶, R²⁷, and R²⁸ is independently chosen from hydrogen and C₁₋₆ alkyl. In certain embodiments, each substituent group is chosen from C₁₋₄ alkyl, C₁₋₄ alkoxy, —OH, and —NH₂.

In certain embodiments of compounds of Formula (II), each of R⁶ and R⁷ is methyl.

In certain embodiments of compounds of Formula (II), each R⁸ is hydrogen.

In certain embodiments of compounds of Formula (II), each of R⁶ and R⁷ is methyl; each R⁸ is hydrogen; and m is chosen from 0, 1, 2, and 3. In certain embodiments of compounds of Formula (II), each of R⁶ and R⁷ is methyl; each R⁸ is hydrogen; and m is 0, m is 1, m is 2, and in certain embodiments, m is 3.

In certain embodiments of compounds of Formula (II), the compound is chosen from:
2-amino-2-methylpropyl [3-(acetylamino)propyl]sulfonate trifluoroacetate;
3-amino-2,2-dimethylpropyl [3-(acetylamino)propyl]sulfonate hydrochloride;
5-amino-2,2-dimethylpentyl [3-(acetylamino)propyl]sulfonate hydrochloride;
   and

a pharmaceutically acceptable salt of any of the foregoing.

Certain embodiments provide a compound of Formula (III):

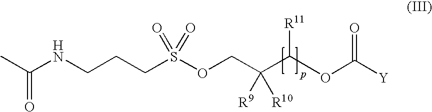

or a pharmaceutically acceptable salt thereof; wherein:

p is chosen from 0, 1, 2, and 3;

Y is chosen from R¹², —OR¹², and —NR¹²₂, wherein:
each R¹² is independently chosen from C₁₋₈ alkyl, substituted C₁₋₈ alkyl, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;

R⁹ and R¹⁰ are independently chosen from C₁₋₄ alkyl and substituted C₁₋₄ alkyl; or R⁹ and R¹⁰ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and

each R¹¹ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl; and

with the proviso that when

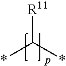

is C₁₋₈ alkyldiyl, and Y is chosen from R¹² and —OR¹²; then R¹² is not chosen from C₁₋₈ alkyl, C₆₋₁₀ aryl, substituted C₆₋₁₀ aryl, C₅₋₁₀ heteroaryl, substituted C₅₋₁₀ heteroaryl, C₇₋₁₈ arylalkyl, and C₆₋₁₈ heteroarylalkyl.

In certain embodiments of a compound of Formula (III), R¹¹ is not substituted C₁₋₈ heteroalkyl. In certain embodiments of a compound of Formula (III), R¹¹ is not substituted C₁₋₈ heteroalkyl, wherein the one or more substituent groups is ═O.

In certain embodiments of a compound of Formula (III), each substituent group is independently chosen from halogen, —OH, —CN, —CF₃, —OCF₃, ═O, —NO₂, C₁₋₆ alkoxy, C₁₋₆ alkyl, —COOR²⁶, —NR²⁷₂, and —CONR²⁸₂; wherein each of R²⁶, R²⁷, and R²⁸ is independently chosen from hydrogen and C₁₋₆ alkyl. In certain embodiments, each substituent group is chosen from C₁₋₄ alkyl, C₁₋₄ alkoxy, —OH, and —NH₂.

In certain embodiments of a compound of Formula (III), Y is R¹². In certain embodiments of a compound of Formula (III) wherein Y is R¹², R¹² is chosen from C₁₋₈ alkyl, C₆₋₁₀ aryl, C₃₋₈ cycloalkyl, C₇₋₁₈ arylalkyl, C₄₋₁₆ cycloalkylalkyl, C₁₋₈ heteroalkyl, C₅₋₁₀ heteroaryl, C₃₋₈ heterocycloalkyl, C₆₋₁₈ heteroarylalkyl, and C₄₋₁₆ heterocycloalkylalkyl. In certain embodiments of a compound of Formula (III) wherein Y is R¹², R¹² is chosen from C₁₋₈ alkyl, C₆₋₁₀ aryl, C₃₋₈ cycloalkyl, C₇₋₁₈ arylalkyl, and C₄₋₁₆ cycloalkylalkyl. In certain embodiments of a compound of Formula (III) wherein Y is R¹², R¹² is chosen from C₁₋₆ alkyl, cyclohexyl, and phenyl.

In certain embodiments of a compound of Formula (III), Y is —OR¹². In certain embodiments of a compound of Formula (III) wherein Y is —OR¹², R¹² is chosen from C₁₋₈ alkyl, C₆₋₁₀ aryl, C₃₋₈ cycloalkyl, C₇₋₁₈ arylalkyl, C₄₋₁₆ cycloalkylalkyl, C₁₋₈ heteroalkyl, C₅₋₁₀ heteroaryl, C₃₋₈ heterocycloalkyl, C₆₋₁₈ heteroarylalkyl, and C₄₋₁₆ heterocycloalkylalkyl. In certain embodiments of a compound of Formula (III) wherein Y is —OR¹², R¹² is chosen from C₁₋₈ alkyl, C₆₋₁₀ aryl, C₃₋₈ cycloalkyl, C₇₋₁₈ arylalkyl, and C₄₋₁₆ cycloalkylalkyl. In certain embodiments of a compound of Formula (III) wherein Y is —OR¹², R¹² is chosen from C₁₋₆ alkyl, cyclohexyl, and phenyl.

In certain embodiments of a compound of Formula (III), Y is —NR¹²₂. In certain embodiments of a compound of Formula (III) wherein Y is —NR¹²₂, R¹² is chosen from C₁₋₈ alkyl, C₆₋₁₀ aryl, C₃₋₈ cycloalkyl, C₇₋₁₈ arylalkyl, C₄₋₁₆ cycloalkylalkyl, C₁₋₈ heteroalkyl, C₅₋₁₀ heteroaryl, C₃₋₈ heterocycloalkyl, C₆₋₁₈ heteroarylalkyl, and C₄₋₁₆ heterocycloalkylalkyl. In certain embodiments of a compound of Formula (III) wherein Y is —NR¹²₂, R¹² is chosen from C₁₋₈ alkyl, C₆₋₁₀ aryl, C₃₋₈ cycloalkyl, C₇₋₁₈ arylalkyl, and C₄₋₁₆ cycloalkylalkyl. In certain embodiments of a compound of Formula (III) wherein Y is —NR¹²₂, each R¹² is independently chosen from C₁₋₆ alkyl.

In certain embodiments of a compound of Formula (III), p is 0, p is 1, p is 2, and in certain embodiments, p is 3. In certain embodiments of a compound of Formula (III), p is chosen from 0, 1, and 2.

In certain embodiments of a compound of Formula (III), each R¹¹ is chosen from hydrogen, C₁₋₆ alkyl, phenyl, and substituted phenyl. In certain embodiments of a compound of Formula (III), each R¹¹ is chosen from hydrogen and C₁₋₄ alkyl. In certain embodiments of a compound of Formula (III), each R¹¹ is hydrogen.

In certain embodiments of a compound of Formula (III), each of R⁹ and R¹¹ is methyl.

In certain embodiments of a compound of Formula (III), p is chosen from 0, 1, and 2; Y is R¹² wherein R¹² is chosen from C₁₋₄ alkyl, cyclohexyl, and phenyl; each R¹¹ is chosen from hydrogen and C₁₋₄ alkyl; and each of R⁹ and R¹⁰ is methyl.

In certain embodiments of a compound of Formula (III), p is chosen from 0, 1, and 2; Y is —OR¹² wherein R¹² is chosen from C₁₋₄ alkyl, cyclohexyl, and phenyl; each R¹¹ is chosen from hydrogen and C₁₋₄ alkyl; and each of R⁹ and R¹⁰ is methyl.

In certain embodiments of a compound of Formula (III), p is chosen from 0, 1, and 2; Y is —NR¹²₂ wherein each R¹² is independently chosen from C₁₋₄ alkyl; each R¹¹ is chosen from hydrogen and C₁₋₄ alkyl; and each of R⁹ and R¹⁰ is methyl.

Certain embodiments provide a compound of Formula (IV):

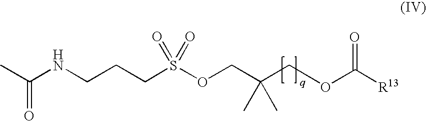

or a pharmaceutically acceptable salt thereof; wherein:

q is chosen from 0, 1, 2, and 3; and

R¹³ is chosen from ethoxy, phenyl, —CH₂NH₂, and C₁₋₆ alkyl.

In certain embodiments of a compound of Formula (IV), q is 0, q is 1, q is 2, and in certain embodiments, q is 3.

In certain embodiments of a compound of Formula (IV), the compound is chosen from:
4-{[3-(acetylamino)propyl]sulfonyloxy}-3,3-dimethylbutyl benzoate;
4-{[3-(acetylamino)propyl]sulfonyloxy}-3,3-dimethylbutyl 2-aminoacetate hydrochloride;
3-{[3-(acetylamino)propyl]sulfonyloxy}-2,2-dimethylpropyl 2-methylpropanoate;
3-{[3-(acetylamino)propyl]sulfonyloxy}-2,2-dimethylpropyl benzoate;
5-{[3-(acetylamino)propyl]sulfonyloxy}-4,4-dimethylpentyl ethoxyformate;
5-{[3-(acetylamino)propyl]sulfonyloxy}-4,4-dimethylpentyl benzoate; and

a pharmaceutically acceptable salt of any of the foregoing.

Certain embodiments provide a compound of Formula (V):

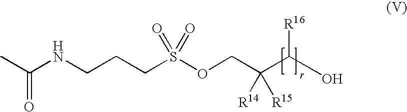

or a pharmaceutically acceptable salt thereof; wherein:

r is chosen from 0, 1, 2, and 3;

R¹⁴ and R¹⁵ are independently chosen from C₁₋₄ alkyl and substituted C₁₋₄ alkyl; or R¹⁴ and R¹⁵ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and

each R¹⁶ is independently chosen from hydrogen, halogen, —OH, —CN, —CF₃, —OCF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

In certain embodiments of a compound of Formula (V), when each of R¹⁴ and R¹⁵ is methyl, and r is 1; then R¹⁶ is not hydrogen.

In certain embodiments of a compound of Formula (V), R¹⁶ is not substituted C₁₋₈ heteroalkyl. In certain embodiments of a compound of Formula (V), R¹⁶ is not chosen from substituted C₁₋₈ heteroalkyl, wherein the one or more substituent groups is ═O.

In certain embodiments of a compound of Formula (V), each substituent group is independently chosen from halogen, —OH, —CN, —CF₃, —OCF₃, ═O, —NO₂, C₁₋₆ alkoxy, C₁₋₆ alkyl, —COOR²⁶, —NR²⁷₂, and —CONR²⁸₂; wherein each R²⁶, R²⁷, and R²⁸ is independently chosen from hydrogen and C₁₋₆ alkyl. In certain embodiments, each substituent group is chosen from C₁₋₄ alkyl, —OH, and —NH₂.

In certain embodiments of a compound of Formula (V), each of R¹⁴ and R¹⁵ is methyl.

In certain embodiments of a compound of Formula (V), each R¹⁶ is hydrogen.

In certain embodiments of a compound of Formula (V), r is chosen from 0, 1, and 2.

In certain embodiments of a compound of Formula (V), each of R¹⁴ and R¹⁵ is methyl; each R¹⁶ is hydrogen; and r is chosen from 0, 1, and 2.

In certain embodiments of a compound of Formula (V), the compound is chosen from:
2-hydroxy-2-methylpropyl[3-(acetylamino)propyl]sulfonate;
4-hydroxy-2,2-dimethylbutyl [3-(acetylamino)propyl]sulfonate;
5-hydroxy-2,2-dimethylpentyl [3-(acetylamino)propyl]sulfonate; and

a pharmaceutically acceptable salt of any of the foregoing.

In certain embodiments of compounds of Formula (I), (III), and (IV), a pharmaceutically acceptable salt is selected from a sodium salt, a potassium salt, a lithium salt, an ammonium salt, a calcium salt, a zinc salt, and a magnesium salt. In certain embodiments of compounds of Formula (I), (III), and (IV), a pharmaceutically acceptable salt is the hydrochloride salt, and in certain embodiments, the sodium salt.

### Synthesis

Compounds disclosed herein may be obtained via the synthetic methods illustrated in Schemes 1-14. Those of ordinary skill in the art will appreciate that a useful synthetic route to the disclosed compounds comprises bonding a substituted neopentyl promoiety bearing a suitable functional group at the neopentyl position of the promoiety to acamprosate, i.e. the sulfonyl chloride of acamprosate, to form a substituted neopentyl sulfonyl ester moiety.

General synthetic methods useful in the synthesis of compounds described herein are available in the art. Starting materials useful for preparing compounds and intermediates thereof and/or practicing methods described herein are commercially available or can be prepared by well-known synthetic methods. Other methods for the synthesis of compounds provided by the present disclosure are either described in the art or will be readily apparent to the skilled artisan in view of the references provided herein and may be used to synthesize the compounds provided by the present disclosure. Accordingly, the methods presented in the schemes are illustrative rather than comprehensive.

### Synthesis of Masked Nitrogen Nucleophiles

Masked nitrogen nucleophile neopentylsulfonic acid prodrugs, intermediates, and precursors of any of the foregoing can be prepared according to general synthetic Schemes 1-5. Neopentyl alcohol may be prepared from commercially available 3,3-dimethyloxirane using the procedures of Mullis et al., J Org. Chem. 1982, 47, 2873-2875 and Roberts et al., Tetrahedron Lett. 1997, 38, 355-358, or following the procedure described in Roberts, et al., U.S. Pat. No. 5,596,095 (WO 96/18609). Neopentyl alcohol may also be prepared by the procedures described by Scheinmann et al., J. Chem. Res. (S) 1993, 414-415, and Flynn et al., J. Org. Chem. 1983, 48, 2424-2426, using pyrrolidin-2-one as the starting material as shown in Scheme 1.

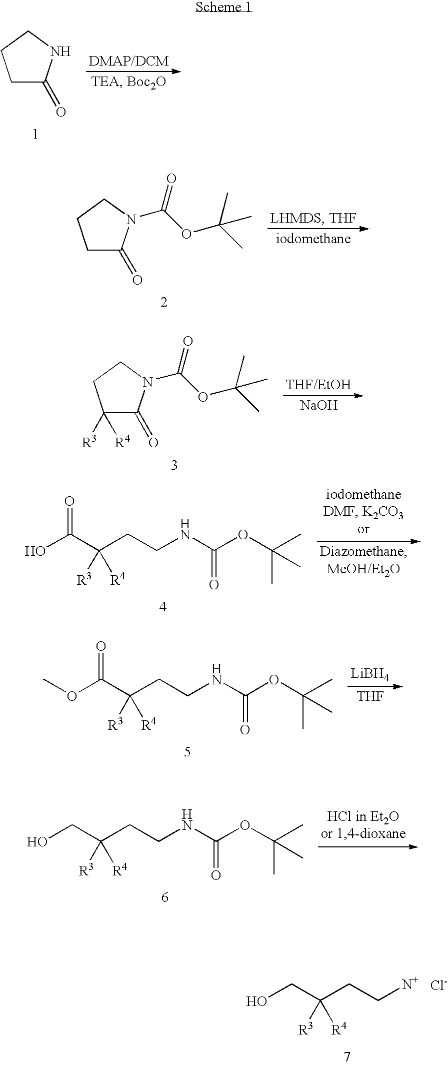

where R³ and R⁴ are as defined herein.

As shown in Scheme 1-pyrrolidin-2-one 1 can be reacted with di-tert-butylpyrocarbonate (Boc₂O) in the presence of 4-(N,N-dimethyl)aminopyridine (DMAP)/dichloromethane (DCM) and a catalytic amount of triethylamine to provide 1-(tert-butoxy)carbonyl-3,3-dimethylpyrrolidin-2-one 2. The Boc-carbonyl protected pyrrolidin-2-one can be 3,3-dialkylated by reacting compound 2 with lithiumhexalkyldisilazide (LHMDS) under a nitrogen atmosphere in tetrahydrofuran (THF) and iodomethane to provide compound 3. The 3,3-dialkylated Boc-carbonyl protected pyrrolidin-2-one ring is opened in a solution of THF/ethanol and sodium hydroxide (NaOH) to provide the corresponding Boc-carbonyl protected free acid. Compound 4 can first be esterifed by reacting with iodomethane in the presence of a base such as potassium carbonate in anhydrous N,N-dimethylformamide (DMF), or alternatively, compound 4 can be reacted in methanol (MeOH) with a slight excess of freshly generated diazomethane in diethylether (Et₂O) to provide the corresponding N-Boc carbonyl protected Neon-B methylester 5. The desired Neon B alcohol 6 can be obtained by reacting methylester 5 in anhydrous tetrahydrofuran (THF) with lithium borohydride (LiBH₄). The N-tertbutyloxycarbonyl protecting group (N-Boc) of alcohol 6 can be removed by reaction with hydrogen chloride in 1,4-dioxane or diethyl ether (Et₂O) to provide the corresponding hydrochloride salt of amino alcohol 7.

As shown in Scheme 2, other Neon-B-type alcohols, i.e. amino alcohols 7 where n is not 2, can be N-Boc-protected to provide the N-Boc protected higher or lower analogs 8 of N-Boc-protected Neon-B-alcohol 7 using the methods A or B shown in Scheme 2.

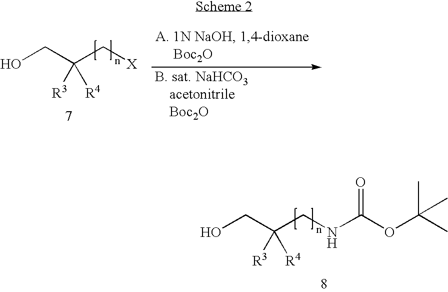

where n is chosen from 0, 1, 2 (for n=2, compound 8 is equivalent to compound 6 in Scheme 1), and 3; and X is chosen from NH₂ and NH₃⁺Cl⁻; and R³ and R⁴ are as defined herein. Neopentyl alcohol 7 can be reacted with di-tert-butyl pyrocarbonate (di-tert-butyldicarbonate, Boc₂O) Boc₂O in a mixture of a 1N aqueous solution of sodium hydroxide (NaOH) and 1,4-dioxane to provide the corresponding N-Boc protected ω-amino-2,2-disubstituted alcohol 8. Alternatively, neopentyl alcohol 7 can be reacted with Boc₂O in a saturated aqueous solution of sodium bicarbonate (NaHCO₃) to provide the corresponding N-Boc protected ω-amino-2,2-disubstituted alcohol 8.

The synthesis of acyloxyalkyl carbamates and acyloxyalkyl carbamate prodrugs is disclosed in Zerangue et al., U.S. Pat. No. 7,351,740, which is incorporated by reference in its entirety. For example, as shown in FIG. 3, cycloxyalkylcarbamate neopentyl alcohols can be prepared by reacting appropriately substituted acyloxyalkyl N-hydroxysuccinimide (NHS) carbonic acid esters 9 with netopentyl alcohols 10, or a suitable derivative thereof, e.g., a hydrochloride salt (X═NH₃⁺Cl⁻), to provide the corresponding N-acyloxyalkyl carbamate protected ω-amino-2,2-disubstituted alcohol 11 as shown in Scheme 3.

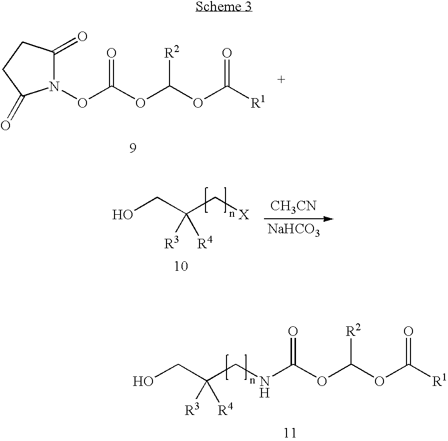

where X is —NH₂; and n, R¹, R², R³, and R⁴ are as defined herein. Acyloxyalkyl N-hydroxy succinimide carbonic acid ester 9 can be reacted with a neopentyl alcohol 10 in acetonitrile and sodium bicarbonate to provide the corresponding acyloxyalkylcarbamate neopentyl alcohol 11. Alternatively, free aminoalcohols may be reacted in a methyl tert-butylether/acetone/water mixture (4:3:1) as disclosed in Zerangue et al., U.S. Pat. No. 7,351,740.

Acyloxyalkylcarbamate neopentyl prodrugs of acamprosate can be prepared as shown in Scheme 4.

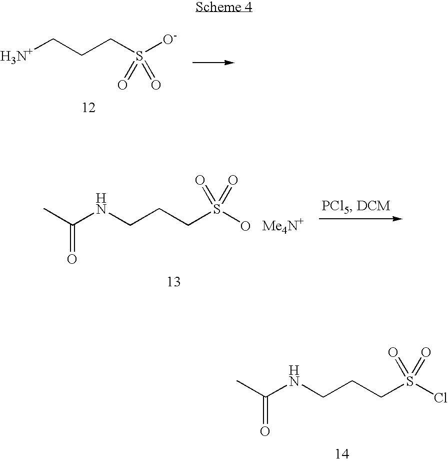

Commercially available homotaurine 12 can be simultaneously N-acetylated and converted to the corresponding tetramethylammonium salt by reacting homotaurine 12 with tetramethylammonium hydroxide (TMAH) and acetic anhydride (Ac₂O) in a mixture of methanol and water to provide acamprosate tetramethylammonium salt 13. Contacting tetramethylammonium salt 13 with phosphorous pentachloride (PCl₅) or other chlorination agent such as sulfuryl chloride (SO₂Cl₂) in a solvent such as dichloromethane (DCM) provides the corresponding sulfonic acid chloride 14 (acamprosate chloride).

N-Acyloxyalkylcarbamate neopentyl prodrugs can be prepared as shown in Scheme 5.

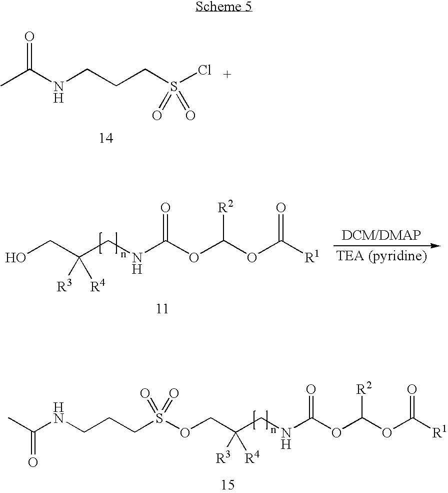

where n, R¹, R², R³ and R⁴ are as defined herein.

Referring to Scheme 5, the sulfonic acid chloride of acamprosate 14 can be reacted with N-acyloxyalkylcarbamate neopentyl alcohol 11 in an appropriate solvent such as dichloromethane (DCM) and in the presence of a suitable base, e.g., triethylamine (TEA) and a catalytic amount of 4-(N,N-dimethyl)aminopyridine (DMAP) to provide the corresponding acamprosate sulfonyl ester 15 of N-acyloxyalkylcarbamate protected ω-amino-2,2-disubstituted alcohols or acamprosate neopentyl prodrug.

N-Boc-protected neopentyl derivatives of acamprosate or derivatives thereof can be prepared as shown in Scheme 6.

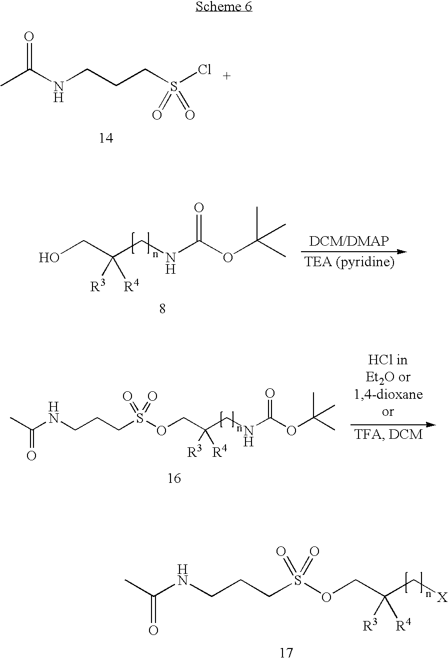

where n, R³, R⁴, and X are as defined herein.

Referring to Scheme 6, sulfonic acid chloride of acamprosate 14 can be reacted with N-Boc-protected neopentyl alcohol 8 under similar conditions as described for the preparation of acamprosate sulfonyl esters 15 of N-acyloxyalkylcarbamate protected ω-amino-2,2-disubstituted alcohols in Scheme 5 to provide the corresponding N-Boc-protected neopentyl sulfonylester of acamprosate 16. The corresponding unprotected neopentyl sulfonylester of acamprosate 17 can be obtained by reacting N-Boc-protected neopentyl sulfonyl ester derivative 16 with a strong acid in an inert solvent, for example, trifluoroacetic acid (TFA) in dichloromethane (DCM) or hydrogen chloride (HCl) in 1,4-dioxane or diethyl ether (Et₂O), to remove the tert-butoxycarbonyl (Boc) protecting group and provide the corresponding unprotected species in either the free amine or N-protonated form, i.e. ammonium, where X is NH₂, NH₃⁺Cl⁻, or NH₃⁺F₃CCO₂⁻; and n, R³, and R⁴ are as defined herein.

### Synthesis of Masked Oxygen Nucleophiles

Masked oxygen nucleophile-based neopentyl sulfonic acid prodrugs, intermediates, and precursors of any of the foregoing can be prepared according to general synthetic Schemes 7-15.

An example of the preparation of O-alkyl protected nucleophiles such as O-benzyl-masked oxygen nucleophiles corresponding to n is 0 is shown in Scheme 7 where R⁹ and R¹⁰ are as defined herein, PG is a protecting group such as benzyl or substituted benzyl, and X is a halogen capable of activating the introduction of protecting group PG. In certain embodiments of Scheme 7, each of R⁹ and R¹⁰ is methyl, and PG is benzyl.

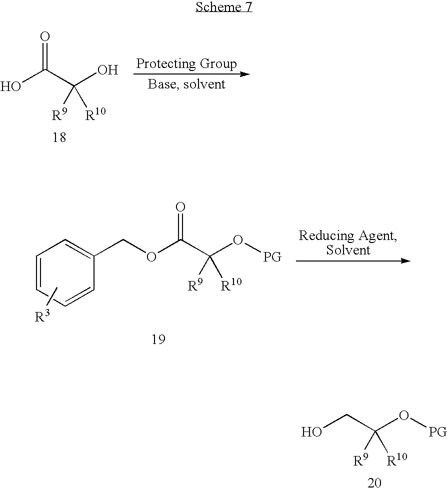

Employing synthetic methods commonly used for similar synthetic transformations, commercially available 2,2-dialkyl-glycolic acid 18 can be benzylated in the presence of a base such as alkali hydrides, i.e. sodium hydride (NaH), or alkali carbonate, e.g., Cs₂CO₃ or K₂CO₃, employing benzylation reagents such as benzyl halides, e.g., benzyl bromide (BnBr), in the presence of an inert solvent such as N,N-dimethylformamide (DMF) or tetrahydrofuran (THF), at a temperature from about 0° C. to about 100° C. to provide bis-benzylated derivative 19. Reduction of the benzyl carboxylate with a suitable reducing agent such as lithium aluminum hydride (LAH) in an appropriate solvent such as tetrahydrofuran (THF) or diethyl ether (Et₂O), at a temperature from about −78° C. to about 0° C. affords the corresponding O-benzylated neopentyl-type derivative 20.

A method for preparing O-mono-acylated and O-mono-alkylated 2,2-bis-substituted propane-1,3-diols 22 as protected nucleophiles, i.e. masked oxygen nucleophiles corresponding to n is 1, is shown in Scheme 8.

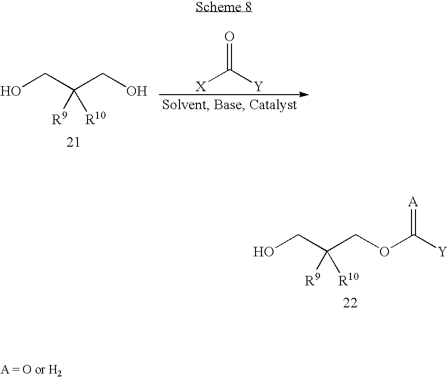

where R⁹ and R¹⁰ are as defined herein. In certain embodiments, each of R⁹ and R¹⁰ is methyl, PG is a protecting group, YC(A), and the activated protecting group is PGX or YC(A)X. Y can be alkyl, alkoxy, or aryl. Depending on the nature of Y, e.g., alkyl or alkoxy, A is oxygen and X is a leaving group such that activated O-protecting group YCAX (or PGX) is, for example, a carboxcylic acid halide, or a alkyl/aryl chloroformate. When Y is (substituted) phenyl and A is two hydrogen atoms, then X is bromo, and the O-protecting group is benzyl or substituted benzyl and the activated protecting group YC(A)X (or PGX) is, for example, benzyl bromide (BnBr).

2,2-Bis-substituted propane-1,3-diols 21 are either commercially available or can be synthesized using standard methods. Employing standard synthetic protocols for the transformation of hydroxyl-functionalities of 2,2-bis-substituted propane-1,3-diols 21, the corresponding mono-acylated derivative 22 can be obtained by reaction with a suitably functionalized activated carboxylic acid or carbonic acid derivatives XC(O)Y. Y is as defined above, and X is a leaving group such as a halide, i.e., chlorine, a carboxylate YCO₂ where (symmetrical anhydride), a lower alkyl carbonic acid monoester such as EtOCO₂ (mixed anhydride), or an O-acylurea such as C₆H₁₁—N═C(—O)NH—C₆H₁₁. Examples of functionalized activated carboxylic acid derivatives include carboxylic acid chlorides such as benzoyl chloride (PhCOCl) and isobutanoyl chloride (iPrCOCl) (2-methyl-propanoyl chloride). An example of an activated carbonic acid derivative is ethyl chlorofommate (EtOCOCl). The reaction can be carried out in the presence of an appropriate base such as a tertiary amine, for example, triethylamine (Et₃N, TEA), diisopropyl ethylamine (iPr₂EtN, DIEA), or pyridine, with or without a nucleophilic acylation catalyst such as 4-(N,N-dimethyl)aminopyridine (DMAP), and in the presence of an inert solvent such as dichloromethane (DCM), tetrahydrofuran (THF), or mixture thereof. The reaction can be carried out at a temperature from about 0° C. to about 60° C.

Williamson's ether syntheses are well known and can be used to form alkyl ethers from alcohols and alkyl halides. Accordingly, the hydroxyl-functionalities of 2,2-bis-substituted propane-1,3-diol 21 can be transformed to the corresponding mono-alkyl or benzyl derivative 22 using functionalized, protected or unprotected, and activated alkyl halides such as benzyl halides, e.g. benzyl bromide (BnBr), or the corresponding sulfonate, in the presence of a base such as an alkali hydride, e.g., sodium hydride (NaH); an alkali carbonate, e.g., Cs₂CO₃ or K₂CO₃; or a tertiary organic base, e.g., triethylamine (Et₃N, TEA) or diisopropyl ethylamine (iPr₂EtN, DIEA); in an inert solvent such as N,N-dimethylformamide (DMF) or tetrahydrofuran (THF), at a temperature from about 0° C. to about 60° C.

It is known that derivatization of symmetrical or unsymmetrical multivalent molecules decorated with more than one of the same functional group, i.e., hydroxyl groups such as in 1,3-diols, provides a statistical mixture of non-, mono-, and bis-functionalized products. The product ratios reflect the regiochemical preference of the functional group towards a certain derivatization agent. In certain embodiments, the mixtures of monofunctionalized 1,3-propane diols 22 can be separated using, for example, silica gel column chromatography or other separation method

Methods for preparing O-mono-acylated or O-mono-alkylated 2,2-bis-substituted butane-1,3-diols 29 as protected nucleophiles, i.e. masked oxygen nucleophiles corresponding to n is 2 are shown in Schemes 9 and 10.

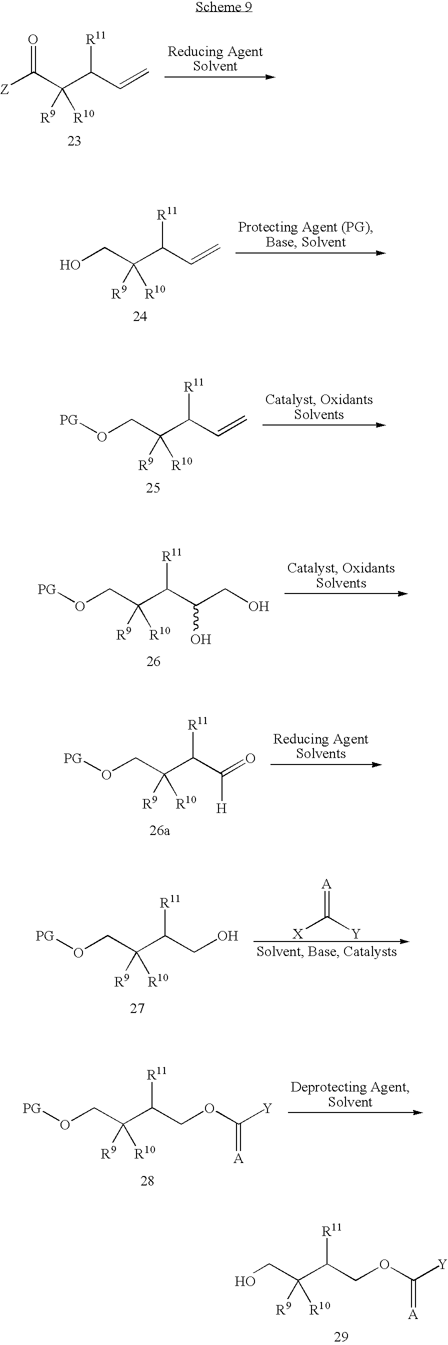

where R⁹, R¹⁰, and R¹¹ are as defined herein; Z is hydroxyl, lower alkoxy, or hydrogen, PG is a protecting group, and YC(A) where Y is alkyl, alkoxy, or aryl. Depending on the nature of Y, i.e., alkyl or alkoxy, A is oxygen and X is a leaving group such that the activated O-protecting group YC(A)X (or PGX) is a carboxylic acid halide, or an alkyl/aryl chloroformate. If Y is, for example, (substituted) phenyl and A is two hydrogen atoms then X is bromo, and the O-protecting group is a benzyl or substituted benzyl group, and the activated protecting group, YC(A)X (or PGX) is, for example, benzyl bromide (BnBr).

2,2-Bis-substituted butane-1,4-diols are either commercially available or can be synthesized using standard methods known in the art. For example, employing standard synthetic protocols, commercially available 2,2-dimethyl-4-pentenoic acid or its C₁₋₆ alkyl ester 23 (Y is OH or C₁₋₆ alkoxy; each of R⁹ and R¹⁰ is methyl; and R¹¹ is hydrogen) can be converted to the corresponding alcohol 24 by reaction with reducing agents such as lithium aluminum hydride (LiAlH₄, LAH) in the presence of an anhydrous inert solvent such as tetrahydrofuran (THF) or diethyl ether (Et₂O), at a temperature from about −78° C. to about 65° C. Alternatively, aldehydes 23, e.g., 2,2-dimethyl-pent-4-enal (Z is hydrogen) can be reduced with boron hydride reagents such as sodium borohydride (NaBH₄) in the presence of alcoholic solvents such as methanol (MeOH) or ethanol (EtOH) at temperatures from about 0° C. to about 25° C. Following standard protocols, the hydroxyl group of the resulting alcohol derivative, 2,2-dialkyl-penten-4-ol 24 can be protected by reacting the alcohol with a protecting agent such as an alkyl- or alkyl/aryl-functionalized chlorosilane, for example, tert-butylchlorodimethylsilane (tert-Bu(Me)₂SiCl, TBDMSCl), in the presence of an organic tertiary base such as imidazole (C₃H₃N₂), triethylamine (Et₃N, TEA), or diisopropyl ethylamine (iPr₂EtN, DIEA) and an inert solvent such as N,N-dimethylformamide (DMF), dichloromethane (DCM), or tetrahydrofuran (THF) at a temperature from about 0° C. to about 25° C. to provide the corresponding 1,1,2,2-tetramethyl-1-silapropane protected intermediate 25. Methods known to those skilled in the art can be used to convert the olefinic double bonds into aldehydes. For example, intermediate 25 can be converted to the corresponding 1,2-diol 26 by reaction with a catalytic amount of a suitable oxidation mixture such as osmium tetroxide (OsO₄) and N-methyl morpholine oxide (NMO) in the presence of a mixture of suitable solvents such as water and acetone in a ratio of approximately 1:1 by volume and at a temperature from about 0° C. to about 40° C. to provide the corresponding 1,2-diol intermediate 26. Employing standard synthetic methods, 1,2-diol 26 can be oxidatively cleaved to the corresponding aldehyde 26a by reaction with a suitable second oxidant such as sodium metaperiodate (NaIO₄) in the presence of a mixture of suitable solvents such as water and ethanol in a ratio of approximately 1:1 by volume and at a temperature from about 0° C. to about 40° C. to provide the corresponding aldehyde intermediate 26a. Aldehyde 26a can be reduced using a reducing agent such as sodium borohydride (NaBH₄) in the presence of an appropriate solvent such as methanol (MeOH) at a temperature from about 0° C. to about 40° C. to provide the corresponding TBDMS-protected neopentyl alcohol 27.

Employing standard synthetic protocols for the transformation of hydroxyl-functionalities, TBDMS-protected neopentyl alcohol 27 can then be converted to the corresponding mono-acylated derivative 28 by reacting with a suitably functionalized activated carboxylic acid, amino acid, or carbonic acid derivative XC(O)Y where Y is as defined above, and X is a leaving group such as a halide, e.g., chlorine; a carboxylate YCO₂ (symmetrical anhydride); a lower alkyl carbonic acid monoester such as EtOCO₂ (mixed anhydride); or an O-acylurea such as C₆H₁₁—N═C(—O)NH—C₆H₁₁). Examples of functionalized activated carboxylic acid derivatives include carboxylic acid chlorides such as benzoyl chloride (PhCOCl) and isobutanoyl chloride (iPrCOCl) (2-methyl-propanoyl chloride). An example of an activated carbonic acid derivative is ethyl chloroformate (EtOCOCl). The reaction can be carried out in the presence of an appropriate base such as a tertiary amine, for example triethylamine (Et₃N, TEA), diisopropyl ethylamine (iPr₂EtN, DIEA), or pyridine, with or without a suitable nucleophilic acylation catalyst such as 4-(N,N-dimethyl)aminopyridine (DMAP), and in the presence of an inert solvent such as dichloromethane (DCM), tetrahydrofuran (THF), or mixture thereof. The reaction can be carried out at a temperature from about 0° C. to about 60° C.

Alternatively, the free hydroxyl group of TBDMS-protected neopentyl alcohol 27 can be esterified with an activated and protected amino acid derivative such as N-Boc-glycine, or others. Following standard methods, protected amino acids can be activated with an activation agent such as dicyclohexylcarbodiimide (DCC) in the presence of an acylation catalyst such as 4-(N,N-dimethyl)aminopyridine (DMAP) in the presence of a solvent such as anhydrous dichloromethane (DCM). The activated amino acid can then be reacted directly with the TBDMS-protected neopentyl alcohol 27 in the same solvent at a temperature from about 0° C. to about 25° C. to provide the corresponding amino acid ester derivative 28 where Y is an amino acid derivative of starting alcohol 27.

Alternatively, using Williamson's ether synthesis the hydroxyl-functionality of the TBDMS-protected neopentyl alcohol 27 can be transformed to the corresponding mono-alkylated derivative 28 (where A is two hydrogens) with a functionalized, protected or unprotected, and activated alkyl halide such as a benzyl halides, e.g., benzyl bromide (BnBr), or the corresponding sulfonate, using an appropriate base such as an alkali hydride, e.g., such as sodium hydride (NaH); an alkali carbonate such as Cs₂CO₃ or K₂CO₃; or a tertiary organic base such as triethylamine (Et₃N, TEA) or diisopropyl ethylamine (iPr₂EtN, DIEA) in the presence of an inert solvent such as N,N-dimethylformamide (DMF), tetrahydrofuran (THF), or mixture thereof, at a temperature from about 0° C. to about 60° C.

O,O′-Bis-protected neopentyl diol 28 can be selectively desilylated by methods known in the art. For example, O,O′-bis-protected neopentyl diol 28 can be reacted with fluoride-based desilylation reagents such as triethylamine trishydrogenfluoride complex (Et₃N.3HF) in the presence of an inert solvent such as tetrahydrofuran (THF), at a temperature from about 25° C. to about 65° C., to provide the corresponding mono-protected 1,4-diol 29.

3,3-Bis-substituted derivatives 30 are also useful starting materials for the preparation of O-mono-acylated or O-mono-alkylated 2,2-bis-substituted butane-1,3-diols 33 that can be used as protected nucleophiles, i.e. masked oxygen nucleophiles corresponding to n is 2, as shown in Scheme 10.

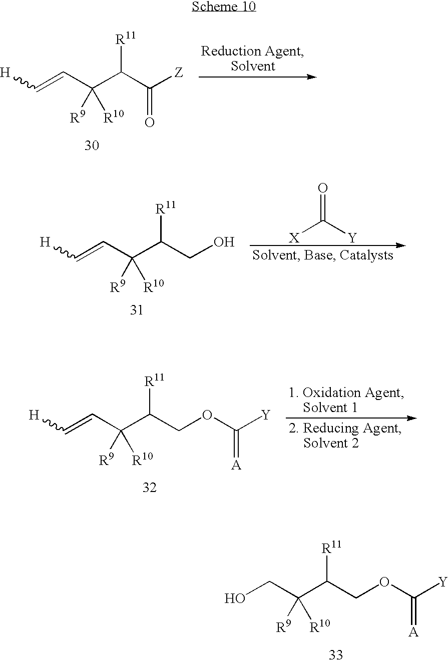

where R⁹, R¹⁰, and R¹¹ are as defined herein; Z is C₁₋₆ alkoxy such as methoxy, PG is a protecting group, YC(A), where Y is alkyl, alkoxy, or aryl. Depending on the nature of Y, i.e., alkyl or alkoxy, A is oxygen and X is a leaving group such that the activated O-protecting group YC(A)X (or PGX) is, for example, a carboxylic acid halide; or an alkyl/aryl chloroformate. When Y is, for example, (substituted) phenyl and A is two hydrogen atoms, then X is bromo, and the O-protecting group is a benzyl or substituted benzyl group and the activated protecting group YC(A)X (or PGX) is, for example, benzyl bromide (BnBr).

3,3-Bis-substituted derivatives 30 are either commercially available or can be synthesized using standard methods. Employing standard synthetic protocols, commercially available methyl 3,3-dimethyl-4-pentenoate 30 (Z is methoxy; each of R⁹ and R¹⁰ is methyl; R¹¹ is hydrogen, and PG is benzyl) can be converted to the corresponding alcohol 31 by reaction with a reducing agent such as lithium aluminum hydride (LiAlH₄, LAH) in the presence of an anhydrous inert solvent such as tetrahydrofuran (THF) or diethyl ether (Et₂O), at a temperature from about −78° C. to about 65° C. Lithium borohydride (LiBH₄) is an alternative reducing agent for this transformation and can be used in the presence of alcohol solvents such as methanol (MeOH) or ethanol (EtOH) at a temperature from about 0° C. to about 25° C. to provide alcohol 31. Alcohol 31 can then be reacted to provide the corresponding O-acylated or O-alkylated derivative 32 using similar reagents, solvents, catalysts, and reaction conditions as described for the synthesis of compounds 28 and 29 in Scheme 9.

Conversion of carbon-carbon double bonds such as in alkene 32 to the corresponding O-protected hydroxymethylene derivative 33 can be achieved by oxidative cleavage of the carbon-carbon double bond followed by reductive work-up of oxygenated intermediates that, depending on the cleavage condition, may or may not be isolated in pure form. For example, alkene 32 can be reacted with an excess of a mixture of oxygen and ozone (O₂/O₃) in the presence of an inert solvent such as dichloromethane (DCM) at a temperature from about −100° C. to about −60° C. The intermediate oxygenated derivative (molonozide) can be converted to the corresponding alcohol 33 by reaction with a reducing agent such as lithium aluminum hydride (LiAlH₄, LAH) in the presence of an anhydrous inert solvent such as tetrahydrofuran (THF) or diethyl ether (Et₂O), at a temperature from about −78° C. to about 65° C. Sodium borohydride (NaBH₄) in an alcoholic solvent such as methanol (MeOH) or ethanol (EtOH) at a temperature from about 0° C. to about 25° C., or borane dimethylsulfide complex (BH₃.Me₂S) in tetrahydrofuran (THF) are alternative reducing agents for this transformation and can be used to provide alcohol 33.

The preparation of functionalized or substituted O-mono-acylated or O-mono-alkylated 2,2-bis-substituted pentane-1,5-diols 36-38 as protected nucleophiles, i.e. masked oxygen nucleophiles corresponding to n is 3, is shown in Scheme 11.

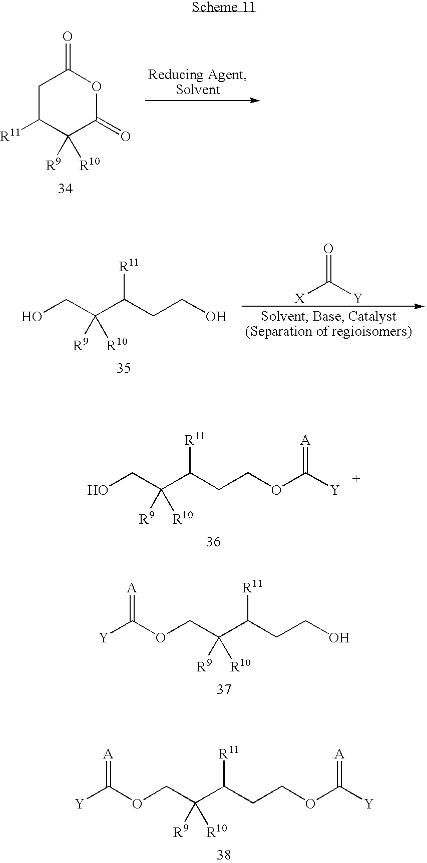

where R⁹, R¹⁰, and R¹¹ are as defined herein; PG is a protecting group YC(A) where Y is either alkyl, alkoxy, or aryl. Depending on the nature of Y, i.e., alkyl or alkoxy, A is oxygen and X is a suitable leaving group such that the activated O-protecting group YC(A)X (or PGX) is, for example, a carboxylic acid halide, or an alkyl/aryl chloroformate. When Y is, for example, (substituted) phenyl and A is two hydrogen atoms, then X is bromo, and the O-protecting group is a benzyl or substituted benzyl group and the activated protecting group YC(A)X (or PGX) is, for example, benzyl bromide (BnBr).

Precursors to functionalized 2,2-bis-substituted pentane-1,4-diol 38 are either commercially available or can be synthesized using standard methods known in the art. In certain embodiments, the starting material 34 is 2,2-dimethylglutaric anhydride and each of R⁹ and R¹⁰ is methyl, R¹¹ is hydrogen, PG is benzyl, and Y is either C₁₋₆ alkoxy such as ethoxy (OEt), aryl such as phenyl (Ph), or C₁₋₆ alkyl such as tert-butyl (tBu), and X is chlorine. In other embodiments, A is two hydrogens, Y is phenyl, and X is chlorine. For example, employing standard synthetic protocols, commercially available 2,2-dimethylglutaric anhydride 34 (each of R⁹ and R¹⁰ is methyl; and R¹¹ is hydrogen) can be converted to the corresponding alcohol 35 by global reduction with a reducing agent such as lithium aluminum hydride (LiAlH₄, LAH) in the presence of an anhydrous inert solvent such as tetrahydrofuran (THF), diethyl ether (Et₂O), or a mixture thereof, at a temperature from about −78° C. to about 65° C. Employing standard synthetic protocols for the transformation of hydroxyl-functionalities, 1,5-diol 35 can be converted to the corresponding mono-O-acylated derivative (A is oxygen) 36, 37, bis-O-acylated derivatives 38, or mixtures thereof, by reacting with a suitably functionalized activated carboxylic or carbonic acid derivative (ZCOY, where Z is a suitable leaving group such as chlorine, and Y is as defined herein) or an activated carbonic acid derivative (ZCOOR¹², where Z is a suitable leaving group such as chlorine, and R¹² is as defined herein). Examples of useful carbonic acid derivatives include carboxylic acid chlorides such as benzoyl chloride (PhCOCl) and pivaloyl chloride (tBuCOCl). An example of a useful carbonic acid derivative is ethyl chloroformate. The reaction can be carried out using an appropriate base such as a tertiary amine, for example triethylamine (Et₃N, TEA), diisopropyl ethylamine (iPr₂EtN, DIEA), or pyridine, with or without a nucleophilic acylation catalyst such as 4-(N,N-dimethyl)aminopyridine (DMAP), and in the presence of an inert solvent such as dichloromethane (DCM) or tetrahydrofuran (THF). The reaction can be carried out at a temperature from about 0° C. to about 60° C.

Alternatively, Williamson's ether syntheses can be used to form alkyl ethers from alcohols and alkyl halides. For example, the hydroxyl-functionalities of 1,5-protected diol 35 can be transformed to the corresponding mono-O-alkylated derivative 36 or 37, bis-O-alkylated derivative 38, or combinations thereof using suitably functionalized, protected or unprotected, and activated alkyl halides including benzyl halides such as benzyl bromide (BnBr), or a sulfonate, employing bases such as an alkali hydride, e.g., sodium hydride (NaH), an alkali carbonate such as Cs₂CO₃ and K₂CO₃, or a tertiary organic base such as triethylamine (Et₃N, TEA) and diisopropyl ethylamine (iPr₂EtN, DIEA) in the presence of an inert solvent such as 4-N,N-dimethylformamide (DMF) or tetrahydrofuran (THF) at a temperature from about 0° C. to about 60° C.

Derivatization of multivalent molecules decorated with more than one of the same functional group such as in 1,5-diol 35 provides mixtures of non-, mono-, and bis-functionalized products. The product ratios reflect the regiochemical preference of the functional groups towards a certain derivatization agent.

In certain embodiments, the mixtures of O-acylation or O-alkylation products 36 or 37 and O,O′-bis-acylation/bis-alkylation product 38 can be used directly and without additional separation, isolation, or purification in subsequent reaction steps where the molecules provided by such steps potentially may be more readily purified or separated to provide regiochemically uniform material.

In certain embodiments, for example, where Y is tert-butyl, in which the products are obtained as mixtures of O-acylated or O-alkylated regioisomers (36 or 37, respectively) and O,O′-bis-acylated or bis-alkylated products 38, the mixtures can be separated using, for example, silica gel column chromatography or other appropriate separation method to provide mono-O-acylated or mono-O-alkylated isomers of defined regiochemistry 36 in highly enriched or pure form. For example, in certain embodiments where each of R⁹ and R¹⁰ is methyl, R¹¹ is hydrogen, A is oxygen, and Y is tert-butyl, the material can be isolated in highly regioisomerially enriched or regioisomerically pure form by silica gel column chromatography.

Following orthogonal trans-protection strategies or variations thereof according to Hashimoto, et al., J. Am. Chem. Soc. 1988, 110, 3670-3672, or other synthetic and protecting methods known in the art, e.g., the Williamson's ether synthesis as described in Schemes 8-10, the purified regioisomer of 36 where each of R⁹ and R¹⁰ is methyl, R¹¹ is hydrogen, A is oxygen, and Y is tert-butyl, can be converted to the corresponding benzyl derivative where each of R⁹ and R¹⁰ is methyl, R¹¹ is hydrogen, A is two hydrogens, and Y is phenyl.

Neopentyl sulfonyl ester prodrugs acamprosate, precursors thereof, and intermediates thereof 41 can be prepared as shown in Scheme 12.

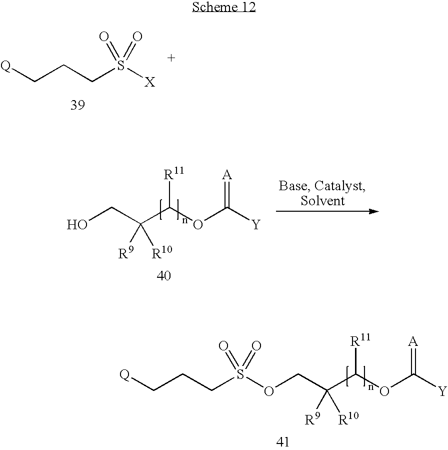

where n, R⁹, R¹⁰, R¹¹, A is O or two hydrogens, Q is NHAc or chloro, and Y are as defined herein.

Referring to Scheme 12, an activated sulfonic acid derivative such as a sulfonyl chloride of a drug having at least one sulfonic acid group 39, e.g., acamprosate, or an activated sulfonic acid derivative such as a chloride of a suitable precursor of a drug having at least one sulfonic acid group can be reacted with an O-functionalized neopentyl alcohol 40 in an appropriate solvent such as dichloromethane (DCM) in the presence of a suitable base such as triethylamine (Et₃N, TEA), pyridine, or diisopropyl ethylamine (iPr₂EtN, DIEA), and in the presence of a nucleophilic catalyst such as 4-(N,N-dimethyl)aminopyridine (DMAP) at a temperature from about −20° C. to about 25° C. to provide the corresponding neopentyl sulfonyl ester 41. When the sulfonylchloride is acamprosate chloride (Q is N-acetylamino) then compound 39 in Scheme 12 is equivalent to compound 14 in Scheme 4. The reaction can be conducted under comparable conditions as described for the preparation of the N-Boc protected or N-acyloxyalkylcarbamate-protected neopentyl sulfonyl esters, i.e., compounds 15 and 16 in Schemes 5 and 6, respectively, using an appropriate solvent such as dichloromethane (DCM) in the presence of a suitable base such as triethylamine (Et₃N, TEA), pyridine, or diisopropyl ethylamine (iPr₂EtN, DIEA), and in the presence of a nucleophilic catalyst such as 4-(N,N-dimethyl)aminopyridine (DMAP) at a temperature from about −20° C. to about 25° C. In certain embodiments, Q is chosen from chlorine and N-acetylamino; X is chlorine; n is chosen from 0, 1, 2, and 3; each of R⁹ and R¹⁰ is methyl; R¹¹ is hydrogen; A is chosen from oxygen and two hydrogens; and Y is chosen from ethoxy (OEt), isopropyl (iPr), phenyl (Ph), and CH₂NHBoc.

Referring to Scheme 13, certain compounds 41 (e.g., Q is chlorine) are precursors for the synthesis of acamprosate prodrugs 42.

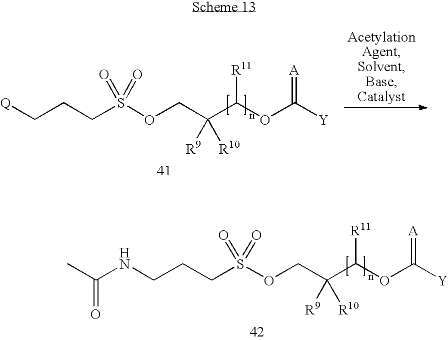

wherein n, R⁹, R¹⁰, R¹¹, A, Y, and Q are as defined herein.

In certain embodiments of compounds 41, where Q is chlorine, each of R⁹ and R¹⁰ is methyl, R¹¹ is hydrogen, A is oxygen or two hydrogens, and Y is aryl such as phenyl (Ph) or alkyl such isopropyl (iPr), the chloro-functionality can be converted to the corresponding N-acetylamino (NHAc) functionality using methods known in the art. For example, compound 41, where Q is chlorine, each of R⁹ and R¹⁰ is methyl, R¹¹ is hydrogen, A is 0 or two hydrogens, and Y is aryl such as phenyl (Ph) or alkyl such as isopropyl (iPr), can be reacted with an azide-nucleophile or salt thereof such as sodium azide (NaN₃), or tetrabutylammonium azide (nBu₄NN₃), in a polar non-protic solvent, for example, anhydrous dimethyl sulfoxide (DMSO), anhydrous N,N-dimethylformamide (DMF), acetonitrile (H₃CCN), and the like, or mixture thereof, at a temperature from about 0° C. to about 100° C., to provide the corresponding organic primary azide.

Azides can be isolated in pure form employing methods such as silica gel column chromatography or can be used directly and without additional isolation or purification in subsequent reactionsteps following aqueous work-up. Primary azides (Q=N₃) can be converted to the corresponding free amine intermediate which can then isolated in pure form as a salt of a mineral acid such as hydrogen chloride (HCl) or an organic acid such as acetic acid (H₃CCO₂H), trifluoroacetic acid (F₃CCO₂H), or mixtures thereof. For azide-containing intermediates in which Q is azido, each of R⁹ and R¹⁰ is methyl, R¹¹ is hydrogen, A is oxygen, and Y is aryl such as phenyl (Ph) or alkyl such isopropyl (iPr), the azide-containing intermediate can be reacted with an azide-reducing agent. An example of an appropriate reducing agent is hydrogen (H₂) in the presence of a catalyst such a palladium on activated carbon. The reaction can be carried out in a solvent such as methanol (MeOH), ethanol (EtOH), ethyl acetate (EtOAc), and the like, or mixtures thereof, under a pressure from about atmospheric pressure to about 100 psi at a temperature from about 0° C. to about 100° C.

Alternatively, the azide functionality can be reduced using metal salts such as stannous chloride (SnCl₂) in a protic solvent such as methanol (MeOH), at a temperature from about 0° C. to about 60° C., or with aryl- or alkyl-phosphines such as triphenylphosphine (Ph₃P) in a solvent mixture such as tetrahydrofuran (THF) and water, at a temperature from about 0° C. to about 60° C. Other appropriate reducing agents and methods can be used for the transformation. The corresponding amine intermediates are provided in either free amine or N-protonated form, i.e. ammonium, where Q is chosen from NH₂, NH₃⁺Cl⁻, NH₃⁺H₃CCO₂⁻, NH₃⁺F₃CO₂⁻, and other suitable salt combinations, or mixtures thereof. Intermediate amine-derivatives where Q is chosen from NH₂, NH₃⁺Cl⁻, NH₃⁺H₃CCO₂⁻, and NH₃⁺F₃CO₂⁻, can either be directly isolated in pure form or can be purified using standard methods. The amines or ammonium salts can be used with or without additional isolation or purification in the next step. The corresponding species in either free amine or N-protonated form can be acetylated employing commonly used synthetic methods to provide the corresponding N-acetylated species. For example, in certain embodiments, where Q is chosen from NH₂, NH₃⁺Cl⁻, NH₃⁺H₃CCO₂⁻, and NH₃⁺F₃CO₂⁻, each of R⁹ and R¹⁰ is methyl, R¹¹ is hydrogen, A is oxygen or two hydrogens, and Y is aryl such as phenyl (Ph) or alkyl such isopropyl (iPr), the free amine or N-protonated forms can be reacted with an acetylation agent such as acetyl chloride (AcCl), acetic anhydride (Ac₂O), or other activated acetylation agent, with or without a nucleophilic acylation catalyst such as 4-(N,N-dimethyl)aminopyridine (DMAP), in a suitable solvent such as dichloromethane (DCM), at a temperature from about −20° C. to about 60° C.

Referring to Scheme 14, in certain embodiments, Q is N-acetylamino (NHAc) (in compound 41); each of R⁹ and R¹⁰ is methyl; R¹¹ is hydrogen; and A is oxygen; and Y is —CH₂NHBoc; the corresponding unprotected derivatives of the amino acid conjugates of neopentyl sulfonylester prodrugs 44 can be obtained by reacting the corresponding N-Boc-protected neopentyl sulfonyl ester derivative 43 with a strong acid in an inert solvent, for example, trifluoroacetic acid in dichloromethane (DCM) or hydrogen chloride (HCl) in 1,4-dioxane, to cleave the tert-butoxycarbonyl (Boc) protecting group to provide the corresponding unprotected species in either free amine or N-protonated form, i.e. ammonium, where X is chosen from NH₂, NH₃⁺Cl⁻, and NH₃⁺F₃CCO₂⁻; and R⁹, R¹⁰, and R¹¹ are as defined herein.

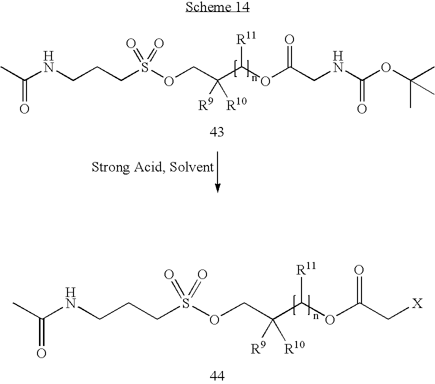

Referring to Scheme 15, in certain embodiments where Q is N-acetylamino (NHAc) in compound 41; each of R⁹ and R¹⁰ is methyl; R¹¹ is hydrogen; and X is NH₂ or NH₃⁺Cl⁻, the corresponding unprotected derivatives of the conjugates of neopentyl sulfonylester prodrugs of acamprosate 46 can be obtained upon reacting a substituted phenyl-protected neopentyl sulfonyl ester derivative 45 with hydrogen and a hydrogenation catalyst such as 5-10% palladium on activated carbon in the presence of a solvent such as ethanol, methanol, ethylacetate, and mixtures thereof under a pressure of about 100 psi at a temperature from about 25° C. to about 60° C.

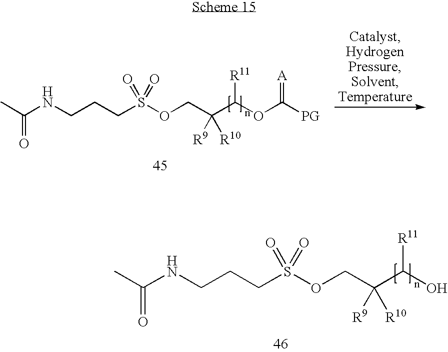

### Pharmaceutical Compositions

Pharmaceutical compositions provided by the present disclosure comprise a compound of Formula (I), Formula (III), and/or Formula (IV) together with a suitable amount of one or more pharmaceutically acceptable vehicles so as to provide a composition for proper administration to a patient. Examples of suitable pharmaceutical vehicles are known in the art.

Pharmaceutical compositions comprising a compound of Formula (I), Formula (III), and/or Formula (IV) may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of compounds of Formula (I), Formula (III), or Formula (IV) or crystalline form thereof and one or more pharmaceutically acceptable vehicles into formulations that can be used pharmaceutically. Proper formulation is dependent upon the route of administration chosen. In certain embodiments, a pharmaceutical composition comprising a compound of Formula (I), Formula (III), or Formula (IV) or crystalline form thereof may be formulated for oral administration, and in certain embodiments for sustained release oral administration. Pharmaceutical compositions provided by the present disclosure may take the form of solutions, suspensions, emulsion, tablets, pills, pellets, capsules, capsules containing liquids, powders, sustained-release formulations, suppositories, emulsions, aerosols, sprays, suspensions, or any other form suitable for administration to a patient.

Pharmaceutical compositions provided by the present disclosure may be formulated in a unit dosage form. A unit dosage form refers to a physically discrete unit suitable as a unitary dose for patients undergoing treatment, with each unit containing a predetermined quantity of at least one compound of Formula (I), Formula (III), or Formula (IV) calculated to produce an intended therapeutic effect. A unit dosage form may be for a single daily dose, for administration 2 times per day, or one of multiple daily doses, e.g., 3 or more times per day. When multiple daily doses are used, a unit dosage may be the same or different for each dose. One or more dosage forms may comprise a dose, which may be administered to a patient at a single point in time or during a time interval.

In certain embodiments, a compound of Formula (I), Formula (III), or Formula (IV) may be incorporated into pharmaceutical compositions to be administered orally. Oral administration of such pharmaceutical compositions may result in uptake of a compound of Formula (I), Formula (III), or Formula (IV) throughout the intestine and entry into the systemic circulation. Such oral compositions may be prepared in a manner known in the pharmaceutical art and comprise at least one compound of Formula (I), Formula (III), or Formula (IV) and at least one pharmaceutically acceptable vehicle. Oral pharmaceutical compositions may include a therapeutically effective amount of at least one compound of Formula (I), Formula (III), or Formula (IV) and a suitable amount of a pharmaceutically acceptable vehicle, so as to provide an appropriate form for administration to a patient.

Pharmaceutical compositions comprising at least one compound of Formula (I), Formula (III), or Formula (IV) may be formulated for immediate release for parenteral administration, oral administration, or for any other appropriate route of administration.

Controlled drug delivery systems may be designed to deliver a drug in such a way that the drug level is maintained within a therapeutically effective window and effective and safe blood levels are maintained for a period as long as the system continues to deliver the drug at a particular rate. Controlled drug delivery may produce substantially constant blood levels of a drug over a period of time as compared to fluctuations observed with immediate release dosage forms. For some drugs, maintaining a constant blood and tissue concentration throughout the course of therapy is the most desirable mode of treatment. Immediate release of drugs may cause blood levels to peak above the level required to elicit a desired response, which may waste the drug and may cause or exacerbate toxic side effects. Controlled drug delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

In certain embodiments, an oral dosage form provided by the present disclosure may be a controlled release dosage form. Controlled delivery technologies can improve the absorption of a drug in a particular region or regions of the gastrointestinal tract.

The appropriate oral dosage form for a particular pharmaceutical composition provided by the present disclosure may depend, at least in part, on the gastrointestinal absorption properties of a compound of Formula (I), Formula (III), or Formula (IV), the stability of a compound of Formula (I), Formula (III), or Formula (IV) in the gastrointestinal tract, the pharmacokinetics of a compound of Formula (I), Formula (III), or Formula (IV), and the intended therapeutic profile. An appropriate controlled release oral dosage form may be selected for a particular compound of Formula (I), Formula (III), or Formula (IV). For example, gastric retention oral dosage forms may be appropriate for compounds absorbed primarily from the upper gastrointestinal tract, and sustained release oral dosage forms may be appropriate for compounds absorbed primarily from the lower gastrointestinal tract. Certain compounds are absorbed primarily from the small intestine. In general, compounds traverse the length of the small intestine in about 3 to 5 hours. For compounds that are not easily absorbed by the small intestine or that do not dissolve readily, the window for active agent absorption in the small intestine may be too short to provide a desired therapeutic effect.

Gastric retention dosage forms, i.e., dosage forms that are designed to be retained in the stomach for a prolonged period of time, may increase the bioavailability of drugs that are most readily absorbed by the upper gastrointestinal tract. For example, certain compounds of Formula (I), Formula (III), or Formula (IV) may exhibit limited colonic absorption, and be absorbed primarily from the upper gastrointestinal tract. Thus, dosage forms that release a compound of Formula (I), Formula (III), or Formula (IV) in the upper gastrointestinal tract and/or retard transit of the dosage form through the upper gastrointestinal tract will tend to enhance the oral bioavailability of the compound of Formula (I), Formula (III), or Formula (IV). The residence time of a conventional dosage form in the stomach is about 1 to about 3 hours. After transiting the stomach, there is approximately a 3 to 5 hour window of bioavailability before the dosage form reaches the colon. However, if the dosage form is retained in the stomach, the drug may be released before it reaches the small intestine and will enter the intestine in solution in a state in which it can be more readily absorbed. Another use of gastric retention dosage forms is to improve the bioavailability of a drug that is unstable to the basic conditions of the intestine.

In certain embodiments, pharmaceutical compositions provided by the present disclosure may be practiced with dosage forms adapted to provide sustained release of a compound of Formula (I), Formula (III), or Formula (IV) upon oral administration. Sustained release oral dosage forms may be used to release drugs over a prolonged time period and are useful when it is desired that a drug or drug form be delivered to the lower gastrointestinal tract. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

Sustained release oral dosage forms may be in any appropriate form for oral administration, such as, for example, in the form of tablets, pills, or granules. Granules can be filled into capsules, compressed into tablets, or included in a liquid suspension. Sustained release oral dosage forms may additionally include an exterior coating to provide, for example, acid protection, ease of swallowing, flavor, identification, and the like.

In certain embodiments, sustained release oral dosage forms may comprise a therapeutically effective amount of a compound of Formula (I), Formula (III), or Formula (IV) and at least one pharmaceutically acceptable vehicle. In certain embodiments, a sustained release oral dosage form may comprise less than a therapeutically effective amount of a compound of Formula (I), Formula (III), or Formula (IV) and a pharmaceutically effective vehicle. Multiple sustained release oral dosage forms, each dosage form comprising less than a therapeutically effective amount of a compound of Formula (I), Formula (III), or Formula (IV) may be administered at a single time or over a period of time to provide a therapeutically effective dose or regimen for treating a disease in a patient. In certain embodiments, a sustained release oral dosage form comprises more than one compound of Formula (I), Formula (III), and/or Formula (IV).

Sustained release oral dosage forms provided by the present disclosure can release a compound of Formula (I), Formula (III), or Formula (IV) from the dosage form to facilitate the ability of the compound of Formula (I), Formula (III), or Formula (IV) to be absorbed from an appropriate region of the gastrointestinal tract, for example, in the small intestine or in the colon. In certain embodiments, sustained release oral dosage forms may release a compound of Formula (I), Formula (III), or Formula (IV) from the dosage form over a period of at least about 4 hours, at least about 8 hours, at least about 12 hours, at least about 16 hours, at least about 20 hours, and in certain embodiments, at least about 24 hours. In certain embodiments, sustained release oral dosage forms may release a compound of Formula (I), Formula (III), or Formula (IV) from the dosage form in a delivery pattern corresponding to about 0 wt % to about 20 wt % in about 0 to about 4 hours; about 20 wt % to about 50 wt % in about 0 to about 8 hours; about 55 wt % to about 85 wt % in about 0 to about 14 hours; and about 80 wt % to about 100 wt % in about 0 to about 24 hours; where wt % refers to the percent of the total weight of the compound in the dosage form. In certain embodiments, sustained release oral dosage forms may release a compound of Formula (I), Formula (III), or Formula (IV) from the dosage form in a delivery pattern corresponding to about 0 wt % to about 20 wt % in about 0 to about 4 hours; about 20 wt % to about 50 wt % in about 0 to about 8 hours; about 55 wt % to about 85 wt % in about 0 to about 14 hours; and about 80 wt % to about 100 wt % in about 0 to about 20 hours. In certain embodiments, sustained release oral dosage forms may release a compound of Formula (I), Formula (III), or Formula (IV) from the dosage form in a delivery pattern corresponding to about 0 wt % to about 20 wt % in about 0 to about 2 hours; about 20 wt % to about 50 wt % in about 0 to about 4 hours; about 55 wt % to about 85 wt % in about 0 to about 7 hours; and about 80 wt % to about 100 wt % in about 0 to about 8 hours.

Sustained release oral dosage forms comprising a compound of Formula (I), Formula (III), or Formula (IV) may provide a concentration of the corresponding drug in the plasma, blood, cerebrospinal fluid, or tissue of a patient over time, following oral administration to the patient. The concentration profile of the drug may exhibit an AUC that is proportional to the dose of the corresponding compound of Formula (I), Formula (III), or Formula (IV).

Regardless of the specific type of controlled release oral dosage form used, a compound of Formula (I), Formula (III), or Formula (IV) may be released from an orally administered dosage form over a sufficient period of time to provide prolonged therapeutic concentrations of the compound of Formula (I), Formula (III), or Formula (IV) in the plasma and/or blood of a patient. Following oral administration, a dosage form comprising a compound of Formula (I), Formula (III), or Formula (IV) may provide a therapeutically effective concentration of the corresponding drug in the plasma and/or blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous time periods during which a therapeutically effective concentration of the drug is maintained may be the same or different. The continuous period of time during which a therapeutically effective plasma concentration of the drug is maintained may begin shortly after oral administration or following a time interval.

An appropriate dosage of a compound of Formula (I), Formula (III), or Formula (IV) or pharmaceutical composition comprising a compound of Formula (I), Formula (III), or Formula (IV) may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

### Uses

Compounds of Formula (I), Formula (III), and Formula (IV) are prodrugs of acamprosate. Thus, compounds of Formula (I), Formula (III), and Formula (IV) may be administered to a patient suffering from any disease including a disorder, condition, or symptom for which acamprosate is known or hereafter discovered to be therapeutically effective. Methods for treating a disease in a patient provided by the present disclosure comprise administering to a patient in need of such treatment a therapeutically effective amount of at least one compound of Formula (I), Formula (III), and/or Formula (IV).

Compounds of Formula (I), Formula (III), and Formula (IV) or pharmaceutical compositions thereof may provide therapeutic or prophylactic plasma and/or blood concentrations of the corresponding drug following oral administration to a patient. The promoiety(ies) of compounds of Formula (I), Formula (III), and Formula (IV) may be cleaved in vivo either chemically and/or enzymatically to release the parent drug. One or more enzymes present in the intestinal lumen, intestinal tissue, blood, liver, brain, or any other suitable tissue of a patient may enzymatically cleave the promoiety of the administered compounds. For example, a promoiety of a compound of Formula (I), Formula (III), and Formula (IV) may be cleaved following absorption of the compound from the gastrointestinal tract (e.g., in intestinal tissue, blood, liver, or other suitable tissue of a mammal). For compounds of Formula (I), Formula (III), and Formula (IV) the masking promoiety is first cleaved enzymatically, chemically, or by both mechanisms to provide a neopentyl promoiety terminated with a nitrogen or oxygen nucleophile. The structures of the oxygen and nitrogen nucleophile metabolic intermediates have the structures of Formula (II) and Formula (V), respectively. The nucleophilic group can then internally cyclize to release acamprosate. Metabolic intermediates of masked nitrogen nucleophile prodrugs of acamprosate have the structure of Formula (II) herein. Metabolic intermediates of masked oxygen nucleophile prodrugs of acamprosate have the structure of Formula (V) herein.

In certain embodiments, compounds of Formula (I), Formula (III), and Formula (IV) may be actively transported across the intestinal endothelium by transporters expressed in the gastrointestinal tract including the small intestine and colon. The drug, e.g., acamprosate, may remain conjugated to the promoiety during transit across the intestinal mucosal barrier to prevent or minimize presystemic metabolism. In certain embodiments, a compound of Formula (I), Formula (III), or Formula (IV) is essentially not metabolized to acamprosate within gastrointestinal enterocytes, but is metabolized to release acamprosate within the systemic circulation, for example in the intestinal tissue, blood/plasma, liver, or other suitable tissue of a mammal. In such embodiments, compounds of Formula (I), Formula (III), and Formula (IV) may be absorbed into the systemic circulation from the small and large intestines either by active transport, passive diffusion, or by both active and passive processes. For example, a promoiety may be cleaved after absorption from the gastrointestinal tract, for example, in intestinal tissue, blood, liver, or other suitable tissue of a mammal.

Compounds of Formula (I), Formula (III), and Formula (IV) may be administered in similar equivalent amounts of acamprosate and using a similar dosing schedule as described in the art for treatment of a particular disease. For example, in a human subject weighing about 70 kg, compounds of Formula (I), Formula (III), and Formula (IV) may be administered at a dose over time having an equivalent weight of acamprosate from about 10 mg to about 10 g per day, and in certain embodiments, an equivalent weight of acamprosate from about 1 mg to about 3 g per day. A dose of a compound of Formula (I), Formula (III), or Formula (IV) taken at any one time can have an equivalent weight of acamprosate from about 1 mg to about 3 g. An acamprosate dose may be determined based on several factors, including, for example, the body weight and/or condition of the patient being treated, the severity of the disease being treated, the incidence of side effects, the manner of administration, and the judgment of the prescribing physician. Dosage ranges may be determined by methods known to one skilled in the art. In certain embodiments, compounds of Formula (I), Formula (III), and Formula (IV) provide a higher oral bioavailability of acamprosate compared to the oral bioavailability of acamprosate when orally administered at an equivalent dose and in an equivalent dosage form. Consequently, a lesser equivalent amount of acamprosate derived from a compound of Formula (I), Formula (III), or Formula (IV) may be orally administered to achieve the same therapeutic effect as that achieved when acamprosate itself is orally administered.

Compounds of Formula (I), Formula (III), and Formula (IV) may be assayed in vitro and in vivo for the desired therapeutic or prophylactic activity prior to use in humans. For example, in vitro assays may be used to determine whether administration of a compound of Formula (I), Formula (III), or Formula (IV) is a substrate of a transporter protein, including transporters expressed in the gastrointestinal tract. Examples of certain assay methods applicable to analyzing the ability of compounds of Formula (I), Formula (III), and Formula (IV) to act as substrates for one or more transporter proteins are disclosed in Zerangue et al., US 2003/0158254. In vivo assays, for example using appropriate animal models, may also be used to determine whether administration of a compound of Formula (I), Formula (III), or Formula (IV) is therapeutically effective. Compounds of Formula (I), Formula (III), and Formula (IV) may also be demonstrated to be therapeutically effective and safe using animal model systems.

In certain embodiments, a therapeutically effective dose of a compound of Formula (I), Formula (III), or Formula (IV) may provide therapeutic benefit without causing substantial toxicity. Toxicity of compounds of Formula (I), Formula (III), and Formula (IV), prodrugs, and/or metabolites thereof may be determined using standard pharmaceutical procedures and may be ascertained by one skilled in the art. The dose ratio between toxic and therapeutic effect is the therapeutic index. A dose of a compound of Formula (I), Formula (III), or Formula (IV) may be within a range capable of establishing and maintaining a therapeutically effective circulating plasma and/or blood concentration of a compound of Formula (I), Formula (III), and Formula (IV) or acamprosate that exhibits little or no toxicity.

Compounds of Formula (I), Formula (III), and Formula (IV) may be used to treat diseases, disorders, conditions, and symptoms of any of the foregoing for which acamprosate is shown to provide therapeutic benefit. Hence, compounds of Formula (I), Formula (III), and Formula (IV) may be used to treat neurodegenerative disorders, psychotic disorders, mood disorders, anxiety disorders, somatoform disorders, movement disorders, substance abuse disorders, binge eating disorder, cortical spreading depression related disorders, tinnitus, sleeping disorders, multiple sclerosis, and pain. The underlying etiology of any of the foregoing diseases being so treated may have a multiplicity of origins.

Further, in certain embodiments, a therapeutically effective amount of one or more compounds of Formula (I), Formula (III), and Formula (IV) may be administered to a patient, such as a human, as a preventative measure against various diseases or disorders. Thus, a therapeutically effective amount of one or more compounds of Formula (I), Formula (III), and Formula (IV) can be administered as a preventative measure to a patient having a predisposition for a neurodegenerative disorder, a psychotic disorder, a mood disorder, an anxiety disorder, a somatoform disorder, a movement disorder, a substance abuse disorder, binge eating disorder, a cortical spreading depression related disorder, tinnitus, a sleeping disorder, multiple sclerosis, or pain.

Substance abuse disorders refer to disorders related to taking a drug of abuse, to the side effects of a medication, and to toxin exposure. Drugs of abuse include alcohol, amphetamines, caffeine, cannabis, cocaine, hallucinogens, inhalants, nicotine, opioids, phencyclidine, or similarly acting arylcyclohexylamines, sedatives, hypnotics, and anxiolytics.

Alcoholism or alcohol dependence is a chronic disorder with genetic, psychosocial, and environmental causes. Alcoholism refers to “ . . . maladaptive alcohol use with clinically significant impairment as manifested by at least three of the following within any one-year period: tolerance; withdrawal; taken in greater amounts or over longer time course than intended; desire or unsuccessful attempts to cut down or control use; great deal of time spent obtaining, using, or recovering from use; social, occupational, or recreational activities given up or reduced; continued use despite knowledge of physical or psychological sequelae.” (Diagnostic and Statistical Manual of Mental Disorders, Fourth Edition, Text Revision, Washington D.C., American Psychiatric Association, 2000 (DSM-IV)). Alcohol use disorders include alcohol dependence and alcohol abuse. Screening tests useful for identifying alcoholism include the Alcohol Dependence Data Questionnaire, the Michigan Alcohol Screening Test, the Alcohol Use Disorders Identification Test, and the Paddington Alcohol Test, and other generally recognized tests for diagnosing alcohol dependence.

Treatment for alcoholism generally includes psychological, social, and pharmacotherapeutic interventions aimed at reducing alcohol-associated problems and usually involves detoxification and rehabilitation phases. Medications useful in the pharmacologic treatment of alcohol dependence include disulfiram and naltrexone.

Studies suggest that modulation of mGluR5 receptors play a role in substance abuse disorders and that mGluR5 receptor antagonists such as MPEP may be useful in treating such conditions including drug abuse disorders.

Acamprosate has been shown to be effective for maintaining abstinence from alcohol in patients with alcohol dependence that are abstinent at the initiation of acamprosate treatment (Scott et al., CNS Drugs 2005, 19(5), 445-464; and Heilig and Egli, Pharmacology & Therapeutics 2006, 11, 855-876) and as such is marketed in the United States for the treatment of alcohol abstinence as Campral® (Forest Laboratories and Merck KGaA). Typical acamprosate doses range from about 1-2 gm per day to achieve a steady-state plasma concentration of about 370-640 ng/mL, which occurs at about 3-8 hours post-dose (Overman et al., Annals Pharmacotherapy 2003, 37, 1090-1099; Paille et al., Alcohol. 1995, 30, 239-47; and Pelc et al., Br. Psychiatry 1997, 171, 73-77) with a recommended dose of Campral® being two to three 333 mg tablets taken three times daily.

The efficacy of compounds of Formula (I), Formula (III), and Formula (IV) and compositions thereof for treating alcohol dependency may be assessed using animal models of alcoholism and using clinical studies. Animal models of alcoholism are known. Clinical protocols for assessing the efficacy of a compound of Formula (I), Formula (III), and Formula (IV) for treating alcoholism are known.

The effect of acamprosate on relapse in other substances of abuse has not been extensively studied; however administration of 100 mg/kg acamprosate for 3 days attenuated relapse-like behavior in cocaine conditioned mice (Mcgeehan and Olive, Behav Pharmacol 2006, 17(4), 363-7). Studies suggest that modulation of mGluR5 receptors play a role in substance abuse disorders and that mGluR5 receptor antagonists such as MPEP may be useful in treating such conditions including drug abuse disorders and nicotine abuse disorders. Therefore, acamprosate may have applicability in treating other substance abuse disorders, including narcotic abuse disorders and nicotine abuse disorders.

Binge eating disorder is characterized by recurrent episodes of distressing, uncontrollable eating of excessively large amounts of food without the inappropriate compensatory weight loss behaviors of bulimia nervosa or anorexia nervosa (DSM-IV, Fourth Ed., Text Revision, Washington D.C., American Psychiatric Assoc., 2000). The pathophysiology of binge eating disorders is unknown. Binge eating disorder is associated with psychopathology such as compulsive, impulsive, and affective disorders, medical comorbidity, especially obesity, impaired quality of life, and disability. Emotional cues such as anger, sadness, boredom, and anxiety can trigger binge eating. Impulsive behavior and certain other emotional problems can be more common in people with binge eating disorder. Antidepressant medications, including tricyclic antidepressants, selective serotonin re-uptake inhibitors, as well as some of certain antidepressants, have shown evidence of some therapeutic value in binge eating disorder (Bello and Jajnal, Brain Res Bulletin 2006, 70, 422-429; Buda-Levin et al., Physiology & Behavior 2005, 86, 176-184; and Han et al., Drug Alcohol Dependence 2007, prepublication no. DAD-3137, 5 pages).

The efficacy of compounds of acamprosate prodrugs and compositions for treating binge eating may be assessed using animal models of binge eating and using clinical studies. Animal models of binge eating are known. Clinical protocols useful for assessing the efficacy of an acamprosate prodrug for treating binge eating are also known.

In certain embodiments, compounds of Formula (I), Formula (III), and Formula (IV) can be used to treat tinnitus. Tinnitus is the perception of sound in the absence of acoustic stimulation and often involves sound sensations such as ringing, buzzing, roaring, whistling, or hissing that cannot be attributed to an external sound source. Tinnitus is a symptom associated with many forms of hearing loss and can also be a symptom of other health problems.

Tinnitus can be caused by hearing loss, loud noise, medicine, and other health problems such as allergies, head or neck tumors, cardiovascular disorders such as atherosclerosis, high blood pressure, turbulent blood flow, malformation of capillaries, trauma such as excessive exposure to loud noise, long-term use of certain medications such as salicylates, quinine, cisplatin and certain types of antibiotics, changes to ear bones such as otosclerosis, and jaw and neck injuries. In general, insults or damage to the auditory and somatosensory systems can create an imbalance between inhibitory and excitatory transmitter actions in the midbrain, auditory cortex, and brain stem. This imbalance can cause hyperexcitability of auditory neurons that can lead to the perception of phantom sounds. For acute tinnitus such as tinnitus induced by drugs or loud noises, increased spontaneous firing rates in the auditory nerve fibers have been attributed to reduced levels of central inhibition, presumably by GABA, in central auditory structures leading to neural hyperactivity in the inferior colliculus. Although chronic tinnitus may have a different cause than acute tinnitus, reduced GABA levels have also been implicated.

A recent clinical trial suggests that acamprosate may be effective in treating tinnitus (Azevedo and Figueiredo, Rev Bras Otorrinolaringol 2005, 71(5), 618-23).

Acamprosate prodrugs of Formula (I), Formula (III), and Formula (IV) can be used to treat tinnitus, including preventing, reducing, or eliminating tinnitus and/or the accompanying symptoms of tinnitus in a patient. Treating tinnitus refers to any indicia of success in prevention, reduction, elimination, or amelioration of tinnitus, including any objective or subjective parameter such as abatement, remission, diminishing of symptoms, prevention, or lessening of tinnitus symptoms or making the condition more tolerable to the patient, making the tinnitus less debilitating, or improving a patient's physical or mental well-being. The efficacy of an acamprosate prodrug of Formula (I), Formula (III), or Formula (IV) for treating tinnitus can be assessed using animal models of tinnitus and in clinical results. Methods of evaluating tinnitus in animals and humans are known. The ability of a compound of Formula (I), Formula (III), or Formula (IV) to treat tinnitus in human patients may be assessed using objective and subjective tests such as those described in Bauer and Brozoski, Laryngoscope 2006, 116(5), 675-681. An example of a test used in a clinical context to assess tinnitus treatment outcomes is the Tinnitus Handicap Inventory.

Neurodegenerative diseases are characterized by progressive dysfunction and neuronal death. Neurodegenerative diseases featuring cell death can be categorized as acute, i.e., stroke, traumatic brain injury, spinal cord injury, and chronic, i.e., amyotrophic lateral sclerosis, Huntington's disease, Parkinson's disease, and Alzheimer's disease. Although these diseases have different causes and affect different neuronal populations, they share similar impairment in intracellular energy metabolismNMDA receptor and non-NMDA receptor mediated excitotoxic injury results in neurodegeneration leading to necrotic or apoptotic cell death. Studies also suggest that mGluR5 receptor activity is involved in the etiology of neurodegenerative disorders and that mGluR5 modulators can be useful in treating movement and cognitive dysfunction associated with neurodegenerative disorders, as well as exhibit neuroprotective effects.

Parkinson's disease is a slowly progressive degenerative disorder of the nervous system characterized by tremor when muscles are at rest (resting tremor), slowness of voluntary movements, and increased muscle tone (rigidity). In Parkinson's disease, nerve cells in the basal ganglia, e.g., substantia nigra, degenerate, and thereby reduce the production of dopamine and the number of connections between nerve cells in the basal ganglia. As a result, the basal ganglia are unable to smooth muscle movements and coordinate changes in posture as normal, leading to tremor, incoordination, and slowed, reduced movement (bradykinesia).

Modulators of NMDA receptor activity have shown therapeutic potential in the management of Parkinson's disease, as well as have mGluR5 receptor antagonists. Accordingly, acamprosate may be useful in treating Parkinson's disease.

Studies suggest that agents that NMDA receptor antagonists or mGluR5 receptor antagonists are potentially useful for treating levodopa-induced dyskinesias such as levodopa-induced dyskinesias in Parkinson's disease Fabbrini et al., Movement Disorders 2007, 22(10), 1379-1389; and Mela et al., J Neurochemistry 2007, 101, 483-497). Accordingly, acamprosate prodrugs provided by the present disclosure may be useful in treating a movement disorder such as levodopa-induced dyskinesias in Parkinson's disease.

The efficacy of a compound of Formula (I), Formula (III), or Formula (IV) for treating Parkinson's disease may be assessed using animal models of Parkinson's disease and in clinical studies. Animal models of Parkinson's disease are known. The ability of acamprosate prodrugs to mitigate against L-dopa induced dyskinesias can be assessed using animal models and in clinical trials.

Alzheimer's disease is a progressive loss of mental function characterized by degeneration of brain tissue. In Alzheimer's disease, parts of the brain degenerate, destroying nerve cells and reducing the responsiveness of the maintaining neurons to neurotransmitters. Abnormalities in brain tissue consist of senile or neuritic plaques, e.g., clumps of dead nerve cells containing an abnormal, insoluble protein called amyloid, and neurofibrillary tangles, twisted strands of insoluble proteins in the nerve cell.

Excitotoxic cell death is thought to contribute to neuronal cell injury and death in Alzheimer's diseases and other neurodegenerative disorders. Excitotoxicity is due, at least in part, to excessive acylation of NMDA-type glutamate receptors and the concomitant excessive Ca2+ influx through the receptor's associated ion channel. NMDA receptor antagonists have shown neuroprotective effects in Alzheimer's disease (Lipton, NeuroRx 2004, 1(1), 101-110). As a modulator of the NMDA receptor, acamprosate may have similar effects.

The efficacy of administering a compound of Formula (I), Formula (III), or Formula (IV) for treating Alzheimer's disease may be assessed using animal models of Alzheimer's disease and in clinical studies. Useful animal models for assessing the efficacy of compounds for treating Alzheimer's disease are known.

Huntington's disease is an autosomal dominant neurodegenerative disorder in which specific cell death occurs in the neostriatum and cortex. Onset usually occurs during the fourth or fifth decade of life, with a mean survival at age of onset of 14 to 20 years. Huntington's disease is universally fatal, and there is no effective treatment. Symptoms include a characteristic movement disorder (Huntington's chorea), cognitive dysfunction, and psychiatric symptoms. The disease is caused by a mutation encoding an abnormal expansion of CAG-encoded polyglutamine repeats in the protein, huntingtin.

Neuroprotective effects of NMDA antagonists such as memantine and ketamine in Huntington's disease have been proposed (Murman et al., Neurology 1997, 49(1), 153-161; and Kozachuk, US 2004/0102525).

The efficacy of administering a compound of Formula (I), Formula (III), or Formula (IV) for treating Huntington's disease may be assessed using animal models of Huntington's disease and in clinical studies. Animal models of Huntington's disease are known.

Amyotrophic lateral sclerosis (ALS) is a progressive neurodegenerative disorder characterized by the progressive and specific loss of motor neurons in the brain, brain stem, and spinal cord. ALS begins with weakness, often in the hands and less frequently in the feet that generally progresses up an arm or leg. Over time, weakness increases and spasticity develops characterized by muscle twitching and tightening, followed by muscle spasms and possibly tremors.

A possible cause of ALS is constitutive opening of the calcium pore in glutamate responsive AMPA channels secondary to a failure of RNA editing. Recent work has shown that endogenous polyamines can block the vulnerability of motor neurons to cell death due to calcium influx through Ca²⁺-permeable AMP receptors. Acamprosate is believed to have an action at AMPA receptors similar to that of endogenous polyamines. Accordingly, it has been proposed that acamprosate may be useful in treating ALS (Kast and Altschuler, Med Hypotheses 2007, 69(4), 836-837).

The efficacy of a compound of Formula (I), Formula (III), or Formula (IV) for treating ALS may be assessed using animal models of ALS and in clinical studies. Natural disease models of ALS include mouse models (motor neuron degeneration, progressive motor neuropathy, and wobbler) and the hereditary canine spinal muscular atrophy canine model. Experimentally produced and genetically engineered animal models of ALS can also useful in assessing therapeutic efficacy. Specifically, the SOD1-G93A mouse model is a recognized model for ALS. Examples of clinical trial protocols useful in assessing treatment of ALS are known.

Multiple sclerosis (MS) is an inflammatory autoimmune disease of the central nervous system caused by an autoimmune attack against the isolating axonal myelin sheets of the central nervous system. Demyelination leads to the breakdown of conduction and to severe disease with destruction of local axons and irreversible neuronal cell death. The symptoms of MS are highly varied with each patient exhibiting a particular pattern of motor, sensory, and sensory disturbances. MS is typified pathologically by multiple inflammatory foci, plaques of demyelination, gliosis, and axonal pathology within the brain and spinal cord, all of which contribute to the clinical manifestations of neurological disability. Although the causal events that precipitate MS are not fully understood, evidence implicates an autoimmune etiology together with environmental factors and specific genetic predispositions. Functional impairment, disability, and handicap are expressed as paralysis, sensory and octintive disturbances, spasticity, tremor, lack of coordination, and visual impairment. These symptoms significantly impact the quality of life of the individual.

Involvement of ionotropic glutamate receptor function including the NMDA receptor, AMPA receptor, and kainite receptor are implicated in the pathology of MS). Compounds that modulate the NMDA and AMPA/kainite family of glutamate receptors have shown neuroprotective effects in multiple sclerosis (Killestein et al., J Neurol Sci 2005, 233, 113-115). As a mediator of ionotropic glutamate receptors, acamprosate is potentially useful in treating MS.

Assessment of MS treatment efficacy in clinical trials can be accomplished using tools such as the Expanded Disability Status Scale and the MS Functional Composite as well as magnetic resonance imaging lesion load, biomarkers, and self-reported quality of life). Animal models of MS shown to be useful to identify and validate potential MS therapeutics include experimental autoimmune/allergic encephalomyelitis (EAE) rodent models that simulate the clinical and pathological manifestations of MS.

In certain embodiments, compounds of Formula (I), Formula (III), and Formula (V) or pharmaceutical compositions thereof can be used to treat a psychotic disorder such as, for example, schizophrenia. Other psychotic disorders for which acamprosate prodrugs provided by the present disclosure may be useful include brief psychotic disorder, delusional disorder, schizoaffective disorder, and schizophreniform disorder.

Schizophrenia is a chronic, severe, and disabling brain disorder that affects about one percent of people worldwide, including 3.2 million Americans. Schizophrenia encompasses a group of psychotic disorders characterized by dysfunctions of the thinking process, such as delusions, hallucinations, and extensive withdrawal of the patient's interests form other people. Schizophrenia includes the subtypes of paranoid schizophrenia characterized by a preoccupation with delusions or auditory hallucinations, hebephrenic or disorganized schizophrenia characterized by disorganized speech, disorganized behavior, and flat or inappropriate emotions; catatonic schizophrenia dominated by physical symptoms such as immobility, excessive motor activity, or the assumption of bizarre postures; undifferentiated schizophrenia characterized by a combination of symptoms characteristic of the other subtypes; and residual schizophrenia in which a person is not currently suffering from positive symptoms but manifests negative and/or cognitive symptoms of schizophrenia (DSM-IV-TR classifications 295.30 (Paranoid Type), 295.10 (Disorganized Type), 295.20 (Catatonic Type), 295.90 (Undifferentiated Type), and 295.60 (Residual Type) (Diagnostic and Statistical Manual of Mental Disorders, Fourth Edition, Text Revision, American Psychiatric Association, 2000). Schizophrenia includes these and other closely associated psychotic disorders such as schizophreniform disorder, schizoaffective disorder, delusional disorder, brief psychotic disorder, shared psychotic disorder, psychotic disorder due to a general medical condition, substance-induced psychotic disorder, and unspecified psychotic disorders (DSM-IV-TR). Schizoaffective disorder is characterized by symptoms of schizophrenia as well as mood disorders such as major depression, bipolar mania, or mixed mania, is included as a subtype of schizophrenia.

Symptoms of schizophrenia can be classified as positive, negative, or cognitive. Positive symptoms of schizophrenia include delusion and hallucination, which can be measured using, for example, using the Positive and Negative Syndrome Scale (PANSS). Negative symptoms of schizophrenia include affect blunting, anergia, alogia and social withdrawal, can be measured for example, using the Scales for the Assessment of Negative Symptoms (SANS) (Andreasen, 1983, Scales for the Assessment of Negative Symptoms (SANS), Iowa City, Iowa). Cognitive symptoms of schizophrenia include impairment in obtaining, organizing, and using intellectual knowledge, which can be measured using the Positive and Negative Syndrome Scale-cognitive subscale (PANSS-cognitive subscale) or by assessing the ability to perform cognitive tasks such as, for example, using the Wisconsin Card Sorting Test.

The glutamatergic system has been implicated in the etiology and pathophysiology of schizophrenia and modulators of NMDA receptor activity and mGluR5 receptor activity such as acamprosate have been proposed as potential therapeutic agents for schizophrenia Paz et al., Eur Neuropsychopharmacology 2008, prepublication no. NEUPSY-10085, 14 pages). Accordingly, acamprosate and acamprosate prodrugs provided by the present disclosure may have efficacy in treating the positive, negative, and/or cognitive symptoms of schizophrenia (Kozachuk, US 2004/0102525; and Fogel, U.S. Pat. No. 6,689,816).

The efficacy of compounds of Formula (I), Formula (III), and Formula (IV) and pharmaceutical compositions of any of the foregoing for treating schizophrenia may be determined by methods known to those skilled in the art. For example, negative, positive, and/or cognitive symptom(s) of schizophrenia may be measured before, during, and/or after treating the patient. Reduction in such symptom(s) indicates that a patient's condition has improved. Improvement in the symptoms of schizophrenia may be assessed using, for example, the Scale for Assessment of Negative Symptoms (SANS), Positive and Negative Symptoms Scale (PANSS) and using Cognitive Deficits tests such as the Wisconsin Card Sorting Test (WCST).

The efficacy of Formula (I), Formula (III), and Formula (IV) and pharmaceutical compositions of any of the foregoing may be evaluated using animal models of schizophrenic disorders. For example, conditioned avoidance response behavior (CAR) and catalepsy tests in rats are shown to be useful in predicting antipsychotic activity and EPS effect liability.

In certain embodiments, compounds of Formula (I), Formula (III), and Formula (V) or pharmaceutical compositions thereof can be used to treat a mood disorder such as, for example, a bipolar disorder and a depressive disorder.

### Bipolar Disorder

Bipolar disorder is a psychiatric condition characterized by periods of extreme mood. The moods can occur on a spectrum ranging from depression (e.g., persistent feelings of sadness, anxiety, guilt, anger, isolation, and/or hopelessness, disturbances in sleep and appetite, fatigue and loss of interest in usually enjoyed activities, problems concentrating, loneliness, self-loathing, apathy or indifference, depersonalization, loss of interest in sexual activity, shyness or social anxiety, irritability, chronic pain, lack of motivation, and morbid/suicidal ideation) to mania (e.g., elation, euphoria, irritation, and/or suspicious). Bipolar disorder is defined and classified in DSM-IV-TR. Bipolar disorder includes bipolar I disorder, bipolar II disorder, cyclothymia, and bipolar disorder not otherwise specified. Patients afflicted with this disorder typically alternate between episodes of depression (depressed mood, hopelessness, anhedonia, varying sleep disturbances, difficulty in concentration, psychomotor retardation and often, suicidal ideation) and episodes of mania (grandiosity, euphoria, racing thoughts, decreased need for sleep, increased energy, risk taking behavior).

Inhibitors of glutamate release such as lamotrigine and riluzole, and NMDA antagonists such as memantine and ketamine are being investigated for treating bipolar disorder (Zarate et al., Am J Psychiatry 2004, 161, 171-174; Zarate et al., Biol Psychiatry 2005, 57, 430-432; and Teng and Demetrio, Rev Bras Psiquiatr 2006, 28(3), 251-6).

Treatment of bipolar disorder can be assessed in clinical trials using rating scales such as the Montgomery-Asberg Depression Rating Scale, the Hamilton Depression Scale, the Raskin Depression Scale, Feighner criteria, and/or Clinical Global Impression Scale Score).

Depressive disorders include major depressive disorder, dysthymic disorder, premenstrual dysphoric disorder, minor depressive disorder, recurrent brief depressive disorder, and postpsychotic depressive disorder of schizophrenia (DSM IV).

Studies support the involvement of the glutamatergic system in the pathophsyiology of depression. NMDA receptor antagonists have shown antidepressant effects in animal models and in clinical studies. Modulators of mGluR5 activity have also shown potential efficacy as antidepressants.

The efficacy of compounds provided by the present disclosure for treating depression can be evaluated in animal models of depression such as the forced swim test, the tail suspension test and others, and in clinical trials.

Anxiety is defined and classified in DSM-IV-TR. Anxiety disorders include panic attack, agoraphobia, panic disorder without agoraphobia, agoraphobia without history of panic disorder, specific phobia, social phobia, obsessive-compulsive disorder, posttraumatic stress disorder, acute stress disorder, generalized anxiety disorder, anxiety disorder due to a general medical condition, substance-induced anxiety disorder, and anxiety disorder not otherwise specified.

Neurochemical investigations have linked anxiety to dysfunction in central GABAergic, serotonergic, and noradrenergc systems. Modulators of mGluR5 receptors such as the selective antagonist 2-methyl-6-(phenylethynyl)-pyridine have been shown to be effective in treating anxiety disorders (Lea and Faden, CNS Drug Rev 2006, 12(2), 149-66; and Molina-Hernandez et al., Prog Neuro-Psychopharmacology Biolog Psychiatry 2006, 30, 1129-1135). In particular, acamprosate has been proposed for the treatment of anxiety disorders (Fogel, U.S. Pat. No. 6,689,816).

Useful animal models for assessing treatment of anxiety include fear-potentiated startle, elevated plus-maze, X-maze test of anxiety, and the rat social interaction test. Genetic animal models of anxiety are also known as are other animal models sensitive to anti-anxiety agents.

In clinical trials, efficacy can be evaluated using psychological procedures for inducing experimental anxiety applied to healthy volunteers and patients with anxiety disorders or by selecting patients based on the Structured Clinical interview for DSM-IV Axis I Disorders. One or more scales can be used to evaluate anxiety and the efficacy of treatment including, for example, the Penn State Worry Questionnaire, the Hamilton Anxiety and Depression Scales, the Spielberger State-Trait Anxiety Inventory, and the Liebowitz Social Anxiety Scale.

In certain embodiments, acamprosate prodrugs provided by the present disclosure may be useful in treating somatoform disorders such as somatization disorder, conversion disorder, hypochondriasis, and body dysmorphic disorder.

In certain embodiments, movement disorders include myoclonus, tremor, tics, tardive dyskinesia, movement disorders associated with Parkinson's disease and Huntignton's disease, progressive suprauclear palsy, Shy-Drager syndrome, tics, Tourette's syndrome, chorea and athetosis, spasmodic torticollis, ataxia, restless legs syndrome, and dystonias. Also included in movement disorders is spasticity.

Tardive dyskinesia is a neurological disorder caused by the long-term or high-dose use of dopamine antagonists such as antipsychotics. Tardive dyskinesia is characterized by repetitive, involuntary, purposeless movements such as grimacing, tongue protrusion, lip smacking, puckering and pursing of the lips, and rapid eye blinking, and can also involve rapid movements of the arms, legs, and trunk.

Studies suggest that NMDA receptors are involved in the dyskinesia observed in animal models of tardive dyskinesia and NMDA receptor modulators have to some extent been shown to reverse the effects of neuroleptic induced vacuous chewing movements, an animal model of tardive dyskinesia. Accordingly, acamprosate has been proposed for treating tardive dyskinesia and other movement disorders including tics, Tourette's syndrome, focal dystonias, blepharospasm, and Meige Syndrome (Fogel, U.S. Pat. No. 5,952,389, US 2002/0013366, and US 2006/1028802), and in studies on individual patients has been shown effective in treating tardive dyskinesia, dystonia, and tic at acamprosate doses from about 1,000 mg/day to about 2,000 mg/day.

Efficacy of tardive dyskinesia treatment can be assessed using animal models.

### Spasticity

Spasticity is an involuntary, velocity-dependent, increased resistance to stretch. Spasticity is characterized by muscle hypertonia and displays increased resistance to externally imposed movement with increasing speed of stretch. Spasticity can be caused by lack of oxygen to the brain before, during, or after birth (cerebral palsy); physical trauma (brain or spinal cord injury); blockage of or bleeding from a blood vessel in the brain (stroke); certain metabolic diseases; adrenolekodystrophy; phenylketonuria; neurodegenerative diseases such as Parkinson's disease and amyotrophic lateral sclerosis; and neurological disorders such as multiple sclerosis. Spasticity is associated with damage to the corticospinal tract and is a common complication of neurological disease. Diseases and conditions in which spasticity may be a prominent symptom include cerebral palsy, multiple sclerosis, stroke, head and spinal cord injuries, traumatic brain injury, anoxia, and neurodegenerative diseases. Patients with spasticity complain of stiffness, involuntary spasm, and pain. These painful spasms may be spontaneous or triggered by a minor sensory stimulus, such as touching the patient.

Symptoms of spasticity can include hypertonia (increased muscle tone), clonus (a series of rapid muscle contractions), exaggerated deep tendon reflexes, muscle spasms, scissoring (involuntary crossing of the legs), deformities with fixed joints, stiffness, and/or fatigue caused by trying to force the limbs to move normally. Other complications include urinary tract infections, chronic constipation, fever or other systemic illnesses, and/or pressure sores. The degree of spasticity varies from mild muscle stiffness to severe, painful, and uncontrollable muscle spasms. Spasticity may coexist with other conditions but is distinguished from rigidity (involuntary bidirectional non-velocity-dependent resistance to movement), clonus (self-sustaining oscillating movements secondary to hypertonicity), dystonia (involuntary sustained contractions resulting in twisting abnormal postures), athetoid movement (involuntary irregular confluent writhing movements), chorea (involuntary, abrupt, rapid, irregular, and unsustained movements), ballisms (involuntary flinging movements of the limbs or body), and tremor (involuntary rhythmic repetitive oscillations, not self-sustaining). Spasticity can lead to orthopedic deformity such as hip dislocation, contractures, or scoliosis; impairment of daily living activities such as dressing, bathing, and toileting; impairment of mobility such as inability to walk, roll, or sit; skin breakdown secondary to positioning difficulties and shearing pressure; pain or abnormal sensory feedback; poor weight gain secondary to high caloric expenditure; sleep disturbance; and/or depression secondary to lack of functional independence.

Treatment of spasticity includes physical and occupational therapy such as functional based therapies, rehabilitation, facilitation such as neurodevelopmental therapy, proprioceptive neuromuscular facilitation, and sensory integration; biofeedback: electrical stimulation; and orthoses. Oral medications useful in treating spasticity include baclofen, benzodiazepines such as diazepam, dantrolene sodium; imidazolines such as clonidine and tizanidine; and gabapentin. Intrathecal medications useful in treating spasticity include baclofen. Chemodenervation with local anesthetics such as lidocaine and xylocalne; type A botulinum toxin and type B botulinum toxin; phenol and alcohol injection can also be useful in treating spasticity. Surgical treatments useful in treating spasticity include neurosurgery such as selective dorsal rhizotomy; and orthopedic operations such as contracture release, tendon or muscle lengthening, tendon transfer, osteotomy, and arthrodesis.

Studies suggest that NMDA receptor may play a role in the activity of muscle relaxants and that NMDA receptor antagonists may have therapeutic potential in spasticity (Kornhuber and Quack, Neruosci Lett 1995, 195, 137-139).

The efficacy of a compound of Formula (I), Formula (III), and Formula (IV) for the treatment of spasticity can be assessed using animal models of spasticity and in clinically relevant studies of spasticity of different etiologies. The therapeutic activity may be determined without determining a specific mechanism of action. Animal models of spasticity are known. For example, animal models of spasticity include the mutant spastic mouse; the acute/chronic spinally transected rat and the acute decerebrate rat; primary observation Irwin Test in the rat; and Rotarod Test in the rat and mouse. Other animal models include spasticity induced in rats following transient spinal cord ischemia (; spasticity in mouse models of multiple sclerosis; and spasticity in rat models of cerebral palsy.

The efficacy of compounds of Formula (I), Formula (III), and Formula (IV) may also be assessed in humans using double blind placebo-controlled clinical trials. Clinical trial outcome measures for spasticity include the Ashworth Scale, the modified Ashworth Scale, muscle stretch reflexes, presence of clonus and reflex response to noxious stimuli. Spasticity can be assessed using methods and procedures known in the art such as a combination of clinical examination, rating scales such as the Ashworth Scale, the modified Ashworth scale the spasm frequency scale and the reflex score, biomechanical studies such as the pendulum test, electrophysiologic studies including electromyography, and functional measurements such as the Fugl-Meyer Assessment of Sensorimotor Impairment scale. Other measures can be used to assess spasticity associated with a specific disorder such as the Multiple Sclerosis Spasticity Scale.

Cortical spreading depression (CSD) is a phenomena believed to be involved in the pathogenesis of migraine. During the early phase of CSD, a slow-propagating wave of hyper- then hypo-activity spreads through the cortex, resulting in hyper- then hypo-vascularization. This is followed by a prolonged period of neuronal depression, which is associated with disturbances in nerve cell metabolism and regional reductions in blood flow. CSD may also activate trigeminal nerve axons, which then release neuropeptides, such as substance P, neurokinin A, and CGRP from axon terminals near the meningeal and other blood vessels that produce an inflammatory response in the area around the innervated blood vessels. CSD is also implicated in progressive neuronal injury following stroke and head trauma; and cerebrovascular disease. Glutamate release and subsequent NMDA receptor activation have been implicated in the spread of CSD. NMDA antagonists such as ifenprodil have been shown effective in preventing CSD in the mouse entorhinal cortex and the NMDA receptor antagonist MK-801 was effective in blocking CSD caused by traumatic injury in rat neocortical brain slices. Accordingly, NMDA receptor antagonists that inhibit the release of glutamate in the neuron can potentially prevent CSD and its consequences. For example, (7-chloro-4-hydroxy-3-(3-phenoxy)phenyl-2-(1H)-quinolone, a high affinity antagonist at the glycine site of the NMDA receptor inhibits the initiation and propagation of spreading depression. Other selective NMDA antagonists and an uncompetitive NMDA receptor blocker have shown potential for treating cortical spreading depression migraine (Menniti et al., Neuropharmacology 2000, 39, 1147-1155; and Peeters et al., J Pharmacology and Experimental Therapeutics 2007, 321(2), 564-572). Accordingly, acamprosate prodrugs may be useful in treating cortical spreading depression related disorders such as migraine, cerebral injury, epilepsy, and cardiovascular disease.

Efficacy of acamprosate prodrugs provided by the present disclosure for treating cortical spreading depression can be assessed using animal models of cortical spreading depression.

Migraine is a neurological disorder that is characterized by recurrent attacks of headache, with pain most often occurring on one side of the head, accompanied by various combinations of symptoms such as nausea, vomiting, and sensitivity to light, sound, and odors. The exact mechanism of migraine initiation and progress is not known. Migraine can occur at any time of day or night, but occurs most frequently on arising in the morning. Migraine can be triggered by various factors, such as hormonal changes, stress, foods, lack of sleep, excessive sleep, or visual, auditory, olfactory, or somatosensory stimulation. In general, there are four phases to a migraine: the prodrome, auras, the attack phase, and postdrome. The prodrome phase is a group of vague symptoms that may precede a migraine attack by several hours, or even a few days before a migraine episode. Prodrome symptoms can include sensitivity to light and sound, changes in appetite, fatigue and yawning, malaise, mood changes, and food cravings. Auras are sensory disturbances that occur before the migraine attack in one in five patients. Positive auras include bright or shimmering light or shapes at the edge of the field of vision. Other positive aura experiences are zigzag lines or stars. Negative auras are dark holes, blind spots, or tunnel vision. Patients may have mixed positive and negative auras. Other neurologic symptoms that may occur at the same time as the aura include speech disturbances, tingling, numbness, or weakness in an arm or leg, perceptual disturbances such as space or size distortions, and confusion. A migraine attack usually lasts from 4 to 72 hours and typically produces throbbing pain on one side of the head, pain worsened by physical activity, nausea, visual symptoms, facial tingling or numbness, extreme sensitivity to light and noise, looking pale and feeling cold, and less commonly tearing and redness in one eye, swelling of the eyelid, and nasal congestion. During the attack the pain may migrate from one part of the head to another, and may radiate down the neck into the shoulder. Scalp tenderness occurs in the majority of patients during or after an attack. After a migraine attack, there is usually a postdrome phase, in which patients may feel exhausted, irritable, and/or be unable to concentrate. Other types of migraine include menstrual migraines, opthalmologic migraine, retinal migraine, basilar migraine, familial hemiplegic migraine, and status migrainosus.

It is theorized that persons prone to migraine have a reduced threshold for neuronal excitability, possibly due to reduced activity of the inhibitory neurotransmitter γ-aminobutyric acid (GABA). GABA normally inhibits the activity of the neurotransmitters serotonin (5-HT) and glutamate, both of which appear to be involved in migraine attacks. The excitatory neurotransmitter glutamate is implicated in an electrical phenomenon called cortical spreading depression, which can initiate a migraine attack, while serotonin is implicated in vascular changes that occur as the migraine progresses.

Acamprosate prodrugs provided by the present disclosure or pharmaceutical composition thereof may be administered to a patient after initiation of the migraine. For example, a patient may be in the headache phase of the migraine or the postdrome phase before the prodrug or pharmaceutical composition is administered. Alternatively, acamprosate prodrugs provided by the present disclosure or pharmaceutical composition thereof may be administered to the patient before the migraine starts, such as once the patient senses that a migraine is developing or when the early symptoms of the migraine have begun. Acamprosate prodrugs provided by the present disclosure may also be administered to a patient on an ongoing or chronic basis to treat recurrent or frequent occurrences of migraine episodes.

Migraine may be diagnosed by determining whether some of a person's recurrent headaches meet migraine criteria as disclosed in, for example, see The International Classification of Headache Disorders, 2nd edition, Headache Classification Committee of the International Headache Society, Cephalalgia 2004, 24 (suppl 1), 8-160.

The efficacy of administering at least one compound of Formula (I), Formula (III), and Formula (IV) for treating migraine can be assessed using animal models of migraine and clinical studies. Animal models of migraine are known. For example, to delineate and assess the effectiveness of an acamprosate prodrug provided by the present disclosure, the frequency of migraine attacks, their severity and their accompanying symptoms may be recorded and measured at baseline, and at 3 months, and 6 months, etc., following initiation of treatment. Anti-migraine and cortical-spreading depression activity of compounds provided by the present disclosure may be determined using methods known in the art.

Therapeutic efficacy of a compound of Formula (I), Formula (III), or Formula (IV) or pharmaceutical composition of any of the foregoing for treating migraine may also be determined in various animal models of neuropathic pain or in clinically relevant studies of different types of neuropathic pain. The therapeutic activity may be determined without determining a specific mechanism of action. Animal models for neuropathic pain are known in the art and include, but are not limited to, animal models that determine analgesic activity or compounds that act on the CNS to reduce the phenomenon of central sensitization that results in pain from non-painful or non-noxious stimuli. Other animal models are known in the art, such as hot plate tests, model acute pain and are useful for determining analgesic properties of compounds that are effective when painful or noxious stimuli are present. The progression of migraine is believed to be similar to the progress of epilepsy because an episodic phenomenon underlies the initiation of the epileptic episode and, as such, it is believed that epilepsy animal models may be useful in determining a component of the therapeutic activity of the pharmaceutical composition.

Sleeping disorders include primary sleep disorders such as dysomnias characterized by abnormalities in the amount, quality, or timing of sleep and parasomnias characterized by abnormal behavioral or physiological events occurring in association with sleep, specific sleep stages, or sleep-wake transitions; sleep disorders related to another mental disorder, sleep disorders due to a general medical condition; and substance-induced sleep disorder (DSM-IV). Dysomnias include breathing-related sleep disorders such as obstructive sleep apnea syndrome characterized by repeated episodes of upper-airway obstruction during sleep; central sleep apnea syndrome characterized by episodic cessation of ventilation during sleep without airway obstruction; and central alveolar hypoventilation syndrome characterized by impairment in ventilatory control that results in abnormally low arterial oxygen levels further worsened by sleep.

Sleep apnea is a sleep disorder characterized by pauses in breathing during sleep. Clinically significant levels of sleep apnea are defined as five or more events of any type per hour of sleep time. Sleep apnea can be characterized as central, obstructive, and mixed. In central sleep apnea, breathing is interrupted by the lack of effort. In obstructive sleep apnea, a physical block to airflow despite effort results in interrupted breathing. In mixed sleep apnea, there is a transition from central to obstructive features during the events. Sleep apnea leads to interrupted, poor-quality sleep, nocturnal oxygen desaturation, and a reduction or absence of REM sleep. Sleep apnea may exacerbate or contribute to cardiovascular disease including coronary heart disease, hypertension, ventricular hypertrophy and dysfunction, cardiac arrhythmias, and stroke, by mechanisms such as endothelial damage and dysfunction, increases in inflammatory mediators, increases in prothromobitic factors, increased sympathetic activity, hypoxemia, impaired vagal activity and insulin resistance. Sleep apnea may also contribute to cognitive impairment.

Acamprosate has been shown to improve sleep in patients being treated for alcohol withdrawal (Staner et al., Alcohol Clin Exp Res 2006, 30(9), 1492-9) and preliminary studies suggest that acamprosate at doses of about 1,000 mg/day (333 mg three times per day) may be effective in treating central and obstructive sleep apnea (Hedner et al., WO 2007/032720).

Sleep apnea can be clinically evaluated using polysomnography or oximetry, and/or using tools such as the Epworth Sleepiness Scale and the Sleep Apnea Clinical Score and/or using polysomnographic recording. Animal models of sleep apnea are known and can be useful in assessing the efficacy of acamprosate prodrugs for treating sleep apnea.

Pain includes nociceptive pain caused by injury to bodily tissues and neuropathic pain caused by abnormalities in nerves, spinal cord, and/or brain. Pain includes mechanical allodynia, thermal allodnia, hyperplasia, central pain, peripheral neuropathic pain, diabetic neuropathy, breakthrough pain, cancer pain, deafferentation pain, dysesthesia, fibromyalgia syndrome, hyperpathia, incident pain, movement-related pain, myofacial pain, and paresthesia. Pain can be acute or chronic.

Studies demonstrate the involvement of mGluR5 receptors in nociceptive processes and that modulation of mGluR5 receptor activity can be useful in treating various pain states such as acute pain, persistent and chronic pain inflammatory pain, visceral pain, neuropathic pain, nonioceptive pain, and post-operative pain. NMDA receptor antagonists have also been shown to attenuate central sensitization and hyperplasia in animals and humans.

Neuropathic pain involves an abnormal processing of sensory input usually occurring after direct injury or damage to nerve tissue. Neuropathic pain is a collection of disorders characterized by different etiologies including infection, inflammation, disease such as diabetes and multiple sclerosis, trauma or compression to major peripheral nerves, and chemical or irradiation-induced nerve damage. Neuropathic pain typically persists long after tissue injury has resolved.

An essential part of neuropathic pain is a loss (partial or complete) of afferent sensory function and the paradoxical presence of certain hyperphenomena in the painful area. The nerve tissue lesion may be found in the brain, spinal chord, or the peripheral nervous system. Symptoms vary depending on the condition but are usually the manifestations hyperalgesia (the lowering of pain threshold and an increased response to noxious stimuli), allodynia (the evocation of pain by non-noxious stimuli such as cold, warmth, or touch), hyperpathia (an explosive pain response that is suddenly evoked from cutaneous areas with increased sensory detection threshold when the stimulus intensity exceeds sensory threshold), paroxysms (a type of evoked pain characterized by shooting, electric, shock like or stabbing pain that occurs spontaneously, or following stimulation by an innocuous tactile stimulus or by a blunt pressure), paraesthesia (abnormal but non-painful sensations, which can be spontaneous or evoked, often described as pins and needles), dysesthesia (abnormal unpleasant but not necessarily painful sensation, which can be spontaneous or provoked by external stimuli), referred pain and abnormal pain radiation (abnormal spread of pain), and wind-up like pain and aftersensations (the persistence of pain long after termination of a painful stimulus). Patients with neuropathic pain typically describe burning, lancinating, stabbing, cramping, aching and sometimes vice-like pain. The pain can be paroxysmal or constant. Pathological changes to the peripheral nerve(s), spinal cord, and brain have been implicated in the induction and maintenance of chronic pain. Patients suffering from neuropathic pain typically endure chronic, debilitating episodes that are refractory to current pharmacotherapies and profoundly affect their quality of life. Currently available treatments for neuropathic pain, including tricyclic antidepressants and gabapentin, typically show limited efficacy in the majority of patients (Sindrup and Jensen, Pain 1999, 83, 389-400).

There are several types of neuropathic pain. A classification that relates to the type of damage or related pathophysiology causing a painful neuropathy includes neuropathies associated with mechanical nerve injury such as carpal tunnel syndrome, vertebral disk herniation, entrapment neuropathies, ulnar neuropathy, and neurogentic thoracic outlet syndrome; metabolic disease associated neuropathies such as diabetic polyneuropathy; neuropathies associated with neurotropic viral disease such as herpes zoster and human immunodeficiency virus (HIV) disease; neuropathies associated with neruotoxicity such as chemotherapy of cancer or tuberculosis, radiation therapy, drug-induced neuropathy, and alcoholic neuropathy; neuropathies associated with inflammatory and/or immunolgic mechanisms such as multiple sclerosis, anti-sulfatide antibody neuropathies, neuropathy associated with monoclonal gammopathy, Sjogren's disease, lupus, vasculitic neuropathy, polyclonal inflammatory neuropathies, Guillain-Barre syndrome, chronic inflammatory demyelinating neuropathy, multifocal motor neuropathy, paraneoplastic autonomic neuropathy, ganlinoic acetylcholine receptor antibody autonomic neuropathy, Lambert-Eaton myasthenic syndrome and myasthenia gravis; neuropathies associated with nervous system focal ischemia such as thalamic syndrome (anesthesia dolorosa); neuropathies associated with multiple neurotransmitter system dysfunction such as complex regional pain syndrome (CRPS); neuropathies associated with chronic/neuropathic pain such as osteoarthritis, lower back pain, fibromyalgia, cancer bone pain, chronic stump pain, phantom limb pain, and paraneoplastic neuropathies; neuropathies associated with neuropathic pain including peripheral neuropathies such as postherpetic neuralgia, toxic neuropathies (e.g., exposure to chemicals such as exposure to acrylamide, 3-chlorophene, carbamates, carbon disulfide, ethylene oxide, n-hexane, methyl n-butylketone, methyl bromide, organophosphates, polychlorinated biphenyls, pyriminil, trichlorethylene, or dichloroacetylene), focal traumatic neuropathies, phantom and stump pain, monoradiculopathy, and trigeminal neuralgia; and central neuropathies including ischemic cerebrovascular injury (stroke), multiple sclerosis, spinal cord injury, Parkinson's disease, amyotrophic lateral sclerosis, syringomyelia, neoplasms, arachnoiditis, and post-operative pain; mixed neuropathies such as diabetic neuropathies (including symmetric polyneuropathies such as sensory or sensorimotor polyneuropathy, selective small-fiber polyneuropathy, and autonomic neuropathy; focal and multifocal neuropathies such as cranial neuropathy, limb mononeuropathy, trunk mononeuro-pathy, mononeuropathy multiplex, and asymmetric lower limb motor neuropathy) and sympathetically maintained pain. Other neuropathies include focal neuropathy, glosopharyngeal neuralgia, ischemic pain, trigeminal neuralgia, atypical facial pain associated with Fabry's disease, Celiac disease, hereditary sensory neuropathy, or B₁₂-deficiency; mono-neuropathies, polyneuropathis, hereditary peripheral neuropathies such as Carcot-Marie-Tooth disease, Refsum's disease, Strumpell-Lorrain disease, and retinitis pigmentosa; acute polyradiculoneuropathy; and chronic polyradiculoneuropathy. Paraneoplastic neuropathies include paraneoplastic subacute sensory neuronopathy, paraneoplastic motor neuron disease, paraneoplastic neuromyotonia, paraneoplastic demyelinating neuropathies, paraneoplastic vasculitic neuropathy, and paraneoplastic autonomic insufficiency.

The important role of N-methyl-D-aspartate (NMDA) receptors in the development and maintenance of chronic pain associated with central and peripheral nerve injury is well documented. Consequently, NMDA antagonists have been proposed as potential therapeutics for neuropathic pain. NMDA antagonists of different classes have shown efficacy in preclinical models as well as in patients with chronic pain, including neuropathic pain. Several clinical studies have observed a long-lasting relief in some neuropathic pain patients treated with NMDA antagonists (Pud et al., Pain 1998, 75(2-3), 349-54; Eisenberg et al., J Pain 2007, 8(3), 223-9; Rabben et al., J Pharmacol Exp Ther 1999, 289(2), 1060-1066; Correll et al., Pain Med 2004, 5(3), 263-75; and Harbut et al., US 2005/0148673).

Other diseases or disorders for which NMDA antagonists and mGluR5 antagonists such as acamprosate may be therapeutically useful include neuroprotection in epilepsy (Chapman et al., Neuropharmacol 2000, 39, 1567-1574), cognitive dysfunction (Riedel et al., Neuropharmacol 2000, 39, 1943-1951), Down's syndrome, normal cognitive senescence, meningitis, sepsis and septic encephalophathy, CNS vasculities, leudodystrophies and X-ADL, childbirth and surgical anesthesia, spinal cord injury, hypoglycemia, encephalopathy, tumors and malignancies, cerebellar degenerations, ataxias, bowel syndromes, metabolic bone disease and osteoporosis, obesity, diabetes and pre-diabetic syndromes (Storto et al., Molecular Pharmacology 2006, 69(4), 1234-1241), and gastroesophageal reflux disease (Jensen et al., Eur J Pharmacology 2005, 519, 154-157).

### Administration

Prodrugs of Formula (I), Formula (III), or (IV), pharmaceutically acceptable salts of any of the foregoing, and/or pharmaceutical compositions thereof may be administered orally. Prodrugs of Formula (I), Formula (III), or Formula (IV) and/or pharmaceutical compositions thereof may also be administered by any other convenient route, for example, by infusion or bolus injection, by absorption through epithelial or mucocutaneous linings (e.g., oral mucosa, rectal, and intestinal mucosa, etc.). Administration may be systemic or local. Various delivery systems are known, (e.g., encapsulation in liposomes, microparticles, microcapsules, capsules, etc.) that may be used to administer a compound and/or pharmaceutical composition. Compounds of Formula (I), Formula (III), or Formula (IV) a pharmaceutically acceptable salt of any of the foregoing, or a pharmaceutical composition thereof may be administered by any appropriate route. Examples of suitable routes of administration include, but are not limited to, intradermal, intramuscular, intraperitoneal, intravenous, subcutaneous, intranasal, epidural, oral, sublingual, intracerebral, intravaginal, transdermal, rectal, inhalation, or topical.

In certain embodiments, it may be desirable to introduce prodrugs of Formula (I), Formula (III), or Formula (IV) and/or pharmaceutical compositions thereof into the central nervous system, which may be by any suitable route, including intraventricular, intrathecal, and epidural injection. Intraventricular injection may be facilitated using an intraventricular catheter attached to a reservoir such as an Ommaya reservoir.

The amount of a prodrug of Formula (I), Formula (III), or Formula (IV) that will be effective in the treatment of a disease in a patient will depend, in part, on the nature of the condition and can be determined by standard clinical techniques known in the art. In addition, in vitro or in vivo assays may be employed to help identify optimal dosage ranges. A therapeutically effective amount of prodrug of Formula (I), Formula (III), or Formula (IV) to be administered may also depend on, among other factors, the subject being treated, the weight of the subject, the severity of the disease, the manner of administration, and the judgment of the prescribing physician.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

A dose may be administered in a single dosage form or in multiple dosage forms. When multiple dosage forms are used the amount of compound contained within each dosage form may be the same or different. The amount of a compound of Formula (I), Formula (III), or Formula (IV) contained in a dose may depend on the route of administration and whether the disease in a patient is effectively treated by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, an acamprosate prodrug may exhibit a high therapeutic index. The data obtained from these cell culture assays and animal studies may be used in formulating a dosage range that is not toxic for use in humans. A dose of an acamprosate prodrug provided by the present disclosure may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

### Combination Therapy

In certain embodiments, prodrugs of Formula (I), Formula (III), or Formula (IV) or pharmaceutically acceptable salts of any of the foregoing can be used in combination therapy with at least one other therapeutic agent. Prodrugs of Formula (I), Formula (III), or Formula (IV) and the at least one other therapeutic agent(s) may act additively or, in certain embodiments, synergistically. In certain embodiments, prodrugs of Formula (I), Formula (III), or Formula (IV) may be administered concurrently with the administration of another therapeutic agent. In certain embodiments, prodrugs of Formula (I), Formula (III), or Formula (IV) or pharmaceutically acceptable salts of any of the foregoing may be administered prior or subsequent to administration of another therapeutic agent. The at least one other therapeutic agent may be effective for treating the same or different disease or disorder.

When used to treat a disease or disorder a therapeutically effective amount of one or more compounds of Formula (I), Formula (III) or Formula (IV) may be administered singly, or in combination with other agents including pharmaceutically acceptable vehicles and/or pharmaceutically active agents for treating a disease or disorder, which may be the same or different disease or disorder as the disease or disorder being treated by the one or more compounds of Formula (I), Formula (III), or Formula (IV). A therapeutically effective amount of one or more compounds of Formula (I), Formula (III), or Formula (IV) may be delivered together with a compound disclosed herein or combination with another pharmaceutically active agent.

Methods of the present disclosure include administration of one or more compounds of Formula (I), Formula (III), Formula (IV), or pharmaceutical compositions thereof and another therapeutic agent provided the other therapeutic agent does not inhibit the therapeutic efficacy of the one or more compounds of Formula (I), Formula (III), or Formula (IV) and/or does not produce adverse combination effects.

In certain embodiments, compositions provided by the present disclosure may be administered concurrently with the administration of another therapeutic agent, which can be part of the same pharmaceutical composition as, or in a different composition than that containing the compound provided by the present disclosure. In certain embodiments, a compound of Formula (I), Formula (III), or Formula (IV) may be administered prior or subsequent to administration of another therapeutic agent. In certain embodiments of combination therapy, the combination therapy comprises alternating between administering a composition of Formula (I), Formula (III), or Formula (IV) and a composition comprising another therapeutic agent, e.g., to minimize adverse side effects associated with a particular drug. When a compound of Formula (I), Formula (III), or Formula (IV) is administered concurrently with another therapeutic agent that may produce adverse side effects including, but not limited to, toxicity, the other therapeutic agent may be administered at a dose that falls below the threshold at which the adverse side effect is elicited.

In certain embodiments, a pharmaceutical composition may further comprise substances to enhance, modulate and/or control release, bioavailability, therapeutic efficacy, therapeutic potency, stability, and the like. For example, to enhance therapeutic efficacy a compound of Formula (I), Formula (III), or Formula (IV) may be co-administered with one or more active agents to increase the absorption or diffusion of the compound from the gastrointestinal tract or to inhibit degradation of the drug in the systemic circulation. In certain embodiments, a compound of Formula (I), Formula (III), or Formula (IV) may be co-administered with active agents having a pharmacological effect that enhance the therapeutic efficacy of the drug.

In certain embodiments, compounds of Formula (I), Formula (III), or Formula (IV) or pharmaceutical compositions thereof include, or may be administered to a patient together with, another compound for treating a neurodegenerative disorder, a psychotic disorder, a mood disorder, an anxiety disorder, a somatoform disorder, movement disorder, a substance abuse disorder, binge eating disorder, a cortical spreading depression related disorder, tinnitus, a sleeping disorder, multiple sclerosis, or pain.

In certain embodiments, acamprosate prodrugs provided by the present disclosure and pharmaceutical compositions thereof may be administered to a patient for treating a neurodegenerative disorder in combination with a therapy or another therapeutic agent known or believed to be effective in treating a neurodegenerative disorder. In certain embodiments, a neurodegenerative disorder is chosen from Alzheimer's disease, Parkinson's disease, Huntington's disease, and amyotrophic lateral sclerosis.

Therapeutic agents useful for treating Parkinson's disease include dopamine precursors such levodopa, dopamine agonists such as bromocriptine, pergolide, pramipexole, and ropinirole, MAO-B inhibitors such as selegiline, anticholinergic drugs such as benztropine, trihexyphenidyl, tricyclic antidepressants such as amitriptyline, amoxapine, clomipramine, desipramine, doxepin, imipramine, maprotiline, nortriptyline, protriptyline, amantadine, and trimipramine, some antihistamines such as diphenhydramine; antiviral drugs such as amantadine; and β-blockers such as propranolol.

Useful drugs for treating Alzheimer's disease include rosiglitazone, roloxifene, vitamin E, donepezil, tacrine, rivastigmine, galantamine, and memantine.

Useful drugs for treating symptoms of Huntington's disease include antipsychotics such as haloperidol, chlorpromazine and olanzapine to control hallucinations, delusions and violent outbursts; antidepressants such as fluoxetine, sertraline, and nortryiptyline to control depression and obsessive-compulsive behavior; tranquilizers such as benzodiazepines, paroxetine, venflaxin and beta-blockers to control anxiety and chorea; mood stabilizers such as liethium, valproate, and carbamzepine to control mania and bipolar disorder; and botulinum toxin to control dystonia and jaw clenching. Useful drugs for treating symptoms of Huntington's disease further include selective serotonin reuptake inhibitors (SSRI) such as fluoxetine, paroxetine, sertraline, escitalopram, citalopram, fluvosamine; norepinephrine and serotonin reuptake inhibitors (NSRI) such as venlafaxine and duloxetine, benzodiazepines such as clonazepam, alprazolam, diazepam, and lorazepam, tricyclic antidepressants such as amitriptyline, nortriptyline, and imipramine; and atypical antidepressants such as busipirone, bupriopion, and mirtazepine for treating the symptoms of anxiety and depression; atomoxetine, dextroamphetamine, and modafinil for treating apathy symptoms; amantadine, memantine, and tetrabenazine for treating chorea symptoms; citalopram, atomoxetine, memantine, rivastigmine, and donepezil for treating cognitive symptoms; lorazepam and trazedone for treating insomnia; valproate, carbamazepine and lamotrigine for treating symptoms of irritability; SSRI antidepressants such as fluoxetine, paroxetine, sertaline, and fluvoxamine, NSRI antidepressants such as venlafaxine, and others such as mirtazepine, clomipramine, lomotrigine, gabapentin, valproate, carbamazepine, olanzapine, rispiridone, and quetiapine for treating symptoms of obsessive-compulsive disorder; haloperidol, quetiapine, clozapine, risperidone, olanzapine, ziprasidone, and aripiprazole for treating psychosis; and pramipexole, levodopa and amantadine for treating rigidity.

Useful drugs for treating ALS include riluzole. Other drugs of potential use in treating ALS include memantine, tamoxifen, thalidomide, ceftriaxone, sodium phenyl butyrate, celecoxib, glatiramer acetate, busipirone, creatine, minocycline, coenzyme Q10, oxandrolone, IGF-1, topiramate, xaliproden, and indinavir. Drugs such as baclofen and diazepam can be useful in treating spasticity associated with ALS.

In certain embodiments, acamprosate prodrugs provided by the present disclosure and pharmaceutical compositions thereof may be administered to a patient for treating a psychotic disorder in combination with a therapy or another therapeutic agent known or believed to be effective in treating a psychotic disorder. In certain embodiments a psychotic disorder is schizophrenia.

Examples of antipsychotic agents useful in treating positive symptoms of schizophrenia include, but are not limited to, acetophenazine, alseroxylon, amitriptyline, aripiprazole, astemizole, benzquinamide, carphenazine, chlormezanone, chlorpromazine, chlorprothixene, clozapine, desipramine, droperidol, aloperidol, fluphenazine, flupenthixol, glycine, oxapine, mesoridazine, molindone, olanzapine, ondansetron, perphenazine, pimozide, prochlorperazine, procyclidine, promazine, propiomazine, quetiapine, remoxipride, reserpine, risperidone, sertindole, sulpiride, terfenadine, thiethylperzaine, thioridazine, thiothixene, trifluoperazine, triflupromazine, trimeprazine, and ziprasidone. Examples of typical antipsychotic agents useful for treating positive symptoms of schizophrenia include acetophenazine, chlorpromazine, chlorprothixene, droperidol, fluphenazine, haloperidol, loxapine, mesoridazine, methotrimeprazine, molindone, perphenazine, pimozide, raclopride, remoxipride, thioridazine, thiothixene, and trifluoperazine. Examples of atypical antipsychotic agents useful for treating positive symptoms of schizophrenia include aripiprazole, clozapine, olanzapine, quetiapine, risperidone, sertindole, and ziprasidone.

Other antipsychotic agents useful for treating positive symptoms of schizophrenia include amisulpride, balaperidone, blonanserin, butaperazine, carphenazine, eplavanserin, iloperidone, lamictal, onsanetant, paliperidone, perospirone, piperacetazine, raclopride, remoxipride, sarizotan, sonepiprazole, sulpiride, ziprasidone, and zotepine; serotonin and dopamine (5HT/D2) agonists such as asenapine and bifeprunox; neurokinin 3 antagonists such as talnetant and osanetant; AMPAkines such as CX-516, galantamine, memantine, modafinil, ocaperidone, and tolcapone; and α-amino acids such as D-serine, D-alanine, D-cycloserine, and N-methylglycine. Thus, antipsychotic agents include typical antipsychotic agents, atypical antipsychotic agents, and other compounds useful for treating schizophrenia in a patient, and particularly useful for treating the positive symptoms of schizophrenia.

Examples of agents useful for treating cognitive and/or negative symptoms of schizophrenia include aripiprazole, clozapine, olanzapine, quetiapine, risperidone, sertindole, ziprasidone, asenapine, bifeprunox, iloperidone, lamictal, galantamine, memantine, modafininil, acaperidone, NK3 antagonists such as talnetant and osanetant, AMPAkines, tolcapone, amisulpride, mirtazapine, lamotrigine, idazoxan, neboglamine, sabcomeline, ispronicline, sarcosine, preclamol, L-carnosine, nicotine, raloxifene, pramipexol, escitalopram, estradiol, riluzole, creatine, entacapone, L-threonine, atomoxetine, divalproex sodium, pimozide, provastatin, duloxetine; and NMDA receptor modulators such as glycine, D-serine, and D-cycloserine.

In certain embodiments, pharmaceutical compositions provided by the present disclosure may be co-administered with another drug useful for treating a symptom of schizophrenia or a disease, disorder, or condition associated with schizophrenia and that is not an antipsychotic agent. For example, acamprosate prodrugs may be co-administered with an antidepressant, such as, but not limited to alprazolam, amitriptyline, amoxapine, bupropion, citalopram, clomipramine, desipramine, eoxepin, escitapopram, fluoxetine, fluvoxamine, imipramine, maprotiline, methylphenidate, mirtazapine, nefazodone, nortriptyline, paroxetine, phenelzine, protriptyline, sertraline, tranylcypromine, trazodone, trimipramine, venlafaxine, and combinations of any of the foregoing.

For example, in certain embodiments, an acamprosate prodrug provided by the present disclosure, or pharmaceutical compositions thereof may be administered to a patient for the treatment of schizophrenia in conjunction with a social therapy for treating schizophrenia such as rehabilitation, community support activities, cognitive behavioral therapy, training in illness management skills, participation in self-help groups, and/or psychotherapy. Examples of psychotherapies useful for treating schizophrenia include Alderian therapy, behavior therapy, existential therapy, Gestalt therapy, person-centered therapy, psychoanalytic therapy, rational-emotive and cognitive-behavioral therapy, reality therapy, and transactional analysis.

Other examples of drugs useful for treating psychotic disorders include aripiprazole, loxapine, mesoridazine, quetiapine, reserpine, thioridazine, trifluoperazine, and ziprasidone, chlorpromazine, clozapine, fluphenazine, haloperidol, olanzapine, perphenazine, prochlorperazine, risperidone, and thiothixene.

In certain embodiments, acamprosate prodrugs provided by the present disclosure and pharmaceutical compositions thereof may be administered to a patient for treating a mood disorder in combination with a therapy or another therapeutic agent known or believed to be effective in treating a mood disorder. In certain embodiments, a mood disorder is chosen from a bipolar disorder and a depressive disorder.

Examples of drugs useful for treating bipolar disorder include aripirprazole, verapamil, carbamazepine, clonidine, clonazepam, lamotrigine, olanzapine, quetiapine, fluoxetine, and ziprasidone.

In certain embodiments, acamprosate prodrugs provided by the present disclosure and pharmaceutical compositions thereof may be administered to a patient for treating depression in combination with a therapy or another therapeutic agent known or believed to be effective in treating depression.

Examples of drugs useful for treating depression include tricyclics such as amitriptyline, amoxapine, clomipramine, desipramine, doxepin, imipramine, maprotiline, nortryptyline, protryptyline, and trimipramine; tetracyclics such as maprotiline and mirtazapine; selective serotonin reuptake inhibitors (SSRI) such as citalopram, escitalopram, fluoxetine, fluvoxamine, paroxetine, and sertraline; serotonin and norepinephrine reuptake inhibitors (SNRI) such as venlafaxine and duloxetine; monoamine oxidase inhibitors such as isocarboxazid, phenelzine, selegiline, and tranylcypromine; psychostimulants such as dextroamphetamine and methylphenidate; and other drugs such as bupropion, mirtazapine, nefazodone, trazodone, lithium, and venlafaxine.

In certain embodiments, acamprosate prodrugs provided by the present disclosure and pharmaceutical compositions thereof may be administered to a patient for treating an anxiety disorder in combination with a therapy or another therapeutic agent known or believed to be effective in treating an anxiety disorder.

Examples of drugs for useful treating anxiety disorders include alprazolam, atenolol, busipirone, chlordiazepoxide, clonidine, clorazepate, diazepam, doxepin, escitalopram, halazepam, hydroxyzine, lorazepam, nadolol, oxazepam, paroxetine, prochlorperazine, trifluoperazine, venlafaxine, amitriptyline, sertraline, citalopram, clomipramine, fluoxetine, fluvoxamine, and paroxetine.

In certain embodiments, acamprosate prodrugs provided by the present disclosure and pharmaceutical compositions thereof may be administered to a patient for treating a somatoform disorder in combination with a therapy or another therapeutic agent known or believed to be effective in treating a somatoform disorder.

Examples of drugs useful for treating somatoform disorders include tricyclic antidepressants such as amitriptyline, and serotonin reuptake inhibitors.

In certain embodiments, acamprosate prodrugs provided by the present disclosure and pharmaceutical compositions thereof may be administered to a patient for treating a movement disorder in combination with a therapy or another therapeutic agent known or believed to be effective in treating a movement disorder. In certain embodiments, a movement disorder is selected from tardive dyskinesia and spasticity.

Examples of drugs useful for treating movement disorders include levodopa, mild sedatives such as benzodiazepines including alprazolam, chlordiazepoxide, clonazepam, clorazepate, diazepam, lorazepam, and oxazepam; muscle relaxants such as baclofen, anticholinergic drugs such as trihexyphenidyl and diphenhydramine; antipsychotics such as chlorpromazine, fluphenazine, haloperidol, loxapine, mesoridazine, molindone, perphenazine, pimozide, thioridazine, thiothixene, trifluoperazine, aripiprazole, clozapine, olanzapine, quetiapine, risperidone, and ziprasidone; and antidepressants such as amitriptyline.

Examples of drugs useful for treating tardive dyskinesia include vitamin E, dizocilpine, memantine, clzapine, lorazepam, diazepam, clonazepam, glycine, D-cycloserine valproic acid, amantadine, ifenprodil, and tetrabenazine.

Examples of drugs useful for treating spasticity include baclofen, R-baclofen, diazepam, tizanidine, clonidine, dantrolene, 4-aminopyridine, cyclobenzaprine, ketazolam, tiagabine, and botulinum A toxin. Compounds having activity as α2δ subunit calcium channel modulators such as gabapentin and pregabalin are believed to be useful as antispasticity agents.

In certain embodiments, acamprosate prodrugs provided by the present disclosure and pharmaceutical compositions thereof may be administered to a patient for treating a substance abuse disorder in combination with a therapy or another therapeutic agent known or believed to be effective in treating a substance abuse disorder. In certain embodiments, a substance abuse disorder is chosen from an alcohol abuse disorder, a narcotic abuse disorder, and a nicotine abuse disorder.

Examples of drugs useful for treating alcohol dependency or alcohol abuse disorders include disulfiram, naltrexone, acamprosate, ondansetron, atenolol, chlordiazepoxide, clonidine, clorazepate, diazepam, oxazepam, methadone, topiramate, 1-alpha-acetylmethadol, buprenorphine, bupropion, and baclofen.

Examples of drugs useful for treating opioid abuse disorders include buprenorphine, naloxone, tramadol, methadone, and naltrexone.

Examples of drugs useful for treating cocaine abuse disorders include disulfiram, modafinil, propranolol, baclofen, vigabatrin, and topiramate.

Examples of drugs useful for treating nicotine abuse disorders include bupropion, clonidine, rimonabant, verenicline, and nicotine.

In certain embodiments, acamprosate prodrugs provided by the present disclosure and pharmaceutical compositions thereof may be administered to a patient for treating a cortical spreading depression related disorder in combination with a therapy or another therapeutic agent known or believed to be effective in treating a cortical spreading depression related disorder. In certain embodiments, a cortical spreading depression related disorder is selected from migraine, cerebral injury, epilepsy, and cardiovascular disease.

Drugs useful for treating migraine can prevent a migraine from occurring, abort a migraine that is beginning, or relieve pain during the migraine episode.

Prophylactic migraine treatments reduce the frequency of migraines and include non-steroidal anti-inflammatory agents (NSAIDs), adrenergic beta-blockers, calcium channel blockers, tricyclic antidepressants, selective serotonin reuptake inhibitors, anticonvulsants, NMDA receptor antagonists, angiotensin converting enzyme (ACE) inhibitors, angiotensin-receptor blockers (ARBs), leukotriene-antagonists, dopamine agonists, selective 5HT-ID agonists, selective 5HT-1 F agonists, AMPA/KA antagonists, CGRP (calcitonin gene related peptide) antagonists, NOS (nitric oxide synthase) inhibitors, blockers of spreading cortical depression, and other therapy. Examples of NSAIDs useful for preventing migraine include aspirin, ibuprofen, fenoprofen, flurbiprofen, ketoprofen, mefenamic acid, and naproxen. Examples of adrenergic beta-blockers useful for preventing migraine include acebutolol, atenolol, imilol, metoprolol, nadolol, pindolol, propranolol, and timolol. Examples of calcium channel blockers useful for preventing migraine include amlodipine, diltiazem, dotarizine, felodipine, flunarizine, nicardipine, nifedipine, nimodipine, nisoldipine, and verapamil. Examples of tricyclic antidepressants useful for preventing migraine include amitriptyline, desipramine, doxepin, imipramine, nortriptyline, and protriptyline. Examples of selective serotonin reuptake inhibitors (SSRIs) useful for preventing migraine include fluoxetine, methysergide, nefazodone, paroxetine, sertraline, and venlafaxine. Examples of other antidepressants useful for preventing migraine include bupropion, nefazodone, norepinephrine, and trazodone.

Examples of anticonvulsants (antiepileptics) useful for preventing migraine include divalproex sodium, felbamate, gabapentin, lamotrigine, levetiracetam, oxcarbazepine, tiagabine, topiramate, valproate, and zonisamide. Examples of NMDA receptor antagonists useful for preventing migraine include dextromethorphan, magnesium, and ketamine. Examples of angiotensin converting enzyme (ACE) inhibitors useful for preventing migraine include lisinopril. Examples of angiotensin-receptor blockers (ARBs) useful for preventing migraine include candesartan. Examples of leukotriene-antagonists useful for preventing migraine include zileuton, zafirlukast, montelukast, and pranlukast. Examples of dopamine agonists useful for preventing migraine include α-dihydroergocryptine. Examples of other therapy useful for preventing migraine include botulinum toxin, magnesium, hormone therapies, riboflavin, methylergonovine, cyproheptadine, and phenelzine, and complementary therapies such as counseling/psychotherapy, relaxation training, progressive muscle relaxation, guided imagery, diaphragmatic breathing, biofeedback, acupuncture, and physical and massage therapy.

Acute migraine treatments intended to eliminate or reduce the severity of the headache and any associated symptoms after a migraine has begun include serotonin receptor agonists, such as triptans (5-hydroxytryptophan (5-HT) agonists) including almotriptan, eletriptan, frovatriptan, naratriptan, rizatriptan, sumatriptan, imotriptan, and zolmitriptan; ergotamine-based compounds such as dihydroergotamine and ergotamine; antiemetics such as metoclopramide and prochlorperazine; and compounds that provide analgesic effects.

Other examples of drugs used to treat migraine once started include, acetaminophen-aspirin, caffeine, cyproheptadine, methysergide, valproic acid, NSAIDs such as diclofenac, flurbiprofen, ketaprofen, ketorolac, ibuprofen, indomethacin, meclofenamate, and naproxen sodium, opioids such as codeine, meperidine, and oxycodone, and glucocorticoids including dexamethasone, prednisone and methylprednisolone.

GABA analog prodrugs provided by the present disclosure may also be administered in conjunction with drugs that are useful for treating symptoms associated with migraine such as nausea and vomiting, and depression. Examples of useful therapeutic agents for treating or preventing vomiting include, but are not limited to, 5-HT₃ receptor antagonists such as ondansetron, dolasetron, granisetron, and tropisetron; dopamine receptor antagonists such as prochlorperazine, thiethylperazine, chlorpromazine, metoclopramide, and domperidone; glucocorticoids such as dexamethasone; and benzodiazepines such as lorazepam and alprazolam. Examples of useful therapeutic agents for treating or preventing depression include, but are not limited to, tricyclic antidepressants such as amitryptyline, amoxapine, bupropion, clomipramine, desipramine, doxepin, imipramine, maprotiline, nefazadone, nortriptyline, protriptyline, trazodone, trimipramine, and venlafaxine; selective serotonin reuptake inhibitors such as fluoxetine, fluvoxamine, paroxetine, and setraline; monoamine oxidase inhibitors such as isocarboxazid, pargyline, phenizine, and tranylcypromine; and psychostimulants such as dextroamphetamine and methylphenidate.

Useful drugs for treating cerebral trauma include corticosteroids such as betamethasone, budesonide, cortisone, dexamethasone, hydrocortisone, methylprednisolone, predisolone, prednisone, and triamcinolone, and antithrombotics such as ticlopidine.

Useful drugs for treating epilepsy include acetazolamide, carbamazepine, gabapentin, mephobarbital, felbamate, fosphenytoin, phenytoin, pregabalin, and valproic acid.

In certain embodiments, acamprosate prodrugs provided by the present disclosure and pharmaceutical compositions thereof may be administered to a patient for treating tinnitus in combination with a therapy or another therapeutic agent known or believed to be effective in treating tinnitus.

A second therapeutic agent for treating or preventing tinnitus can have one or more of analgesic, anesthetic, sodium channel blocker, antiedemic, analgesic, and antipyretic properties. Analgesics include, for example, steroidal anti-inflammatory agents, non-steroidal anti-inflammatory agents, selective COX-2 inhibitors, and narcotics. Examples of analgesics include, for example, acetaminophen, amitriptyline, aspirin, buprenorphine, celecoxib, clonidine, codeine, diclofenac, diflunisal, etodolac, fenoprofen, fentanyl, flurbiprofen, hydromorphone, hydroxyzine, ibuprofen, imipramine, indomethacin, ketoprofen, ketorolac, levorphanol, meperidine, methadone, morphine, naproxen, oxycodone, piroxicam, propoxyphene, refecoxib, sulindac, tolmetin, tramadol, valdecoxib, and combinations of any of the foregoing.

In certain embodiments, a compound of the present disclosure or pharmaceutical composition thereof can be administered with a N-methyl-D-aspartate (NMDA) receptor antagonist that binds to the NMDA receptor at the competitive NMDA antagonist binding site, the non-competitive NMDA antagonist binding site within the ion channel, or to the glycine site. Examples of NMDA receptor antagonists include amantadine, D-2-amino-5-phosphonopentanoic acid (D-AP5), 3-((±)2-carboxypiperazin-4-yl)-propyl-1-phosphonic acid (CCP), conantokins, 7-chlorokynurenate (7-CK), dextromethorphan, ifenprodil, ketamine, memantine, dizocilpine, gacyclidine, licostinel, phencyclidine, riluzole, traxoprodil, and combinations of any of the foregoing (Sands, U.S. Pat. No. 5,716,961 and Guitton et al., US 2006/0063802). Other drugs that may be useful in treating tinnitus include baclofen, caroverine, piribedil, nimodipine, clonazepam, and trimetazidine.

An acamprosate prodrug of Formula (I), Formula (III), or Formula (IV), or pharmaceutical composition thereof can also be used in conjunction with non-pharmacological tinnitus therapies such as, for example, avoidance of ototoxic medications, reduced consumption of alcohol, caffeine and nicotine, reduced stress, the use of background noises or maskers, behavioral therapies such as hypnosis, cognitive therapy, biofeedback, tinnitus retraining therapy.

In certain embodiments, acamprosate prodrugs provided by the present disclosure and pharmaceutical compositions thereof may be administered to a patient for treating a sleeping disorder in combination with a therapy or another therapeutic agent known or believed to be effective in treating a sleeping disorder.

Examples of drugs useful for treating sleep apnea include mirtiazapine and modafinil.

In certain embodiments, acamprosate prodrugs provided by the present disclosure and pharmaceutical compositions thereof may be administered to a patient for treating multiple sclerosis in combination with a therapy or another therapeutic agent known or believed to be effective in treating multiple sclerosis.

Examples of drugs useful for treating MS include corticosteroids such as methylprednisolone; IFN-β such as IFN-β1a and IFN-β1b; glatiramer acetate; monoclonal antibodies that bind to the very late antigen-4 (VLA-4) integrin such as natalizumab; immunomodulatory agents such as FTY 720 sphinogosie-1 phosphate modulator and COX-2 inhibitors such as BW755c, piroxicam, and phenidone; and neuroprotective treatments including inhibitors of glutamate excitotoxicity and iNOS, free-radical scaventers, and cationic channel blockers; memantine; AMPA antagonists such as topiramate; and glycine-site NMDA antagonists.

In certain embodiments, acamprosate prodrugs provided by the present disclosure and pharmaceutical compositions thereof may be administered to a patient for treating pain in combination with a therapy or another therapeutic agent known or believed to be effective in treating pain. In certain embodiments, the pain is neuropathic pain.

Examples of drugs useful for treating pain include opioid analgesics such as morphine, codeine, fentanyl, meperidine, methadone, propoxyphene, levorphanol, hydromorphone, oxycodone, oxymorphone, and pentazocine; nonopioid analgesics such as aspirin, ibuprofen, ketoprofen, naproxen, and acetaminophen; nonsteroidal anti-inflammatory drugs such as aspirin, choline magnesium trisalicylate, diflunisal, salsalate, celecoxib, rofecoxib, valdecoxib, diclofenac, etodolac, fenoprofen, flubiprofen, ibuprofen, indomethacin, ketoprofen, ketorolac, meclofanamate, mefenamic acid, meloxicam, nabumetone, naproxen, oxaprozin, piroxicam, sulindac, and tometin; and other drugs such as amitriptyline, desipramine, gabapentin, carbamazepine, phenyloin, clonazepam, divalproex, lamotrigine, topiramate, oxcarbazepine, divalproex, butorphanol, tramadol, valdecoxib, vicoprofen, pentazocine, propoxyhene, fenoprofen, piroxicam, indometnacin, hydroxyzine, buprenorphine, benzocaine, clonidine, flurbiprofen, and meperidine.

The weight ratio of compounds of Formula (I), Formula (III), or Formula (IV) to a second therapeutic agent may be varied and may depend upon the effective dose of each agent. A therapeutically effective dose of each compound will be used. Thus, for example, when a compound of Formula (I), Formula (III), or Formula (IV) is combined with another therapeutic agent, the weight ratio of the compound provided by the present disclosure to the second therapeutic agent can be from about 1000:1 to about 1:1000, and in certain embodiments, from about 200:1 to about 1:200.

Combinations of compounds of Formula (I), Formula (III), or Formula (IV) and a second therapeutic agent may also be within the aforementioned range, but in each case, an effective dose of each active compound can be used. In such combinations a compound of Formula (I), Formula (III), or Formula (IV) and second therapeutic agent may be administered separately or in conjunction. In addition, administration of one agent may be prior to, concurrent with, or subsequent to the administration of another therapeutic agent(s). Accordingly, compounds of Formula (I), Formula (III), or Formula (IV) may be used alone or in combination with other therapeutic agents that are known to be beneficial in treating the same disease being treated with the compound of Formula (I), Formula (III), or Formula (IV) or other therapeutic agents that affect receptors or enzymes that either increase the efficacy, safety, convenience, or reduce unwanted side effects or toxicity of the compound of Formula (I), Formula (III), or Formula (IV). Compounds of Formula (I), Formula (III), or Formula (IV) and the other therapeutic agent may be co-administered, either in concomitant therapy or in a fixed combination. The additional therapeutic agent may be administered by the same or different route than the route used to administer a compound of Formula (I), Formula (III), or Formula (IV) or pharmaceutical composition of any of the foregoing.

### EXAMPLES

The following examples describe in detail synthesis of compounds of Formula (I)-(V), properties of compounds of Formula (I)-(V), and uses of compounds of Formula (I)-(V). It will be apparent to those skilled in the art that many modifications, both to materials and methods, may be practiced without departing from the scope of the disclosure.

### Description 1

### General Experimental Protocols

All reagents and solvents were purchased from commercial suppliers and used without further purification or manipulation.

Proton NMR spectra (400 MHz) were recorded on a Varian AS 400

NMR spectrometer equipped with an autosampler and data processing computation. CDCl₃ (99.8% D), DMSO-d⁶ (99.9% D), or MeOH-d⁴ (99.8+% D) were used as solvents unless otherwise noted. The CHCl₃, DMSO-d⁵, or MeOH-d³ solvent signals were used for calibration of the individual spectra. Analytical thin layer chromatography (TLC) was performed using Whatman, Schleicher & Schuell TLC. MK6F silica gel plates (2.5×7.5 cm, 250 μm layer thickness). Dyeing or staining reagents for TLC detection and visualization were prepared using methods known in the art. Ozonolysis reactions were performed using a Welsbach Standard T-series ozone generator. A belt-driven Parr hydrogenation apparatus, Model No. 3911 EA from Parr Instrument, Co. was used for high-pressure hydrogenations Analytical LC/MS was performed on a Waters 2790 separation module equipped with a Waters Micromass QZ mass spectrometer, a Waters 996 photodiode detector, and a Merck Chromolith UM2072-027 or Phenomenex Luna C-18 analytical column. Mass-guided preparative HPLC purification of final compounds was performed on an instrument equipped with a Waters 600 controller, ZMD Micromass spectrometer, a Waters 2996 photodiode array detector, and a Waters 2700 Sample Manager. Acetonitrile/water gradients containing 0.05% formic acid were used as eluent in both analytical and preparative HPLC experiments. Compound isolation from aqueous solvent mixtures, e.g., acetonitrile/water/0.05% formic acid, was accomplished through primary lyophilization (freeze drying) of the frozen solutions under reduced pressure at room temperature using manifold freeze dryers such as Heto Drywinner DW 6-85-1, Heto FD4, or VIRTIS Freezemobile 25 ES equipped with high vacuum pumps. Optionally and if the isolated compound had ionizable functional groups such as an amino group or a carboxylic acid, the lyophilization process was conducted in the presence of a slight excess of one molar (1.0 M) hydrochloric acid to yield the purified compounds as the corresponding hydrochloride salts (HCl-salts) or the corresponding protonated free carboxylic acids. Chemical names were generated with the Chemistry 4-D Draw Pro Version 7.01c (Draw Chemical Structures Intelligently© 1993-2002) from ChemInnovation Software, Inc., San Diego, USA).

### Example 1

### Preparation of (tert-butoxy)-N-(4-hydroxy-3,3-dimethylbutyl) carboxamide (Neopentyl Alcohol) (1)

Neopentyl alcohol was prepared from commercially available 3,3-dimethyloxirane following the procedures of Mullis, et al., J. Org. Chem. 1982, 47, 2873-2875 and Roberts, et al., Tetrahedron Lett. 1997, 38, 355-358, or following the procedure described in Roberts, et al., U.S. Pat. No. 5,596,095 (WO 96/18609). Neopentyl alcohol was more readily prepared by adapting procedures, or variations thereof, described by Scheinmann, et al., J. Chem. Res. (S) 1993, 414-415, and Flynn, et al., J. Org. Chem. 1983, 48, 2424-2426 using pyrrolidin-2-one as the starting material.

### Step A: 1-(tert-Butoxy)carbonyl-pyrrolidin-2-one (1a)

Adapting procedures or variations thereof according to Scheinmann, et al., J. Chem. Res. (S), 1993, 414-415, and Flynn, et al., J. Org. Chem. 1983, 48, 2424-2426, pyrrolidin-2-one (4.25 g, 50.0 mmol) was reacted in the presence of 610 mg (5.0 mmol, 10 mol %) 4-dimethylaminopyridine (DMAP) and 10.95 mL (7.59 g, 75.0 mmol) of triethylamine with 11.95 g (55.0 mmol) di-tert-butylpyrocarbonate (Boc₂O) in 50 mL of anhydrous dichloromethane. After aqueous work-up, the product was purified by silica gel chromatography using a mixture of ethyl acetate (EtOAc)/hexane (Hxn) (2:3) as eluent to yield 8.89 g (96% yield) of the title compound (1a) as a pale yellow oil. R_{f}=0.55 (EtOAc/Hxn=1:1). The analytical data agreed with that given for the title compound (1a) in the literature. Alternatively, the title compound (1a) can also be obtained from commercial suppliers.

### Step B: 1-(tert-Butoxy)carbonyl-3,3-dimethylpyrrolidin-2-one (1b)

Following the procedure according to Scheinmann, et al., J. Chem. Res. (S), 1993, 414-415, lithiumhexamethyldisilazide (LHMDS) was prepared prior to use from hexamethyldisilazane (58.0 mL, 44.4 g, 275 mmol) and n-butyllithium (nBuLi) (1.6 M in hexane, 169 mL, 270 mmol). After evaporation, the nitrogen base was dissolved in 250 mL of anhydrous tetrahydrofurane. In dry Schlenk glassware 1-(tert-butoxy)carbonyl-pyrrolidin-2-one (1a) (18.52 g, 100 mmol) was 3,3-dimethylated at −78° C. (dry ice/acetone) under nitrogen with iodomethane (37.4 mL, 85.2 g, 600 mmol) in 100 mL of anhydrous tetrahydrofuran (THF). After aqueous work-up, the product was purified by silica gel column chromatography using a mixture of ethyl acetate/hexane (1:9) as eluent to provide 9.55 g (45% yield) of the title compound (1b) as a pale yellow oil. R_{f}=0.15 (EtOAc/hexane=1:9). The analytical data agreed with that given for the title compound (1b) in the literature.

### Step C: 4-(tert-Butoxy)carbonylamino-2,2-dimethylbutanoic Acid (1c)

Following the procedure according to Scheinmann, et al., J. Chem. Res. (S), 1993, 414-415, 1-(tert-butoxy)carbonyl-3,3-dimethylpyrrolidin-2-one (1b) (9.5 g, 44.5 mmol) in ca. 70 mL of a mixture of THF and ethanol (1:1) was reacted with a solution of 9.1 g (228 mmol) of sodium hydroxide in water at room temperature for more than 12 h (TLC control). After aqueous work-up, 8.80 g (85% yield) of the title compound (1c) was obtained as a colorless solid. The compound was used in the next step without further purification. R_{f}=0.35 (EtOAc/hexane=1:2). ¹H NMR (400 MHz, DMSO-d⁶): δ=1.08 (s, 6H), 1.36 (s, 9H), 1.54-1.60 (m, 2H), 2.83-2.92 (m, 2H), 6.74 (br. t, J=5.6 Hz, 1H), 12.04 (br. s, 1H) ppm. MS (ESI) m/z 231.03 (M+H)⁺, 254.08 (M+Na)⁺, 230.09 (M−H)⁻. The analytical data agreed with that given for the title compound (1c) in the literature.

### Step D: Methyl (4-tert-butoxy)carbonylamino-2,2-dimethylbutanoate (1d)

4-(tert-Butoxy)carbonylamino-2,2-dimethylbutanoic acid (1c) (8.33 g, 36.02 mmol) was esterified using 4.48 mL (10.23 g, 72.0 mmol) of iodomethane in the presence of 14.93 g (108.0 mmol) of potassium carbonate in 100 mL of anhydrous DMF. After aqueous work-up and purification by silica gel chromatography using a mixture of ethyl acetate/hexane (1:3) as eluent, ca. 9 g (quant.) of the title compound (1d) was obtained as a yellow oil. R_{f}=0.55 (EtOAc/Hxn=1:1). ¹H NMR (400 MHz, CDCl₃): δ=1.21 (s, 6H), 1.44 (s, 9H), 1.72-1.79 (m, 2H), 3.08-3.17 (m, 2H), 3.68 (s, 3H), 4.43-4.55 (br. m, 1H) ppm. MS (ESI) m/z 246.01 (M+H)⁺, 267.99 (M+Na)⁺.

Alternatively, a diethyl ether solution of diazomethane was prepared prior to use according to common synthetic procedures known to those skilled in the art from a reaction mixture of N-nitrosourea-n-methylurea 5.15 g (50.0 mmol) and sodium hydroxide (20.0 g, 500.0 mmol) in 30 mL of water at 0° C. in a glass beaker. 1.16 g (5.05 mmol) of 4-(tert-butoxy)carbonylamino-2,2-dimethylbutanoic acid (1c), dissolved in 25 mL of methanol, was reacted at 0° C. with a slight excess of diazomethane in diethylether (dropwise titration until yellow color persisted for several seconds; TLC control). Excess diazomethane was removed with a slight excess of a dilute aqueous solution of acetic acid. The solvent was removed under reduced pressure using a rotary evaporator to yield 1.24 g (quant.) of the title compound (1d) as a colorless oil.

### Step E: (tert-Butoxy)-N-(4-hydroxy-3,3-dimethylbutyl)carboxamide (1)

8.5 g (35 mmol) of methyl (4-tert-butoxy)carbonylamino-2,2-dimethylbutanoate (1d) was dissolved under an atmosphere of nitrogen in dry Schlenk glassware in 35 mL of anhydrous THF. A solution of lithium borohydride (1.53 g, 70 mmol) in 35 mL of anhydrous tetrahydrofuran (ca. 2 M) was added dropwise to the solution of the methyl ester at room temperature. The reaction was monitored by TLC. 100 mL of ethanol was added and the reaction mixture stirred for an additional 12 h at room temperature. After the starting material was completely consumed, the reaction was quenched by addition of a 10% (w/v) solution of citric acid in water. THF was removed under reduced pressure using a rotary evaporator and the crude residue was diluted with ethylacetate. After extraction (twice), the combined organic extracts were washed with water and brine, dried over magnesium sulfate, filtered, and the solvents removed under reduced pressure using a rotary evaporator. The residue was purified by silica gel chromatography using a mixture of ethyl acetate/hexane (1:1) as eluent to provide 7.02 g (96% yield) of the title compound (1). R_{f}=0.22 (EtOAc/Hxn=1:1). ¹H NMR (400 MHz, CDCl₃): δ=0.91 (s, 6H), 1.45 (s, 9H), 1.46-1.51 (m, 2H), 3.10-3.16 (m, 2H), 3.36 (s, 2H) ppm. MS (ESI) m/z 240.03 (M+Na)⁺. The analytical data agreed with that given in the literature.

### Example 2

### 4-Amino-2,2-dimethylbutan-1-ol Hydrochloride (2)

A 250 mL round bottomed flask equipped with a magnetic stir bar was charged with 7.02 g (32.3 mmol) of (tert-butoxy)-N-(4-hydroxy-3,3-dimethylbutyl)carboxamide (1). 50 mL of anhydrous diethylether was added followed by 35 mL of a 4 M solution of hydrogen chloride (HCl) in 1,4-dioxane (140 mmol). The reaction mixture was stirred at room temperature for 12 h, additional diethylether added, the colorless precipitate filtered off, and the filter residue washed with diethylether and dried in high vacuum to provide 4.00 g (81% yield) of the title compound (2) as a colorless hygroscopic solid. The compound was used in the next steps without further purification. ¹H NMR (400 MHz, DMSO-d⁶): δ=0.81 (s, 6H), 1.46-1.51 (m, 2H), 2.70-2.80 (m, 2H), 3.08 (s, 2H), 4.75 (br. s, 1H), 7.94 (br. s, 3H) ppm. MS (ESI) m/z 117.95 (M+H)⁺.

### Example 3

### 3,3-Dimethylpyrrolidine Hydrochloride (3)

### Step A: tert-Butyl 3,3-dimethylpyrrolidinecarboxylate (3a)

Adapting a procedure by Ezquerra, et al., J. Org. Chem. 1994, 59, 4327-4331, to a solution of 1.174 g (5.53 mmol) of 1-(tert-butoxy)carbonyl-3,3-dimethylpyrrolidin-2-one (1b) in 30 mL of anhydrous THF in an oven dried 250 mL round bottomed flask equipped with a magnetic stirring bar and a rubber septum, 6.64 mL of a one molar (1M) solution of lithium triethylborohydride (LiBHEt₃/Superhydride) (6.64 mmol) in tetrahydrofuran (THF) was added dropwise under a nitrogen atmosphere at −78° C. After stirring for 30 minutes the reaction mixture was quenched by addition of 10 mL of a saturated aqueous solution of sodium bicarbonate (NaHCO₃) followed by gradual warming to room temperature. 1 mL of a 30 w-% aqueous solution of hydrogen peroxide (H₂O₂) (9.7 mmol) was added and the reaction mixture stirred for additional 30 minutes. The organic solvents were evaporated under reduced pressure using a rotary evaporator and the residual aqueous layer was diluted with dichloromethane (CH₂Cl₂/DCM). After phase separation, the aqueous phase was extracted three more times with dichloromethane and the combined organic extracts were dried over magnesium sulfate (MgSO₄) and filtered. The solvents were removed under reduced pressure using a rotary evaporator to yield the title compound (3a) as a colorless oil, which was used without further purification in the next step.

A 100 mL round bottomed flask equipped with a magnetic stirring bar and a rubber septum was charged under an atmosphere of nitrogen with a solution of the hemiaminal in 30 mL of anhydrous dichloromethane and 910 μL of triethylsilane (TES) (663 mg, 5.7 mmol). The mixture was cooled to −78° C. 790 μL of boron trifluoride etherate (BF₃.Et₂O) (885 mg, 6.27 mmol) was added dropwise. After 30 minutes, an additional 910 μL of triethylsilane (TES) (663 mg, 5.7 mmol) and 790 μL of BF₃.Et₂O (885 mg, 6.27 mmol) were added. The mixture was stirred for an additional two hours at this temperature, following which the reaction mixture was quenched by adding 8 mL of a saturated aqueous solution of sodium bicarbonate (NaHCO₃) and water followed by gradual warming to room temperature. The cooled reaction mixture was diluted with dichloromethane. After phase separation, the aqueous phase was extracted three more times with dichloromethane and the combined organic extracts were washed with brine, dried over magnesium sulfate (MgSO₄), filtered, and the solvents removed under reduced pressure using a rotary evaporator to yield a colorless oil. The crude material was purified by silica gel column chromatography using mixtures of ethyl acetate (EtOAc) and hexane (Hxn) as eluent (EtOAc/Hxn=1:9→EtOAc/Hxn=1:6) to provide 1 g (90% yield) of the title compound (2a) as a colorless liquid. The purified material contained trace amounts of TES-dimer (hexaethylsilane) that was subsequently removed under high vacuum. The residual material was used directly in the next step without further manipulation. R_{f}=0.50 (EtOAc/Hxn=1:6). ¹H NMR (400 MHz, CDCl₃): δ=1.07 (s, 6H), 1.47 (s, 9H), 1.59-1.66 (m, 2H), 3.07 (d, J=25.2 Hz, 2H), 3.39 (dt, J=23.2, 6.8 Hz, 2H) ppm. MS (ESI) m/z=200.21 (M+H)⁺.

### Step B: 3,3-Dimethylpyrrolidine hydrochloride (3)

A 100 mL round bottomed flask equipped with a magnetic stirring bar, a stainless steel needle connected to a hydrogen chloride (HCl) cylinder, and a perforated polyethylene cap, was charged with 1 g (5 mmol) of tert-butyl 3,3-dimethylpyrrolidinecarboxylate (3a). The material was dissolved in 20 mL of anhydrous diethyl ether and a gentle stream of gaseous HCl was carefully bubbled through the solution at room temperature for two hours. The reaction mixture was then stirred for an additional two hours. The solvent was removed under reduced pressure using a rotary evaporator followed by high vacuum drying overnight to provide 598 mg (80% yield, two steps) of the title compound (3) as colorless solid. R_{f}=0.33 [DCM/10 vol-% MeOH+2 vol-% triethylamine (TEA)]. ¹H NMR (400 MHz, DMSO-d⁶): δ=1.08 (s, 6H), 1.65-1.71 (m, 2H), 2.80-2.85 (m, 2H), 3.16-3.25 (m, 2H), 9.38 (br.m, 2H) ppm. MS (ESI) m/z=100.10 (M+H)⁺.

### Description 2

### General Procedure for the N-Boc-Protection of ω-Amino-2,2-Dimethylalcohols

A 250 mL round bottomed flask equipped with a magnetic stirring bar was charged with 10.0 mmol of the ω-amino-2,2-dimethylalcohol or the corresponding hydrochloride salt. The reagent was dissolved at room temperature in a mixture of 25 mL of a 1N aqueous solution of sodium hydroxide (NaOH) and 25 mL of 1,4-dioxane. Alternatively, the reagent was dissolved in 10 mL of a saturated aqueous solution of sodium bicarbonate (NaHCO₃) and 20 mL of acetonitrile. Di-tert-butyl-dicarbonate (Boc₂O) (2.62 g, 12.0 mmol) was added at room temperature and the reaction mixture stirred overnight at room temperature. The reaction mixture was then diluted with ethyl acetate (100 mL) and acidified with 50 mL of a 1N aqueous solution of hydrogen chloride (HCl). After separation of the phases, the organic phase was washed with brine, dried over magnesium sulfate (MgSO₄), filtered, and the solvents removed under reduced pressure using a rotary evaporator to yield the N-Boc-protected ω-amino-2,2-dimethylalcohol as a colorless viscous oil or colorless solid of sufficient purity to be used in subsequent steps and without further isolation and purification.

### Example 4

### (tert-Butoxy)-N-(1-hydroxy-2-methylpropyl)carboxamide (4)

Following the general procedure for the N-Boc-protection of ω-amino-2,2-dimethylalcohols of Description 2, 1.783 g (20.0 mmol) of 2-amino-2-methyl-1-propanol was reacted with 3.274 g (15.0 mmol) of di-tert-butyl-dicarbonate in a mixture of 20 mL of a saturated aqueous solution of sodium bicarbonate (NaHCO₃) and 40 mL of acetonitrile to provide 1.937 g (68% yield) of the title compound (4) as a colorless solid of sufficient purity to be used in subsequent steps without further isolation and purification. ¹H NMR (400 MHz, CDCl₃): δ=1.26 (s, 6H), 1.44 (s, 9H), 3.59 (d, J=6.0 Hz, 2H), 4.00-4.20 (br. m, 1H), 4.64-4.72 (br. m, 1H) ppm. MS (ESI) m/z 212.92 (M+Na)⁺.

### Example 5

### (tert-Butoxy)-N-(3-hydroxy-2,2-dimethylpropyl)carboxamide (5)

Following the general procedure for the N-Boc-protection of ω-amino-2,2-dimethylalcohols of Description 2, 2.064 g (20.0 mmol) of 3-amino-2,2-dimethyl-1-propanol was reacted with 3.274 g (15.0 mmol) of di-tert-butyl-dicarbonate in a mixture of 20 mL of a saturated aqueous solution of sodium bicarbonate (NaHCO₃) and 40 mL of acetonitrile to provide ca. 2.877 g (94% yield) of the title compound (5) as a colorless solid of sufficient purity to be used in subsequent steps without further isolation and purification. ¹H NMR (400 MHz, CDCl₃): δ=0.86 (s, 6H), 1.45 (s, 9H), 2.92-2.98 (br. m, 2H), 3.20 (s, 2H), 4.82-4.94 (br. m, 1H) ppm. MS (ESI) m/z 226.01 (M+Na)⁺.

### Example 6

### (tert-Butoxy)-N-(5-hydroxy-4,4-dimethylpentyl)carboxamide (6)

Following the general procedure for the N-Boc-protection of ω-amino-2,2-dimethylalcohols of Description 2, 1.312 g (10.0 mmol) of 5-amino-2,2-dimethyl-1-pentanol was reacted with 2.62 g (12.0 mmol) of di-tert-butyl-dicarbonate in a mixture of 25 mL of a 1N aqueous solution of sodium hydroxide (NaOH) and 25 mL of 1,4-dioxane to provide ca. 2.6 g (quant.) of the title compound (6) as a colorless, viscous oil of sufficient purity to be used in subsequent steps without further isolation and purification. ¹H NMR (400 MHz, CDCl₃): δ=0.87 (s, 6H), 1.21-1.28 (m, 2H), 1.40-1.48 (m, 11H), 3.08 (br. q, J=6.4 Hz, 2H), 3.30 (s, 2H), 3.70 (s, 1H), 4.65 (br. m, 1H) ppm. MS (ESI) m/z 254.18 (M+Na)⁺.

### Description 3

### General Procedure for Synthesis of Acyloxyalkyl Carbamates

A screw-capped 40 mL glass vial equipped with a magnetic stir bar was charged with ω-amino-2,2-dimethylalcohol or its corresponding hydrochloride (2.0 mmol) and 20 mL of acetonitrile. An appropriately substituted acyloxyalkyl N-hydroxysuccinimide carbonic acid ester (2.0 mmol) was added either as a solid or dissolved in a small volume of solvent (for oily materials). 5 mL of a saturated aqueous sodium bicarbonate (NaHCO₃) solution was added and the reaction mixture stirred for ca. 12 hours at room temperature. Alternatively, free aminoalcohols may be reacted in a methyl tert-butylether (MTBE)/acetone/water mixture (4:3:1) as disclosed in Zerangue et al., U.S. Pat. No. 7,351,740.

Upon completion of the reaction, the mixture was diluted with ethyl acetate and 10 mL of 1N aqueous hydrochloric acid (HCl) added. After vigorous mixing followed by phase separation, the aqueous layer was extracted once more with ethyl acetate and the combined organic extracts washed with saturated aqueous sodium bicarbonate solution and brine. The combined organic extracts were dried over magnesium sulfate (MgSO₄), filtered, and the solvents evaporated under reduced pressure using a rotary evaporator. The resulting residue was either of sufficient purity to be used without further isolation and purification, or, alternatively the acyloxyalkyl carbamate compounds were purified by silica gel chromatography using an ethyl acetate/hexane mixture as eluent followed by removal of the solvents under reduced pressure using a rotary evaporator to yield a colorless viscous oil or solid.

For analytically pure samples, the residue was dissolved in a mixture of 60% (v/v) acetonitrile/water. The solution was filtered through a 0.2 μm nylon syringe filter and purified by mass-guided preparative HPLC. After lyophilization, the pure acyloxyalkylcarbamate compounds were obtained as colorless oils or solids.

### Example 7

### [N-(2-Hydroxy-tert-butyl)carbamoyloxy]ethyl 2-methylpropanoate (7)

Following the general procedure of Description 3,2-amino-2-methyl-1-propanol (1.783 g, 20.0 mmol), dissolved in 40 mL of acetonitrile and 20 mL of a saturated aqueous solution of NaHCO₃, was reacted with [(2,5-dioxopyrrolidinyl)oxycarbonyloxy]ethyl 2-methylpropanoate (4.10 g, 15.0 mmol). After aqueous work-up, 3.68 g (quant.) of the title compound (7) was obtained as a colorless viscous oil. The material was of sufficient purity to be used without further isolation and purification in the next step. ¹H NMR (400 MHz, CDCl₃): δ=1.17 (d, J=7.2 Hz, 3H), 1.18 (d, J=6.8 Hz, 3H), 1.28 (s, 3H), 1.30 (s, 3H), 1.46 (d, J=5.2 Hz, 3H), 2.54 (heptet, J=7.2 Hz, 1H), 3.14-3.26 (br. m, 1H), 3.55 (dd, J=10.8, 5.6 Hz, 1H), 3.67 (dd, J=10.4, 4.8 Hz, 1H), 4.09 (br. s, 1H), 6.74 (q, J=5.6 Hz, 1H) ppm. MS (ESI) m/z 247.93 (M+H)⁺, 269.98 (M+Na)⁺.

### Example 8

### [N-(3-Hydroxy-2,2-dimethylpropyl)carbamoyloxy]ethyl 2-methylpropanoate (8)

Following the general procedure of Description 3,3-amino-2,2-dimethyl-1-propanol (1.032 g, 10.0 mmol), dissolved in 20 mL of acetonitrile and 10 mL of a saturated aqueous solution of NaHCO₃, was reacted with [(2,5-dioxopyrrolidinyl)oxycarbonyloxy]ethyl 2-methylpropanoate (2.0 g, 7.32 mmol). After aqueous work-up, 1.91 g (quant.) of the title compound (8) was obtained as a colorless viscous oil. The material was of sufficient purity to be used without further isolation and purification in the next step. ¹H NMR (400 MHz, CDCl₃): δ=0.87 (s, 3H), 0.88 (s, 3H), 1.16 (d, J=7.2 Hz, 3H), 1.18 (d, J=6.8 Hz, 3H), 1.47 (d, J=5.2 Hz, 3H), 2.53 (heptet, J=6.8 Hz, 1H), 2.76-2.90 (br. m, 1H), 3.00 (dd, J=14.0, 6.8 Hz, 1H), 3.09 (dd, J=14.4, 6.8 Hz, 1H), 3.22 (d, J=10.8 Hz, 1H), 3.26 (d, J=11.6 Hz, 1H), 5.16-5.23 (br. m, 1H), (br. s, 1H), 6.78 (q, J=5.6 Hz, 1H) ppm. MS (ESI) m/z 262.02 (M+H)⁺, 284.03 (M+Na)⁺.

### Example 9

### [N-(4-Hydroxy-3,3-dimethylbutyl)carbamoyloxy]ethyl 2-methylpropanoate (9)

Following the general procedure of Description 3,4-amino-2,2-dimethylbutan-1-ol hydrochloride (1.34 g, 8.7 mmol) dissolved in 30 mL of acetonitrile and 15 mL of a saturated aqueous NaHCO₃ solution, was reacted with [(2,5-dioxopyrrolidinyl)oxycarbonyloxy]-ethyl 2-methylpropanoate (2.73 g, 10.0 mmol). After aqueous work-up, 2.39 g (quant.) of the title compound (9) was obtained as a colorless solid. The material was of sufficient purity to be used without further isolation and purification in the next step. R_{f}=0.46 (EtOAc/Hxn=1:1). ¹H NMR (400 MHz, DMSO-d⁶): δ=0.79 (s, 6H), 1.059 (s, 3H), 1.064 (s, 3H), 1.29-1.35 (m, 2H), 1.46 (d, J=5.6 Hz, 3H), 2.49 (heptet, J=6.8 Hz, 1H), 2.93-3.01 (m, 2H), 3.06 (d, J=5.2 Hz, 2H), 4.52 (t, J=5.2 Hz, 1H), 6.63 (q, J=5.6 Hz, 1H), 7.32 (br. t, 5.6 Hz) ppm. MS (ESI) m/z 276.13 (M+H)⁺.

### Example 10

### [N-(4-Hydroxy-3,3-dimethylbutyl)carbamoyloxy]ethyl Benzoate (10)

Following the general procedure of Description 3,4-amino-2,2-dimethylbutan-1-ol hydrochloride (600 mg, 3.9 mmol) dissolved in 30 mL of acetonitrile and 10 mL of a saturated aqueous NaHCO₃ solution, was reacted with 2,5-dioxopyrrolidinyl (phenylcarbonyloxyethoxy)formate (1.30 g, 4.3 mmol). After aqueous work-up the product was purified by silica gel column chromatography using a mixture of ethyl acetate/hexane (EtOAc/Hxn=1:1) as an eluent to provide 1.1 g (92% yield) of the title compound (10) as a colorless, viscous liquid. R_{f}=0.47 (EtOAc/Hxn=1:1). ¹H NMR (400 MHz, CDCl₃): δ=0.90 (s, 6H), 1.51 (m, 2H), 1.59 (d, J=5.2 Hz, 3H), 3.18-3.24 (m, 2H), 5.12 (br. s, 1H), 7.06 (q, J=5.2 Hz, 1H), 7.04-7.44 (m, 2H), 7.54-7.58 (m, 1H), 8.02-8.04 (m, 2H). MS (ESI) m/z 310.19 (M+H)⁺.

### Example 11

### [N-(5-Hydroxy-4,4-dimethylpentyl)carbamoyloxy]ethyl 2-methylpropanoate (11)

Following the general procedure of Description 3,5-amino-2,2-dimethylpentan-1-ol (1.312 g, 10.0 mmol) dissolved in 20 mL of acetonitrile and 10 mL of a saturated aqueous NaHCO₃ solution, was reacted with [(2,5-dioxopyrrolidinyloxy)oxycarbonyl-oxy]ethyl 2-methylpropanoate (1.366 g, 5.0 mmol). After aqueous work-up, 1.339 g (93% yield) of the title compound (11) was isolated as a colorless, viscous oil. The material was of sufficient purity to be used without further isolation and purification in the next step. R_{f}=0.42 (EtOAc/Hxn=1:1). ¹H NMR (400 MHz, CDCl₃): δ=0.86 (s, 6H), 1.15-1.17 (m, 6H), 1.23-1.27 (m, 2H), 1.5-1.44 (m, 5H), 2.52 (heptet, 1H), 3.13-3.18 (m, 2H), 3.30 (s, 2H), 4.94 (br. m, 1H), 7.06 (q, J=5.2 Hz, 1H) ppm. MS (ESI) m/z 312.15 (M+Na)⁺.

### Example 12

### [N-(5-Hydroxy-4,4-dimethylpentyl)carbamoyloxy]methyl Benzoate (12)

Following the general procedure of Description 3,5-amino-2,2-dimethylpentan-1-ol (1.312 g, 10.0 mmol) dissolved in 20 mL of acetonitrile and 10 mL of a saturated aqueous NaHCO₃ solution, was reacted with 2,5-dioxopyrrolidinyl (phenylcarbonyloxymethoxy) formate (1.47 g, 5.0 mmol). After aqueous work-up, 1.106 g (72% yield) of the title compound (12) was isolated as a yellowish viscous oil. The material was used without further purification. R_{f}=0.34 (ethyl acetate/hexane=1:1). ¹H NMR (400 MHz, CDCl₃): δ=0.86 (s, 6H), 1.24-1.28 (m, 2H), 1.46-1.53 (m, 2H), 3.17-3.22 (m, 2H), 3.30 (s, 2H), 5.07 (br. m, 1H), 5.97 (s, 2H), 7.06 (q, J=5.2 Hz, 1H), 7.42-7.46 (m, 2H), 7.55-7.60 (m, 1H), 8.06-8.08 (m, 2H) ppm. MS (ESI) m/z 332.13 (M+Na)⁺.

### Example 13

### Synthesis of N-[3-(Chlorosulfonyl)propyl]acetamide (13)

### Step A: Tetramethylammonium N-acetylhomotaurate (13a)

Tetramethylammonium N-acetylhomotaurate was synthesized adapting procedures disclosed in Durlach, U.S. Pat. No. 4,355,043 and U.S. Pat. No. 4,199,601, and DE 3019350. A 250 mL round bottomed flask equipped with a magnetic stir bar was charged with 3-amino-1-propanesulfonic acid (5.0 g, 36 mmol) and 20 mL of water. To the stirred solution, 13.0 g (36.0 mmol) of tetramethylammonium hydroxide ((CH₃)₄NOH, TMAH) (25 w-% in water) was added. The solution was stirred at room temperature for 1 hour and 4.1 mL of acetic anhydride (4.39 g, 43 mmol) was added. The mixture was stirred overnight at ca. 40° C. (oil bath) to ensure complete conversion. The resulting solution was extracted twice with 30 mL of diethyl ether or tert-butyl methyl ether (MTBE) and residual methanol in the aqueous phase was removed under reduced pressure using a rotary evaporator. The product was isolated from the residual water in the solution by lyophilization to yield 9.1 g (quant.) of the title compound (13a) as a colorless powder that was used without further purification after additional drying under high vacuum. ¹H NMR (400 MHz, D₂O): δ=1.88-1.95 (m, 2H), 1.97 (s, 3H), 2.88-2.92 (m, 2H), 3.16 (s, 12H), 3.27 (m, 2H) ppm. MS (ESI) m/z 180.04 (M−H)⁻.

### Step B: N-[3-(Chlorosulfonyl)propyl]acetamide (13)

A 500 mL round bottomed flask equipped with a magnetic stir bar was charged with tetramethylammonium N-acetylhomotaurate (13a) (9.1 g, 36 mmol), phosphorus pentachloride (7.9 g, 37 mmol), and anhydrous dichloromethane (200 mL). The solution was heated to reflux and reacted overnight. The resulting mixture was washed twice with water (100 mL) and brine (100 mL). The organic layer was dried over MgSO₄, filtered, and the solvents removed by evaporation under reduced pressure using a rotary evaporator to provide 4.6 g (65% yield) of the title compound (13) as a slightly yellow, viscous liquid. The crude material was of sufficient purity to be used in the next step. ¹H NMR (400 MHz, CDCl₃): δ=2.07 (s, 3H), 2.23-2.32 (m, 2H), 3.46-3.51 (m, 2H), 3.76-3.80 (m, 2H) ppm. MS (ESI) m/z 200.01 (M+H)⁺.

### Description 4

### General Procedure for Synthesis of Acamprosate Neopentyl Sulfonylester Prodrugs

A 100 mL round bottomed flask equipped with a magnetic stir bar was charged under an atmosphere of nitrogen with N-[3-(chlorosulfonyl)propyl]acetamide (13) (600 mg, 3.0 mmol), the corresponding functionalized neopentyl alcohol (3.0 mmol), and dichloromethane (15 mL). The reaction mixture was cooled to ca. 0° C. (ice bath) and to the solution was added 418 μL of triethylamine (304 mg, 3.0 mmol,) or 243 μL of pyridine (237 mg, 3.0 mmol), and 4-(N,N-dimethylamino)pyridine (DMAP) (367 mg, 3.0 mmol). The reaction mixture was stirred overnight with gradual warming to room temperature. Upon completion of the reaction, dichloromethane was evaporated and the residue was diluted with ethyl acetate and water. The aqueous layer was acidified with an aqueous one normal (1 N) hydrogen chloride (HCl) solution. After vigorous mixing followed by phase separation, the aqueous layer was extracted twice more with ethyl acetate. The combined organic extracts were successively washed with a saturated aqueous sodium hydrogencarbonate (NaHCO₃) solution, brine, and dried over magnesium sulfate (MgSO₄). After filtration, the solvent was evaporated under reduced pressure using a rotary evaporator. The residue was dissolved in a mixture of ca. 60% (v/v) acetonitrile/water and the solution was filtered through a 0.2-μm nylon syringe filter and purified by mass-guided preparative HPLC. After lyophilization of the solvents, pure acamprosate neopentyl sulfonylester prodrugs were obtained as colorless oils or solids. Alternatively, acamprosate neopentyl sulfonylester prodrugs were purified by silica gel chromatography using ethyl acetate/hexane or ethyl acetate/methanol mixtures as eluent followed by removal of the solvents under reduced pressure using a rotary evaporator.

### Example 14

### 2-[(tert-Butoxy)carbonylamino]-2-methylpropyl [3-(acetylamino)propyl]sulfonate (14)

Following the general procedure for the synthesis of acamprosate neopentyl sulfonylester prodrugs of Description 4, N-[3-(chlorosulfonyl)propyl]acetamide (13) (ca. 800 mg, 4.0 mmol) dissolved in 20 mL of dichloromethane was reacted with (tert-butoxy)-N-(1-hydroxy-2-methylpropyl)carboxamide (4) (757 mg, 4.0 mmol) in the presence of 558 μL of triethylamine (405 mg, 4.0 mmol) and 489 mg (4.0 mmol) of DMAP. After purification using mass-guided preparative HPLC, 19 mg (1.3% yield) of the title compound (14) was obtained as a colorless, viscous oil. ¹H NMR (400 MHz, CDCl₃): δ=1.34 (s, 6H), 1.45 (s, 9H), 2.02 (s, 3H), 2.04-2.12 (m, 2H), 3.15-3.21 (m, 2H), 3.42 (q, J=6.4 Hz, 2H), 4.31 (s, 2H), 4.60 (br. m, 1H), 5.99 (br.m, 1H) ppm. MS (ESI) m/z 375.05 (M+Na)⁺.

### Example 15

### 2-Amino-2-methylpropyl [3-(acetylamino)propyl]sulfonate Trifluoroacetate (15)

2-[(tert-Butoxy)carbonylamino]-2-methylpropyl [3-(acetylamino)propyl]sulfonate (14) (18.8 mg, 0.046 mmol) was dissolved in 1 mL of dichloromethane. To this solution was added 1 mL of neat trifluoroacetic acid (TFA). The mixture was stirred at room temperature for several hours. Upon completion of the reaction, the solvent and excess acid was removed under reduced pressure using a rotary evaporator to provide 16 mg (quant.) of the title compound (15) as a colorless oil. The product was of sufficient purity to be used directly without further purification for in vitro assaying. MS (ESI) m/z 253.01 (M+H)⁺, 275.09 (M+Na)⁺.

### Example 16

### [N-(2-{[3-(Acetylamino)propyl]sulfonyloxy}-tert-butyl)carbamoyloxy]ethyl 2-methylpropanoate (16)

Following the general procedure for the synthesis of acamprosate neopentyl sulfonylester prodrugs of Description 4, N-[3-(chlorosulfonyl)propyl]-acetamide (13) (ca. 800 mg, 4.0 mmol) dissolved in 20 mL of dichloromethane was reacted with [N-(2-hydroxy-tert-butyl)carbamoyloxy]ethyl 2-methylpropanoate (7) (989 mg, 4.0 mmol) in the presence of 558 μL of triethylamine (405 mg, 4.0 mmol) and 489 mg (4.0 mmol) of DMAP. Following purification by mass-guided preparative HPLC, 209 mg (18% yield) of the title compound (16) was obtained as a colorless powder. M.p.: 75.0-82.3° C. ¹H NMR (400 MHz, DMSO-d⁶): δ=1.06 (d, J=6.8 Hz, 6H), 1.207 (s, 3H), 1.210 (s, 3H), 1.38 (d, J=5.2 Hz, 3H), 1.74-1.84 (m, 5H), 2.49 (heptet, J=7.6 Hz, 1H), 3.08-3.16 (m, 2H), 3.26-3.33 (m, 2H), 4.15-4.25 (m, 2H), 6.01 (q, J=5.2 Hz, 1H), 7.49 (s, 1H), 7.91 (t, J=5.6 Hz, 1H) ppm. MS (ESI) m/z 411.08 (M+H)⁺, 433.03 (M+Na)⁺.

### Example 17

### 3-[(tert-Butoxy)carbonylamino]-2,2-dimethylpropyl [3-(acetylamino)propyl]sulfonate (17)

Following the general procedure for the synthesis of acamprosate neopentyl sulfonylester prodrugs of Description 4, N-[3-(chlorosulfonyl)propyl]-acetamide (13) (ca. 1.4 g, 7.0 mmol) dissolved in 12 mL of dichloromethane was reacted with (tert-butoxy)-N-(3-hydroxy-2,2-dimethylpropyl)carboxamide (5) (610 mg, 3.0 mmol) in the presence of 650 μL of pyridine (633 mg, 8.0 mmol) and 489 mg (4.0 mmol) of DMAP. Following purification by silica gel column chromatography using mixtures of ethyl acetate (EtOAc) and methanol (MeOH) (100% EtOAc→10 vol-% MeOH/EtOAc), 201 mg (20% yield) of the title compound (17) was obtained as a pale yellow oil. R_{f}=0.42 (EtOAc/MeOH=95:5). ¹H NMR (400 MHz, DMSO-d⁶): δ=0.85 (s, 6H), 1.39 (s, 9H), 1.76-1.85 (m, 5H), 2.86 (d, J=6.4 Hz, 2H), 3.13 (d, J=6.0 Hz, 2H), 3.27-3.40 (m, 2H), 3.87 (s, 2H), 6.90 (br. t., J=6.0 Hz, 1H), 7.91 (br. t., J=4.8 Hz, 1H) ppm. MS (ESI) m/z 367.15 (M+H)⁺, 389.17 (M+Na)⁺, 365.17 (M−H).

### Example 18

### 3-Amino-2,2-dimethylpropyl [3-(acetylamino)propyl]sulfonate Hydrochloride (18)

3-[(tert-Butoxy)carbonylamino]-2,2-dimethylpropyl [3-(acetylamino)propyl]sulfonate (17) (190 mg, 0.52 mmol) was dissolved in 2 mL of dichloromethane. To this solution was added 1 mL of neat trifluoroacetic acid (TFA). The mixture was stirred at room temperature for five hours. Upon completion of the reaction, the solvent and excess acid were removed under reduced pressure using a rotary evaporator. The product was purified by mass-guided preparative HPLC. 1 mL of an aqueous 1N hydrogen chloride (HCl) solution was added to the combined fractions from the preparative HPLC purification. Following lyophilization, 105 mg (67% yield) of the title compound (18) was obtained as a colorless, brittle solid. ¹H NMR (400 MHz, DMSO-d⁶): δ=1.00 (s, 6H), 1.75-1.86 (m, 5H), 2.74 (br. q, J=6.4 Hz, 2H), 3.14 (q, J=5.6 Hz, 2H), 3.36-3.43 (m, 2H), 4.07 (s, 2H), 8.05 (br. t, J=5.6 Hz, 1H), 8.12 (br. s, 3H) ppm. MS (ESI) m/z 267.18 (M+H)⁺, 289.12 (M+Na)⁺.

### Example 19

### [N-(3-{[3-(Acetylamino)propyl]sulfonyloxy}-2,2-dimethylpropyl)carbamoyloxy]ethyl 2-methylpropanoate (19)

Following the general procedure for the synthesis of acamprosate neopentyl sulfonylester prodrugs of Description 4, N-[3-(chlorosulfonyl)propyl]-acetamide (13) (700 mg, 3.5 mmol) dissolved in 6 mL of dichloromethane was reacted with [N-(3-hydroxy-2,2-dimethylpropyl)carbamoyloxy]ethyl 2-methylpropanoate (8) (392 mg, 1.5 mmol) in the presence of 325 μL of pyridine (316 mg, 4.0 mmol) and 244 mg (2.0 mmol) of DMAP. After purification by silica gel column chromatography using mixtures of ethyl acetate (EtOAc) and methanol (MeOH) (100% EtOAc→4 vol-% MeOH/EtOAc), 102 mg (12% yield) of the title compound (19) was obtained as a pale-yellow oil. R_{f}=0.42 (EtOAc/MeOH=95:5). ¹H NMR (400 MHz, DMSO-d⁶): δ=0.86 (s, 6H), 1.059 (d, J=7.2 Hz, 3H), 1.062 (d, J=7.2 Hz, 3H), 1.40 (d, J=5.6 Hz, 3H), 1.63-1.72 (m, 1H), 1.80 (s, 3H), 2.40-2.49 (m, 2H), 2.86-2.98 (m, 2H), 3.09-3.16 (m, 2H), 3.28-3.35 (m, 2H), 3.89 (s, 2H), 6.64 (q, J=5.2 Hz, 1H), 7.54 (t, J=6.4 Hz, 1H), 7.92 (t, J=5.2 Hz, 1H) ppm. MS (ESI) m/z 425.15 (M+H)⁺, 447.23 (M+Na)⁺.

### Example 20

### 4-[(tert-Butoxy)carbonylamino]-2,2-dimethylbutyl[3-(acetylamino)propyl]sulfonate (20)

Following the general procedure for the synthesis of acamprosate neopentyl sulfonylester prodrugs of Description 4, N-[3-(chlorosulfonyl)propyl]-acetamide (13) (270 mg, 1.35 mmol) dissolved in 10 mL of dichloromethane was reacted with (tert-butoxy)-N-(4-hydroxy-3,3-dimethylbutyl)carboxamide (1) (350 mg, 1.6 mmol) in the presence of 279 μL of triethylamine (202 mg, 2.0 mmol) and 92 mg (0.75 mmol) of DMAP. Following purification by mass-guided preparative HPLC, 140 mg (23% yield) of the title compound (20) was obtained as a colorless solid. ¹H NMR (400 MHz, CDCl₃): δ=1.00 (s, 6H), 1.44 (s, 9H), 1.51-1.55 (m, 2H), 2.00 (s, 3H), 2.03-2.12 (m, 2H), 3.10-3.20 (m, 4H), 3.41 (q, J=6.4 Hz, 2H), 3.90 (s, 2H). MS (ESI) m/z 381.18 (M+H)⁺.

### Example 21

### 4-Amino-2,2-dimethylbutyl [3-(acetylamino)propyl]sulfonate Hydrochloride (21)

4-[(tert-Butoxy)carbonylamino]-2,2-dimethylbutyl[3-(acetylamino)-propyl]sulfonate (20) (270 mg, 0.7 mmol) was dissolved in 10 mL of dichloromethane. To this solution was added 10 mL of neat trifluoroacetic acid (TFA). The mixture was stirred at room temperature for several hours. Upon completion of the reaction, the solvent and excess acid were removed under reduced pressure using a rotary evaporator. The product was purified by mass-guided preparative HPLC. 1 mL of an aqueous 1N hydrogen chloride (HCl) solution was added to the combined fractions from the preparative HPLC purification. Following lyophilization, 200 mg (90% yield) of the title compound (21) was obtained as a colorless solid. ¹H NMR (400 MHz, D₂O): δ=0.96 (s, 6H), 1.62-1.67 (m, 2H), 1.90-2.02 (m, 2H), 2.98-3.02 (m, 2H), 3.25-3.28 (m, 2H), 3.34-3.38 (m, 2H), 4.00 (s, 2H). MS (ESI) m/z 281.20 (M+H)⁺.

### Example 22

### [N-(4-{[3-(Acetylamino)propyl]sulfonyloxy}-3,3-dimethyl-butyl)carbamoyloxy]ethyl 2-methylpropanoate (22)

Following the general procedure for the synthesis of acamprosate neopentyl sulfonylester prodrugs of Description 4, N-[3-(chlorosulfonyl)propyl]acetamide (13) (2.6 g, 13 mmol) dissolved in 30 mL of dichloromethane was reacted with [N-(4-hydroxy-3,3-dimethylbutyl)carbamoyloxy]ethyl 2-methylpropanoate (9) (2.35 g, 8.5 mmol) in the presence of 2.37 mL of triethylamine (1.72 g, 17.0 mmol) and 553 mg (4.54 mmol) of DMAP. After isolation and purification by silica gel column chromatography using mixtures of ethyl acetate (EtOAc) and methanol (MeOH) as eluent (1 vol-% MeOH/EtOAc→9 vol-% MeOH/EtOAc), 1.11 g (30% yield) of the title compound (22) was obtained as colorless, viscous liquid. R_{f}=0.50 (EtOAc/MeOH=9:1). ¹H NMR (400 MHz, CDCl₃): δ=1.01 (s, 6H), 1.17 (d, J=7.2 Hz, 6H), 1.46 (d, J=5.2 Hz, 3H), 1.55-1.59 (m, 2H), 2.01 (s, 3H), 2.05-2.13 (m, 2H), 2.54 (heptet, J=7.2 Hz, 1H), 3.17-3.24 (m, 4H), 3.38-3.44 (m, 2H), 3.91 (s, 2H), 4.98 (br. m, 1H), 6.20 (br. s, 1H), 6.79 (q, J=5.2 Hz, 1H) ppm. MS (ESI) m/z 439.16 (M+H)⁺, 461.14 (M+Na)⁺.

### Example 23

### [N-(4-{[3-(Acetylamino)propyl]sulfonyloxy}-3,3-dimethylbutyl)-carbamoyloxy]ethyl Benzoate (23)

Following the general procedure for the synthesis of acamprosate neopentyl sulfonylester prodrugs of Description 4, N-[3-(chlorosulfonyl)propyl]acetamide (13) (900 mg, 4.5 mmol) dissolved in 20 mL of dichloromethane was reacted with [N-(4-hydroxy-3,3-dimethylbutyl)carbamoyloxy]ethyl benzoate (10) (1.33 g, 4.3 mmol) in the presence of 697 μL of triethylamine (506 mg, 5.0 mmol) and 61 mg (0.4 mmol) of DMAP. After isolation and purification using mass-guided preparative HPLC, 500 mg (25% yield) of the title compound (23) was obtained as colorless, viscous liquid. R_{f}=0.58 (EtOAc/MeOH=9:1). ¹H NMR (400 MHz, CDCl₃): δ=1.00 (s, 6H), 1.55-1.60 (m, 2H), 1.61 (d, J=5.6 Hz, 3H), 1.99 (s, 3H), 2.03-2.11 (m, 2H), 3.15-3.22 (m, 4H), 3.32-3.42 (m, 2H), 3.91 (s, 2H), 4.98 (t, J=5.6 Hz, 1H), 6.19 (br. s, 1H), 7.06 (q, J=5.2 Hz, 1H), 7.42-7.46 (m, 2H), 7.55-7.59 (m, 1H), 8.01-8.03 (m, 2H) ppm. MS (ESI) m/z 473.16 (M+H)⁺.

### Example 24

### 5-[(tert-Butoxy)carbonylamino]-2,2-dimethylpentyl [3-(acetylamino)propyl]sulfonate (24)

Following the general procedure for the synthesis of acamprosate neopentyl sulfonylester prodrugs of Description 4, N-[3-(chlorosulfonyl)propyl]-acetamide (13) (600 mg, 3.0 mmol) dissolved in 10 mL of dichloromethane was reacted with (tert-butoxy)-N-(5-hydroxy-4,4-dimethylpentyl)carboxamide (6) (462 mg, 2.0 mmol) in the presence of 558 μL of triethylamine (405 mg, 4.0 mmol) and 122 mg (1.0 mmol) of DMAP. After isolation and purification using mass-guided preparative HPLC followed by an additional purification by silica gel column chromatography using mixtures of ethyl acetate (EtOAc), hexane (Hxn), and methanol (MeOH) as eluent (EtOAc/hexane=2:1→100% EtOAc→5 vol-% MeOH/EtOAc), 142 mg (18% yield) of the title compound (24) was obtained as colorless, viscous liquid. R_{f}=0.55 (EtOAc/MeOH=9:1). ¹H NMR (400 MHz, CDCl₃): δ=0.96 (s, 6H), 1.28-1.34 (m, 2H), 1.40-1.52 (m, 1H), 2.01 (s, 3H), 2.04-2.12 (br. m, 2H), 3.10 (q, J=6.4 Hz, 2H), 3.14-3.20 (m, 2H), 3.41 (q, J=6.8 Hz, 2H), 3.89 (s, 2H), 4.76 (br. m, 1H), 6.18 (br. m, 1H). MS (ESI) m/z 395.20 (M+H)⁺, 417.23 (M+Na)⁺.

### Example 25

### 5-Amino-2,2-dimethylpentyl [3-(acetylamino)propyl]sulfonate Hydrochloride (25)

5-[(tert-Butoxy)carbonylamino]-2,2-dimethylpentyl [3-(acetylamino)-propyl]sulfonate (24) (120 mg, 0.304 mmol) was dissolved in 2.0 mL of dichloromethane. To this solution was added 1.0 mL of neat trifluoroacetic acid. The mixture was stirred at room temperature for four hours. Upon completion of the reaction, the solvent and excess acid were removed under reduced pressure using a rotary evaporator. The product was purified by mass-guided preparative HPLC. 1 mL of an aqueous 1N HCl solution was added to the combined fractions from the preparative HPLC purification. Following lyophilization, 83 mg (83% yield) of the title compound (25) was obtained as a colorless, waxy solid. ¹H NMR (400 MHz, DMSO-d⁶): δ=0.89 (s, 6H), 1.24-1.32 (m, 2H), 1.48-1.58 (m, 2H), 1.75-1.84 (m, 5H), 2.67-2.77 (m, 2H), 3.13 (q, J=5.6 Hz, 2H), 3.33-3.39 (m, 2H), 3.87 (s, 2H), 8.06 (br. s, 3H), 8.15 (br. t, J=6.0 Hz, 1H) ppm. MS (ESI) m/z 294.81 (M+H)⁺.

### Example 26

### [N-(5-{[3-(Acetylamino)propyl]sulfonyloxy}-4,4-dimethylpentyl)carbamoyloxy]ethyl 2-methylpropanoate (26)

Following the general procedure for the synthesis of acamprosate neopentyl sulfonylester prodrugs of Description 4, N-[3-(chlorosulfonyl)propyl]-acetamide (13) (ca. 600 mg, 3.0 mmol) dissolved in 10 mL of dichloromethane was reacted with [N-(5-hydroxy-4,4-dimethylpentyl)carbamoyloxy]ethyl 2-methylpropanoate (11) (579 mg, 2.0 mmol) in the presence of 558 μL of triethylamine (405 mg, 4.0 mmol) and 122 mg (1.0 mmol) of DMAP. After purification by mass-guided preparative HPLC followed by silica gel column chromatography using mixtures of ethyl acetate (EtOAc), hexane (Hxn), and methanol (MeOH) as eluent (EtOAc/Hxn=2:1→100% EtOAc→5 vol-% MeOH/EtOAc), 100 mg (11% yield) of the title compound (26) was obtained as a colorless, viscous oil. R_{f}=0.65 (EtOAc/MeOH=9:1). ¹H NMR (400 MHz, CDCl₃): δ=0.96 (s, 6H), 1.17 (d, J=6.4 Hz, 6H), 1.29-1.35 (m, 2H), 1.47-1.54 (m, 5H), 2.01 (s, 3H), 2.03-2.13 (m, 2H), 2.54 (heptet, J=7.2 Hz, 1H), 3.13-3.21 (m, 4H), 3.41 (q, J=6.4 Hz, 2H), 3.89 (s, 2H), 5.13 (br. t., J=6.0 Hz, 1H), 6.08-6.18 (br. t, 1H), 6.79 (q, J=5.6 Hz, 1H) ppm. MS (ESI) m/z 453.19 (M+H)⁺, 475.15 (M+Na)⁺.

### Example 27

### [N-(5-{[3-(Acetylamino)propyl]sulfonyloxy}-4,4-dimethylpentyl)carbamoyloxy]methyl Benzoate (27)

Following the general procedure for the synthesis of acamprosate neopentyl sulfonylester prodrugs of Description 4, N-[3-(chlorosulfonyl)propyl]-acetamide (13) (ca. 400 mg, 2.0 mmol) dissolved in 8 mL of dichloromethane was reacted with [N-(5-hydroxy-4,4-dimethylpentyl)carbamoyloxy]methyl benzoate (12) (390 mg, 1.26 mmol) in the presence of 418 μL of triethylamine (304 mg, 3.0 mmol) and 100 mg (0.82 mmol) of DMAP. After purification by mass-guided preparative HPLC followed by silica gel column chromatography using mixtures of ethyl acetate (EtOAc), hexane (hxn), and methanol (MeOH) as eluent (EtOAc/Hxn=2:1→100% EtOAc→10 vol-% MeOH/EtOAc), 204 mg (34% yield) of the title compound (27) was obtained as a colorless, viscous oil. R_{f}=0.66 (EtOAc/MeOH=9:1). ¹H NMR (400 MHz, CDCl₃): δ=0.95 (s, 6H), 1.29-1.36 (m, 2H), 1.45-1.56 (m, 2H), 2.01 (s, 3H), 2.05-2.11 (m, 2H), 3.12-3.24 (m, 4H), 3.39 (q, J=6.4 Hz, 2H), 3.88 (s, 2H), 5.44 (br. t., J=5.6 Hz, 1H), 5.97 (s, 2H), 6.08 (br. t., J=6.4 Hz, 1H), 7.41-7.48 (m, 2H), 7.56-7.62 (m, 1H), 8.04-8.10 (m, 2H) ppm. MS (ESI) m/z 473.25 (M+H)⁺, 495.15 (M+H)⁺.

### Example 28

### 2-Hydroxy-2-methylpropyl [3-(acetylamino)propyl]sulfonate (28)

### Step A: Phenylmethyl 2-methyl-2-(phenylmethoxy)propanoate (28a)

1.56 g (15 mmol) of 2-hydroxy-2-methylpropanoic acid was dissolved in 150 mL of anhydrous DMF. To the stirred solution was added sodium hydride (0.79 g, 33 mmol) and benzyl bromide (5.6 g, 3.9 mL, 33 mmol). The reaction was monitored by thin layer chromatography (TLC). After the starting material was completely consumed, the reaction was quenched by addition of a 1N HCl solution and then extracted with diethyl ether (twice). The combined organic extracts were washed with water and brine, dried over magnesium sulfate, filtered, and the solvents removed under reduced pressure using a rotary evaporator. The residue was purified by silica gel chromatography using a mixture of ethyl acetate/hexane (1:9) as eluent to provide 4.0 g (94% yield) of the title compound (28a). R_{f}=0.78 (EtOAc/Hxn=1:4). ¹H NMR (400 MHz, CHCl₃): δ=1.56 (s, 6H), 4.46 (s, 2H), 5.22 (s, 2H), 7.28-7.38 (m, 10H) ppm.

### Step B: 2-Methyl-2-(phenylmethoxy)propan-1-ol (28b)

To a solution of phenylmethyl 2-methyl-2-(phenylmethoxy)propanoate (28a) (4.0 g, 14 mmol) in 100 mL THF, LAH solution (1M in THF, 20 mL) was added slowly at −78° C. The mixture was stirred overnight. The reaction was quenched with 1.2 mL of water, 2.4 mL of 10% NaOH aqueous solution and 1.2 mL of water successively. The white precipitate was filtered off and the solvent was removed under reduced pressure. The residue was purified by silica gel chromatography using a mixture of ethyl acetate/hexane (1:2) as eluent to provide 1.7 g (40% yield) of the title compound (28b). R_{f}=0.31 (EtOAc/Hxn=1:4). ¹H NMR (400 MHz, CHCl₃): δ=1.30 (s, 6H), 2.09 (t, J=6.0 Hz, 1H), 3.58 (d, J=6.0 Hz, 2H), 4.46 (s, 2H), 7.28-7.37 (m, 10H) ppm.

### Step C: 2-Methyl-2-(phenylmethoxy)propyl [3-(acetylamino)propyl]sulfonate (28c)

Following the general procedure for the synthesis of acamprosate neopentyl sulfonylester prodrugs of Description 4, N-[3-(chlorosulfonyl)propyl]acetamide (13) (ca. 1.9 g, 15 mmol) dissolved in 100 mL of dichloromethane was reacted with 2-methyl-2-(phenylmethoxy)propan-1-ol (35) (900 mg, 5.0 mmol) in the presence of 2.1 mL of triethylamine (1.51 g, 15 mmol) and 180 mg (1.5 mmol) of DMAP. The reaction was quenched by addition of a 1N HCl solution and then extracted with DCM. The combined organic extracts were washed with water and brine, dried over magnesium sulfate, filtered, and the solvents removed under reduced pressure using a rotary evaporator. The residue was purified by silica gel chromatography using a mixture of ethyl acetate/methanol (4:1) as eluent to provide 0.15 g (8.7% yield) of the title compound (28c). R_{f}=0.33 (EtOAc). ¹H NMR (400 MHz, CHCl₃): δ=1.34 (s, 6H), 1.85 (s, 3H), 1.92-1.97 (m, 2H), 3.11 (dd, J=7.2 Hz, 2H), 3.19 (q, J=6.0 Hz, 2H), 4.12 (s, 2H), 4.45 (s, 2H), 5.96 (br. m, 1H), 7.24-7.34 (m, 5H) ppm. MS (ESI) m/z 343.96 (M+1)⁺.

### Step D: 2-Hydroxy-2-methylpropyl [3-(acetylamino)propyl]sulfonate (28)

A high-pressure reaction vessel was charged with 2-methyl-2-(phenylmethoxy)propyl [3-(acetylamino)propyl]sulfonate (28c) (0.15 g, 0.44 mmol), 10% Pd—C (70 mg) and 4 mL of ethanol. The mixture was subjected to hydrogenolysis for over 8 hrs. The residue was purified by silica gel chromatography using a mixture of ethyl acetate/methanol (4:1) as eluent to provide 0.9 g (8.7% yield) of the title compound (28). ¹H NMR (400 MHz, CHCl₃): δ=1.27 (s, 6H), 1.96 (s, 3H), 2.02-2.09 (m, 2H), 3.22 (dd, J=7.2 Hz, 2H), 3.35 (q, J=6.8 Hz, 2H), 4.02 (s, 2H), 6.63 (br. m, 1H) ppm. MS (ESI) m/z 253.98 (M+1)⁺.

### Example 29

### 3-{[3-(Acetylamino)propyl]sulfonyloxy}-2,2-dimethylpropyl 2-methylpropanoate (29)

### Step A: 3-Hydroxy-2,2-dimethylpropyl 2-methylpropanoate (29a)

In a 250 mL round bottomed flask equipped with a magnetic stirring bar, 1.06 mL of isobutyryl chloride (1.07 g, 10.0 mmol), 1.68 mL of pyridine (1.58 g, 3.1 mmol), and 244 mg (2.0 mmol) of 4-(N,N-dimethylamino) pyridine (DMAP) were added to a stirred solution of 3.13 g (30.0 mmol) of commercially available neopentyl glycol (2,2-dimethyl 1,3-propandiol) in 60 mL of anhydrous dichloromethane (DCM) at ca. 0° C. (ice bath). The reaction mixture was stirred overnight with gradual warming to room temperature. After the starting material was completely consumed, the reaction was quenched by the addition of a one normal (1 N) aqueous solution of hydrogen chloride (HCl), and the reaction mixture then extracted twice with DCM. The combined organic extracts were washed with a saturated aqueous solution of sodium hydrogencarbonate (NaHCO₃) and brine, dried over anhydrous magnesium sulfate (MgSO₄), filtered, and the solvents removed under reduced pressure using a rotary evaporator to provide ca. 1.50 g (86% yield) of the title compound (329a) as a colorless oil. The material obtained was of sufficient purity to be used in the next step without further purification of isolation. ¹H NMR (400 MHz, CDCl₃): δ=0.94 (s, 6H), 1.20 (d, J=6.8 Hz, 6H), 2.16-2.20 (br. m, 1H), 2.60 (heptet, J=7.2 Hz, 1H), 3.27-3.29 (m, 2H), 3.94 (s, 2H) ppm.

### Step B: 3-{[3-(acetylamino)propyl]sulfonyloxy}-2,2-dimethylpropyl 2-methylpropanoate (29)

Following the general procedure for the synthesis of acamprosate neopentyl sulfonylester prodrugs of Description 4, N-[3-(chlorosulfonyl)propyl]acetamide (13) (ca. 700 mg, 3.5 mmol) dissolved in 6 mL of dichloromethane (DCM) was reacted with 3-hydroxy-2,2-dimethylpropyl 2-methylpropanoate (29a) (261 mg, 1.5 mmol) in the presence of 325 μL of pyridine (316 mg, 4.0 mmol) and 244 mg (2.0 mmol) of DMAP. After aqueous work-up and purification by mass-guided preparative HPLC, 39 mg (7.7%) of the title compound (29) was obtained as a colorless, viscous oil. R_{f}=0.48 (EtOAc/MeOH=95:5). ¹H NMR (400 MHz, DMSO-d⁶): δ=0.94 (s, 6H), 1.11 (d, J=6.8 Hz, 6H), 1.74-1.84 (m, 5H), 2.56 (heptet, J=6.4 Hz, 1H), 3.10-3.16 (m, 2H), 3.30-3.36 (m, 2H), 3.83 (s, 2H), 3.87 (s, 2H), 7.91 (br. t, J=5.6 Hz, 1H) ppm. MS (ESI) m/z 338.16 (M+H)⁺, 360.18 (M+Na)⁺.

### Example 30

### 3-{[3-(Acetylamino)propyl]sulfonyloxy}-2,2-dimethylpropyl Benzoate (30)

### Step A: 3-Hydroxy-2,2-dimethylpropyl benzoate (30a)

To a stirred solution of 3.13 g (30.0 mmol) of commercially available neopentyl glycol (2,2-dimethyl 1,3-propandiol) in 60 mL of anhydrous dichloromethane (DCM) in a 250 mL round bottomed flask equipped with a magnetic stirring bar was added at ca. 0° C. (ice bath) 1.16 mL of benzoyl chloride (1.41 g, 10.0 mmol), 1.68 mL of pyridine (1.58 g, 3.1 mmol), and 244 mg (2.0 mmol) of 4-(N,N-dimethylamino) pyridine (DMAP). The reaction mixture was stirred overnight with gradual warming to room temperature. After the starting material was completely consumed, the reaction was quenched by the addition of a one normal (1 N) aqueous solution of hydrogen chloride (HCl), and the reaction mixture then extracted twice with DCM. The combined organic extracts were washed with a saturated aqueous solution of sodium hydrogencarbonate (NaHCO₃) and brine, dried over anhydrous magnesium sulfate (MgSO₄), filtered, and the solvents removed under reduced pressure using a rotary evaporator to yield ca. 2.3 g (quant.) of the title compound (30a) as a colorless oil. The material obtained was of sufficient purity to be used in the next step without further purification of isolation. R_{f}=0.19 (EtOAc/Hxn=1:6). ¹H NMR (400 MHz, CDCl₃): δ=1.04 (s, 6H), 2.05-2.15 (br. m, 1H), 3.39-3.41 (m, 2H), 4.20 (s, 2H) ppm. (ESI) m/z 209.17 (M+H)⁺, 230.90 (M+Na)⁺.

### Step B: 3-{[3-(Acetylamino)propyl]sulfonyloxy}-2,2-dimethylpropyl benzoate (30)

Following the general procedure for the synthesis of acamprosate neopentyl sulfonylester prodrugs of Description 4, N-[3-(chlorosulfonyl)propyl]acetamide (13) (ca. 700 mg, 3.5 mmol) dissolved in 6 mL of dichloromethane (DCM) was reacted with 3-hydroxy-2,2-dimethylpropyl benzoate (30a) (412 mg, 1.5 mmol) in the presence of 558 μL of triethylamine (405 mg, 4.0 mmol) and 244 mg (2.0 mmol) of DMAP. After aqueous work-up and purification by silica gel column chromatography using mixtures of ethylacetate (EtOAc), hexane (Hxn), and methanol (MeOH) as eluent (EtOAc/Hxn=4:1→EtOAc/Hxn=9:1→100% EtOA EtOAc/MeOH=95:5), 321 mg (70%) of the title compound (31) was obtained as an almost colorless, viscous oil. R_{f}=0.32 (EtOAc). ¹H NMR (400 MHz, DMSO-d⁶): δ=1.05 (s, 6H), 1.74-1.83 (m, 5H), 3.06-3.13 (m, 2H), 3.31-3.37 (m, 2H), 4.09 (s, 2H), 4.10 (s, 2H), 7.50-7.56 (m, 2H), 7.63-7.69 (m, 1H), 7.88 (br. t, J=5.2 Hz, 1H), 7.97-8.02 (m, 2H) ppm. MS (ESI) m/z 372.17 (M+H)⁺, 394.13 (M+Na)⁺, 370.20 (M−H)⁻.

### Example 31

### 3-Hydroxy-2,2-dimethylpropyl [3-(acetylamino)propyl]-sulfonate (31)

### Step A: 3-Phenylmethoxy-neopentylol (31a)

Adapting a procedure or a variation thereof according to Effenberger, et al., Tetrahedron: Asymmetry 1995, 6, 271-282, a 1000 mL round-bottomed flask equipped with a magnetic stirring bar and a rubber septum was charged under a nitrogen atmosphere with 2.40 g of a 60 wt-% suspension of sodium hydride (NaH) in mineral oil (1.44 g, 60.0 mmol). The hydride was suspended in 50 mL of hexane and the supernatant was decanted, and the residue was dried under reduced pressure. Six-hundred (600) mL of anhydrous tetrahydrofuran (THF) were added under a nitrogen atmosphere and the suspension was cooled to ca. 0° C. (ice bath). 6.24 g (60 mmol) of commercially available neopentyl glycol (2,2-dimethyl-1,3-propandiol) was added and the reaction mixture was stirred for one hour at this temperature until the hydrogen evolution subsided. 5.9 mL of benzyl bromide (8.5 g, 50.0 mmol) was added to the stirred reaction mixture. The reaction mixture was stirred overnight with gradual warming to room temperature and the solvent was then partially removed under reduced pressure using a rotary evaporator. The reaction was quenched by addition of a one normal (1 N) aqueous solution of hydrogen chloride (HCl) and the product was extracted with ethyl acetate. The combined organic extracts were washed with water and brine, dried over anhydrous magnesium sulfate (MgSO₄), filtered, and the solvents were removed under reduced pressure using a rotary evaporator. The residue was purified by silica gel chromatography using a mixture of ethyl acetate (EtOAc) and hexane (Hxn) as eluent (EtOAc/Hxn=1:4) to provide 6.0 g (62% yield) of the title compound (31a) as a colorless liquid. R_{f}=0.34 (EtOAc/Hxn=1:4). ¹H NMR (400 MHz, CDCl₃): δ=0.953 (s, 6H), 2.62 (br. t, J=5.6 Hz, 1H), 3.34 (s, 2H), 3.47 (d, J=5.6 Hz, 2H), 4.54 (s, 2H), 7.27-7.38 (m, 5H) ppm. MS (ESI) m/z 195.10 (M+H)⁺, 217.10 (M+Na)⁺. The analytical data was consistent with the data given in the literature.

### Step B: 2,2-Dimethyl-3-(phenylmethoxy)propyl [3-(acetylamino)propyl]-sulfonate (31b)

Following the general procedure for the synthesis of acamprosate neopentyl sulfonylester prodrugs of Description 4, N-[3-(chlorosulfonyl)propyl]acetamide (13) (ca. 3.66 g, ca. 50% purity, ca. 10.0 mmol) dissolved in 40 mL of dichloromethane (DCM) was reacted with 3-phenylmethoxy-neopentylol (31a) (3.88 g, 20.0 mmol) in the presence of 2.79 mL of triethylamine (2.02 g, 20.0 mmol) and 2.44 g (20.0 mmol) of DMAP. After aqueous work-up and purification by silica gel column chromatography using mixtures of ethyl acetate (EtOAc) and hexane (Hxn) as eluent (EtOAc/Hxn=2:1→EtOAc/Hxn=3:1→EtOAc/Hxn=4:1→EtOAc/Hxn=6:1→EtOAc/Hxn=9:1), 2.21 g (62% yield) of the title compound (31b) was obtained as an almost colorless, viscous oil. R_{f}=0.35 (EtOAc). ¹H NMR (400 MHz, CDCl₃): δ=1.01 (s, 6H), 1.96 (s, 3H), 1.99-2.07 (m, 2H), 3.09-3.14 (m, 2H), 3.26 (s, 2H), 3.35 (q, J=6.4 Hz, 2H), 4.05 (s, 2H), 4.51 (s, 2H), 5.66-5.72 (br. m, 1H), 7.28-7.38 (m, 5H) ppm. MS (ESI) m/z 358.11 (M+H)⁺, 380.06 (M+Na)⁺, 355.95 (M−H)⁻.

### Step C: 3-Hydroxy-2,2-dimethylpropyl [3-(acetylamino)propyl]sulfonate (31)

Caution: The use of a safety shield and other appropriate safety measures for this reaction are highly recommended.

A thick-walled high-pressure reaction vessel was charged with 1.14 g (3.18 mmol) of 2,2-dimethyl-3-(phenylmethoxy)propyl [3-(acetylamino)propyl]sulfonate (31b), 800 mg of 10 wt-% of palladium on activated carbon (10% Pd/C-catalyst) and 15 mL of anhydrous methanol (MeOH). The atmosphere in the reaction vessel was exchanged to hydrogen by three evacuation and refill cycles using a Parr hydrogenation apparatus. The hydrogenolytic cleavage of the benzyl group was carried out overnight at room temperature at a pressure of ca. 50 psi. After the starting material was completely consumed, the catalyst was filtered off through a short plug of Celite® and the filter plug was washed with anhydrous methanol (MeOH). The solvent was partially removed under reduced pressure using a rotary evaporator, and the solution was additionally filtered through a 0.2 μM nylon syringe filter to remove residual Celite® and catalyst particles before final evaporation under reduced pressure using a rotary evaporator. The title compound (31) thus obtained was of sufficient purity to be used without further purification or isolation. R_{f}=0.38 (EtOAc/MeOH=9:1). ¹H NMR (400 MHz, DMSO-d⁶): δ=0.86 (s, 6H), 1.74-1.85 (m, 5H), 3.09-3.16 (m, 2H), 3.17 (d, J=5.2 Hz, 2H), 3.27-3.34 (m, 2H), 3.92 (s, 2H), 4.76 (t, J=5.6 Hz, 1H), 7.91 (br. t, J=5.6 Hz, 1H) ppm. MS (ESI) m/z 268.13 (M+H)⁺, 290.07 (M+Na)⁺, 266.10 (M−H)⁻.

### Alternative and One-Step Synthesis of 3-Hydroxy-2,2-dimethylpropyl [3-(acetylamino)propyl]sulfonate (31)

Following the general procedure for the synthesis of acamprosate neopentyl sulfonylester prodrugs of Description 4, N-[3-(chlorosulfonyl)propyl]acetamide (13) (ca. 2.0 mg, 10.0 mmol) dissolved in 50 mL of dichloromethane (DCM) was reacted with commercially available neopentyl glycol (2,2-dimethylpropan-1,3-diol) (10.42 g, 100 mmol) in the presence of 1.67 mL of triethylamine (1.21 g, 12.0 mmol) and 1.47 g (12.0 mmol) of DMAP. After aqueous work-up and purification by silica gel column chromatography using mixtures of ethyl acetate (EtOAc) and methanol (MeOH) as eluent (100% EtOAc→EtOAc/MeOH=95:5→EtOAc/MeOH=9:1→EtOAc/MeOH=85:15), 348 mg (13% yield) of the title compound (31) was obtained as an almost colorless, viscous oil. The analytical data was consistent with the data for the material obtained using a mono-protected neopentyl glycol derivative.

### Example 32

### 4-{[3-(Acetylamino)propyl]sulfonyloxy}-3,3-dimethylbutyl Benzoate (32)

### Step A: 2,2-Dimethyl-pent-4-en-1-ol (32a)

Adapting a procedure or a variation thereof according to Chen, et al., J. Am. Chem. Soc, 2003, 125, 6697-6704, an oven dried 500 mL round bottomed flask equipped with a magnetic stirring bar and a pressure equalized addition funnel was charged under a nitrogen atmosphere with 5.00 g (39.0 mmol) of commercially available 2,2-dimethyl-4-pentenoic acid. The acid was dissolved in 100 mL of anhydrous tetrahydrofuran (THF) and cooled to ca. 0° C. (ice bath). Forty (40) mL of a one molar (1 M) solution of lithium aluminum hydride (LAH) in THF was slowly added at this temperature and the reaction mixture was stirred overnight with slow warming to room temperature followed by subsequent cooling to ca. 0° C. (ice bath). Subsequent and careful addition of 2.6 mL of water, 5.2 mL of a 10 wt-% aqueous solution of sodium hydroxide (NaOH), and 2.6 mL of water resulted in a colorless precipitate that was filtered off. The filter residue was washed with ethyl acetate (EtOAc) and the combined filtrates were dried over anhydrous magnesium sulfate (MgSO₄), filtered, and the solvents were evaporated under reduced pressure using a rotary evaporator to provide 4.05 g (91% yield) of the title compound (32a) as a colorless oil. R_{f}=0.30 (EtOAc/Hxn=1:9). ¹H NMR (400 MHz, CDCl₃): δ=0.91 (s, 6H), 2.04 (d, J=8.0 Hz, 2H), 3.34 (d, J=4.8 Hz, 2H), 5.01-5.10 (m, 2H), 5.79-5.91 (m, 1H) ppm. MS (ESI) m/z 114.86 (M+H)⁺. The analytical data was consistent with the proposed structure and with the data reported in the literature.

### Step B: 1-(2,2-Dimethylpent-4-enyloxy)-1,1,2,2-tetramethyl-1-silapropane (32b)

Adapting a procedure or a variation thereof according to Chen, et al., J. Am. Chem. Soc, 2003, 125, 6697-6704, 2.2 g (19.3 mmol) of 2,2-dimethylpent-4-en-1-ol (32a) was dissolved in 100 mL of dichloromethane (DCM) under a nitrogen atmosphere in a 250 mL round bottomed flask equipped with a magnetic stirring bar. The solution was cooled to ca. 0° C. (ice bath) and 3.2 g (21.2 mmol) of solid tert-butyldimethyl chlorosilane (TBDMSCI) and 1.44 g (21.2 mmol) of solid imidazole was added to the stirred solution. The reaction mixture was stirred at this temperature, and the reaction monitored by thin layer chromatography. Upon the completion of the reaction, the colorless precipitate was filtered off and the solvent was removed under reduced pressure using a rotary evaporator. The residue was purified by silica gel chromatography using hexane as eluent to provide 3.1 g (71% yield) of the title compound (32b) as a colorless oil. R_{f}=0.89 (EtOAc/Hxn=1:13). ¹H NMR (400 MHz, CDCl₃): δ=0.05 (s, 6H), 0.85 (s, 6H), 0.92 (s, 9H), 2.00 (d, J=7.6 Hz, 2H), 3.24 (s, 2H), 4.98-5.03 (m, 2H), 5.76-5.86 (m, 1H) ppm. The analytical data was consistent with the proposed structure and with the data reported in the literature.

### Step C: rac-4,4-Dimethyl-5-(1,1,2,2-tetramethyl-1-silapropoxy)pentane-1,2-diol (32c)

Adapting a procedure or a variation thereof according to Pappo, et al., J. Org. Chem. 1956, 21, 478-479, a solution of 0.5 g (19.3 mmol) of 1-(2,2-dimethylpent-4-enyloxy)-1,1,2,2-tetramethyl-1-silapropane (32b) in 10 mL of a mixture of water/acetone (1:1 v/v) was reacted with 2.7 mL of a 2.5 wt-% solution of osmium tetroxide (OsO₄) in tert-butanol in the presence of 0.45 g (3.3 mmol) N-methyl morpholine oxide (NMO). The reaction was monitored by thin layer chromatography. After the starting material was completely consumed, the reaction was quenched by addition of a 10 wt-% aqueous solution of sodium hydrogensulfite (NaHSO₃) and the product was then extracted twice with ethyl acetate (EtOAc). The combined organic extracts were washed with water and brine, dried over anhydrous magnesium sulfate (MgSO₄), filtered, and the solvents were removed under reduced pressure using a rotary evaporator. The residue was purified by silica gel chromatography using ethyl acetate (EtOAc) and hexane (Hxn) as eluent (EtOAc/Hxn=1:2) to provide 400 mg (70% yield) of the title compound (32c) as a colorless oil. R_{f}=0.51 (EtOAc/Hxn=1:1). ¹H NMR (400 MHz, CDCl₃): δ=0.10 (s, 6H), 0.88 (s, 3H), 0.92 (s, 9H), 0.95 (s, 3H), 1.24-1.51 (m, 2H), 3.40 (s, 2H), 3.43-3.55 (br. m, 2H), 3.78-3.83 (br. m, 1H) ppm. MS (ESI) m/z 263.16 (M+H)⁺. Closely related diols [O-(tert-butyldiphenylsilyl) (TBDPS) or O-benzyl (Bn) protecting group instead of O-(tert-butyldimethylsilyl) protecting group)] are disclosed in Trudeau, et al., J. Org. Chem. 2005, 70, 9538-9544.

### Step D: 3,3-Dimethyl-4-(1,1,2,2-tetramethyl-1-silapropoxy)butan-1-ol (32d)

Adapting a procedure or a variation thereof according to Marschner, et al., J. Org. Chem. 1995, 60, 5224-5235, to a stirred solution of 2.5 g (9.5 mmol) of rac-4,4-dimethyl-5-(1,1,2,2-tetramethyl-1-silapropoxy)pentane-1,2-diol (33c) in 50 mL of a mixture of water and ethanol (1:1 v/v) in a 100 mL round-bottomed flask equipped with a magnetic stirring bar was added 4.0 g, (18.7 mmol) of sodium meta-periodate (NaIO₄). The reaction was monitored by thin layer chromatography. After the starting material was completely consumed, the reaction was quenched by addition of a 10 wt-% aqueous solution of sodium thiosulfate (Na₂S₂O₃) and the reaction mixture was extracted with ethyl acetate (EtOAc). The combined organic extracts were washed with water and brine, dried over anhydrous magnesium sulfate (MgSO₄), filtered, and the solvents removed under reduced pressure using a rotary evaporator. The crude residue used directly in the next step without further isolation and characterization procedures and was dissolved in 100 mL of methanol. 0.33 g (8.7 mmol) of solid sodium borohydride (NaBH₄) was added. Upon completion of the reaction, the mixture was quenched by addition of a one normal (1 N) aqueous solution of hydrogen chloride (HCl) and the reaction mixture was twice extracted with dichloromethane (DCM). The combined organic extracts were washed with brine, dried over anhydrous magnesium sulfate (MgSO₄), filtered, and the solvents were removed under reduced pressure using a rotary evaporator. The residue was purified by silica gel chromatography using ethyl acetate (EtOAc) and hexane (Hxn) as eluent (EtOAc/Hxn=1:4) to provide 1.8 g (82% yield over two steps) of the title compound (32d) as a colorless oil. R_{f}=0.67 (EtOAc/Hxn=1:2). ¹H NMR (400 MHz, CDCl₃): δ=0.09 (s, 6H), 0.90 (s, 6H), 0.92 (s, 9H), 1.56 (t, J=6.0 Hz, 2H), 3.35 (s, 2H), 3.65 (t, J=6.0 Hz, 2H) ppm.

### Step E: 3,3-Dimethyl-4-(1,1,2,2-tetramethyl-1-silapropoxy)butyl benzoate (32e)

To a stirred solution of 0.6 g (2.6 mmol) of 3,3-dimethyl-4-(1,1,2,2-tetramethyl-1-silapropoxy)butan-1-ol (32d) in 20 mL of anhydrous dichloromethane (DCM) in a 100 mL round bottomed flask equipped with a magnetic stirring bar was added 360 μL of benzoyl chloride (436 mg, 3.1 mmol), 432 μL of triethylamine (314 mg, 3.1 mmol) and 38 mg (0.31 mmol) of 4-(N,N-dimethylamino) pyridine (DMAP). The reaction mixture was stirred overnight at room temperature. After the starting material was completely consumed, the reaction was quenched by addition of a one normal (1 N) aqueous solution of hydrogen chloride (HCl), and the reaction mixture then extracted twice with diethyl ether. The combined organic extracts were washed with a saturated aqueous solution of sodium hydrogencarbonate (NaHCO₃) and brine, dried over anhydrous magnesium sulfate (MgSO₄), filtered, and the solvents removed under reduced pressure using a rotary evaporator. The residue was purified by silica gel chromatography using a mixture of ethyl acetate (EtOAc) and hexane (Hxn) as eluent (EtOAc/Hxn=1:8) to provide 0.7 g (94% yield) of the title compound (32e) as a colorless oil. R_{f}=0.84 (EtOAc/Hxn=1:4). ¹H NMR (400 MHz, CDCl₃): δ=0.06 (s, 6H), 0.92 (s, 9H), 0.96 (s, 6H), 1.77 (t, J=7.2 Hz, 2H), 3.32 (s, 2H), 4.40 (t, J=7.2 Hz, 2H), 7.41-7.46 (m, 2H), 7.53-7.57 (m, 1H), 8.02-8.04 (m, 2H) ppm.

### Step F: 4-Hydroxy-3,3-dimethylbutyl benzoate (32f)

Adapting a procedure, or a variation thereof, according to Pirrung, et al., Bioorg. Med. Chem. Lett. 1994, 4, 1345-1346, a solution of 0.7 g (2.3 mmol) of 3,3-dimethyl-4-(1,1,2,2-tetramethyl-1-silapropoxy)butyl benzoate (33e) in 15 mL of anhydrous tetrahydrofuran (THF) was reacted with 945 μL (935 mg, 5.8 mmol) of triethylamine trihydrofluoride. The reaction mixture was gradually warmed from room temperature to ca. 50° C. (oil bath) until the reaction was completed as determined by thin layer chromatography. After the starting material was completely consumed, the reaction mixture was diluted with water and then extracted with ethyl acetate. The combined organic extracts were washed with brine, dried over anhydrous magnesium sulfate (MgSO₄), filtered, and the solvents removed under reduced pressure using a rotary evaporator to provide 0.43 g (84% yield) of the title compound (32f) as a colorless oil. R_{f}=0.27 (EtOAc/Hxn=1:4). ¹H NMR (400 MHz, CDCl₃): δ=1.01 (s, 6H), 1.80 (t, J=7.2 Hz, 2H), 3.43 (s, 2H), 4.42 (t, J=7.2 Hz, 2H), 7.26-7.46 (m, 2H), 7.53-7.58 (m, 1H), 8.01-8.04 (m, 2H) ppm.

### Step G: 4-{3-(Acetylamino)propyl]sulfonyloxy}-3,3-dimethylbutyl benzoate (32)

Following the general procedure for the synthesis of acamprosate neopentyl sulfonylester prodrugs of Description 4, N-[3-(chlorosulfonyl)propyl]acetamide (13) (0.59 g, 2.96 mmol) dissolved in 10 mL of dichloromethane was reacted with 4-hydroxy-3,3-dimethylbutyl benzoate (32f) (0.33 g, 1.49 mmol) in the presence of 0.41 mL of triethylamine (0.30 g, 2.96 mmol) and 76 mg (0.62 mmol) of DMAP. After purification by mass-guided preparative HPLC, 300 mg (52% yield) of the title compound (32) was obtained as a colorless oil. ¹H NMR (400 MHz, CDCl₃): δ=1.06 (s, 6H), 1.82 (t, J=7.2 Hz, 2H), 1.97 (s, 3H), 2.02-2.09 (m, 2H), 3.15-3.18 (m, 2H), 3.35-3.40 (m, 2H), 3.96 (s, 2H), 4.38 (t, J=7.2 Hz, 2H), 6.28-6.35 (br. m, 1H), 7.40-7.44 (m, 2H), 7.52-7.56 (m, 1H), 7.98-8.00 (m, 2H) ppm. MS (ESI) m/z 386.13 (M+H)⁺.

### Example 33

### 4-{3-(Acetylamino)propyl]sulfonyloxy}-3,3-dimethylbutyl-2-aminoacetate Hydrochloride (33)

### Step A: 3,3-Dimethyl-4-(1,1,2,2-tetramethyl-1-silapropoxy)butyl 2-[(tert-butoxy)carbonylamino]acetate (33a)

To a stirred solution of 3,3-dimethyl-4-(1,1,2,2-tetramethyl-1-silapropoxy)butan-1-ol (32d) (0.58 g, 2.5 mmol) in 20 mL of anhydrous dichloromethane (DCM) in a 100 mL round-bottomed flask equipped with a magnetic stirring bar and a polyethylene cap was added 0.52 g (3.0 mmol) tert-butoxycarbonyl glycine (BOC-Gly-OH) and 4-(N,N-dimethylamino)pyridine (DMAP) (40 mg, 0.33 mmol). 619 mg (3.0 mmol) of N,N′-dicyclohexylcarbodiimide (DCC)was added to the stirred reaction mixture at room temperature. The reaction was stirred overnight at this temperature. After the starting material was completely consumed, the colorless precipitate was filtered off and the filter residue was washed with diethyl ether (Et₂O). The solution was then diluted with water and then extracted twice with Et₂O. The combined organic extracts were washed with brine, dried over anhydrous magnesium sulfate (MgSO₄), filtered, and the solvents removed under reduced pressure using a rotary evaporator. The residue was purified by silica gel chromatography using a mixture of ethyl acetate (EtOAc) and hexane (Hxn) as eluent (EtOAc/Hxn=1:4) to provide 0.9 g (92% yield) of the title compound (33a) as a colorless oil. R_{f}=0.70 (EtOAc/Hxn=1:2). ¹H NMR (400 MHz, CDCl₃): δ=0.04 (s, 6H), 0.89 (s, 6H), 0.90 (s, 9H), 1.46 (s, 9H), 1.63 (t, J=7.6 Hz, 2H), 3.25 (s, 2H), 3.89 (d, J=5.6 Hz, 2H), 4.22 (t, J=7.6 Hz, 2H), 5.00-5.10 (br. m, 1H) ppm. MS (ESI) m/z 390.26 (M+H)⁺.

### Step B: 4-Hydroxy-3,3-dimethylbutyl 2-[(tert-butoxy)carbonylamino]acetate (33b)

Adapting a procedure or a variation thereof according to Pirrung, et al., Bioorg. Med. Chem. Lett. 1994, 4, 1345-1346, to a stirred solution of 3,3-dimethyl-4-(1,1,2,2-tetramethyl-1-silapropoxy)butyl 2-[(tert-butoxy)carbonylamino]acetate (33a) (0.9 g, 2.3 mmol) in 15 mL of anhydrous tetrahydrofuran (THF) was added 652 μL of triethylamine trihydrofluoride (645 mg, 4.0 mmol). The reaction was stirred overnight at 50° C. (oil bath). After the starting material was completely consumed, the reaction was diluted with water and then extracted with ethyl acetate. The organic extracts were washed with brine, dried over anhydrous magnesium sulfate (MgSO₄), filtered, and the solvents removed under reduced pressure using a rotary evaporator to provide 0.4 g (63% yield) of the title compound (33b) as a colorless oil. R_{f}=0.27 (EtOAc/Hxn=1:2). ¹H NMR (400 MHz, CDCl₃): δ=0.94 (s, 6H), 1.46 (s, 9H), 1.67 (t, J=7.2 Hz, 2H), 3.35 (s, 2H), 3.89 (d, J=5.6 Hz, 2H), 4.24 (t, J=7.2 Hz, 2H), 5.00-5.10 (br. m, 1H) ppm. MS (ESI) m/z 298.09 (M+Na)⁺.

### Step C: 4-{3-(Acetylamino)propyl]sulfonyloxy}-3,3-dimethylbutyl 2-[(tert-butoxy)carbonylamino]acetate (33c)

Following the general procedure for the synthesis of acamprosate neopentyl sulfonylester prodrugs of Description 4, N-[3-(chlorosulfonyl)propyl]acetamide (13) (ca. 1.1 g, 5.5 mmol) dissolved in 20 mL of anhydrous dichloromethane (DCM) was reacted with 4-hydroxy-3,3-dimethylbutyl 2-[(tert-butoxy)carbonylamino]acetate (33b) (0.63 g, 2.3 mmol) in the presence of 0.80 mL of triethylamine (0.58 g, 5.7 mmol) and 61 mg (0.5 mmol) of DMAP. After aqueous work-up and purification by mass-guided preparative HPLC, 400 mg (40% yield) of the title compound (33c) was obtained as a colorless oil. ¹H NMR (400 MHz, CDCl₃): δ=1.93 (s, 6H), 1.46 (s, 9H), 1.71 (t, J=6.8 Hz, 2H), 2.01 (s, 3H), 2.05-2.12 (m, 2H), 3.14-3.21 (m, 2H), 3.39-3.44 (m, 2H), 3.89-3.94 (m, 4H), 4.23 (t, J=6.8 Hz, 2H), 5.08-5.16 (br. m, 1H), 6.08-6.18 (br. m, 1H) ppm. MS (ESI) m/z 439.10 (M+H)⁺.

### Step D: 4-[3-(Acetylamino)propyl]sulfonyloxy)-3,3-dimethylbutyl 2-amino acetate hydrochloride (33)

In a 20 mL screw-capped vial equipped with a magnetic stirring bar, 4-{3-(acetylamino)propyl]sulfonyloxy}-3,3-dimethylbutyl 2-[(tert-butoxy)carbonylamino]-acetate (33c) (0.4 g, 0.91 mmol) dissolved in 5 mL of anhydrous dichloromethane (DCM) was reacted 5 mL of trifluoroacetic acid for 2 hours at room temperature. The solvents were then removed under reduced pressure using a rotary evaporator. The residue was purified by mass-guided preparative HPLC and lyophilized in the presence of a slight excess of aqueous hydrogen chloride [by addition of one normal (1 N)] to give 200 mg (59% yield) of the title compound (33) as a colorless, viscous oil. ¹H NMR (400 MHz, Methanol-d⁴): δ=1.02 (s, 6H), 1.71-1.80 (m, 2H), 1.94-2.01 (m, 5H), 3.19-3.30 (m, 4H), 3.82-4.01 (m, 4H), 4.29-4.34 (m, 2H) ppm. MS (ESI) m/z 339.13 (M+H)⁺.

### Example 34

### 4-Hydroxy-2,2-dimethyl [3-(acetylamino)propyl]sulfonate (34)

### Step A: 3,3-Dimethylpent-4-en-1-ol (34a)

Following a procedure or an adaption thereof given by Wei, et al., Tetrahedron 1998, 54, 12623-12630, a dry 250 mL round-bottomed flask equipped with a magnetic stirring bar, an addition funnel, and a rubber septum was charged under a nitrogen atmosphere with 5.48 g (38.5 mmol) of methyl 3,3-dimethylpent-4-enoate. The material was dissolved in 70 mL of anhydrous tetrahydrofuran (THF) and the solution was cooled to ca. 0° C. (ice bath). 38.5 mL (38.5 mmol) of a one molar (1 M) solution of lithium aluminum hydride (LAH) in diethyl ether was added drop wise at this temperature and the reaction mixture was stirred overnight with warming to room temperature. After cooling to ca. 0° C. (ice bath), 2.35 mL of water, 4.70 mL of an aqueous solution of sodium hydroxide (10 wt-%), and 2.35 mL of water were carefully added (initially vigorous hydrogen evolution) and the resulting colorless precipitate was filtered off. The filter residue was washed with dichloromethane (DCM) and the combined filtrates were dried over magnesium sulfate (MgSO₄). After filtration and evaporation of the solvents under reduced pressure using a rotary evaporator, 2.93 g (66% yield) of the title compound (34a) was obtained as a colorless liquid that was of sufficient purity to be used in the next step without further purification. R_{f}=0.48 (EtOAc/Hxn=1:3). ¹H NMR (400 MHz, CDCl₃): δ=1.04 (s, 6H), 1.63 (t, J=7.2 Hz, 2H), 3.65 (t, J=6.8 Hz, 2H), 4.92-4.99 (m, 2H), 5.79-5.99 (m, 1H) ppm. The analytical data was consistent with the proposed structure and with the data given in the literature.

### Step B: 3,3-Dimethyl-1-(phenylmethoxy)pent-4-en (34b)

Following a procedure or an adaption thereof given by Wei, et al., Tetrahedron 1998, 54, 12623-12630, a dry 250 mL round-bottomed flask equipped with a magnetic stirring bar and a rubber septum was charged under a nitrogen atmosphere with 11.10 g of a 60 wt-% suspension in mineral oil (27.5 mmol) of sodium hydride (NaH). The material was washed with hexane, decanted, and the residue was dried under reduced pressure. The material was suspended in 50 mL of anhydrous dimethylformamide (DMF) and a solution of 3,3-dimethylpent-4-en-1-ol (34a) in 10 mL of anhydrous DMF was slowly added. After hydrogen evolution ceased, 3.56 mL (5.13 g, 30.0 mmol) of benzyl bromide was added and the reaction mixture was stirred overnight at 60° C. (oil bath). The reaction mixture was then diluted with ethyl acetate, washed with a 1.0 M aqueous solution of hydrogen chloride (HCl), a saturated aqueous solution of hydrogen carbonate (NaHCO₃) and brine, dried over magnesium sulfate (MgSO₄), filtered, and evaporated under reduced pressure to yield a yellow oil. The crude material was purified by silica gel column chromatography using a mixture of ethyl acetate (EtOAc) and hexane (Hxn) as eluent to provide 3.54 g (68% yield) of the title compound (34b) as a colorless liquid. R_{f}=0.57 (EtOAc/Hxn=1:9). ¹H NMR (400 MHz, CDCl₃): δ=1.04 (s, 6H), 1.69 (t, J=7.6 Hz, 2H), 3.49 (t, J=6.8 Hz, 2H), 4.48 (s, 2H), 4.89-4.96 (m, 2H), 5.76-5.84 (m, 1H), 7.27-7.36 (m, 5H) ppm. MS (ESI) m/z 205.16 (M+H)⁺. The analytical data was consistent with the proposed structure and with the data given in the literature.

### Step C: 3-Methyl-1-(phenylmethoxy)-3-(1,2,4-trioxolan-3-yl)butane (34c)

Caution: In general, ozonides must be handled with care because they may decompose explosively.

Following a procedure or variation thereof according to Srikrishna, et al., Tetrahedron 2000, 56, 8189-8195, a dry 250 mL round-bottomed flask equipped with a magnetic stirring bar and a multiply perforated polyethylene cap with a stainless steel needle was charged with 2.46 g (12.02 mmol) of 3,3-dimethyl-1-(phenylmethoxy)pent-4-en (34b). The material was dissolved in 100 mL of anhydrous dichloromethane (DCM) and cooled to −78° C. (dry ice/acetone). A mixture of ozone in oxygen was passed through the solution for ca. 30 minutes (the reaction mixture did not turn blue). The reaction was monitored by thin layer chromatography and after the starting material was consumed, excess ozone was removed by a nitrogen purge. The solvent was removed under reduced pressure using a rotary evaporator to yield a pale yellow oil which was purified by silica gel column chromatography using a mixture of ethyl acetate (EtOAc) and hexane (Hxn) (EtOAc/Hxn=1:9) as eluent to yield ca. 3.03 g (quant.) the title compound (34c) as a colorless oil. R_{f}=0.52 (EtOAc/Hxn=1:9). ¹H NMR (400 MHz, CDCl₃): δ=1.022 (s, 3H), 1.032 (s, 3H), 1.74 (t, J=7.2 Hz, 2H), 3.49 (t, J=7.6 Hz, 2H), 4.51 (s, 2H), 4.88 (s, 1H), 4.99 (s, 1H), 5.23 (s, 1H), 7.31-736 (m, 5H) ppm. The analytical data was consistent with the proposed structure.

### Step D: 2,2-Dimethyl-4-(phenylmethoxy)butan-1-ol (34d)

A dry 500 mL round-bottomed flask equipped with a magnetic stirring bar, addition funnel, and a rubber septum was charged under a nitrogen atmosphere with 3.03 g (12.02 mmol) of 3-methyl-1-(phenylmethoxy)-3-(1,2,4-trioxolan-3-yl)butane (34c). The material was dissolved in 40 mL of anhydrous tetrahydrofuran (THF) and the solution was cooled to ca. 0° C. (ice bath). Fifteen (15) mL (15 mmol) of a one molar (1 M) solution of lithium aluminum hydride in diethyl ether was added drop wise at this temperature and the reaction mixture was stirred for three hours with warming to room temperature. After cooling to ca. 0° C. (ice bath) 0.975 mL of water, 1.95 mL of an aqueous solution of sodium hydroxide (10 wt-%), and 0.975 mL of water was carefully added (initially vigorous hydrogen evolution!) and the resulting colorless precipitate was filtered off. The filter residue was washed with dichloromethane (DCM) and the combined filtrates were dried over magnesium sulfate (MgSO₄). After filtration and evaporation of the solvents under reduced pressure using a rotary evaporator, a pale-yellow oil was obtained. Purification by silica gel chromatography using mixtures of ethyl acetate (EtOAc) and hexane (Hxn) as eluent (EtOAc/Hxn=1:4→1:2) provided 1.52 g (61% yield) of the title compound (34d) as a colorless oil. R_{f}=0.31 (EtOAc/Hxn=1:4). ¹H NMR (400 MHz, CDCl₃): δ=0.92 (s, 6H), 1.59-1.63 (m, 2H), 3.15-3.23 (br. m, 1H), 3.30 (d, J=7.2 Hz, 2H), 3.53-3.58 (m, 2H), 4.53 (s, 2H), 7.27-7.39 (m, 5H) ppm. MS (ESI) m/z 208.97 (M+H)⁺, 230.93 (M+Na)⁺. The analytical data was consistent with the proposed structure and with the data given in the literature.

### Step E: 2,2-Dimethyl-4-(phenylmethoxy)butyl [3-(acetylamino)propyl]-sulfonate (34e)

Following the general procedure for the synthesis of acamprosate neopentyl sulfonylester prodrugs of Description 4, N-[3-(chlorosulfonyl)propyl]acetamide (13) (ca. 2.50 g, 12.52 mmol), dissolved in 40 mL of dichloromethane, was reacted with 2,2-dimethyl-4-(phenylmethoxy)butan-1-ol (34d) (1.52 g, 7.28 mmol) in the presence of 2.09 μL of triethylamine (1.52 g, 15.0 mmol) and 1.83 g (15.0 mmol) of DMAP. After purification by silica gel column chromatography using mixtures of ethyl acetate (EtOAc) and methanol (MeOH) as eluent (EtOAc→EtOAc/MeOH=95:5), 1.06 g (39% yield) of the title compound (34e) was obtained as a yellow oil. R_{f}=0.32 (EtOAc). ¹H NMR (400 MHz, CDCl₃): δ=1.02 (s, 6H), 1.67 (t, J=6.4 Hz, 2H), 1.98 (s, 3H), 1.99-2.07 (m, 2H), 3.07-3.12 (m, 2H), 3.37 (q, J=6.4 Hz, 2H), 3.56 (t, J=6.4 Hz, 2H), 3.98 (s, 2H), 4.49 (s, 2H), 5.71-5.77 (br. m, 1H), 7.27-7.38 (m, 5H) ppm. MS (ESI) m/z 372.07 (M+H)⁺, 394.08 (M+Na)⁺, 370.10 (M−H)⁻.

### Step F: 4-Hydroxy-2,2-dimethyl [3-(acetylamino)propyl]sulfonate (34)

A 100 mL round bottomed flask equipped with a magnetic stirring bar, a three-way stopcock, and a hydrogen-filled balloon (15 psi) was charged with 372 mg (1.0 mmol) of 2,2-dimethyl-4-(phenylmethoxy)butyl [3-(acetylamino)propyl]sulfonate (34e), 300 mg of 10 wt-% palladium on activated carbon, and 5 mL of anhydrous ethanol (EtOH). The atmosphere was exchanged to hydrogen (H₂) with three evacuation-refill cycles and the reaction mixture was stirred at room temperature for ca. 90 minutes. The reaction course was monitored by thin layer chromatography. The reaction mixture was then filtered over Celite®, the filter residue was washed with EtOH, and the combined filtrates were evaporated under reduced pressure using a rotary evaporator. The residue was dissolved in dichloromethane (DCM), filtered through a 0.2 μM nylon syringe filter, and the solvent was removed under reduced pressure using a rotary evaporator to provide 258 mg (95% yield) of the title compound (34) as a colorless oil. R_{f}=0.34 (EtOAc/MeOH=95:5). ¹H NMR (400 MHz, CDCl₃): δ=1.03 (s, 6H), 1.63 (t, J=6.8 Hz, 2H), 1.81 (br. m, 1H), 2.01 (s, 3H), 2.06-2.14 (m, 2H), 3.15-3.20 (m, 2H), 3.40 (q, J=6.4 Hz, 2H), 3.75 (t, J=6.8 Hz, 2H), 4.00 (s, 2H), 5.84-5.91 (br. m, 1H) ppm. MS (ESI) m/z 282.06 (M+H)⁺, 304.07 (M+Na)⁺.

### Example 35

### 2,2-Dimethylpentane-1,5-diol (35)

Adapting a procedure or a variation thereof according to Hashimoto, et al., J. Am. Chem. Soc. 1988, 110, 3670-3672; Ishii, et al., J. Org. Chem., 1988, 53, 5549-5552; and Nishimura, et al., J. Org. Chem., 1999, 64, 6750-6755, a dry 500 mL round bottomed flask equipped with a magnetic stirring bar and a pressure-equalizing addition funnel was charged under a nitrogen atmosphere with 1.42 g (37.50 mmol) of lithium aluminum hydride (LAH). The material was suspended in 70 mL of anhydrous tetrahydrofuran (THF) and the suspension cooled to ca. 0° C. (ice bath). At this temperature, a solution of 3.85 g (27.1 mmol) of commercially available 3,3-dimethyl-3H-4,5-dihydropyran-2,6-dione (3,3-dimethyl glutaric acid anhydride) in 30 mL of a anhydrous THF was added drop wise and the reaction mixture was stirred overnight with gradual warming to room temperature. The reaction mixture was then cooled to ca. 0° C. (ice bath) and 2.44 mL of water, 4.88 mL of an aqueous solution of sodium hydroxide (10 wt-%), and 2.44 mL of water were carefully added (Note: Initially vigorous evolution of hydrogen gas!) and the resulting colorless precipitate filtered off. The filter residue was washed with dichloromethane (DCM) or THF and the combined filtrates were dried over anhydrous magnesium sulfate (MgSO₄). After filtration and evaporation of the solvents under reduced pressure using a rotary evaporator, 3.60 g (quant.) of the title compound (35) was obtained as a colorless, viscous liquid that was of sufficient purity to be used without further purification in the next step. ¹H NMR (400 MHz, CDCl₃): δ=0.90 (s, 6H), 1.29-1.36 (m, 2H), 1.50-1.60 (m, 2H), 1.60-1.80 (br. m, 2H), 3.31-3.36 (br. m, 2H), 3.62-3.68 (m, 2H) ppm. MS (ESI) m/z: 133.04 (M+H)⁺. The analytical data was consistent with the proposed structure and with the data given in the literature.

### Description 5

### General Procedure for the Conversion of 2,2-Dimethylpentane-1,5-diol to a Carboxylic Acid or Carbonic Acid Ester

Adapting a procedure or a variation thereof according to Hashimoto, et al., J. Am. Chem. Soc. 1988, 110, 3670-3672; and Breton, et al., Tetrahedron Lett. 1997, 38, 3825-3828, a dry 500 mL round bottomed flask equipped with a magnetic stirring bar and a pressure equalizing addition funnel closed with a rubber septum was charged under a nitrogen atmosphere with 3.97 g (30.0 mmol) of 2,2-dimethylpentane-1,5-diol (35). One-hundred (100) mL of anhydrous dichloromethane (DCM) and 2.67 mL (2.61, 33.0 mmol) of anhydrous pyridine were added. The solution was cooled to ca. 0° C. (ice bath) and a solution of 30.0 mmol of an appropriate carboxylic acid chloride or alkyl- or aryl-chloroformate in 20 mL of anhydrous DCM was very slowly added at this temperature. The reaction mixture was stirred overnight with gradual warming to room temperature and the solvents were removed under reduced pressure using a rotary evaporator. The reaction mixture was diluted with 200 mL of ethyl acetate and 50 mL of a one molar (1.0 M) aqueous solution of hydrogen chloride (HCl). After phase separation, the organic phase was washed with a saturated aqueous solution of sodium hydrogencarbonate (NaHCO₃) and brine, dried over anhydrous magnesium sulfate (MgSO₄), filtered and the solvents were evaporated under reduced pressure using a rotary evaporator. The crude residue was purified from reaction side products (regioisomers and/or bis-esterified species) by silica gel chromatography using mixtures of ethyl acetate (EtOAc) and hexane (Hxn) as eluent to provide the target compound, typically as a clear oil. In some instances, the reaction mixture was used directly in the next step after aqueous work-up.

### Example 36

### 5-Hydroxy-4,4-dimethylpentyl ethoxyformate (36)

Following the general procedure for the conversion of 1,5-diol to a carboxylic acid or carbonic acid ester of Description 5, 661 mg (5.0 mmol) of 2,2-dimethylpentane-1,5-diol (35) was reacted in 10 mL of anhydrous dichloromethane (DCM) with a solution of 478 μL (543 mg, 5.0 mmol) of ethyl chloroformate in 5 mL of anhydrous DCM and in the presence of 425 μL (416 mg, 5.25 mmol) of anhydrous pyridine. After aqueous work-up, ca. 1 g of a colorless, viscous oil was obtained consisting of mixture of the title compound (36), the regioisomeric 5-hydroxy-2,2-dimethylpentyl ethoxyformate, and the corresponding bis-ethoxyformate in a ratio of approximately 2:1:3 (by LC/MS). The material was used without further purification or isolation in the next step. R_{f}=0.23 (EtOAc/Hxn=1:4). ¹H NMR (400 MHz, CDCl₃): δ=0.87 (s, 6H), 1.28-1.34 (m, 2H), 1.30 (t, J=7.2 Hz, 3H), 1.60-1.69 (m, 2H), 1.72-1.88 (br. m, 1H), 3.30 (br. s, 2H), 4.10 (t, J=7.2 Hz, 2H), 4.17 (t, J=7.2 Hz, 2H) ppm. MS (ESI) m/z: 204.90 (M+H)⁺. The analytical data was consistent with the proposed structure.

### Example 37

### 5-Hydroxy-4,4-dimethylpentyl Benzoate (37)

Following the general procedure for the conversion of 1,5-diol to a carboxylic acid or carbonic acid mono ester of Description 5, 661 mg (5.0 mmol) of 2,2-dimethylpentane-1,5-diol (35) was reacted in 10 mL of anhydrous dichloromethane (DCM) with a solution of 580 μL (703 mg, 5.0 mmol) benzoyl chloride in 5 mL of anhydrous DCM and in the presence of 425 μL (416 mg, 5.25 mmol) of anhydrous pyridine. After aqueous work-up, ca. 1 g of a colorless, viscous oil was obtained consisting of mixture of the title compound (37), the regioisomeric 5-hydroxy-2,2-dimethylpentyl benzoate, and the corresponding bis-benzoate in a ratio of approximately 3:1:2 (by LC/MS). The material was used without further purification or isolation in the next step. R_{f}=0.28 (EtOAc/Hxn=1:4). ¹H NMR (400 MHz, CDCl₃): δ=0.93 (s, 6H), 1.37-1.45 (m, 2H), 1.59-1.64 (br. m, 1H), 1.73-1.81 (m, 2H), 3.37 (br. s, 2H), 4.32 (t, J=6.4 Hz, 2H), 7.40-7.48 (m, 2H), 7.50-7.60 (m, 1H), 8.01-8.07 (m, 2H) ppm. MS (ESI) m/z: 236.90 (M+H)⁺. The analytical data was consistent with the proposed structure. A closely related analog, 5-hydroxy-4,4-dimethylpentyl 4-methylbenzoate, is described by Funabashi, et al., WO 2002/092606.

### Example 38

### 5-Hydroxy-4,4-dimethylpentyl 2,2-dimethylpropanate (38)

Following the general procedure for the conversion of 1,5-diol to a carboxylic acid or carbonic acid mono ester of Description 5, 3.97 g (30.0 mmol) of 2,2-dimethylpentane-1,5-diol (35) was reacted in 100 mL of anhydrous dichloromethane (DCM) with a solution of 3.70 mL (3.62 g, 30.0 mmol) of 2,2-dimethylpropanoyl chloride (pivaloyl chloride) in 20 mL of anhydrous DCM and in the presence of 2.67 mL (2.61 g, 33.0 mmol) of anhydrous pyridine. After aqueous work-up, ca. 6.5 g of a colorless, viscous oil was obtained consisting of mixture of the title compound (38), the regioisomeric 5-hydroxy-2,2-dimethylpentyl 2,2-dimethylpropanoate, and the corresponding bis-pivaloate in a ratio of approximately 6:1:2 (by LC/MS). The material was purified by silica gel column chromatography using ethyl acetate (EtOAc) and hexane (Hxn) mixtures as eluent (EtOAc/Hxn=1:9→EtOAc/Hxn=1:6) to provide 3.04 g (47% yield) of the title compound (38) as a colorless, viscous oil and as a single regioisomer. R_{f}=0.34 (EtOAc/Hxn=1:4). ¹H NMR (400 MHz, CDCl₃): δ=0.90 (s, 6H), 1.21 (s, 9H), 1.25-1.33 (m, 2H), 1.34-1.40 (br. m, 1H), 1.57-1.66 (m, 2H), 3.34 (br. s, 1H), 4.05 (t, J=6.8 Hz, 2H) ppm. MS (ESI) m/z: 217.01 (M+H)⁺, 238.90 (M+Na)⁺. The analytical data was consistent with the proposed structure.

### Example 39

### 2,2-Dimethyl-5-(phenylmethoxy)pentan-1-ol (39)

### Step A: rac-5-(2H-3,4,5,6-Tetrahydropyran-2-yloxy)-4,4-dimethylpentyl 2,2-dimethylpropanoate (39a)

Adapting a procedure or a variation thereof according to Hashimoto, et al., J. Am. Chem. Soc. 1988, 110, 3670-3672; Bernardy, et al., J. Org. Chem 1979, 44, 1438-1447; and Miyashita, et al., J. Org. Chem. 1979, 42, 3772-3774, a dry 250 mL round bottomed flask equipped with a magnetic stirring bar and a rubber septum was charged with 3.04 g (14.1 mmol) of 5-hydroxy-4,4-dimethylpentyl 2,2-dimethylpropanate (38) and 60 mL of anhydrous dichloromethane (DCM). 12.73 mL (11.82 g, 141.0 mmol) of dihydropyran was added and the reaction mixture was cooled to ca. 0° C. (ice bath). Twenty-seven (27) mg (0.14 mmol) of para-toluenesulfonic acid monohydrate (TsOH.H₂O) was added and the reaction mixture was stirred for 3 hours with gradual warming to room temperature. The reaction mixture was then diluted with diethyl ether and the organic phase was successively washed with a saturated aqueous sodium hydrogencarbonate (NaHCO₃) solution and brine. The solution was dried over anhydrous magnesium sulfate (MgSO₄), filtered, and the solvent was evaporated under reduced pressure using a rotary evaporator. The crude reaction product was purified by silica gel chromatography using an ethyl acetate (EtOAc) and hexane (Hxn) mixture as eluent (EtOAc/Hxn=1:6) to provide 4.04 g (96% yield) of the title compound (39a) as a colorless liquid. R_{f}=0.67 (EtOAc/Hxn=1:6). ¹H NMR (400 MHz, CDCl₃): δ=0.91 (s, 3H), 0.92 (s, 3H), 1.22 (s, 9H), 1.30-1.38 (m, 2H), 1.48-1.74 (m, 7H), 1.79-1.89 (m, 1H), 3.01 (d, J_{vic}=9.2 Hz, 1H), 3.47 (d, J_{vic}=9.2 Hz, 1H), 3.48-3.54 (m, 1H), 3.81-3.88 (m, 1H), 4.04 (t, J=6.8 Hz, 2H), 4.54-4.57 (m, 1H) ppm. MS (ESI) m/z: 301.0 (M+H)⁺, 322.9 (M+Na)⁺. The analytical data was consistent with the proposed structure.

### Step B: rac-5-(2H-3,4,5,6-Tetrahydropyran-2-yloxy)-4,4-dimethylpentan-1-ol (39b)

Adapting a procedure, or a variation thereof, according to Hashimoto, et al., J. Am. Chem. Soc. 1988, 110, 3670-3672; Nicolaou, et al., J. Am. Chem. Soc. 1990, 112, 3693-3695; and Gassman, et al., J. Org. Chem. 1979, 42, 918-920, a dry 250 mL flask equipped with a magnetic stirring bar and a rubber septum was charged with 4.04 g (13.4 mmol) of rac-5-(2H-3,4,5,6-tetrahydropyran-2-yloxy)-4,4-dimethylpentyl 2,2-dimethylpropanoate (39a). The compound was dissolved in 50 mL of anhydrous methanol (MeOH) and 2.18 g (40.3 mmol) of solid sodium methoxide (NaOCH₃) was added. The reaction mixture was stirred overnight at 65° C. (oil bath). After the starting material was completely consumed, the solvent was partially evaporated under reduced pressure using a rotary evaporator and the residual methanolic solution was diluted with water and dichloromethane (DCM). After phase separation, the aqueous phase was extracted five times with DCM and the combined organic extracts were washed with brine, dried over anhydrous magnesium sulfate (MgSO₄), filtered, and the solvents were evaporated under reduced pressure using a rotary evaporator. The crude reaction product was purified by silica gel chromatography using ethyl acetate (EtOAc) and hexane (Hxn) mixtures as eluent (EtOAc/Hxn=1:4→EtOAc/Hxn=1:3 →EtOAc/Hxn=1:2) to provide 2.67 g (92% yield) of the title compound (39b) as a colorless liquid. R_{f}=0.38 (EtOAc/Hxn=1:2). ¹H NMR (400 MHz, CDCl₃): δ=0.92 (s, 3H), 0.93 (s, 3H), 1.31-1.41 (m, 3H), 1.49-1.65 (m, 6H), 1.67-1.75 (m, 1H), 1.79-1.88 (m, 1H), 3.02 (d, J=9.2 Hz, 1H), 3.49 (d, J=9.2 Hz, 1H), 3.48-3.54 (m, 1H), 3.60-3.67 (br. m, 2H), 3.82-3.89 (m, 1H), 4.54-4.57 (m, 1H) ppm. MS (ESI) m/z: 217.0 (M+H)⁺, 238.9 (M+Na)⁺. The analytical data was consistent with the proposed structure.

### Step C: rac-2-[2,2-Dimethyl-5-(phenylmethoxy)pentyloxy]-2H-3,4,5,6-tetrahydropyran (39c)

A dry 250 mL flask equipped with a magnetic stirring bar and a rubber septum was charged under a nitrogen atmosphere with 560 mg (14.0 mmol) of a 60 wt-% suspension of sodium hydride (NaH) in mineral oil. Mineral oil was removed using two washing/decanting cycles of 25 mL of anhydrous hexane. The washed NaH was dried in vacuum and subsequently suspended in 25 mL of anhydrous N,N-dimethylformamide (DMF). The suspension was heated to 65° C. (oil bath) and a solution of 2.67 g (8.87 mmol) of rac-5-(2H-3,4,5,6-tetrahydropyran-2-yloxy)-4,4-dimethylpentan-1-ol (39b) in 10 mL of anhydrous DMF was added dropwise. The reaction mixture was stirred at this temperature until the hydrogen gas evolution subsided (ca. one hour). Neat benzyl bromide (1.66 mL, 2.40 g, 14.0 mmol) was then added and the reaction mixture was stirred overnight at this temperature. The reaction mixture was carefully quenched with water to destroy excess NaH and was further diluted with water and methyl tert-butyl ether (MTBE). After phase separation, the aqueous phase was extracted twice more with MTBE and the combined organic extracts were washed with brine, dried over anhydrous magnesium sulfate (MgSO₄), filtered, and the solvents were evaporated under reduced pressure using a rotary evaporator. The crude reaction product was purified by silica gel chromatography using an ethyl acetate (EtOAc) and hexane (Hxn) mixture as eluent (EtOAc/Hxn=1:9) to provide 2.34 g (86% yield) of the title compound (39c) as a colorless liquid. R_{f}=0.53 (EtOAc/Hxn=1:6). ¹H NMR (400 MHz, CDCl₃): δ=0.92 (s, 3H), 0.93 (s, 3H), 1.30-1.37 (m, 2H), 1.48-1.74 (m, 7H), 1.78-1.90 (m, 1H), 3.01 (d, J=9.2 Hz, 1H), 3.43-3.54 (m, 4H), 3.81-3.88 (m, 1H), 4.51 (s, 2H), 4.54-4.57 (m, 1H), 7.30-7.39 (m, 5H) ppm. MS (ESI) m/z: 307.0 (M+H)⁺. The analytical data was consistent with the proposed structure.

### Step D: 2,2-Dimethyl-5-(phenylmethoxy)pentan-1-ol (39)

Adapting a procedure or a variation thereof according to Hashimoto, et al., J. Am. Chem. Soc., 1988, 110, 3670-3672; and Miyashita, et al., J. Org. Chem., 1979, 42, 3772-3774, a dry 250 mL round bottomed flask equipped with a magnetic stirring bar and a rubber septum was charged with 2.34 g (7.64 mmol) of rac-2-[2,2-dimethyl-5-(phenylmethoxy)pentyloxy]-2H-3,4,5,6-tetrahydropyran (39c) and 60 mL of anhydrous ethanol (EtOH). Two-hundred fifty-one (251) mg (1.0 mmol) of pyridinium para-toluenesulfonate (PPTS) was added and the reaction mixture was stirred at 60° C. (oil bath). The reaction was monitored by thin layer chromatography. After three hours at this temperature, the starting material was completely consumed and the ethanol was partially evaporated under reduced pressure using a rotary evaporator. The residual solution was diluted with methyl tert-butyl ether (MTBE) and an aqueous one molar solution (1.0 M) of hydrogen chloride (HCl). After phase separation, the organic phase was washed with water, a saturated aqueous solution of sodium hydrogencarbonate (NaHCO₃), and brine. The solution was dried over anhydrous magnesium sulfate (MgSO₄), filtered, and the solvent was evaporated under reduced pressure using a rotary evaporator. After removal of transacetalization by-products under high vacuum, 1.59 g (94% yield) of the title compound (39) was obtained as an almost colorless liquid. The reaction product was of sufficient purity to be used in the next step without further purification or isolation. R_{f}=0.45 (EtOAc/Hxn=1:2). ¹H NMR (400 MHz, CDCl₃): δ=0.89 (s, 6H), 1.30-1.36 (m, 2H), 1.57-1.65 (m, 3H), 3.31-3.34 (br. m, 2H), 3.47 (t, J=6.4 Hz, 2H), 4.52 (s, 2H), 7.30-7.37 (m, 5H) ppm. MS (ESI) m/z: 223.0 (M+H)⁺, 245.0 (M+Na)⁺. The analytical data was consistent with the proposed structure.

### Example 40

### 5-{[3-(Acetylamino)propyl]sulfonyloxy}-4,4-dimethylpentyl ethoxyformate (40)

### Step A: 5-[(3-Chloropropyl)sulfonyloxy]-4,4-dimethylpentyl ethoxyformate (40a)

Following the general procedure for coupling of neopentyl promoieties (neopentylalcohols) of Description 4,3-chloropropylsulfonyl chloride (973 μL, 1.42 g, 8.0 mmol) dissolved in 30 mL of dichloromethane was reacted with crude a regioisomeric mixture (ca. 2:1) of 5-hydroxy-4,4-dimethylpentyl ethoxyformate (36) (ca. 1.0 g, ca. 4.9 mmol) in the presence of 1.12 mL of triethylamine (810 mg, 8.0 mmol) and 977 mg (8.0 mmol) of DMAP. After aqueous work-up, ca. 1.6 g of the crude reaction product was selectively hydrolyzed in a mixture of 25 mL methanol and 15 mL of a saturated aqueous solution of sodium hydrogencarbonate. After aqueous work up and purification by silica gel column chromatography using mixtures of ethyl acetate (EtOAc) and hexane (Hxn) as eluent (EtOAc/Hxn=1:9→EtOAc/Hxn=1:6), 879 mg (quant.) of the title compound (40a) was obtained as a pale-yellow, clear oil in a regioisomeric ratio of ca. 6:1 (738 mg, 86% yield for the desired regioisomer). R_{f}=0.39 (EtOAc/Hxn=1:4). ¹H NMR (400 MHz, CDCl₃): δ=0.91 (s, 6H), 1.33 (t, J=7.2 Hz, 3H), 1.37-1.43 (m, 2H), 1.63-1.72 (m, 2H), 2.31-2.38 (m, 2H), 3.28-3.34 (m, 2H), 3.69-3.73 (m, 2H), 3.93 (s, 2H), 4.13 (t, J=6.8 Hz, 2H), 4.20 (q, J=7.2 Hz, 2H) ppm. MS (ESI) m/z: 345.0 (M+H)⁺, 367.0 (M+Na)⁺.

### Step B: 5-[(3-Azidopropyl)sulfonyloxy]-4,4-dimethylpentyl ethoxyformate (40b)

Adapting a procedure or a variation thereof according to de la Mora, et al., Tetrahedron Lett. 2001, 42, 5351-5353; and De Kimpe, et al., Tetrahedron 1997, 53, 3693-3706, a dry 100 mL round bottomed flask equipped with a magnetic stirring bar and a rubber septum was charged with 879 mg of the regioisomeric mixture (ca. 6:1) from Step A [738 mg, 2.14 mmol of 5-[(3-chloropropyl)sulfonyloxy]-4,4-dimethylpentyl ethoxyformate (40a)] and 10 mL of anhydrous dimethylsulfoxide (DMSO). 650 mg of sodium azide (NaN₃) was added to the solution and the reaction mixture was stirred at 55° C. (oil bath) until thin layer chromatography indicated that the starting material was completely consumed (ca. 3 hours). The reaction mixture was then diluted with 50 mL of water and 150 mL of methyl tert-butyl ether (MTBE), and the phases were separated. The aqueous phase was extracted twice more with MTBE and the combined organic extracts were washed with a saturated aqueous solution of sodium hydrogencarbonate and brine. The solution was dried over anhydrous magnesium sulfate (MgSO₄), filtered, and the solvent was evaporated under reduced pressure using a rotary evaporator to provide 750 mg (99% yield) of the title compound (40b) as a light-yellow oil. The undesired regioisomer decomposed quantitatively under the reaction conditions to afford regioisomerically pure reaction product. The crude reaction product was of sufficient purity to be used in the next step without further isolation and purification. R_{f}=0.22 (MTBE/Hxn=1:2). ¹H NMR (400 MHz, CDCl₃): δ=0.99 (s, 6H), 1.33 (t, J=7.2 Hz, 3H), 1.37-1.42 (m, 2H), 1.63-1.71 (m, 2H), 2.09-2.17 (m, 2H), 3.19-3.24 (m, 2H), 3.51-3.55 (m, 2H), 3.92 (s, 2H), 4.09-4.16 (m, 4H) ppm. MS (ESI) m/z: 352.09 (M+H)⁺, 374.05 (M+Na)⁺.

### Step C: 5-[(3-Aminopropyl)sulfonyloxy]-4,4-dimethylpentyl ethoxyformate Acetate (40c)

A 250 mL round bottomed flask equipped with a magnetic stirring bar and a three-way stopcock and a hydrogen-filled balloon (15 psi) was charged with 750 mg (2.13 mmol) of 5-[(3-azidopropyl)sulfonyloxy]-4,4-dimethylpentyl ethoxyformate (40b), 422 mg of 10 wt-% palladium on activated carbon, and 25 mL of anhydrous ethanol (EtOH). The atmosphere was exchanged to hydrogen (H₂) using three evacuation-refill cycles and the reaction mixture was stirred at room temperature. The reaction was monitored by thin layer chromatography and after ca. four hours the reaction mixture was filtered over Celite®, and the filter residue was washed with EtOH. 137 μL (144 mg, 2.40 mmol) of glacial acetic acid was added to transform the free amine into its corresponding acetate salt and to prevent acyl transfer reactions. The combined filtrates were evaporated under reduced pressure using a rotary evaporator. The residue was dissolved in dichloromethane (DCM), filtered through a 0.2 μM nylon syringe filter to remove traces of the heterogeneous catalyst, and the solvent was removed under reduced pressure using a rotary evaporator to yield 820 mg (quant.) of the title compound (40c) as an almost colorless, viscous oil. The material thus obtained was of sufficient purity to be used without further purification or isolation in the next step. MS (ESI) m/z: 326.08 (M+H)⁺, 348.03 (M+Na)⁺.

### Step D: 5-{[3-(Acetylamino)propyl]sulfonyloxy}-4,4-dimethylpentyl ethoxyformate (40)

A 250 mL round bottomed flask equipped with a magnetic stirring bar and a rubber septum was charged with 820 mg (2.13 mmol) of 5-[(3-aminopropyl)sulfonyloxy]-4,4-dimethylpentyl ethoxyformate acetate (40c) and dissolved in 20 mL of anhydrous dichloromethane (DCM). Thirty-one (31) mg (0.25 mmol) of (4-N,N-dimethylamino)pyridine (DMAP) was added and the solution was cooled to ca. 0° C. (ice bath). At this temperature, 236 μL (255 mg, 2.35 mmol) of acetic anhydride (Ac₂O) and 697 μL (506 mg, 5.0 mmol) of triethylamine (TEA) was added and the reaction mixture was stirred overnight with gradual warming to room temperature. The solvent was then evaporated under reduced pressure using a rotary evaporator and the residue was dissolved in 50 mL of ethyl acetate (EtOAc). The organic phase was successively washed with a one normal aqueous solution of hydrogen chloride (HCl), a saturated aqueous solution of sodium hydrogencarbonate (NaHCO₃), and brine. After drying over anhydrous magnesium sulfate (MgSO₄) and filtration, the solvent was evaporated under reduced pressure using a rotary evaporator. The residue was purified by silica gel chromatography using ethyl acetate (EtOAc) as eluent to yield 454 mg of a colorless viscous oil. Following mass-guided preparative HPLC purification, 229 mg (29% yield) of the title compound (40) was obtained as a pale-yellow, clear, viscous oil. R_{f}=0.23 (EtOAc). ¹H NMR (400 MHz, CDCl₃): δ=0.98 (s, 6H), 1.32 (t, J=6.8 Hz, 3H), 1.35-1.41 (m, 2H), 1.62-1.71 (m, 2H), 2.00 (s, 3H), 2.04-2.12 (m, 2H), 3.14-3.19 (m, 2H), 3.39-3.45 (m, 2H), 3.90 (s, 2H), 4.12 (t, J=6.4 Hz, 2H), 4.20 (q, J=7.2 Hz, 2H), 5.89-5.95 (br. m, 1H) ppm. MS (ESI) m/z: 368.14 (M+H)⁺, 390.03 (M+Na)⁺.

### Example 41

### 5-{[3-(Acetylamino)propyl]sulfonyloxy}-4,4-dimethylpentyl Benzoate (41)

### Step A: 5-[(3-Chloropropyl)sulfonyloxy]-4,4-dimethylpentyl benzoate (41a)

Following the general procedure for coupling of neopentyl promoieties (neopentylalcohols) of Description 4,3-chloropropylsulfonyl chloride (973 μL, 1.42 g, 8.0 mmol) dissolved in 30 mL of dichloromethane was reacted with a crude regioisomeric mixture (ca. 3:1) of 5-hydroxy-4,4-dimethylpentyl benzoate (37) (ca. 1.2 g, ca. 5.0 mmol) in the presence of 1.12 mL of triethylamine (810 mg, 8.0 mmol) and 977 mg (8.0 mmol) of DMAP. Ca. 1.7 g of the crude reaction product was selectively hydrolyzed in a mixture of 25 mL methanol and 15 mL of a saturated aqueous solution of sodium hydrogencarbonate. After aqueous work up and purification by silica gel column chromatography using mixtures of ethyl acetate (EtOAc) and hexane (Hxn) as eluent (EtOAc/Hxn=1:6→EtOAc/Hxn=1:4→EtOAc/Hxn=1:2), 879 mg (quant.) of the title compound (41a) was obtained as a clear, yellow oil in a regioisomeric ratio of ca. 10:1 (799 mg, 93% yield for the desired regioisomer). R_{f}=0.23 (EtOAc/Hxn=1:6). ¹H NMR (400 MHz, CDCl₃): δ=1.02 (s, 6H), 1.44-1.50 (m, 2H), 1.74-1.83 (m, 2H), 2.30-2.37 (m, 2H), 3.28-3.36 (m, 2H), 3.67-3.70 (m, 2H), 3.96 (s, 2H), 4.32 (t, J=6.4 Hz, 2H), 7.42-7.48 (m, 2H), 7.54-7.59 (m, 1H), 8.02-8.06 (m, 2H) ppm. MS (ESI) m/z: 377.0 (M+H)⁺, 399.0 (M+Na)⁺.

### Step B: 5-[(3-Azidopropyl)sulfonyloxy]-4,4-dimethylpentyl benzoate (41b)

Adapting a procedure or a variation thereof according to de la Mora, et al., Tetrahedron Lett. 2001, 42, 5351-5353; and De Kimpe, et al., Tetrahedron 1997, 53, 3693-3706, a dry 100 mL round bottomed flask equipped with a magnetic stirring bar and a rubber septum was charged with 879 mg of the regioisomeric mixture (ca. 10:1) from Step A [800 mg, 2.12 mmol of 5-[(3-chloropropyl)sulfonyloxy]-4,4-dimethylpentyl benzoate (41a)] and 10 mL of anhydrous dimethylsulfoxide (DMSO). To the solution, 650 mg of sodium azide (NaN₃) was added and the reaction mixture was stirred at 55° C. (oil bath) until thin layer chromatography indicated that the starting material was completely consumed (ca. 3 hours). The reaction mixture was diluted with 50 mL of water and 150 mL of methyl tert-butyl ether (MTBE) and the phases were separated. The aqueous phase was extracted twice more with MTBE and the combined organic extracts were washed with a saturated aqueous solution of sodium hydrogencarbonate and brine. The solution was dried over anhydrous magnesium sulfate (MgSO₄), filtered, and the solvent was evaporated under reduced pressure using a rotary evaporator to provide 814 mg (99% yield) of the title compound (41b) as a slightly yellow oil. The undesired regioisomer decomposed quantitatively under the reaction conditions to afford regioisomerically pure reaction product. The crude reaction product was of sufficient purity to be used in the next step without further isolation and purification s. R_{f}=0.27 (EtOAc/Hxn=1:4). ¹H NMR (400 MHz, CDCl₃): δ=1.02 (s, 6H), 1.44-1.49 (m, 2H), 1.74-1.82 (m, 2H), 2.07-2.16 (m, 2H), 3.18-3.23 (m, 2H), 3.48-3.52 (m, 2H), 3.95 (s, 2H), 4.32 (t, J=6.4 Hz, 2H), 7.42-7.48 (m, 2H), 7.54-7.59 (m, 1H), 8.02-8.06 (m, 2H) ppm. MS (ESI) m/z: 384.1 (M+H)⁺, 406.1 (M+Na)⁺.

### Step C: 5-[(3-Aminopropyl)sulfonyloxy]-4,4-dimethylpentyl benzoate acetate (41c)

A 250 mL round bottomed flask equipped with a magnetic stirring bar and a three-way stopcock and a hydrogen-filled balloon (15 psi) was charged with 814 mg (2.12 mmol) of 5-[(3-azidopropyl)sulfonyloxy]-4,4-dimethylpentyl benzoate (41b), 421 mg of 10 wt-% palladium on activated carbon and 25 mL of anhydrous ethanol (EtOH). The atmosphere was exchanged to hydrogen (H₂) with three evacuation-refill cycles and the reaction mixture was stirred at room temperature. The reaction was monitored by thin layer chromatography and after ca. four hours the reaction mixture was filtered over Celite®, and the filter residue washed with EtOH. 137 μL (144 mg, 2.40 mmol) of glacial acetic acid was added to transform the free amine into its corresponding acetate salt and to prevent acyl transfer reactions. The combined filtrates were evaporated under reduced pressure using a rotary evaporator. The residue was dissolved in dichloromethane (DCM), filtered through a 0.2 μM nylon syringe filter to remove traces of the heterogeneous catalyst, and the solvent was removed under reduced pressure using a rotary evaporator to yield 882 mg (quant.) of the title compound (41c) as an almost colorless, viscous oil. The material thus obtained was of sufficient purity to be used in the next step without further purification or isolation. MS (ESI) m/z: 358.1 (M+H)⁺, 380.1 (M+Na)⁺.

### Step D: 5-{[3-(Acetylamino)propyl]sulfonyloxy}-4,4-dimethylpentyl benzoate (41)

A 250 mL round bottomed flask equipped with a magnetic stirring bar and a rubber septum was charged with 882 mg (2.11 mmol) of 5-[(3-aminopropyl)sulfonyloxy]-4,4-dimethylpentyl benzoate acetate (41c) and dissolved in 20 mL of anhydrous dichloromethane (DCM). Thirty-one (31) mg (0.25 mmol) of (4-N,N-dimethylamino)pyridine (DMAP) was added and the solution was cooled to ca. 0° C. (ice bath). At this temperature, 236 μL (255 mg, 2.35 mmol) of acetic anhydride (AC₂O) and 697 μL (506 mg, 5.0 mmol) of triethylamine (TEA) was added and the reaction mixture was stirred overnight with gradual warming to room temperature. The solvent was evaporated under reduced pressure using a rotary evaporator and the residue was dissolved in 50 mL of ethyl acetate (EtOAc). The organic phase was successively washed with a one normal aqueous solution of hydrogen chloride (HCl), a saturated aqueous solution of sodium hydrogencarbonate, and brine. After drying over anhydrous magnesium sulfate (MgSO₄) and filtration, the solvent was evaporated under reduced pressure using a rotary evaporator. The residue was purified by mass-guided preparative HPLC to provide 431 mg (51% yield) of the title compound (41) as pale-yellow, clear, viscous oil. R_{f}=0.26 (EtOAc). ¹H NMR (400 MHz, CDCl₃): δ=1.01 (s, 6H), 1.43-1.48 (m, 2H), 1.73-1.82 (m, 2H), 2.00 (s, 3H), 2.04-2.12 (m, 2H), 3.14-3.19 (m, 2H), 3.38-3.44 (m, 2H), 3.94 (s, 2H), 4.32 (t, J=6.4 Hz, 2H), 5.84-5.91 (br. m, 1H), 7.43-7.48 (m, 2H), 7.54-7.59 (m, 1H), 8.01-8.05 (m, 2H) ppm. MS (ESI) m/z: 400.1 (M+H)⁺, 422.1 (M+Na)⁺.

### Example 42

### 5-Hydroxy-2,2-dimethylpentyl [3-(acetylamino)propyl]sulfonate (42)

### Step A: 2,2-Dimethyl-5-(phenylmethoxy)pentyl (3-chloropropyl)sulfonate (42a)

Following the general procedure for coupling of neopentyl promoieties (neopentylalcohols) of Description 4,3-chloropropylsulfonyl chloride (1.71 mL, 2.48 g, 14.0 mmol) dissolved in 60 mL of dichloromethane was reacted with 2,2-dimethyl-5-(phenylmethoxy)pentan-1-ol (39) (1.59 g, 7.15 mmol) in the presence of 1.95 mL of triethylamine (1.42 g, 14.0 mmol) and 1.71 g (14.0 mmol) of DMAP. After purification by silica gel column chromatography using mixtures of ethyl acetate (EtOAc) and hexane (Hxn) as eluent (EtOAc/Hxn=1:6→EtOAc/Hxn=1:5→EtOAc/Hxn=1:4), 2.14 g (82% yield) of the title compound (42a) was obtained as a pale-yellow, clear oil. R_{f}=0.31 (EtOAc/Hxn=1:6). ¹H NMR (400 MHz, CDCl₃): δ=0.99 (s, 6H), 1.36-1.42 (m, 2H), 1.58-1.66 (m, 2H), 2.29-2.36 (m, 2H), 3.25-3.32 (m, 2H), 3.47 (t, J=6.4 Hz, 2H), 3.65-3.70 (m, 2H), 3.94 (s, 2H), 4.51 (s, 2H), 7.28-7.38 (m, 5H) ppm. MS (ESI) m/z: 363.1 (M+H)⁺, 385.1 (M+Na)⁺.

### Step B: 2,2-Dimethyl-5-(phenylmethoxy)pentyl (3-azidopropyl)sulfonate (42b)

Adapting a procedure or a variation thereof according to de la Mora, et al., Tetrahedron Lett. 2001, 42, 5351-5353; and De Kimpe, et al., Tetrahedron 1997, 53, 3693-3706, a dry 250 mL round bottomed flask equipped with a magnetic stirring bar and a rubber septum was charged with 2.14 g (5.89 mmol) of 2,2-dimethyl-5-(phenylmethoxy)pentyl (3-chloropropyl)sulfonate (42a) and 20 mL of anhydrous dimethylsulfoxide (DMSO). 650 mg of sodium azide (NaN₃) was added to the solution and the reaction mixture was stirred at 55° C. (oil bath) until thin layer chromatography indicated that the starting material was completely consumed (ca. 4 hours). The reaction mixture was diluted with 100 mL of water and 250 mL of methyl tert-butyl ether (MTBE) and the phases were separated. The aqueous phase was extracted twice more with MTBE and the combined organic extracts were washed with a saturated aqueous solution of sodium hydrogencarbonate and brine. The solution was dried over anhydrous magnesium sulfate (MgSO₄), filtered, and the solvent was evaporated under reduced pressure using a rotary evaporator to yield 2.18 g (quant.) of the title compound (42b) as a slightly yellow oil. The crude reaction product was of sufficient purity to be used in the next step without further isolation and purification. R_{f}=0.35 (EtOAc/Hxn=1:4). ¹H NMR (400 MHz, CDCl₃): δ=0.98 (s, 6H), 1.36-1.42 (m, 2H), 1.57-1.66 (m, 2H), 2.07-2.15 (m, 2H), 3.17-3.22 (m, 2H), 3.45-3.51 (m, 4H), 3.93 (s, 2H), 4.51 (s, 2H), 7.27-7.38 (m, 5H) ppm. MS (ESI) m/z: 370.1 (M+H)⁺, 392.1 (M+Na)⁺.

### Step C: 2,2-Dimethyl-5-(phenylmethoxy)pentyl (3-aminopropyl)sulfonate (42c)

Adapting a procedure or a variation thereof according to Nagarajan, et al., J. Org. Chem. 1987, 52, 5044-5046; and Pillard, et al., Tetrahedron Lett. 1984, 25, 1555-1556, a dry 250 mL round bottomed flask equipped with a magnetic stirring bar and a rubber septum was charged under a nitrogen atmosphere with 2.18 g (5.89 mmol) of 2,2-dimethyl-5-(phenylmethoxy)pentyl (3-azidopropyl)sulfonate (42b) and 25 mL of tetrahydrofuran (THF). To the solution was added 1.70 g (6.48 mmol) of triphenylphosphine (PPh₃) and 116 μL (I 16 mg, 6.48 mmol) of water. The reaction mixture was stirred at room temperature for 18 hours. Thin layer chromatography indicated that the starting material was completely consumed. The solvent was evaporated under reduced pressure using a rotary evaporator to yield the title compound (42c) as a yellow oil and the residue was used in the next step without further purification or isolation. MS (ESI) m/z: 370.1 (M+H)⁺, 392.1 (M+Na)⁺.

### Step D: 2,2-Dimethyl-5-(phenylmethoxy)pentyl [3-(acetylamino)propyl]sulfonate (42d)

A 250 mL round bottomed flask equipped with a magnetic stirring bar and a rubber septum was charged with the material obtained from Step C [ca. 2.03 g (5.89 mmol) of 2,2-dimethyl-5-(phenylmethoxy)pentyl (3-aminopropyl)sulfonate (42c)] and the material was dissolved in 25 mL of anhydrous dichloromethane (DCM). 122 mg (1.0 mmol) of (4-N,N-dimethylamino)pyridine (DMAP) was added and the solution was cooled to ca. 0° C. (ice bath). At this temperature, 668 μL (722 mg, 7.07 mmol) of acetic anhydride (Ac₂O) and 1.026 mL (734 mg, 7.07 mmol) of triethylamine (TEA) were added and the reaction mixture was stirred overnight with gradual warming to room temperature. The solvent was evaporated under reduced pressure using a rotary evaporator and the residue was dissolved in 100 mL of ethyl acetate (EtOAc). The organic phase was successively washed with a one normal aqueous solution of hydrogen chloride (HCl), a saturated aqueous solution of sodium hydrogencarbonate, and brine. After drying over anhydrous magnesium sulfate (MgSO₄) and filtration, the solvent was evaporated under reduced pressure using a rotary evaporator. Excess triphenylphosphine oxide was partially removed by titruation of the residue with methyl tert-butylether. After removal of the solvent under reduced pressure using a rotary evaporator, the residue was purified by silica gel chromatography using ethyl acetate (EtOAc) and hexane (Hxn) mixtures as containing one vol-% of glacial acetic acid (HOAc) as a co-solvent (EtOAc/Hxn=2:1→EtOAc/Hxn=4:1→EtOAc/Hxn=4:1→EtOAc/Hxn=7:1) to provide 1.12 g (49% yield) of the title compound (42d) as yellow, clear, viscous oil and with another fraction contaminated with triphenylphosphine oxide. R_{f}=0.29 (EtOAc/Hxn=4:1+1 vol-% HOAc). ¹H NMR (400 MHz, CDCl₃): δ=0.98 (s, 6H), 1.36-1.42 (m, 2H), 1.57-1.66 (m, 2H), 1.98 (s, 3H), 2.03-2.11 (m, 2H), 3.12-3.17 (m, 2H), 3.35-3.41 (m, 2H), 3.48 (t, J=6.4 Hz, 2H), 3.91 (s, 2H), 4.51 (s, 2H), 5.74-5.83 (br. m, 1H), 7.27-38 (m, 5H) ppm. MS (ESI) m/z: 386.1 (M+H)⁺, 408.0 (M+Na)⁺.

### Step E: 5-Hydroxy-2,2-dimethylpentyl [3-(acetylamino)propyl]sulfonate (42)

A 100 mL round bottomed flask equipped with a magnetic stirring bar, a three-way stopcock, and a hydrogen-filled balloon (15 psi), was charged with 1.12 g (2.91 mmol) of 2,2-dimethyl-5-(phenylmethoxy)pentyl [3-(acetylamino)propyl]sulfonate (42d), 522 mg of 10 wt-% palladium on activated carbon, 100 μL of glacial acetic acid (HOAc), and 25 mL of anhydrous ethanol (EtOH). The atmosphere was exchanged to hydrogen (H₂) with three evacuation-refill cycles and the reaction mixture was stirred overnight at room temperature. The reaction was monitored by thin layer chromatography. The reaction mixture was then filtered over Celite®, the filter residue was washed with EtOH, and the combined filtrates were evaporated under reduced pressure using a rotary evaporator. The residue was dissolved in dichloromethane (DCM), filtered through a 0.2 μM nylon syringe filter, and the solvent was removed under reduced pressure using a rotary evaporator to provide 800 mg (93% yield) of the title compound (42) as a colorless oil. R_{f}=0.45 (EtOAc/MeOH=10:1+1 vol-% HOAc). ¹H NMR (400 MHz, CDCl₃): δ=0.98 (s, 6H), 1.36-1.42 (m, 2H), 1.51-1.59 (m, 2H), 2.01 (s, 3H), 2.05-2.14 (m, 2H), 3.15-3.20 (m, 2H), 3.38-3.44 (m, 2H), 3.62-3.67 (m, 2H), 3.92 (s, 2H), 5.90-5.96 (br. m, 1H) ppm. MS (ESI) m/z 296.0 (M+H)⁺, 318.0 (M+Na)⁺.

### Example 43

### Bioavailability of Acamprosate Following Oral Administration of Acamprosate Prodrugs to Rats

Rats were obtained commercially and were pre-cannulated in the jugular vein. Animals were conscious at the time of the experiment. All animals were fasted overnight and until 4 hours post-dosing of a prodrug of Formula (I), Formula (III), or Formula (IV).

Rat blood samples (0.3 mL/sample) were collected from all animals prior to dosing and at different time-points up to 24 h post-dose into tubes containing EDTA. Two aliquots (100 μL each) were quenched with 300 μL methanol and stored at −20° C. prior to analysis.

To prepare analysis standards, 90 μL of rat blood was quenched with 300 μL methanol followed by 10 μL of spiking standard and/or 20 μL of internal standard. The sample tubes were vortexed for at least 2 min and then centrifuged at 3400 rpm for 20 min. The supernatant was then transferred to an injection vial or plate for analysis by LC-MS-MS.

To prepare samples for analysis, 20 μL of internal standard was added to each quenched sample tube. The sample tubes were vortexed for at least 2 min and then centrifuged at 3400 rpm for 20 min. The supernatant was then transferred to an injection vial or plate for analysis by LC-MS-MS.

LC-MS-MS analysis was performed using an API 4000 equipped with Agilent 1100 HPLC and a Leap Technologies autosampler. The following HPLC column conditions were used: HPLC column: Thermal-Hypersil-Keystone C18, 4.6×100 mm, 5 μm; mobile phase A: 0.1% formic acid in water; mobile phase B: 0.1% formic acid in acetonitrile; flow rate: 1.2 mL/min; gradient: 99% A/1% B at 0.0 min; 99% A/1% B at 0.5 min; 5% A/95% B at 1.8 min; 5% A/95% B at 3.5 min; 99% A/1% B at 3.6 min; and 99% A/1% B at 9.0 min. Acamprosate was monitored in negative ion mode. The LOQ was 0.004 μg/mL. The standard curve range was 0.004 to 10 μg/mL. Prodrug was monitored in positive ion mode. The LOQ and standard curve range was the same as for acamprosate.

Non-compartmental analysis was performed using WinNonlin software (v.3.1 Professional Version, Pharsight Corporation, Mountain View, Calif.) on individual animal profiles. Summary statistics on major parameter estimates was performed for Cₘₐₓ (peak observed concentration following dosing), Tₘₐₓ (time to maximum concentration is the time at which the peak concentration was observed), AUC₍₀₋ₜ₎ (area under the plasma concentration-time curve from time zero to last collection time, estimated using the log-linear trapezoidal method), AUC_{(0-∞)}, (area under the plasma concentration time curve from time zero to infinity, estimated using the log-linear trapezoidal method to the last collection time with extrapolation to infinity), and t_{1/2,z} (terminal half-life).

Acamprosate or acamprosate prodrug was administered by oral gavage to groups of four to six adult male Sprague-Dawley rats (about 250 g). Animals were conscious at the time of the experiment. Acamprosate or acamprosate prodrug was orally administered in 3.4% Phosal at a dose of 70 mg-equivalents acamprosate per kg body weight.

The oral bioavailability (F %) of acamprosate was determined by comparing the area under the acamprosate concentration vs time curve (AUC) following oral administration of an acamprosate prodrug with the AUC of the acamprosate concentration vs time curve following intravenous administration of acamprosate on a dose normalized basis. Compounds 22 and 32 exhibited an acamprosate oral bioavailability at least about 5 times greater than the acamprosate oral bioavailability following oral administration of an equivalent dose of acamprosate itself.

### Description 6

### Use of Clinical Trials to Assess the Efficacy of Acamprosate Prodrugs for Maintaining Abstinence from Alcohol

The efficacy of an acamprosate prodrug for treating alcoholism can be assessed using a randomized, double-blind, double-dummy, placebo-controlled trial. Patients aged 18 to 65 years meeting DSM IV criteria for alcohol dependence and having a history of alcohol dependence for at least 12 months are selected for the study. Patients are required to have undergone detoxification and have had five or more days of abstinence from alcohol before commencing treatment. Patients having a body weight of less than 60 kg receive an equivalent of 1332 mg/day (two 333 mg tablets in the morning and one at midday and in the evening) or placebo, and patients having a bodyweight of greater than 50 kg receive an acamprosate equivalent of 1998 mg/day (two 333 mg tablets in the morning, midday and evening) or placebo. Other acamprosate equivalent doses may be appropriate depending upon the pharmacokinetics of a particular acamprosate prodrug.

Primary and secondary outcome measures include commonly accepted subjective measures (based mainly on self-reported data) of continuous abstinence rate (CAR, i.e., the percentage of patients completely abstinent throughout the entire treatment and/or follow-up period), cumulative abstinence duration (CAD), the proportion of the total time that CAD represented (CADP, i.e. CAD as a proportion of the total treatment duration) and/or time to first drink (TFD). Surrogate biologcial markers of relapse such as γ-glutamyl transferase, carbohydrate-deficient transferrin, AST and ALT levels, and mean corpuscular volume can also be determined. Efficacy of acamprosate prodrugs in the maintenance of abstinence in patients with alcohol dependence is reflected in an increased CAR, CADP, and TFD compared to patients receiving placebo.

### Description 7

### Use of Animal Models to Assess the Efficacy of Acamprosate Prodrugs for Treating Alcohol Withdrawal

Withdrawal Seizure-Prone (WSP) and Withdrawal Seizure-Resistant (WSR) mice are used to assess the efficacy of acamprosate prodrugs for treating alcohol withdrawal. Mice are made dependent on ethanol via 72 h of chronic ethanol vapor inhalation. On day 1, mice are weighted, injected with a loading dose of ethanol and pyrazole HCl (Pyr), an alcohol dehydrogenase inhibitor, and placed into ethanol vapor chambers. Controls are placed into air chambers and receive Pyr only. At 24 and 48 h, Pyr boosters are administered to both the experimental and control groups. Blood ethanol concentrations (BECs) for ethanol groups are measured and the ethanol vapor concentrations adjusted to equate ethanol exposure between lines. Mean BECs are maintained between approximately 1.0-2.0 mg/mL, depending upon the effects of the test compound being studied. After 72 h, all mice are removed from the chambers to initiate withdrawal, and ethanol treated mice have blood samples drawn for BEC determinations.

Following removal from the ethanol or air chambers, mice are scored hourly for handling-induced convulsion (HIC). Scoring is initiated 1 h after removal from ethanol and hourly over the next 12-15 h and again at 24 h. If animals do not return to baseline HIC levels by 25 h, an additional score is obtained at 48 h. The scale such as the following is used (0—no convulsion after a gently 180° spin; 1—only facial grimace after gentle 180° spin; 2—tonic convulsion elicited by gently 180° spin; 3—tonic-clonic convulsion after 180° spin; 4—tonic convulsion when lifted by tail, no spin; 5—tonic-clonic convulsion when lifted by tail, no spin; 6—severe tonic-clonic convulsion when lifted by tail, no spin; and 7—severe tonic-clonic convulsion elicited before lifting by the tail). The area under the curve is calculated and used to quantitatively evaluate withdrawal severity. An additional index of withdrawal severity is the peak HIC score, calculated by identifying the highest HIC for each individual mouse and averaging this score with the two adjacent scores. Data are analyzed by appropriate statistical methods.

### Description 8

### Animal Model for Assessing Therapeutic Efficacy of Acamprosate Prodrugs for Treating Tinnitus

### Unilateral Noise Trauma

The efficacy of acamprosate prodrugs of Formula (I), Formula (III), and Formula (IV) for treating tinnitus can be assessed using animal models of tinnitus in which unilateral noise trauma is used to induce tinnitus (Bauer and Brozoski, J Assoc Res Otolarynology 2001, 2(1), 54-64; and Guitton et al., US 2006/0063802). Long-Evans rats are first behaviorally acclimated to lever-press for food pellets and then conditioned to respond in a distinctive and standard way to auditory test stimuli. After conditioning, the animals are separated into groups and exposed to unilateral noise trauma for 0, 1, or 2 hours. Animals are anesthetized, placed in a stereotaxic head frame, and unilaterally exposed once to narrowband noise with a peak intensity of 105 dB centered at 16 kHz for 0, 1, or 2 hours before or after behavioral training and testing. The animals are then administered an acamprosate prodrug and suppression of the conditioned response determined and compared to a control group not exposed to noise trauma.

### Sodium Salicylate-Induced Sound Experience

An animal model developed for short-term, acute induced phantom auditory sensations in rats can be used to evaluate acamprosate prodrugs for treating tinnitus. Salicylate-induced animal models of tinnitus are known.

Female albino rats (Wistar, aged 8-20 weeks) are trained and tested on five consecutive days per week. Training and testing takes place in a commercial conditioning chamber (rat shuttle box, TSE) adapted for the study. Electrical stimuli (0.1-0.5 mA, 100 V, 0.5 s) can be supplied via a shockable floor ground. A resting platform with a mechanical sensor is mounted on one side of the cage, covering the shockable floor and serving as a resting location for the animal. The cage is separated by a wall into two short hallways. At both ends of the hallways, within a recess, small amounts of fluid can be given to an animal, gravity-advanced and controlled by flow resistance- and vibration-muted magnetic shutter valves. A typical open time is 0.5 s, resulting in a reward drop of ca. 20 μL, supplied to an animal via a curved metal drinking cannula. Reward drops not taken up by the animal are drained off into a reservoir unreachable by the rat. Photo sensors registered the visits of an animal at the feeder recesses. All sensors are monitored on a computer screen and a top-mounted USB camera provided pictures of the entire floor dimensions of the cage interior.

Auditory stimuli are generated and presented over three broadened speakers mounted vertically in the cage. A continuous white noise can be plated on the central loudspeaker switched off and on with a 100 ms ramp. In parallel to the white noise sound, a pure tone (cue tone, 8 kHz, 70 dB SPL, 200 ms length, 25 ms ramp, repeated five times with 300 ms pause) could be presented over loudspeakers mounted directly over the left and right feeder recesses.

Animals are trained on auditory stimuli for 30-60 min/day for 5 days/week. Training session length is adapted to the animal's activity. Always 15-18 h prior to behavioral testing (experimental session), the drinking water is withdrawn. The conditioned rats are divided into two groups (one animal per cage for either group). Animals from the first group receive an intraperitoneal injection of sodium salicylate (350 mg/kg bw) while animals from the second group receive an intraperitoneal injection of an equivalent volume of saline. Animals from either group are tested on the same day in a semi-random order exactly 3 h after injection. During the experimental session electrical stimuli are omitted. Four minutes after the start of a session the sugar water reward is stopped and the behavioral performances are recorded from 12-16 min and subsequently analyzed. Within the next 2-5 days rats receive the same training as before the experiment. On the next experimental day animals from the group previously treated with salicylate are injected with saline or test compound and tested again.

Frequencies of feeder access action of a rat are calculated for periods of sound and periods of silence separately (accesses/min) and normalized (SA activity ratio). The difference of silence activity ratios (ΔSA ratio) is determined as the silence activity ratio of an animal tested after salicylate injection less the silence activity ratio of the same animal after saline injection. Data is analyzed using appropriate statistical methods.

During the training procedure, animals are conditioned to discriminate between periods of sound and periods of silence using auditory cues.

To induce phantom auditory sensations, animals are injected with salicylate (350 mg/kg bw) or an equivalent volume of saline and tested 3 h later. The SA ratio of animals treated with salicylate is significantly higher than the SA ratio for animals treated with saline.

Test compounds can be administered and their ability to reverse the effects of the salicylate induced phantom auditory sensations determined. Compounds that reduce the increase in the SA ratio following in the salicylate treated animals can have potential in treating tinnitus.

### Description 9

### Method for Assessing Therapeutic Efficacy of Acamprosate Prodrugs for Treating Tinnitus in Humans

The efficacy of acamprosate prodrugs of Formula (I), Formula (III), and Formula (IV) for treating tinnitus in humans can be assessed using methods known in the art.

Patients are screened using pre-established inclusion and exclusion criteria and selected for their ability to perform a psychophysical loudness matching task using pure tones and broad-band noise (BBN). Examples of inclusion criteria include, for example, age, type of tinnitus, e.g., continuous or pulsed, duration of tinnitus, Tinnitus Handicap Questionnaire (THQ) score >30, Beck Depression Index (BDI)<13, and criterion performance on loudness matching task using a 1 KHz standard.

Following screening, selection and enrollment, tinnitus is evaluated before and after an acamprosate prodrug is administered to a patient. Hearing thresholds are evaluated using an objective stimulus loudness match and a tinnitus loudness matching procedure.

Prior to enrollment, subjects are screened for proficiency in a psychophysical matching task. In the objective stimulus loudness matching procedure, subjects match a binaural 1 KHz standard tone at 20 dB sensation levels to each of five binaural comparison stimuli (BBN, 0.5, 1, 2, and 4 KHz). The loudness match is obtained using a forced two-choice procedure. Each trial begins with the simultaneous presentation of a visual cue and the 1 KHz standard followed by the presentation of the second visual cue and the comparison stimulus. Subjects are instructed to indicate whether the standard and comparison stimuli sound the “same” or “different” in loudness by clicking an on-screen button. An ascending-descending method of limits procedure is used. Subjects are screened using this loudness-matching test and are required to meet inclusion criteria of efficiency (completion time ≦1 h) and reliability (standard deviation of match levels ≦5 dB).

The tinnitus loudness matching procedure differs from the objective stimulus loudness matching procedure in that the initial presentation on each trial is a null presentation during which an on-screen message instructs subjects to listen closely to their tinnitus. During this initial 1-sec cue subjects are instructed to use their perception of tinnitus as the standard stimulus. Subjects are instructed to click a “same loudness” button when the loudness of the comparison stimulus matches the loudness of their tinnitus. The presentation order of the comparison stimuli (BBN, 0.5, 1, 2, and 4 KHz) is randomized, and each ascending and descending stimulus series is repeated once, for a total of four tinnitus loudness matches at each of the five comparison stimuli. The intensities of the loudness-match points are recorded and converted to sensation levels of tinnitus loudness using the hearing threshold determined in each session for the comparison stimuli. Psychoacoustically determined tinnitus loudness is reported as dB HL of the maximum sensation-level match obtained within a session.

Assessment sessions are performed at the initiation of the study and at intervals during the study. Subjects can be given placebo only, an acamprosate prodrug only, a variable including escalating or deescalating dose of an acamprosate prodrug, or a combination of placebo and acamprosate prodrug during the course of a study. The duration of the study can be a few hours, days, weeks, months, or years.

Primary outcome measures are psychoacoustically determined tinnitus loudness and perceived tinnitus handicap. Tinnitus handicap can be determined using the Tinnitus Handicap Questionnaire, which provides a global score and subscores related to emotional, functional, and cognitive aspects of tinnitus. Secondary outcome measures include general health and quality of life factors determined using, for example, the General Health Survey Short form (RAND 36-Item Health Survey, 1.0, Rand Health, Santa Monica, Calif.) and the Tinnitus Experience Questionnaire, a set of seven scaled questions that evaluate the experiential sensory features of tinnitus. Other questionnaires for assessing tinnitus can be used.

### Description 10

### Animal Model for Assessing Therapeutic Efficacy of Acamprosate Prodrugs for Treating Sleep Apnea

Sprague-Dawley rats are anesthetized and a surgical incision of the scalp is made to allow bilateral implantation of stainless steel screws into the frontal and parietal bones of the skull for electroencephalogram (EEG) recording. Bilateral wire electrodes are placed into the nuchal muscles for electromyogram (EMG) recording. The skin is then sutured and the animals allowed at least 7 days for recovery. Respirations are recorded by placing each rat inside a single chamber plethysmograph. The plethysmograph chamber is flushed with room air at a constant regulated flow rate of 2 L/min. EEG, EMG and respirations are continuously recorded. Sleep apneas are defined as cessation of respiratory effort for at least 2.5 s. The effects of recording hour, sleep state, and acamprosate prodrug administration are analyzed using appropriate statistical methods.

### Description 11

### Study for Assessing the Therapeutic Efficacy of Acamprosate Prodrugs for Treating Sleep Apnea in Humans

Inclusion criteria are an apnea-hypopnea index (AHI) exceeding 20 based on self-rated sleep duration at previous unattended ventilatory screening or an AHI exceeding 25 in a previous polysomnographic (PSG) recording. A double blind, randomized, placebo-controlled cross-over study comparing the effects of an acamprosate prodrug and placebo is used. Each patient undergoes a complete PSG recording for habituation at night 1. Patients are randomized to receive acamprosate prodrug on night 2 and placebo on night 3, or vice versa. Night 2 is scheduled within 1-21 days after night 1 and night 3 within 7-28 days after night 1 to provide a minimum of 7 days between night 2 and night 3 washout. A complete PSG recording, physical examination, and recording of ECG is performed in an identical manner at all study nights. Blood samples are obtained in the morning after study nights for hematology and clinical chemistry. Adverse events are determined by active questioning. AHI, the number of obstructive apneic/hyponeic events per time, is the primary efficacy variable. Secondary efficacy variables are REM AHI, non-REM AHI, apnea index (AI), hypopnea index (HI), oxygen desaturation index (ODI), minimum overnight oxygen saturation, sleep stage distribution arousal index, REM sleep and slow wave sleep latency, safety and tolerability. An obstructive apnea is defined as loss of nasal pressure accompanied by paradoxical respiratory movements for >10 s. An obstructive hypopnea is defined as a >50% reduction of the nasal pressure signal, but accompanied by chest wall paradoxical motion through most of inspiration for >10 s. Events without respiratory movements are classified as central apneas.

### Description 12

### Animal Models for Assessing Therapeutic Efficacy of Acamprosate Prodrugs for Treating Parkinson's Disease

### MPTP Induced Neurotoxicity

MPTP, or 1-methyl-4-phenyl-1,2,3,6-tetrahydropyridine is a neurotoxin that produces a Parkinsonian syndrome in both man and experimental animals. Studies of the mechanism of MPTP neurotoxicity show that it involves the generation of a major metabolite, MPP⁺, formed by the activity of monoamine oxidase on MPTP. Inhibitors of monoamine oxidase block the neurotoxicity of MPTP in both mice and primates. The specificity of the neurotoxic effects of MPP⁺ for dopaminergic neurons appears to be due to the uptake of MPP⁺ by the synaptic dopamine transporter. Blockers of this transporter prevent MPP⁺ neurotoxicity. MPP⁺ has been shown to be a relatively specific inhibitor of mitochondrial complex I activity, binding to complex I at the retenone binding site and impairing oxidative phosphorylation. In vivo studies have shown that MPTP can deplete striatal ATP concentrations in mice. It has been demonstrated that MPP⁺ administered intrastriatally to rats produces significant depletion of ATP as well as increased lactate concentration confined to the striatum at the site of the injections. Compounds that enhance ATP production can protect against MPTP toxicity in mice.

A prodrug of Formula (I), Formula (III), or Formula (IV) is administered to mice or rats for three weeks before treatment with MPTP. MPTP is administered at an appropriate dose, dosing interval, and mode of administration for 1 week before sacrifice. Control groups receive either normal saline or MPTP hydrochloride alone. Following sacrifice the two striate are rapidly dissected and placed in chilled 0.1 M perchloric acid. Tissue is subsequently sonicated and aliquots analyzed for protein content using a fluorometer assay. Dopamine, 3,4-dihydroxyphenylacetic acid (DOPAC), and homovanillic acid (HVA) are also quantified. Concentrations of dopamine and metabolites are expressed as nmol/mg protein.

Prodrugs of Formula (I), Formula (III), and Formula (IV) that protect against DOPAC depletion induced by MPTP, HVA, and/or dopamine depletion are neuroprotective and therefore can be useful for the treatment of Parkinson's disease.

### Haloperidol-Induced Hypolocomotion

The ability of a compound to reverse the behavioral depressant effects of dopamine antagonists such as haloperidol in rodents and is considered a valid method for screening drugs with potential antiparkinsonian effects. Hence, the ability of prodrugs of Formula (I), Formula (III), and Formula (IV) to block haloperidol-induced deficits in locomotor activity in mice can be used to assess both in vivo and potential anti-Parkinsonian efficacy.

Mice used in the experiments are housed in a controlled environment and allowed to acclimatize before experimental use. 1.5 h before testing, mice are administered 0.2 mg/kg haloperidol, a dose that reduces baseline locomotor activity by at least 50%. A test compound is administered 5-60 min prior to testing. The animals are then placed individually into clean, clear polycarbonate cages with a flat perforated lid. Horizontal locomotor activity is determined by placing the cages within a frame containing a 3×6 array of photocells interfaced to a computer to tabulate beam interrupts. Mice are left undisturbed to explore for 1 h, and the number of beam interruptions made during this period serves as an indicator of locomotor activity, which is compared with data for control animals for statistically significant differences.

6-Hydroxydopamine Animal Model

The neurochemical deficits seen in Parkinson's disease can be reproduced by local injection of the dopaminergic neurotoxin, 6-hydroxydopamine (6-OHDA) into brain regions containing either the cell bodies or axonal fibers of the nigrostriatal neurons. By unilaterally lesioning the nigrostriatal pathway on only one-side of the brain, a behavioral asymmetry in movement inhibition is observed. Although unilaterally-lesioned animals are still mobile and capable of self maintenance, the remaining dopamine-sensitive neurons on the lesioned side become supersensitive to stimulation. This is demonstrated by the observation that following systemic administration of dopamine agonists, such as apomorphine, animals show a pronounced rotation in a direction contralateral to the side of lesioning. The ability of compounds to induce contralateral rotations in 6-OHDA lesioned rats has been shown to be a sensitive model to predict drug efficacy in the treatment of Parkinson's disease.

A 2 cm long incision is made along the midline of the scalp and the skin retracted and clipped back to expose the skull. A small hole is then drilled through the skull above the injection site. In order to lesion the nigrostriatal pathway, the injection cannula is slowly lowered to position above the right medial forebrain bundle at −3.2 mm anterior posterior, −1.5 mm medial lateral from the bregma, and to a depth of 7.2 mm below the duramater. Two minutes after lowering the cannula, 6-OHDA is infused at a rate of 0.5 μL/min over 4 min, to provide a final dose of 8 μg. The cannula is left in place for an additional 5 min to facilitate diffusion before being slowly withdrawn. The skin is closed, the animal removed from the sterereotaxic frame, and returned to its housing. The rats are allowed to recover from surgery for two weeks before behavioral testing.

Rotational behavior is measured using a rotameter system having stainless steel bowls (45 cm dia×15 cm high) enclosed in a transparent Plexiglas cover around the edge of the bowl and extending to a height of 29 cm. To assess rotation, rats are placed in a cloth jacket attached to a spring tether connected to an optical rotameter positioned above the bowl, which assesses movement to the left or right either as partial (45°) or full (360°) rotations.

To reduce stress during administration of a test compound, rats are initially habituated to the apparatus for 15 min on four consecutive days. On the test day, rats are given a test compound, e.g., a prodrug of Formula (I), Formula (III), or Formula (IV). Immediately prior to testing, animals are given a subcutaneous injection of a subthreshold dose of apomorphine, and then placed in the harness and the number of rotations recorded for one hour. The total number of full contralatral rotations during the hour test period serves as an index of antiparkinsonian drug efficacy.

### L-Dopa Induced Dyskinesia

The ability of acamprosate prodrugs to mitigate the effects of L-dopa induced dyskinesia can be assessed using animal models.

Male, Sprague-Dawley rats (250-300 g) are housed and maintained under standard conditions.

Reserpine (4 mg/kg) is administered under light isofluorane anesthesia. Eighteen hours following reserpine administration, the animals are placed into observation cages. Behavior is assessed using an automated movement detection system that includes dual layers of rectangular grids of sensors containing an array of 24 infrared beams surrounding the cage. Each beam break is registered as an activity count and contributes to the assessment of a variety of different behavioral parameters depending on the location of the event and the timing of successive beam breaks. These parameters include: (1) horizontal activity, a measure of the number of beams broken on the lower level; and (2) vertical activity, a measure of beams broken on the upper level.

In one experiment, immediately prior to commencing behavioral assessments, rats are injected with a combination of L-dopa methyl ester and carbidopa (or benserazide). In another study, to assess the effects of acamprosate prodrugs on L-dopa induced activity, animals are randomly assigned to groups. In each group, immediately following L-dopa/carbidopa administration, vehicle or acamprosate prodrug is administered. The behavior of normal, non-resperine-treated, animals is also assessed. Behavior of the animals in the different groups is monitored for at least 4 hours. Acamprosate prodrugs that reduce the L-dopa-induced locomotion in the reserpine-treated rats are potentially useful in treating Parkinson's disease and/or the symptoms associated with Parkinson's disease.

### Description 13

### Use of Clinical Trials to Assess the Efficacy of Acamprosate Prodrugs for Treating Parkinson's Disease

The following clinical study may be used to assess the efficacy of a compound in treating Parkinson's disease.

Patients with idiopathic PD fulfilling the Queen Square Brain Bank criteria with motor fluctuations and a defined short duration GABA analog response (1.5-4 hours) are eligible for inclusion. Clinically relevant peak dose dyskinesias following each morning dose of their current medication are a further pre-requisite. Patients are also required to have been stable on a fixed dose of treatment for a period of at least one month prior to starting the study. Patients are excluded if their current drug regime includes slow-release formulations of L-Dopa, COMT inhibitors, selegiline, anticholinergic drugs, or other drugs that could potentially interfere with gastric absorption (e.g. antacids). Other exclusion criteria include patients with psychotic symptoms or those on antipsychotic treatment, patients with clinically relevant cognitive impairment, defined as MMS (Mini Mental State) score of less than 24, risk of pregnancy, Hoehn & Yahr stage 5 in off-status, severe, unstable diabetes mellitus, and medical conditions such as unstable cardiovascular disease or moderate to severe renal or hepatic impairment. Full blood count, liver, and renal function blood tests are taken at baseline and after completion of the study.

A randomized, double blind, and cross-over study design is used. Each patient is randomized to the order in which either L-dopa or one of the two dosages of test compound, e.g., an acamprosate prodrug, is administered in a single-dose challenge in double-dummy fashion in three consecutive sessions. Randomization is by computer generation of a treatment number, allocated to each patient according to the order of entry into the study. All patients give informed consent.

Patients are admitted to a hospital for an overnight stay prior to administration of test compound the next morning on three separate occasions at weekly intervals. After withdrawal of all antiparkinsonian medication from midnight the previous day, test compound is administered at exactly the same time in the morning in each patient under fasting conditions.

Patients are randomized to the order of the days on which they receive placebo or test compound. The pharmacokinetics of a test compound can be assessed by monitoring plasma acamprosate concentration over time. Prior to administration, a 22 G intravenous catheter is inserted in a patient's forearm. Blood samples of 5 ml each are taken at baseline and 15, 30, 45, 60, 75, 90, 105, 120, 140, 160, 180, 210, and 240 minutes after administering a test compound or until a full off state has been reached if this occurs earlier than 240 minutes after drug ingestion. Samples are centrifuged immediately at the end of each assessment and stored deep frozen until assayed. Plasma acamprosate levels are determined by high-pressure liquid chromatography (HPLC). On the last assessment additional blood may be drawn for routine hematology, blood sugar, liver, and renal function.

For clinical assessment, motor function is assessed using the United Parkinson's Disease Rating Scale motor score and BrainTest, which is a tapping test performed with a patient's more affected hand on the keyboard of a laptop computer. These tests are carried out at baseline and then immediately following each blood sample until patients reach their full on-stage, and thereafter at 3 intervals of 20 min, and 30 min intervals until patients reach their baseline off-status. Once patients reach their full on-state, video recordings are performed three times at 20 min intervals. The following mental and motor tasks, which have been shown to increase dyskinesia, are monitored during each video session: (1) sitting still for 1 minute; (2) performing mental calculations; (3) putting on and buttoning a coat; (4) picking up and drinking from a cup of water; and (5) walking. Videotapes are scored using, for example, versions of the Goetz Rating Scale and the Abnormal Involuntary Movements Scale to document a possible increase in test compound induced dyskinesia.

Occurrence and severity of dyskinesia is measured with a Dyskinesia Monitor. The device is taped to a patient's shoulder on their more affected side. The monitor records during the entire time of a challenging session and provides a measure of the frequency and severity of occurring dyskinesias.

Results can be analyzed using appropriate statistical methods.

### Description 14

### Use of Clinical Trials to Assess the Efficacy of Acamprosate Prodrugs for Treating Levodopa-Induced Dyskinesias in Parkinson's Disease

A double-blind placebo-controlled clinical trial such as that described by Goetz et al., Movement Disorders 2007, 22(2), 179-186 can be used to assess the efficacy of an acamprosate prodrug for treating levodopa-induced dyskinesias in Parkinson's disease.

Patients are 30 years of age or older with Parkinson's disease and received levodopa treatment at a stable (at least 4 weeks) and optimized dose. Following enrollment, patients are randomized and receive either placebo or an appropriate dose and regimen of acamprosate prodrug. Levodopa doses are maintained at the baseline level. At appropriate intervals during the study, patients are evaluated for periods during the day characterized by sleep, off, on-without dyskinesias, on-with non-troublesome dyskinesias, and on-with troublesome dyskinesia. The primary outcome is change from baseline in on-time without dyskinesia. Various dyskinesia rating scales such as, for example, the Abnormal Involuntary Movement Scale, Unified Parkinson's Disease Rating Scale (UPDRS) Motor examination (Part III), or UPDRS Activities of Daily Living assessment (Part III) can also be used. Measures of safety such as frequency and severity of reported adverse events, changes in vital signs, laboratory test results, including ACTH-suppression testing of cortisol levels and electrocardiogram can also be determined.

### Description 15

### Animal Model for Assessing Therapeutic Efficacy of Acamprosate Prodrugs for Treating Alzheimer's Disease

Heterozygous transgenic mice expressing the Swedish AD mutant gene, hAPPK670N, M671L (Tg2576) are used as an animal model of Alzheimer's disease. Beginning at 9 months of age, mice are divided into two groups. The first two groups of animals receive increasing doses of an acamprosate prodrug, over six weeks. The remaining control group receives daily saline injections for six weeks.

Behavioral testing is performed at each drug dose using the same sequence over two weeks in all experimental groups: (1) spatial reversal learning, (2) locomotion, (3) fear conditioning, and (4) shock sensitivity. This order is selected to minimize interference among testing paradigms.

Acquisition of the spatial learning paradigm and reversal learning are tested during the first five days of test compound administration using a water T-. Mice are habituated to the water T-maze during days 1-3, and task acquisition begins on day 4. On day 4, mice are trained to find the escape platform in one choice arm of the maze until 6 to 8 correct choices are made on consecutive trails. The reversal learning phase is then conducted on day 5. During the reversal learning phase, mice are trained to find the escape platform in the choice arm opposite from the location of the escape platform on day 4. The same performance criterion and inter-trial interval are used as during task acquisition.

Large ambulatory movements are assessed to determine that the results of the spatial reversal learning paradigm are not influenced by the capacity for ambulation. After a rest period of two days, horizontal ambulatory movements, excluding vertical and fine motor movements, are assessed in a chamber equipped with a grid of motion-sensitive detectors on day 8. The number of movements accompanied by simultaneous blocking and unblocking of a detector in the horizontal dimension are measured during a one-hour period.

The capacity of an animal for contextual and cued memory is tested using a fear conditioning paradigm beginning on day 9. Testing takes place in a chamber that contains a piece of absorbent cotton soaked in an odor-emitting solution such as mint extract placed below the grid floor. A 5-min, 3 trial 80 db, 2800 Hz tone-foot shock sequence is administered to train the animals on day 9. On day 10, memory for context is tested by returning each mouse to the chamber without exposure to the tone and foot shock, and recording the presence or absence of freezing behavior every 10 seconds for 8 minutes. Freezing is defined as no movement, such as ambulation, sniffing or stereotypy, other than respiration.

On day 11, the response of the animal to an alternate context and to the auditory cue is tested. Coconut extract is placed in a cup and the 80 dB tone is presented, but no foot shock is delivered. The presence or absence of freezing in response to the alternate context is then determined during the first 2 minutes of the trial. The tone is then presented continuously for the remaining 8 minutes of the trial, and the presence or absence of freezing in response to the tone is determined. On day 12, the animals are tested to assess their sensitivity to the conditioning stimulus, i.e., foot shock. Following the last day of behavioral testing, animals are anesthetized and the brains removed, post-fixed overnight, and sections cut through the hippocampus. The sections are stained to image β-amyloid plaques (.

Data are analyzed using appropriate statistical methods.

### Description 16

### Animal Model for Assessing Therapeutic Efficacy of Acamprosate Prodrugs for Treating Huntington's Disease

### Neuroprotective Effects in a Transgenic Mouse Model of Huntington's Disease

Transgenic HD mice of the N171-82Q strain and non-transgenic littermates are treated with a prodrug of Formula (I), Formula (III), and Formula (IV) or a vehicle from 10 weeks of age. The mice are placed on a rotating rod (“rotarod”). The length of time at which a mouse falls from the rotarod is recorded as a measure of motor coordination. The total distance traveled by a mouse is also recorded as a measure of overall locomotion. Mice administered prodrugs of Formula (I), Formula (III), and Formula (IV) that are neuroprotective in the N171-82Q transgenic HD mouse model remain on the rotarod for a longer period of time and travel further than mice administered vehicle.

### Malonate Model of Huntington's Disease

A series of reversible and irreversible inhibitors of enzymes involved in energy generating pathways has been used to generate animal models for neurodegenerative diseases such as Parkinson's and Huntington's diseases. In particular, inhibitors of succinate dehydrogenase, an enzyme that impacts cellular energy homeostasis, has been used to generate a model for Huntington's disease. The enzyme succinate dehydrogenase plays a central role in both the tricarboxylic acid cycle as well as the electron transport chain in mitochondria. Malonate is a reversible inhibitor of succinate dehydrogenase. Intrastriatal injections of malonate in rats have been shown to produce dose dependent striatal excitotoxic lesions that are attenuated by both competitive and noncompetitive NMDA antagonists. For example, the glutamate release inhibitor, lamotrigine, also attenuates the lesions. Co-injection with succinate blocks the lesions, consistent with an effect on succinate dehydrogenase. The lesions are accompanied by a significant reduction in ATP levels as well as a significant increase in lactate levels in yl)o as shown by chemical shift resonance imaging. The lesions produce the same pattern of cellular sparing, which is seen in Huntington's disease, supporting malonate challenge as a useful model for the neuropathologic and neurochemical features of Huntington's disease.

To evaluate the effect of acamprosate prodrugs of Formula (I), Formula (III), and Formula (IV) in this malonate model for Huntington's disease, a prodrug of Formula (I), Formula (III), and Formula (IV) is administered at an appropriate dose, dosing interval, and route, to male Sprague-Dawley rats. A prodrug is administered for two weeks prior to the administration of malonate and then for an additional week prior to sacrifice. Malonate is dissolved in distilled deionized water and the pH adjusted to 7.4 with 0.1 M HCl. Intrastriatal injections of 1.5 μL of 3 μmol malonate are made into the left striatum at the level of the Bregma 2.4 mm lateral to the midline and 4.5 mm ventral to the dura. Animals are sacrificed at 7 days by decapitation and the brains quickly removed and placed in ice cold 0.9% saline solution. Brains are sectioned at 2 mm intervals in a brain mold. Slices are then placed posterior side down in 2% 2,3,5-tiphenyltetrazolium chloride. Slices are stained in the dark at room temperature for 30 min and then removed and placed in 4% paraformaldehyde pH 7.3. Lesions, noted by pale staining, are evaluated on the posterior surface of each section. The measurements are validated by comparison with measurements obtained on adjacent Nissl stain sections. Compounds exhibiting a neuroprotective effect and therefore potentially useful in treating Huntington's disease show a reduction in malonate-induced lesions.

### Description 17

### Animal Model for Assessing Therapeutic Efficacy of Acamprosate Prodrugs for Treating Amyotrophic Lateral Sclerosis

A murine model of SOD1 mutation-associated ALS has been developed in which mice express the human superoxide dismutase (SOD) mutation glycine→alanine at residue 93 (SOD1). These SOD1 mice exhibit a dominant gain of the adverse property of SOD, and develop motor neuron degeneration and dysfunction similar to that of human ALS. The SOD1 transgenic mice show signs of posterior limb weakness at about 3 months of age and die at 4 months. Features common to human ALS include astrocytosis, microgliosis, oxidative stress, increased levels of cyclooxygenase/prostaglandin, and, as the disease progresses, motor neuron loss.

Studies are performed on transgenic mice overexpressing human Cu/Zn-SOD G93A mutations ((B6SJL-TgN(SOD1-G93A) 1 Gur)) and non-transgenic B6/SJL mice and their wild litter mates. Mice are housed on a 12-hr day/light cycle and (beginning at 45 d of age) allowed ad libitum access to either test compound-supplemented chow, or, as a control, regular formula cold press chow processed into identical pellets. Genotyping can be conducted at 21 days of age. The SOD1 mice are separated into groups and treated with a test compound, e.g., an acamprosate prodrug, or serve as controls.

The mice are observed daily and weighed weekly. To assess health status mice are weighed weekly and examined for changes in lacrimation/salivation, palpebral closure, ear twitch and pupillary responses, whisker orienting, postural and righting reflexes and overall body condition score. A general pathological examination is conducted at the time of sacrifice.

Motor coordination performance of the animals can be assessed by one or more methods known to those skilled in the art. For example, motor coordination can be assessed using a neurological scoring method. In neurological scoring, the neurological score of each limb is monitored and recorded according to a defined 4-point scale: 0—normal reflex on the hind limbs (animal will splay its hind limbs when lifted by its tail); 1—abnormal reflex of hind limbs (lack of splaying of hind limbs weight animal is lifted by the tail); 2—abnormal reflex of limbs and evidence of paralysis; 3—lack of reflex and complete paralysis; and 4—inability to right when placed on the side in 30 seconds or found dead. The primary end point is survival with secondary end points of neurological score and body weight. Neurological score observations and body weight are made and recorded five days per week. Data analysis is performed using appropriate statistical methods.

The rotarod test evaluates the ability of an animal to stay on a rotating dowel allowing evaluation of motor coordination and proprioceptive sensitivity. The apparatus is a 3 cm diameter automated rod turning at, for example, 12 rounds per min. The rotarod test measures how long the mouse can maintain itself on the rod without falling. The test can be stopped after an arbitrary limit of 120 sec. Should the animal fall down before 120 sec, the performance is recorded and two additional trials are performed. The mean time of 3 trials is calculated. A motor deficit is indicated by a decrease of walking time.

In the grid test, mice are placed on a grid (length: 37 cm, width: 10.5 cm, mesh size: 1×1 cm²) situated above a plane support. The number of times the mice put their paws through the grid is counted and serves as a measure for motor coordination.

The hanging test evaluates the ability of an animal to hang on a wire. The apparatus is a wire stretched horizontally 40 cm above a table. The animal is attached to the wire by its forepaws. The time needed by the animal to catch the string with its hind paws is recorded (60 sec max) during three consecutive trials.

Electrophysiological measurements (EMG) can also be used to assess motor activity condition. Electromyographic recordings are performed using an electromyography apparatus. During EMG monitoring mice are anesthetized. The measured parameters are the amplitude and the latency of the compound muscle action potential (CMAP). CMAP is measured in gastrocnemius muscle after stimulation of the sciatic nerve. A reference electrode is inserted near the Achilles tendon and an active needle placed at the base of the tail. A ground needle is inserted on the lower back of the mice. The sciatic nerve is stimulated with a single 0.2 msec pulse at supramaximal intensity (12.9 mA). The amplitude (mV) and the latency of the response (ms) are measured. The amplitude is indicative of the number of active motor units, while distal latency reflects motor nerve conduction velocity.

The efficacy of test compounds can also be evaluated using biomarker analysis. To assess the regulation of protein biomarkers in SOD1 mice during the onset of motor impairment, samples of lumbar spinal cord (protein extracts) are applied to ProteinChip Arrays with varying surface chemical/biochemical properties and analyzed, for example, by surface enhanced laser desorption ionization time of flight mass spectrometry. Then, using integrated protein mass profile analysis methods, data is used to compare protein expression profiles of the various treatment groups. Analysis can be performed using appropriate statistical methods.

### Description 18

### Assessing Therapeutic Efficacy of Acamprosate Prodrugs for Treating Cortical Spreading Depression

It has been hypothesized that cortical spreading depression emanating from a site of injury causes secondary damage in the “penumbra” by disrupting ion homeostasis and producing demands on neurons already in a compromised state. Focal CNS injury or ischemia also results in an induction of the immediate early gene c-fos. c-fos induction spreads throughout the injured hemisphere by a process that appears to be dependent on cortical spreading depression. c-fos induction is inhibited by NMDA receptor antagonists. Thus, both cortical spreading depression and c-fos induction are NMDA receptor activated processes associated with CNS injury and may be components of the cascade leading to neuron death.

NMDA-induced increase in fos immunoreactivty in mice is determined according to the following protocol. Male CF-1 mice (20 to 25 g) are administered varying doses of an acamprosate prodrug or vehicle. Thirty min later, animals receive intraperitioneal administration of NMDA (75 mg/kg) or vehicle. Sixty min later, animals are terminally anesthetized, brains are removed to ice and immersed for 1 h in 2% paraformaldehyde in phosphate buffered saline, and transferred to 15% sucrose in phosphate buffered saline, incubated overnight, and then frozen at −80° C. Coronal sections through the hippocampal region are taken, washed, and incubated with a sheep anti-fos polyclonal antibody (OA-11-824) for 18 h at 4° C. Sections are washed with phosphate buffered saline and then incubated with biotinylated rabbit anti-sheep antibody for 2 h. After 3 washes in phosphate buffered saline, sections are incubated in Vector ABC solution for 1 h at 25° C., washed 3 times, stained for glucose oxidase, and mounted. Each section is photographed and the intensity of fos-like immunoreactivity in the dentate gyrus is analyzed.

CNS trauma-induced c-fos mRNA induction in rats is determined according to the following protocol. Male Sprague-Dawley rats (200-250 g) are administered different doses of an acamprosate prodrug or vehicle. After 30 min, animals are anesthetized and a burr hole drilled over the right frontal parietal cortex 3 mm anterior and 3 mm lateral to bregma. An 18-gauge needle is inserted through the hole for 2 min to a depth of about 3 mm into the cortex. After a 60 min recovery animals are sacrificed, the brains removed, and cortices dissected and frozen in liquid nitrogen. Changes in c-fos mRNA expression following needle injury are quantified using procedures known in the art.

To assess the effects of acamprosate prodrugs on electrically-induced cortical spreading depression in rats, male Sprague-Dawley rats (275-325 g) are anaesthetized. The spontaneously breathing animals are fixed in a stereotaxic frame, a craniotomy is drilled over the parietal cortex, and the dura is removed. Two saline-filled glass recording microelectrodes each containing a Ag/AgCl wire are inserted into the parietal cortex at a depth of about 1 mm and 1.5-2.0 mm apart along the sagital plane using a micromanipulator. Two saline filled cannulae each containing a Ag/AgCl wire are inserted under the skin of the animal to serve as reference electrodes. Cortical spreading depression is induced in the parietal cortex using a bipolar stimulating electrode placed at 90° to the frontal recording electrode and positioned so that the electrode visibly touches but does not depress the cortex. Electrocortical stimulation consists of a train of 5 ms pulses at 40 Hz lasting for 2 s. The threshold stimulation for cortical spreading depression determined by varying the current. Once the threshold current has been determined, the current is increased by 20% for experimental measurements. DC potentials are recorded at 10 min intervals for four control stimulations. An acamprosate prodrug is then administered. DC potentials are again recorded at 10 min intervals. The speed of cortical spreading depression expansion is calculated from the latency difference of the negative DC shift at the rostral and caudal electrodes.

### Description 19

### Animal Models to Assess the Efficacy of Acamprosate Prodrugs for Treating Migraine

Therapeutic activity of acamprosate prodrugs provided by the present disclosure may be determined in various animal models of neuropathic pain or in clinically relevant studies of different types of neuropathic pain. Animal models for neuropathic pain are known in the art and include animal models that determine analgesic activity or compounds that act on the CNS to reduce the phenomenon of central sensitization that results in pain from nonpainful or nonnoxious stimuli. Other animal models that are known in the art, such as hot plate tests, model acute pain, are useful for determining analgesic properties of compounds that are effective when painful or noxious stimuli are present. The progression of migraines is believed to be similar to the progression of epilepsy (because an episodic phenomenon underlies the initiation of the epileptic episode) and, as such, it is believed that epilepsy animal models may be useful in determining efficacy in treating migraine.

### Analgesic Activity

The following test can be used to evaluate the analgesic activity of an acamprosate prodrug. Test compound is administered orally to mice. Morphine is administered as a reference substance at 64 mg/kg to mice under the same experimental conditions. A vehicle is administered to mice as a control substance under the same experimental conditions. Test compound, morphine, or vehicle is administered to the mice in a blind study. Sixty minutes after the test compound, morphine, or vehicle is administered, the mice are placed onto a hot metal plate maintained at 54° C. and surrounded by a Plexiglass cylinder. The time taken for the mice to lick their feet is an index of analgesic activity. Effective analgesics increase the latency or amount of time to licking. Latency to the first foot lick is measured, up to a maximum time of 30 sec to prevent tissue damage to the mice.

### Hyperreflexia and Flexor Reflex Tests

Assessment of hyperreflexia, pain, and muscle tone in chronic spinally transected rats is performed using male albino Holtzman-derived rats weighing 270-530 gm. The rats are housed independently and have continuous access to food and water throughout the experiments. Animals are anesthetized. Rats are placed in a stereotaxic frame and anesthesia is maintained. An incision is made so that the paraspinal muscles can be retracted and a laminectomy performed between T6-T9. A one- to two-millimeter portion of the spinal cord is removed by evacuation and replaced with gel foam to reduce bleeding, after which the incision is closed in layers.

Following the transection, rats are placed in a room in which the ambient temperature is raised to about 27° C. to maintain body temperature. On the following morning post-surgery, the hindquarters of the spinalized rats are bathed and their urine expressed manually by applying pressure to their bladders. Experiments are conducted between 21 and 28 days after surgery. For the first two weeks post-surgery, 0.25 mL of an antibiotic is administered to the rats to prevent bladder infection. A topical antibiotic is applied to any part of the skin that shows signs of decubitus lesions. Within approximately two weeks, all animals regain bladder control and are no longer given antibiotic treatment. Assessment of hyperreflexia and flexor reflex is performed before and after treatment with test compound so that each animal serves as its own control.

Initial assessment of hyperreflexia is performed by rating the hyperreflexia response elicited with an innocuous stimulus, such as a metal probe. A metal probe is pressed against the lower abdomen at four specific sites. The response is evaluated for each of four trials using a scale ranging from zero (no response in all four trials) to four (a maximum, tonic-clonic reaction elicited in all four trials). All scores, pre- and post-treatment, are transformed to indicate the percent of hyperreflexia, pain, or muscle tone. The data is analyzed using appropriate statistical methods.

After determining hyperreflexia before drug treatment, test compound is administered to the rats.

Polysynaptic flexor-reflex responses, elicited by stimuli that activate high-threshold afferents, are recorded as EMG activity from the ipsilateral hamstring muscle. Supramaximal electric shocks are applied to the hindpaw and recording electrodes are placed in the biceps femoris semitendinosus muscle. Five sets of stimuli are made at each time point. The flexor reflex is recorded, in periods with and without test compound, every 30 min once a stable baseline response is achieved. The data at time zero represent pre-treatment control values. The responses are determined in spinalized rats by observing the flexor-reflex response before treatment and at each of 30, 60, 90, and 120 min following administration of test compound, baclofen (10 mg/kg sc), or vehicle (water, 12 ml/kg po). Efficacy is indicated when a test compound is shown to reduce the magnitude of the flexor-reflex responses in a chronic spinalized rat at all time points with similar efficacy to baclofen, the positive control.

### Cutaneous Hypersensitivity Test

The effects of a test compound on nociceptive activation of the trigeminovascular system is determined using an animal model of migraine. A pharmaceutical composition comprising a test compound is administered to cats. To serve as positive and negative controls, a vehicle control is administered to the cats. Efficacy is indicated for compounds that inhibit trigeminovascular activation compared to the trigeminovascular activation in the cats that receive the vehicle.

### Yawning

Yawning is a behavior that has been linked to activation of dopaminergic neurotransmission. Yawning is part of a behavioral syndrome occurring in most patients during a migraine attack. Blockage of quinipirole-induced yawning in rats has been used as an animal model to study the potential antagonism of migraine symptoms.

Male Sprague Dawley rats are acclimatized for 12 days before testing and at the time of the study. The rats are housed in standard size steel cages with four animals per cage and are maintained on a 12 hour light/dark schedule. Test compound or vehicle is administered 15 min before the dopamine D2 agonist quinipirole in vehicle or the vehicle alone is administered to the animals. The animals are then placed individually in 6 in×6 in plexiglass observation cages and the number of yawns is counted for the subsequent 30 min. The data is analyzed by an appropriate statistical method.

The dopamine D2 agonist quinipirole can produce an average of 13-15 yawns per 30 minutes while no yawning behavior is typically observed in vehicle treated animals. Compounds that inhibit quinipirole-induced yawning may be efficacious in treating migraine.

### Animal Model of Dural Protein Extravasation

The following animal model can be employed to determine the ability of an acamprosate prodrug to inhibit protein extravasation, an exemplary functional assay of the neuronal mechanism of migraine.

Rats or guinea pigs are anesthetized and placed in a stereotaxic frame with the incisor bar set at −3.5 mm for rats or −4.0 mm for guinea pigs. Following a midline sagital scalp incision, two pairs of bilateral holes are drilled through the skull (6 mm posteriorly, 2.0 and 4.0 mm laterally in rats; 4 mm posteriorly and 3.2 and 5.2 mm laterally in guinea pigs, with all coordinates referenced to bregma). Pairs of stainless steel stimulating electrodes, insulated except at the tips are lowered through the holes in both hemispheres to a depth of 9 mm (rats) or 10.5 mm (guinea pigs) from dura.

Test compound is administered. About 7 min later a fluorescent dye (e.g., Evans Blue) is administered. The fluorescent dye complexes with proteins in the blood and functions as a marker for protein extravasation. Ten (10) min post-injection of the test compound, the left trigeminal ganglion is stimulated for 3 minutes at a current intensity of 1.0 mA (5 Hz, 4 msec duration) with a potentiostat/galvanostat. Fifteen minutes following stimulation, the animals are killed and exsanguinated with 20 mL of saline. The top of the skull is removed to facilitate collection of the dural membranes. Dural membrane samples are removed from both hemispheres, rinsed with water, and spread flat on microscopic slides. Once dried, the tissues are coverslipped with a 70% glycerol/water solution. A fluorescence microscope equipped with a grating monochromator and a spectrophotometer is used to quantify the amount of fluorescent dye in each sample.

The extravasation induced by the electrical stimulation of the trigeminal ganglion is an ipsilateral effect (i.e. occurs only on the side of the dura in which the trigeminal ganglion is stimulated). This allows the other (unstimulated) half of the dura to be used as a control. The ratio of the amount of extravasation in the dura from the stimulated side, over the amount of extravasation in the unstimulated side, is calculated. Control animals dosed with only saline, yield, for example, a ratio of about 2.0 in rats and about 1.8 in guinea pigs. In contrast, a compound that effectively prevents the extravasation in the dura from the stimulated side yields a ratio of about 1.0. Dose-response curves can be generated for a test compound and the dose that inhibits the extravasation by 50% (ID₅₀) or 100% (ID₁₀₀) can be determined.

### Amygdala Kindling Model

A relationship has been reported between migraine, affective illness and epilepsy. Although the three disorders are distinct, they all are paroxysmal dysregulations of the nervous system that partially overlap in their pharmacology. The kindling model for complex-partial seizures is based on the progressive development of seizures combined with electroencephalographic (EEG) paroxysmal patterns induced by repeated initially subconvulsive electrical stimulation of limbic structures, e.g., the basolateral nucleus of the amygdala. Once established, the phenomenon persists for months. Since the amygdala-kindled seizures in animals share numerous characteristics with complex-partial seizures in humans, it is a useful animal model of complex partial seizures. An advantage of using the amygdala kindling model is that both behavioral and EEG parameters of the partial and generalized seizures can be measured. Furthermore, the amygdala kindling model is reported to be appropriate for studying diseases such as migraine, affective illness, and epilepsy which increase in severity over time and in a manner which is related to the number of symptomatic episodes.

Rats are obtained at an age of 11-12 weeks (body weight 180-200 gm). Rats are maintained separately in plastic cages at controlled temperature (23° C.) and humidity (about 50% RH) with a 12-h light cycle. The rats receive standard diet and tap water ad libitum.

For implantation of stimulation and recording electrodes, rats are anesthetized and receive stereotaxic implantation of one bipolar electrode in the right basolateral amygdala. Coordinates for electrode implantation are AP-2.2 mm, L-4.8 mm, V-8.5 mm. All coordinates are measured from bregma. Skull screws serve as the reference electrode. The electrode assembly is attached to the skull by dental acrylic cement. After a postoperative period of 2 weeks, constant current stimulations (500 μA, 1 ms, monophasic square-wave pulses, 50/sec for 1 sec) are delivered to the amygdala at intervals of 1/day until ten stage 5 seizures are elicited. The electrical susceptibility of the stimulated region (threshold for induction of afterdischarges) is recorded on the first day of the experiment (initial afterdischarge threshold) as well as after kindling acquisition (with an interval of at least 4 days after the tenth stage 5 seizure) using an ascending staircase procedure. The initial current intensity is 1 μA, and the current intensity is increased in steps of about 20% of the previous current at intervals of 1 min until an afterdischarge of at least 3 sec duration is elicited. In addition to afterdischarge threshold, the following parameters of kindled seizures are measured in fully-kindled rats after stimulation with the afterdischarge threshold current: seizure severity is classified as follows: 1—immobility, eye closure, twitching of vibrissae, sniffing, facial clonus; 2—head nodding associated with more severe facial clonus; 3—clonus of one forelimb; 4—rearing, often accompanied by bilateral forelimb clonus; and 5—rearing with loss of balance and falling accompanied by generalized clonic seizures. Seizure duration 1 is the duration of limbic (stage 1-2) and/or motor seizures (stage 3-5). Seizure duration 2 includes the time of limbic and/or motor seizures plus the adjacent time of immobility. Afterdischarge duration 1 (ADD 1) is the time of spikes in the EEG recorded from the site of stimulation with a frequency of at least 1/sec. Afterdischarge duration 2 (ADD 2) is the total time of spikes occurring in the EEG including those, which followed the ADD 1 with lower frequency and amplitude.

Test compound is administered to the prepared animals. Control experiments are performed 2-3 days before each test compound experiment. For control determinations, rats receive vehicle (e.g., saline) with the pretreatment time of the respective test compound experiment. For all test compound experiments, at least 4 days are interposed between successive administrations in order to avoid alterations in drug potency due to cumulation or tolerance. Data is analyzed using appropriate statistical methods.

In addition to recordings of anticonvulsant parameters, kindled rats can be observed for adverse effects in order to estimate a therapeutic index. Tests include open field observations, rotarod test, and body temperature. Tests used to evaluate adverse effects are performed in the same manner in control and test compound experiments at two different times, immediately before application of a test compound or vehicle and 13 min after application.

The rotarod test is carried out with a rod of 6 cm diameter and rotation speed of 8 rpm. Neurological deficit is indicated by inability of the animals to maintain their equilibrium for at least 1 min on the rotating rod. Rats are trained prior to the rotarod evaluation to maintain their balance on the rod. After treatment with a test compound or vehicle, rats that are not able to maintain their equilibrium on the rod for three subsequent 1 min attempts are considered to exhibit neurological deficit.

In addition to these quantitative estimations of neurological deficit, behavioral alterations after administration of test compound are noted in the cage and after placing the animals in an open field of 90-100 cm diameter. Muscle tone is estimated by palpation of the abdomen. The extent of deficits in behavior after administration of a test compound is determined by a rating system. Animals are taken out of the cage, placed in an open field, observed for about 1 minute and rated separately for ataxia, abducted hindlimbs, reduced righting, flat body posture, circling, Straub tail, piloerection, hypolocomotion and hyperlocomotion (abdominal muscle tone is evaluated by palpation at the end of the period of observation). All other parameters except ataxia are scored from 0 to 3: 0—absent; 1—equivocal; 2—present; 3—intense. For ataxia: 1—slight ataxia in hind-legs (tottering of the hind quarters); 2—more pronounced ataxia with dragging of hind legs; 3—further increase of ataxia and more pronounced dragging of hind legs; 4—marked ataxia, animals lose balance during forward locomotion; 5—very marked ataxia with frequent loss of balance during forward locomotion; and 6—permanent loss of righting reflexes, but animal still attempts to move forward. Rectal body temperature is measured. Body weight of the animals is recorded once daily before a test compound is administered. Data is analyzed by an appropriate statistical method. The ability of a test compound to increase the electrical threshold for induction of afterdischarges, decrease the severity of seizures, reduce seizure duration, and reduce total afterdischarge duration suggests efficacy in treating migraine.

### Description 20

### Use of Clinical Trials to Assess the Efficacy of Acamprosate Prodrugs for Treating Migraine

The efficacy of a compound of Formula (I), Formula (III), and Formula (IV) in treating migraine may be assessed using a randomized, double blind, placebo-controlled, parallel group, clinical trial. The primary objective of the study is to evaluate the safety and efficacy of a test compound vs placebo in the treatment of recurrent episodes of migraine based on change from the baseline phase to the double-blind phase in the monthly (28 days) migraine episode rate. The secondary objectives are to evaluate the effect of treatment with a test compound versus placebo in migraine patients on percentage of subjects responding to treatment (50% or more reduction in monthly migraine episode rate) and change from the baseline phase to the double-blind phase in migraine days per month, average migraine duration, rescue medication use, average severity of migraine headache, average severity of migraine associated symptoms (nausea, vomiting, photophobia, phonophobia), to provide safety and efficacy data for the comparison a dose of a test compound in the treatment of migraine, and to evaluate the effect of treatment with a dose of a test compound versus placebo in migraine patients on migraine-specific measures of health-related quality of life (HRQL) and SF-36 quality-of-life measures, as well as the correlation between HRQL and migraine frequency.

The clinical trial is a randomized, double blind, placebo controlled, parallel-group, multicenter study to evaluate the efficacy and safety of one or more doses of a test compound versus placebo in migraine prophylaxis. Patients are randomized into treatment groups. The patients must have been diagnosed with migraine for at least twelve months, with or without aura, as defined by the International Headache Society (HIS). The IHS diagnostic criteria differ from the definition of a migraine period utilized in this study for evaluation of efficacy. For the purposes of this study a migraine period is defined as the twenty-four hour period starting with the onset of painful migraine symptoms, or aura with successful abortive/rescue treatment. Any recurrence during the twenty-four hour period is considered part of the initial episode. If the migraine pain persists beyond the twenty-four hour period, for the purposes of this study, this is considered a new episode.

There are four phases in the clinical trial: Baseline, Core Double-Blind, Blinded Extension, and Taper/Exit. The Baseline Phase lasts up to 42 days and includes two periods: Washout and Prospective Baseline. At Baseline Visit I (screening), patients are evaluated to ensure that they meet inclusion/exclusion criteria. In addition, a three-month retrospective headache history is recorded. During each of the three months prior to Visit 1, patients should have had no more than 8 migraines and no more than 15 total headache days (migraine plus other headache types). Eligible patients then undergo other study procedures and are given a headache/rescue medication record. Patients maintain this record from Visit I throughout their participation in the clinical trial, documenting the occurrence of any headaches, or auras, as well as the duration, severity, and symptomatology of any migraine attacks. Patients also record the use of any abortive/rescue medication taken for the relief of migraine pain and associated symptoms, or during an aura to prevent migraine pain or relieve symptoms. In addition, for each migraine attack, patients answer the questions on the headache record regarding work loss and productivity. If at the start of the trial, eligible patients are on any prophylactic medication to treat their migraines, they enter a Washout Period of up to 14 days to taper from these medications. This washout is concluded by the time the patient enters the Prospective Baseline Period, 28 days prior to Visit 2 (randomization).

At Baseline Visit 2 (Day 1), headache/rescue medication record information is reviewed. To be eligible for randomization into the trial a patient must have had 3 to 12 migraine episodes but no greater than 15 (migraine and non-migraine), headache days during the 28 days prior to Visit 2.

In the Core Double-Blind Phase, patients who complete the Baseline Phase and meet the entry criteria (including Prospective Baseline Period migraine/headache rate) are randomized into treatment groups representing one or more doses of test compound or placebo. The Core Double-Blind Phase has two periods: Titration and Maintenance.

The Titration Period immediately follows the Baseline Phase and extends for eight weeks (56 days). During this period, patients randomized to test compound are started at an initial dose and the daily dose is increased weekly until the assigned dose is achieved (or maximum tolerated dose, whichever is less). From the third week of Titration until the end of the Maintenance Period, a maximum of two dose level reductions are permitted for unacceptable tolerability problems. If a patient is still in the Titration Period, after a dose reduction, rechallenge is attempted to approach the patient's assigned dose, and, if unsuccessful, the dose is reduced again to the original reduced dose. Patients who have already had their study medication dose decreased by two levels, and are still experiencing unacceptable tolerability problems, which warrant additional dose reductions, exit the study, or enter the Open Label Extension Phase, where their dose is further adjusted. Clinic visits occur on, for example, Day 29 (Visit 3) and Day 57 (Visit 4/End of Titration).

During the 18-week Maintenance Period, patients remain on the dose of test compound reached at the end of the Titration Period (the assigned dose or the maximum tolerated dose). If a patient experiences unacceptable tolerability problems, the dose is reduced, but only to the point that there are no more than two dose reductions for the entire Core Phase (Titration plus Maintenance). No rechallenge is permitted during the Maintenance Period, so a patient continues on the reduced dose for the remainder of the period. Patients who have already had their study medication dose decreased by two levels, and are still experiencing unacceptable tolerability problems, which would warrant additional dose reductions, exit the study. Clinic visits occur, for example, on Day 83 (Visit 5), Day 113 (Visit 6), Day 141 (Visit 7) and Day 183 (Visit 8/Core Double-Blind Final Visit or Early Withdrawal).

Patients are considered to have completed the Core Double-Blind Phase if they complete all 26 weeks of the Phase (8 weeks of Titration and 18 weeks of Maintenance) without prematurely discontinuing study medication. Only patients who complete all 26 weeks of the Core Phase have the option of entering the Blinded Extension Phase.

During the Blinded Extension Phase, patients remain on test compound at the same dose they achieve during the Core Phase for six months, or until they withdraw. During this phase, patients are not permitted to adjust the dose of test compound. Patients are seen quarterly during this phase (Visits 10 and 11/Blinded Extension Final Visit). Patients are considered to have completed the Blinded Extension Phase if they complete all six months of the Phase without prematurely discontinuing the test compound.

In the Taper/Exit Phase, patients exiting the study are tapered from study medication. If a patient exits the study during the Core Double-Blind Phase (Titration or Maintenance Period), he or she is tapered from study medication in a blinded fashion. The length of the taper is as long as seven weeks, but varied according to the dose the patient achieves. Patients who exit the study during the Blinded Extension Phase are tapered from their medication following the recommended taper schedule.

Physical examinations (including height) and neurologic examinations are performed at the beginning and end of the study. A baseline electrocardiogram is performed at the beginning of the study. Vital signs and weight are recorded at each clinic visit. Adverse events are recorded. Quality of Life assessments are performed at intervals, for example, Visits 2 (Day 1), 4 (Day 57/Exit from Titration), 6 (Day 113) and 8 (Day 183/Core Double-Blind Final Visit/Early Withdrawal). Health Care Resource Use information is recorded at intervals, for example, Visits 3 through 8. The occurrence of any headaches or auras, severity and symptomatology of any migraine headaches, and the use of rescue medication is transcribed from a patient's headache record to their case record form at each visit.

Efficacy evaluations are based on information recorded on the subject's headache/rescue medication record and Health-Related Quality of Life assessments. On the headache/rescue medication record the patients documented the following throughout his/her study participation: occurrence and duration of headaches (and auras if no headache pain develops), severity of migraine pain and associated symptoms, as well as the use of medication taken to relieve migraine pain or symptoms (or taken during an aura to relieve symptoms or prevent migraine pain). Health-Related Quality of Life (HRQL) assessments are completed at specified intervals throughout the study. The Migraine-Specific Quality of Life questionnaire (MSQ), and the Medical Outcomes Study Short Form-36 (SF-36) can be used to assess HRQL.

The primary efficacy criterion is the reduction in migraine episodes per month (28 days) during the Core Double-Blind Phase compared to the 28 day Prospective Baseline Period. Secondary efficacy criteria include the percentage of patients responding to treatment (50% or more reduction in the monthly (28 day) migraine episode rate) and reduction from the Prospective Baseline Period to the Core Double-Blind Phase in migraine days per month, monthly rate of all types of headaches, average migraine duration, rescue medication use, average severity of migraine headache, and average severity of migraine-associated symptoms (nausea, vomiting, photophobia, phonophobia). Also included in the secondary efficacy criteria is the effect of treatment with test compound versus placebo on migraine-specific measures of health-related quality of life (HRQL) and SF-36 quality-of-life measures, as well as the correlation between HRQL and migraine frequency. The Medical Outcomes Study Short Form-36 (SF-36) is the most frequently used generic measure of HRQL in migraine patients and has been used in studies of migraine. The SF-36 is a 36-item questionnaire measuring eight domains. The SF-36 has been shown to be reliable and valid in a wide variety of patient populations as well as for migraine patients. The migraine specific quality of life questionnaire (MSQ) can also be administered. The MSQ is a disease-specific instrument developed to assess quality of life relating to migraine. The MSQ has been used in published clinical trials of migraine therapy and has demonstrated evidence of reliability, validity, and responsiveness.

### Description 21

### Animal Model for Assessing Therapeutic Efficacy of Acamprosate Prodrugs for Treating Schizophrenia

### Morris Water Maze

The Morris Water Maze (MWM) is used as a well-validated hippocampus dependent test of visual-spatial memory. The MWM tests the ability of an animal to locate a hidden platform submerged under water by using extra-maze cues from the test environment. Rats are trained in a pool 1.8 m in diameter and 0.6 m high, containing water at about 26° C. A 10 cm square transparent platform is hidden in a constant position 1 cm below the water level in the pool. Only distal visuo-spatial cues are available to the rats for location of the submerged platform. The rats are given trials to find the hidden platform. The escape latency, i.e., the time required by the rats to find and climb onto the platform, is recorded for up to 120 s. Each rat is allowed to remain on the platform for 30 s, after which it is removed to its home cage. If the rat did not find the platform within 120 s, it is manually placed on the platform and returned to its home cage after 30 s.

Male Sprague-Dawley rats weighing 150-200 g are used. Ten days before the beginning of the experiments, the rats are handled once daily to reduce experimental stress. Acamprosate prodrug or control is administered to the rats for three consecutive days before behavioral testing. On each day of behavioral testing the rats are injected with either haloperidol or saline 30 min before behavioral assessment.

### PCP-Induced Hyperactivity Model

Male C57Bl/6J mice are used. Mice are received at 6-weeks of age. Upon receipt, mice are assigned unique identification numbers (tail marked) and are group housed with 4 mice/cage in OPTI mouse ventilated cages. All animals remain housed in groups of four during the study. All mice are acclimated to the colony room for at least two weeks prior to testing and are subsequently tested at an average age of 8 weeks of age. During the period of acclimation, mice and rats are examined on a regular basis, handled, and weighed to assure adequate health and suitability.

Test compounds are prepared and administered according to the following procedures. An acamprosate prodrug is dissolved in sterile injectable water and administered i.p. at a dose volume of 10 mL/kg at 60 min prior to PCP injection. The amount of acamprosate prodrug administered can range, for example, from 0.01 mg/kg to 100 mg/kg. As a positive control, clozapine (1 mg/kg) is dissolved in 10% DMSO and administered i.p. at a dose volume of 10 mL/kg at 30 min prior to PCP injection. PCP (5 mg/kg) is dissolved in sterile injectable water and administered i.p. at a dose volume of 10 mL/kg.

The Open Filed (OF) test is used to assess both anxiety and locomotor behavior. The open field chambers are Plexiglas square chambers (27.3×27.3×20.3 cm) surrounded by infrared photobeams (16×16×16) to measure horizontal and vertical activity. The analysis is configured to divide the open field into a center and periphery zone. Distance traveled is measured from horizontal beam breaks as a mouse moves, and rearing activity is measured from vertical beam breaks.

Mice are acclimated to the activity experimental room for at least 1 h to prior to testing. Eight animals are tested in each run. Mice are injected with water or acamprosate prodrug, placed in holding cages for 30 min, and then in the OF chamber for 30 min, removed from the OF chamber and injected with either water or PCP and returned to the OF chambers for a 60-minute session. A different group of mice are injected with either 10% DMSO or clozapine and placed in the OF chamber for 30 min, removed from the OF chamber and injected with PCP (5 mg/kg), and returned to the OF chambers for a 60-minute session.

Data is analyzed by analysis of variance (ANOVA) followed by post-hoc comparisons with Fisher Tests when appropriate. Baseline activity is measured during the first 30 min of the test prior to PCP injection. PCP-induced activity is measured during the 60 min following PCP injection. Statistical outliers that fall above or below 2 standard deviations from the mean are removed from the final analysis. An effect is considered significant if p<0.05.

### Auditory Startle and Prepulse Inhibition of Startle (PPI)

Young, adult male C57Bl/6J mice are used in this study. Mice are received at 6-weeks of age. Upon receipt, mice are assigned unique identification numbers (tail marked) and are group housed in standard mouse cages. For testing, animals are randomly assigned across treatment groups and balanced by PPI chamber.

Acoustic startle measures an unconditioned reflex response to external auditory stimulation. PPI consisting of an inhibited startle response (reduction in amplitude) to an auditory stimulation following the presentation of a weak auditory stimulus or prepulse, has been used as a tool for the assessment of deficiencies in sensory-motor gating, such as those seen in schizophrenia. Mice are placed in the PPI chamber (Med Associates) for a 5 min session of white noise (70 dB) habituation. A test session begins immediately after the 5 min acclimation period. The session starts with a habituation block of 6 presentations of the startle stimulus alone, followed by 10 PPI blocks of 6 different types of trials. Trial types are: null (no stimuli), startle (120 dB), startle plus prepulse (4, 8 and 12 dB over background noise i.e., 74, 78 or 82 dB) and prepulse alone (82 dB). Trial types are presented at random within each block. Each trial begins with a 50 ms null period during which baseline movements are recorded. There is a subsequent 20 ms period during which prepulse stimuli are presented and responses to the prepulse measured. Following a 100 ms pause, the startle stimuli are presented for 40 ms and responses are recorded for 100 ms from startle onset. Responses are sampled every ms. The inter-trial interval is variable with an average of 15 s (range from 10 to 20 s). In startle alone trials the basic auditory startle is measured and in prepulse plus startle trials the amount of inhibition of the normal startle is determined and expressed as a percentage of the basic startle response (from startle alone trials), excluding the startle response of the first habituation block.

For the normal mouse-PPI portion of the study, C57BL/6J mice are treated with vehicle, haloperidol or acamprosate prodrug and placed back in their holding cages. Thirty min following administration of vehicle or haloperidol and 60 min following injection of vehicle or acamprosate prodrug, normal mouse-PPI testing commences.

For the PCP-PPI portion of the study, C57BL/6J mice are treated with vehicle, clozapine, or acamprosate prodrug and returned to their holding cages. Thirty min later, all treatment groups are injected with vehicle or PCP. Thirty min following vehicle or PCP injection, PPI testing commences.

### Description 22

### Animal Model for Assessing Therapeutic Efficacy of Acamprosate Prodrugs for Treating Anxiety

The elevated plux-maze test can be used to assess the effects of test compounds on anxiety. A plus-maze is consists of two open arms (50×10 cm) and two closed arms (50×10×40 cm). The arms extend from a central platform (10×10 cm) and are raised 50 cm. Each mouse is placed at the center of the maze facing a closed arm and is allowed to explore the maze for 5 min. The time spent in the open arms and the time spent in the closed arms is monitored, and the percent of time spent in the open arms determined. Increased time spent in the open arms indicates an anxiolytic effect for the test condition. A test that measures spontaneous locomotor activity such as measurement in an activity cage can be used to determine whether the test compound also affects locomotor activity. It is desirable that a compound exhibiting an anxiolytic effect not decrease locomotor activity.

### Description 23

### Animal Models of Depression

### Forced Swim Test in Rats

Male Wistar rats weighting 230-270 g are acclimated to the colony room for a minimum of 1 week, handled daily for at least 4 days and habituated to saline injections for 2 days before the experiments.

Two glass cylinders (20 cm dia×40 cm height) are separated by black opaque partitions and filled with water at about 24° C. to a depth of 30 cm. At this depth a rat cannot stand on the cylinder bottom. The water level is 10 cm from the top. Water is changed before each animal is placed into the water tank. An experimental session consists of two trials. During the conditioning trial, rats are gently placed into the cylinders for 15 min. After the trial, rats are dried and placed into a warm cage with the paper towels for 10-15 min before being returned to their home cages. Twenty-four hours later, for the test trial, animals are placed again into the cylinders for a 5-min test session. Tests are video taped for subsequent quantitative behavioral analysis. The frequency and/or total duration are calculated for each of the following categories: passive/immobile behavior (floating is scored when an animal remains in the water with all four limbs motionless, except for occasional alternate movements of paws and tail necessary to prevent sinking and to keep head/nose above the water); active/mobile behaviors (swimming characterized by rigorous movements with all four legs; paddling characterized by floating with rhythmical simultaneous kicks and occasional pushes off the wall to give speed and direction to the drift), including escape-oriented behaviors (climbing characterized by intense movements with all four limbs, with the two forepaws breaking the surface of the water and being directed against the walls of the cylinder; diving characterized by movements towards the bottom of the cylinder with the head of the rat below its hind limbs), and self-directed behaviors (headshakes, vigorous headshakes to get water off the snout and eyes; wiping, rubbing water away from the snout). In addition, at the end of each test trial, fecal boli are counted. A test compound, control, or positive control (e.g., imipramine) is administered prior to the test.

### Tail Suspension Test in Mice

Mice are housed in standard laboratory cages and acclimated. Mice are moved from the housing room to the testing area in their home cages and allowed to adapt to the new environment for at least 1 h before testing. Immobility is induced by tail suspension. Mice are hung individually on a paper adhesive tape, 65 cm above a tabletop. Tape is placed approximately 1 cm from the tip of the tail. Animals are allowed to hang for 6 min and the duration of immobility is recorded. Mice are considered immobile only when hanging passively and completely motionless. Mice from these experiments are used one week later in locomotor activity studies. A test compound, control, or positive control (e.g., imipramine) is administered prior to the test.

### Locomotor Activity

The spontaneous locomotor activity of mice is measured in photoresistor actometers (circular cages, 25 cm in dia, 15 cm high, two light sources, two photoresistors), in which the animals are placed individually 1 h after administration of a test compound. The number of crossings of light beams is measured during the first 30 min of the experimental session. The first measurement is performed 6 min after placing an animal into the actometer.

### Description 24

### Animal Model for Assessing Therapeutic Efficacy of Acamprosate Prodrugs for Treating Tardive Dyskinesia

Vacuous chewing movements (VCM) are a rodent model of TD. In this model, animals are treated chronically with antipsychotics and their vacuous chewing motions are assessed by observation. The model has been shown to be sensitive to differential effects of typical and atypical antipsychotics and potential anti-dyskinetic agents.

Rats are housed in a controlled environment and allowed to acclimatize prior to testing. In order to limit neuroleptic-induced weight gain, food consumption is restricted to 15 g per animal per day. Rats are weighed biweekly throughout the study.

For two weeks prior to administration of test compound, animals are handled daily and habituated to the animal colony and the procedures related to drug administration and video recording. Subsequently (week 0), rats undergo a behavior video recording session following which they are randomized to a haloperidol treatment and a control group. The rats in the treatment group receive an intramuscular injection in the thigh muscles with haloperidol decanoate. The control rats are similarly injected with an equal volume of phosphate buffered saline (PBS). The haloperidol decanoate and saline injections are repeated every four weeks, for 20 weeks. Additional behavior video recording sessions are performed at weeks 12, 20 and 24 (i.e., 4 weeks after the last (fifth) injection). During the injection procedures, rats are handheld with minimal restraint.

On the basis of the results of the behavior assessment performed 24 weeks after the first haloperidol injection (i.e., baseline day), the haloperidol-treated rats are assigned to 10 subject-each treatment groups having an equal mean frequency of observed VCM episodes. One week later (i.e., test day), the groups are randomized to receive either 0.5 mL PBS (vehicle) or acamprosate prodrug in 0.5 mL PBS. Rats undergo a 30-150 min video recorded behavior assessment session following administration. Two weeks after the test day (i.e., post-test day), the video recorded behavior assessment session is repeated to investigate longer-term effects of the experimental treatments.

The videotapes are scored. A VCM episode is defined as a bout of vertical deflections of the lower jaw, which may be accompanied by contractions of the jaw musculature.

### Description 25

### Animal Model for Assessing Therapeutic Efficacy of Acamprosate Prodrugs for Treating Spasticity

The mutant spastic mouse is a homozygous mouse that carries an autosomal recessive trait of genetic spasticity characterized by a deficit of glycine receptors throughout the central nervous system. The mouse is normal at birth and subsequently develops a coarse tremor, abnormal gait, skeletal muscle rigidity, and abnormal righting reflexes at two to three weeks of age. Assessment of spasticity in the mutant spastic mouse can be performed using electrophysiological measurements or by measuring the righting reflex (any righting reflex over one second is considered abnormal), tremor (holding mice by their tails and subjectively rating tremor), and flexibility.

Models of acute spasticity including the acute decerebrate rat, the acute or chronic spinally transected rat, and the chronically spinal cord-lesioned rat. The acute models, although valuable in elucidating the mechanisms involved in the development of spasticity, have come under criticism due to the fact that they are acute. The animals usually die or have total recovery from spasticity. The spasticity develops immediately upon intervention, unlike the spasticity that evolves in the human condition of spasticity, which most often initially manifests itself as a flaccid paralysis. Only after weeks and months does spasticity develop in humans. Some of the more chronic-lesioned or spinally transected models of spasticity do postoperatively show flaccid paralysis. At approximately four weeks post-lesion/transection, the flaccidity changes to spasticity of variable severity. Although all of these models have their own particular disadvantages and lack of true representation of the human spastic condition, they are shown useful in developing treatments for spasticity in humans. Many of these models have also made use of different species, such as cats, dogs, and primates. Baclofen, diazepam, and tizanidine, effective antispastic agents in humans, are effective on different parameters of electrophysiologic assessment of muscle tone in these models.

The Irwin Test is used to detect physiological, behavioral, and toxic effects of a test substance, and indicates a range of dosages that can be used for later experiments. Typically, rats (three per group) are administered the test substance and are then observed in comparison with a control group given vehicle. Behavioral modifications, symptoms of neurotoxicity, pupil diameter, and rectal temperature are recorded according to a standardized observation grid derived from that of Irwin. The grid contains the following items: mortality, sedation, excitation, aggressiveness, Straub tail; writhes, convulsions, tremor, exopthalmos, salivation, lacrimation, piloerection, defecation, fear, traction, reactivity to touch, loss of righting reflexes, sleep, motor incoordination, muscle tone, stereotypes, head-weaving, catalepsy, grasping, ptosis, respiration, corneal reflex, analgesia, abnormal gait, forepaw treading, loss of balance, bead twitches, rectal temperature, and pupil diameter. Observations are performed at 15, 30, 60, 120, and 180 minutes following administration of a test compound, and also 24 hours later.

In the Rotarod Test rats or mice are placed on a rod rotating at a speed of eight turns per minute. The number of animals that drop from the rod before three minutes is counted and the drop-off times are recorded (maximum: 180 sec). Diazepam, a benzodiazepine, can be administered at 8 mg/kg, i.p., as a reference substance.

### Description 26

### Animal Model for Assessing Therapeutic Efficacy of Acamprosate Prodrugs for Treating Multiple Sclerosis

Experiments are conducted on female C57BL/6 mice aged 4-6 weeks weighing 17-20 g. Experimental autoimmune encephalomyelitis (EAE) is actively induced using ≧95% pure synthetic myelin oligodendrocyte glycoprotein peptide 35-55 (MOG35-55, MEVGWYRSPFSRVVHLYRNGK). Each mouse is anesthetized and receives 200 μg of MOG peptide and 15 μg of Saponin extract from Quilija bark emulsified in 100 μL of phosphate-buffered saline. A 25 μL volume is injected subcutaneously over four flank areas. Mice are also intraperitoneally injected with 200 ng of pertussis toxin in 200 μL of PBS. A second, identical injection of pertussis toxin is given after 48 h.

An acamprosate prodrug is administered at varying doses. Control animals receive 25 μL of DMSO. Daily treatment extends from day 26 to day 36 post-immunization. Clinical scores are obtained daily from day 0 post-immunization until day 60. Clinical signs are scored using the following protocol: 0, no detectable signs; 0.5, distal tail limpness, hunched appearance and quiet demeanor; 1, completely limp tail; 1.5, limp tail and hindlimb weakness (unsteady gait and poor grip with hindlimbs); 2, unilateral partial hindlimb paralysis; 2.5, bilateral hindlimb paralysis; 3, complete bilateral hindlimb paralysis; 3.5, complete hindlimb paralysis and unilateral forelimb paralysis; 4, total paralysis of hindlimbs and forelimbs.

Inflammation and demyelination are assessed by histology on sections from the CNS of EAE mice. Mice are sacrificed after 30 or 60 days and whole spinal cords are removed and placed in 0.32 M sucrose solution at 4° C. overnight. Tissues are prepared and sectioned. Luxol fast blue stain is used to observe areas of demyelination. Haematoxylin and eosin staining is used to highlight areas of inflammation by darkly staining the nuclei of mononuclear cells. Immune cells stained with H&E are counted in a blinded manner under a light microscope. Sections are separated into gray and white matter and each sector is counted manually before being combined to give a total for the section. T cells are immunolabelled with anti-CD3+ monoclonal antibody. After washing, sections are incubated with goat anti-rat HRP secondary antibody. Sections are then washed and counterstained with methyl green. Spenocytes isolated from mice at 30 and 60 days post-immunization are treated with lysis buffer to remove red blood cells. Cells are then resuspended in PBS and counted. Cells at a density of about 3×106 cells/mL are incubated overnight with 20 μg/mL of MOG₃₅₋₅₅ peptide. Supernatants from stimulated cells are assayed for IFN-γ protein levels using an appropriate mouse IFN-γ immunoassay system.

### Description 27

### Animal Models of Pain

### Inflammatory Pain—Formalin Test

A formalin assessment test is used. Fifty μL of a 5% formalin solution is injected subcutaneously into the dorsal aspect of the right hind paw and the rats are then individually placed into clear observation cages. Rats are observed for a continuous period of 60 min or for periods of time corresponding to phase I (from 0 to 10 min following formalin injection) and phase II (from 30 to 50 min following formalin injection) of the formalin test (Abbott et al., Pain 1995, 60, 91-102). The number of flinching behaviors of the injected paw is recorded using a sampling technique in which each animal is observed for one 60-sec period during each 5-min interval. Test compound is administered 30 min or other appropriate interval prior to formalin injection.

### Inflammatory Pain—Carrageenan-Induced Acute Thermal Hyperalgesia and Edema

Paw edema and acute thermal hyperalgesia are induced by injecting 100 μL of a 1% solution of λ-carrageenan in physiological saline into the plantar surface of the right hind paw. Thermal hyperalgesia is determined 2 h following carrageenan injection, using a thermal paw stimulator. Rats are placed into plastic cubicles mounted on a glass surface maintained at 30° C. and a thermal stimulus in the form of radiant heat emitted form a focused projection bulb is then applied to the plantar surface of each hind paw. The stimulus current is maintained at 4.50±0.05 amp, and the maximum time of exposure is set at 20.48 sec to limit possible tissue damage. The elapsed time until a brisk withdrawal of the hind paw from the thermal stimulus is recorded automatically using photodiode motion sensors. The right and left hind paw of each rat is tested in three sequential trials at about 5-min intervals. Carrageenan-induced thermal hyperalgesia of paw withdrawal latency (PWLₜₕₑᵣₘₐₗ) is calculated as the mean of the two shortest latencies. Test compound is administered 30 min before assessment of thermal hyperalgesia.

The volume of paw edema is measured using water displacement with a plethysmometer 2 h following carrageenan injection by submerging the paw up to the ankle hairline (approx. 1.5 cm). The displacement of the volume is measured by a transducer and recorded. Test compound is administered at an appropriate time following carrageenan injection, such as for example, 30 min or 90 min.

### Visceral Pain

Thirty min following administration of test compound, mice receive an injection of 0.6% acetic acid in sterile water (10 mL/kg, i.p.). Mice are then placed in table-top Plexiglass observation cylinders (60 cm high×40 cm diameter) and the number of constrictions/writhes (a wave of mild constriction and elongation passing caudally along the abdominal wall, accompanied by a slight twisting of the trunk and followed by bilateral extension of the hind limbs) is recorded during the 5-20 min following acetic acid injection for a continuous observation period of 15 min.

### Neuropathic Pain—Spinal Nerve Ligation

Rats receive unilateral ligation of the lumbar 5 (L5) and lumbar 6 (L6) spinal nerves. The left L5 and L6 spinal nerves of the rat are isolated adjacent to the vertebral column and tightly ligated with a 5-0 silk suture distal to the dorsal root ganglia, and care is taken to avoid injury of the lumbar 4 (L4) spinal nerve. Control rats undergo the same procedure but without nerve ligation. All animals are allowed to recover for at least 1 week and not more than 3 weeks prior to assessment of mechanical allodynia. Mechanical allodynia is measure using calibrated von Frey filaments. Rats are placed into inverted plastic containers (20×12.5×20 cm) on top of a suspended wire mesh grid and acclimated to the test chamber for 20 min. The von Frey filaments are presented perpendicularly to the plantar surface of the selected hind paw, and then held in this position for approximately 8 s with enough force to cause a slight bend in the filament. Positive responses include an abrupt withdrawal of the hind paw from the stimulus or flinching behavior immediately following removal of the stimulus. A 50% paw withdrawal threshold (PWT) is determined. Rats with a PWT ≦5.0 g are considered allodynic and utilized to test the analgesic activity of a test compound. The test compound can be administered 30 min prior to the assessment of mechanical allodynia.

### Neuropathic Pain—Chronic Constriction Injury of the Sciatic Nerve

A model of chronic constriction injury of the sciatic nerve-induced neuropathic pain is used. The right common sciatic nerve is isolated at mid-thigh level and loosely ligated by four chromic gut (4-0) ties separated by an interval of 1 mm. Control rats undergo the same procedure but without sciatic nerve constriction. All animals are allowed to recover for at least 2 weeks and for no more than 5 weeks prior to testing of mechanical allodynia. Allodynic PWT is assessed in the animals as described for animals with spinal nerve ligation. Only rats with a PWT ≦5.0 g are considered allodynic and utilized to evaluate the analgesic activity of a test compound. Test compound is administered 30 min or other appropriate time prior to the assessment of mechanical allodynia.

### Neuropathic Pain—Vincristine-Induced Mechanical Allodynia

A model of chemotherapy-induced neuropathic pain is produced by continuous intravenous vincristine infusion (Nozaki-Taguchi et al., Pain 2001, 93, 69-76). Anesthetized rats undergo a surgical procedure in which the jugular vein is catheterized and a vincristine-primed pump is implanted subcutaneously. Fourteen days of intravenous infusion of vincristine (30 μg/kg/day) results in systemic neuropathic pain of the animal. Control animals undergo the same surgical procedure, with physiological saline infusion. PWT of the left paw is assessed in the animals 14 days post-implantation as described for the spinal nerve ligation model. Test compound is administered 30 min prior to the test for mechanical allodynia in rats with PWT≦5.00 g before treatment.

### Post-Operative Pain

A model of post-operative pain is performed in rats. The plantar aspect of the left hind paw is exposed through a hole in a sterile plastic drape, and a 1-cm longitudinal incision is made through the skin and fascia, starting 0.5 cm from the proximal edge of the heel and extending towards the toes. The plantaris muscle is elevated and incised longitudinally leaving the muscle origin and insertion points intact. After hemostasis by application of gently pressure, the skin is apposed with two mattress sutures using 5-0 nylon. Animals are then allowed to recover for 2 h following surgery, at which time mechanical allodynia and thermal hyperalgesia are assessed.

Effects of test compound on mechanical allodynia are assessed 30 min following administration, with PWT being examined in these animals for both the injured and non-injured paw as described for the spinal nerve ligation model with the von Frey filament systematically pointing towards the medial side of the incision. In a separate experiment, the effects of test compound on thermal hyperalgesia are assessed 30 min following administration of test compound, with PWLₜₕₑᵣₘₐₗ being determined as described for the carrageen-induced thermal hyperalgesia model with the thermal stimulus applied to the center of the incision of the paw planter aspect.

### Description 28

### Animal Model for Assessing Therapeutic Efficacy of Acamprosate Prodrugs for Treating Binge Eating

Thirty 2-month old male Sprague Dawley rats are individually housed in a temperature- and humidity-controlled vivarium under a 12:12 light:dark cycle. Three days after being introduced into the vivarium, rats are given overnight access to a bowl of vegetable shortening. The rats are then divided into three groups of ten matched for two-day average chow intake, overnight shortening intake, and body weight.

The groups and different test phases are designed to test the effects of acamprosate prodrug under different shortening access conditions. In phase 1, rats maintained on a feeding protocol that promotes infrequent, large binges (B group) are compared to rats maintained on feeding protocols that promote no binges (FM and C groups). In phase 2, rats maintained for an extended period of time on the infrequent, large binge protocol (B group) are compared to rats that have just started the same binge protocol (FM and C groups). In phase 3, rats maintained on the feeding protocol that promotes infrequent, large binges (B group) are compared to rats on a feeding protocol that promotes more frequent, smaller binges (FM and C groups).

The three groups are maintained as follows: Binge (B): The (B) rats have continuous access to chow and water. In addition, they are given 2-h access to a separate bowl of vegetable shortening every Monday, Wednesday, and Friday (MWF), during the 2 h prior to no light. During the 2-h shortening access period, the chow and water remain available. This protocol results in infrequent, large episodes of binge-type eating in male rats. This protocol models the intermittent excessive eating behavior that characterizes binge eating. The B rats are maintained on this protocol throughout all phases of the study. Fat-Matched (FM): The rats in group FM are given the same proportions of chow and shortening that the Binge (B) groups consume except that the shortening is mixed into the chow, which is provided continuously. The proportions of chow and shortening consumed by the Binge group each week are determined, and the FM group is provided with a fat-matched chow mixed to that proportion the following week. The FM group has free access to the FM chow and water. The FM group is included to control for possible neural or behavioral effects of dietary fat. The FM group is maintained on the FM chow throughout all phases of the study. During phase 1, the FM group only has access to the FM chow. During phase 2, the FM group has access to a separate bowl of vegetable shortening for 2-h on MWF each week, in addition to the continuously available FM chow. During phase 3, the FM group has 2-h access to the vegetable shortening every day, in addition to the continuously available FM chow. This daily protocol results in more frequent, smaller episodes of binge-type eating. Chow/change (C): The rats in group C have continuous access to the regular chow and water through all phases of the study. During the first phase, the C group only has access to the regular chow diet. During the second phase, the C group has access to a separate bowl of vegetable shortening for 2-h on MWF each week in addition to the continuously available regular chow. During the third phase, the C group has 2-h access to the vegetable shortening every day in addition to the continuously available regular chow.

The effects of acamprosate prodrugs effects are determined during each of the three phases of the study. In phase 1, the effects of acamprosate prodrug are determined on binge-type consumption of vegetable shortening and on consumption of the regular and FM chow diets. Rats are on their respective diets for about 6 weeks prior to the initiation of acamprosate prodrug testing. In phase 2, the effects of acamprosate prodrug are assessed in rats that are bingeing for a relatively long (B group: three months) or short (FM and C groups: 1 day) period of time (all groups have MWF 2-h access to shortening in addition to their assigned regular or FM chow). In phase 3, the effects of acamprosate prodrug are assessed under conditions of infrequent (B: 2-h MWF) and more frequent (FM and C groups: 2-h daily) shortening access. The FM and C rats are on the daily shortening access schedule for ten days before the first acamprosate prodrug administration in phase 3. Acamprosate prodrug is not tested in rats with continuous access to a bowl of shortening due to the low 2-h intakes that are generated on that protocol under non-food-deprived conditions. A dose and regimen of acamprosate prodrug is administered as appropriate for the objectives of the study.

Acamprosate prodrug is administered at an appropriate time prior to the shortening access period. Chow is removed during the 30-min pretreatment period. Shortening and/or chow are weighted and placed into the cage at the beginning of the test period, e.g., 2-h, and then re-weighted at the end of the test period. The data is analyzed using appropriate statistical methods.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

What is claimed is:

1. A compound of Formula (I): 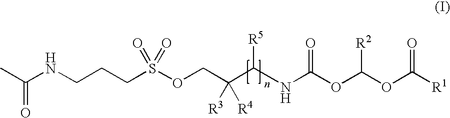
or a pharmaceutically acceptable salt thereof, wherein:
n is chosen from 0, 1, 2, and 3;
R¹ is chosen from C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;
R² is chosen from hydrogen, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ beterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;
R³ and R⁴ are independently chosen from C₁₋₄ alkyl and substituted C₁₋₄ alkyl; or R³ and R⁴ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and
each R⁵ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

2. The compound of claim 1, wherein R¹ is chosen from C₁₋₆ alkyl, C₁₋₆ alkoxy, phenyl, and substituted phenyl.

3. The compound of claim 1, wherein R² is chosen from hydrogen and C₁₋₆ alkyl.

4. The compound of claim 1, wherein each of R³ and R⁴ is methyl.

5. The compound of claim 1, wherein each R⁵ is hydrogen.

6. The compound of claim 1, wherein n is chosen from 0, 1, and 2.

7. The compound of claim 1, wherein R¹ is chosen from C₁₋₆ alkyl, C₁₋₆ alkoxy, phenyl, and substituted phenyl; R² is chosen from hydrogen and C₁₋₆ alkyl; each of R³ and R⁴ is methyl; each R⁵ is hydrogen; and n is chosen from 0, 1, and 2.

8. The compound of claim 1, wherein the compound is chosen from:
[N-(4-{[3-(acetylamino)propyl]sulfonyloxy}-3,3-dimethylbutyl)carbamoyloxy]ethyl 2-methylpropanoate;
[N-(4-{[3-(acetylamino)propyl]sulfonyloxy}-3,3-dimethylbutyl)carbamoyloxy]ethyl benzoate;
[N-(5-{[3-(acetylamino)propyl]sulfonyloxy}-4,4-dimethylpentyl)carbamoyloxy]ethyl 2-methylpropanoate;
[N-(5-{[3-(acetylamino)propyl]sulfonyloxy}-4,4-dimethylpentyl)carbamoyloxy]methyl benzoate;
[N-(3-{[3-(acetylamino)propyl]sulfonyloxy}-2,2-dimethylpropyl)carbamoyloxy]ethyl 2-methylpropanoate;
[N-(2-{[3-(acetylamino)propyl]sulfonyloxy}-tert-butyl)carbamoyloxy]ethyl 2-methylpropanoate; and
a pharmaceutically acceptable salt of any of the foregoing.

9. A compound of Formula (II): 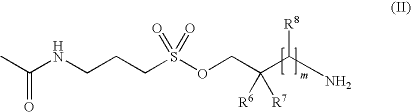
or a pharmaceutically acceptable salt thereof; wherein:
m is chosen from 0, 1, 2, and 3;
R⁶ and R⁷ are independently chosen from C₁₋₄ alkyl and substituted C₁₋₄ alkyl; or R⁶ and R⁷ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and
each R⁸ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

10. The compound of claim 9, wherein each of R⁶ and R⁷ is methyl.

11. The compound of claim 9, wherein each R⁸ is hydrogen.

12. The compound of claim 9, wherein each of R⁶ and R⁷ is methyl; each R⁸ is hydrogen; and m is chosen from 0, 1, 2, and 3.

13. The compound of claim 9, wherein the compound is chosen from:
2-amino-2-methylpropyl [3-(acetylamino)propyl]sulfonate trifluoroacetate;
3-amino-2,2-dimethylpropyl [3-(acetylamino)propyl]sulfonate hydrochloride;
4-amino-2,2-dimethylbutyl [3-(acetylamino)propyl]sulfonate hydrochloride;
5-amino-2,2-dimethylpentyl [3-(acetylamino)propyl]sulfonate hydrochloride;
and
a pharmaceutically acceptable salt of any of the foregoing.

14. A compound of Formula (III): 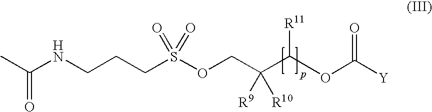
or a pharmaceutically acceptable salt thereof; wherein:
p is chosen from 0, 1, 2, and 3;
Y is chosen from R¹², —OR¹², and —NR¹²₂, wherein:
each R¹² is independently chosen from C₁₋₈ alkyl, substituted C₁₋₈ alkyl, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl;
R⁹ and R¹⁰ are independently chosen from C₁₋₄ alkyl and substituted C₁₋₄ alkyl; or R⁹ and R¹⁰ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and
each R¹¹ is independently chosen from hydrogen, halogen, —OH, —CN, —CF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl; and
with the proviso that when 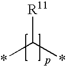
is C₁₋₈ alkyldiyl, and Y is chosen from R¹² and —OR¹²; then R¹² is not chosen from C₁₋₈ alkyl, C₆₋₁₀ aryl, substituted C₆₋₁₀ aryl, C₅₋₁₀ heteroaryl, substituted C₅₋₁₀ heteroaryl, C₇₋₁₈ arylalkyl, and C₆₋₁₈ heteroarylalkyl.

15. A compound of Formula (IV): 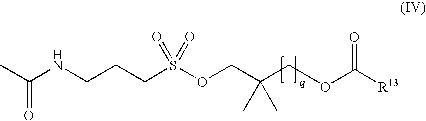
or a pharmaceutically acceptable salt thereof or; wherein:
q is chosen from 0, 1, 2, and 3; and
R¹³ is chosen from ethoxy, phenyl, —CH₂NH₂, and C₁₋₆ alkyl.

16. The compound of claim 15, wherein the compound is chosen from:
4-{[3-(acetylamino)propyl]sulfonyloxy}-3,3-dimethylbutyl benzoate;
4-{[3-(acetylamino)propyl]sulfonyloxy}-3,3-dimethylbutyl 2-aminoacetate hydrochloride;
3-{[3-(acetylamino)propyl]sulfonyloxy}-2,2-dimethylpropyl 2-methylpropanoate;
3-{[3-(acetylamino)propyl]sulfonyloxy}-2,2-dimethylpropyl benzoate;
5-{[3-(acetylamino)propyl]sulfonyloxy}-4,4-dimethylpentyl ethoxyformate;
5-{[3-(acetylamino)propyl]sulfonyloxy}-4,4-dimethylpentyl benzoate; and
a pharmaceutically acceptable salt of any of the foregoing.

17. A compound of Formula (V): 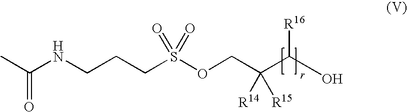
or a pharmaceutically acceptable salt thereof; wherein:
r is chosen from 0, 1, 2, and 3;
R¹⁴ and R¹⁵ are independently chosen from C₁₋₄ alkyl and substituted C₁₋₄ alkyl; or R¹⁴ and R¹⁵ together with the carbon to which they are bonded form a ring chosen from a C₃₋₁₀ cycloalkyl, substituted C₃₋₁₀ cycloalkyl, C₃₋₁₀ heterocycloalkyl, and substituted C₃₋₁₀ heterocycloalkyl ring; and
each R¹⁶ is independently chosen from hydrogen, halogen, —OH, —CN, —CF₃, —OCF₃, ═O, —NO₂, C₁₋₈ alkyl, substituted C₁₋₈ alkyl, C₁₋₈ alkoxy, substituted C₁₋₈ alkoxy, C₆₋₁₀ aryl, substituted C₆₋₁₀ aryl, C₃₋₁₀ cycloalkyl, substituted C₃₋₁₀ cycloalkyl, C₇₋₁₈ arylalkyl, substituted C₇₋₁₈ arylalkyl, C₄₋₁₈ cycloalkylalkyl, substituted C₄₋₁₈ cycloalkylalkyl, C₁₋₈ heteroalkyl, substituted C₁₋₈ heteroalkyl, C₅₋₁₀ heteroaryl, substituted C₅₋₁₀ heteroaryl, C₃₋₁₀ heterocycloalkyl, substituted C₃₋₁₀ heterocycloalkyl, C₆₋₁₈ heteroarylalkyl, substituted C₆₋₁₈ heteroarylalkyl, C₄₋₁₈ heterocycloalkylalkyl, and substituted C₄₋₁₈ heterocycloalkylalkyl.

18. The compound of claim 17, wherein each of R¹⁴ and R¹⁵ is methyl.

19. The compound of claim 17, wherein each R¹⁶ is hydrogen.

20. The compound of claim 17, wherein r is chosen from 0, 1, and 2.

21. The compound of claim 17, wherein each of R¹⁴ and R¹⁵ is methyl; each R¹⁶ is hydrogen; and r is chosen from 0, 1, and 2.

22. The compound of claim 17, wherein the compound is chosen from:
2-hydroxy-2-methylpropyl[3-(acetylamino)propyl]sulfonate;
4-hydroxy-2,2-dimethylbutyl [3-(acetylamino)propyl]sulfonate;
5-hydroxy-2,2-dimethylpentyl [3-(acetylamino)propyl]sulfonate; and
a pharmaceutically acceptable salt of any of the foregoing.

23. A pharmaceutical composition comprising a compound of any one of claims 1, 14, and 15, and at least one pharmaceutically acceptable vehicle.

24. The pharmaceutical composition of claim 23, comprising an amount of said compound effective for treating a disease is chosen from a neurodegenerative disorder, a psychotic disorder, a mood disorder, an anxiety disorder, a somatoform disorder, movement disorder, a substance abuse disorder, binge eating disorder, a cortical spreading depression related disorder, sleeping disorder, tinnitus, multiple sclerosis, and pain.

25. The pharmaceutical composition of claim 23, wherein the pharmaceutical composition is a sustained release oral dosage formulation.

26. A method of treating a disease in a patient comprising administering to a patient in need of such treatment the compound of any one of claims 1, 14, and 15; wherein the disease is chosen from a neurodegenerative disorder, a psychotic disorder, a mood disorder, an anxiety disorder, a somatoform disorder, movement disorder, a substance abuse disorder, binge eating disorder, a cortical spreading depression related disorder, sleeping disorder, tinnitus, multiple sclerosis, and pain.

27. A method of treating a disease in a patient comprising administering to a patient in need of such treatment the pharmaceutical composition of claim 23; wherein the disease is chosen from a neurodegenerative disorder, a psychotic disorder, a mood disorder, an anxiety disorder, a somatoform disorder, movement disorder, a substance abuse disorder, binge eating disorder, a cortical spreading depression related disorder, sleeping disorder, tinnitus, multiple sclerosis, and pain.
